SEQUENCE LISTING (466 sequences)

SEQ ID NO: 1 - protein, 2377 aa, Cryptococcus neoformans
MTTALTFGGGLFKDNTKFDIDMRGTADGAVNGGNIPNSQSQKRKRASPSPEIESEEDGDDWYEIDYIADSRVIRRKGRQILQYLIHWAGYAVHERTWEDEDGIGGEDCALVQEFYRKNPGKPRLSPSSVRKEVKLARMVEVVITTRRIDGKSRAASSTDQPSPHRLGITSPQANNIGGEDPNPSLTRRPVRSTVSEIAKRPTSKKVHPNKKCKASSDDESDFVFEEGEWDEDEDDDNDVDFRSSEDDEDDEQERSAEEPESDEEIIKPAKKTKSSLPKAKLRPKPANLGGFVTGVRPLNQGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKTDWEEIPQEEYKGRGSSISMTNEKRTPTLTVKSNPFKRSSSWALASSFRSKKRSMEARDAEGVSDDDENSELSDII

SEQ ID NO: 2 - DNA, 7131 bp, Cryptococcus neoformans
  source 1..7131: mol_type = genomic DNA
atgactactgcacttacatttggcggcggtcttttcaaagacaacaccaagttcgacattgacatgaggggaacagcagacggagccgtaaatgggggaaatatcccaaatagtcagtcacagaaaagaaaacgtgcgtctccttcgccggagatagagtcagaagaggatggcgacgactggtatgaaattgactacattgccgattcgcgagttatcagaagaaaaggacgccaaatcttacagtatctcatccactgggccggctatgctgtgcacgagagaacgtgggaggacgaggatggtattggaggagaagactgtgcacttgttcaagaattttatcgaaagaatcccggaaaaccaaggctttcgccctcttctgtcaggaaagaagtaaaattagcgcgcatggtagaggtcgtcatcactacgcggaggatcgacggaaagagcagagccgcctcctcaacagaccagccatcccctcatcgccttggcataacaagtccccaagcgaacaacattggtggggaagacccgaatccatccctcacaaggcgccctgttcggtctaccgtctcggaaatagccaagagaccaacttcaaagaaggtgcatccgaataaaaagtgtaaggcgtctagcgatgatgaaagcgacttcgtctttgaagaaggtgaatgggacgaggacgaggacgatgacaatgatgtggatttcagatcgagtgaagatgatgaagatgatgaacaggagagatctgcggaggagcctgaaagcgatgaggaaataataaaacctgcgaaaaagacaaagtcttctttgcccaaggcgaaattacgtcccaaaccagcgaatcttgggggtttcgtcacaggtgtgagacctctcaaccagggactggatatcaaagcggctgtaaggaacatgagtgacgatctacctccaataagcgacatcgaagccatgttcgatcaccttgtttccaggatcccggacatagttgaactggtcaggcagctgaatgggcgaaaactccgggtagctacaatgtgctccggcaccgagtcccctcttcttgctctcaacatgatcgcaaaagcaatcaaagctcaacatggccttacattggcgtttgaacacgttttttcttgtgagattgaaccattcaagcaggcttatattgagcgcaacttcacccctcctatcctattcagggatgtaactgaactgggaaagaaaagagctcatacagcgtatggctcgatggttgacgtacctggtgatgttgatatacttattgctggtacttcctgcgtcgattactccaatttgaataacgtccagcaggatattgacgccaatggcgaatctggaaggacctttcgtggcatgttacaatgggtcaaaaagcatcagcccccaatcgtcattttggaaaacgtctgtaacgcgccatgggataaggttgttgaatactttggacagattgattatgacgctcagtatacacgacttgatactaaagagttttatattccccatactcgaactcgagtttaccttttcgccactccctcatcttctgaatcagacgatcttccggagaaatgggcccagaccgtgaaggatcttcgtcgcccatggtcttcaccgttcgaagcattcttacttcatacagatgatcccaacatacatcgcgctcgtctcgaattggcctctgcgcgcgcgcagacagatggtacttctcgcaaaactaccgactggaatagatgcgaatcaagacatcagcgtgctagacaagacgaagccctgggcttattgagacctctgacttcctggcaagaagcgggtgtgtgtaagggactggattggacatggaatgactggctacttgcccagacagaaagggtagtcgatctcttggagatttccaccttgcggatggcaaaggacggcattgactctggtttcaaagcttgcatttggaatgtcagtcaaaacgttgatcgacagaccgggtcgtcaaagacagcccttgcaccttgcctgacacccaacatgattccctgggtcactatccgtggtggccctgtcactggacgcgaggctctagcacttcaaggcatccccgttcgagagctccttcttacaagtgagaacgaagatcaattggctgatctggcgggcaatgccatgaccaccactgttgtcggttctgcaatgattgctgcgctcaaggtggcatgtcacaagatcaccgagggcgctaaccctgaaaaagaagccgctttgattcttgaaaaggaagcagttgacgatgaacaagttgctaaccggatcatcggcgaagattatctcgagcatcatgatctcgaccttgccaaagtcaccaaatccaatctatctgagatcctcgatttggcttgtcgaagttcaaggcactgtcaatgtgaagggcagtctggcactgctccgaacatacttgagtgtcaagagtgcagttatcgtgcctgtaaatcgtgtggaggcagacctgagcatgtctatgcgccttgtgccaatcagagggttgagccagcggaatttgagaaaagatttaagggcttgcttcccatgcgggtacggattgctggcttaactgaccaatgtctaaatgctgtcaggaaagcagctgaaaagtctaacaaaggcagcgttaatgataatgattggcaattatggagtacagcacttctagaaggaatacacgatgctgaattcaggttccgatatctcaaaagacaaagcacctggacggcagtttacgaagctcgccgtgctatgcttagtcttgtccttcgcaatcaaataccggaatggcgactgaccatcaaggcaccggcatccgagccgaataacagccaacttcgagcacttcttcttcatccggtagctcgacttcaaattgatatcgccggccaagatgtcctgtgcggtccttgggaactttgcatcccctctatgaagactatcgacattgagattacgggtaagggcgaattgttgccttcttggcaagcttcgcttggtctccaaggtcccttcgccaacaccactcgatggtccgaagtcgaaatttccctccaagcggaagatgagaatacgcttgacagaaaattatctggtacctatcagcttcttccccgatgcggtcaggcaatgtcatctcttcataagaagaggcccgatctttcggacgatggtcttcctcagctctactttttccttgatcctaccaggtgtggtgaatcccgtgaagacagatatgtattttctacaagtactgaaagattggactatggcacggagcggcctgtgattgctcgattggattcccactggcgggaaggcaatgagaaacagaggaaggtgaagctggatgtctctggcgcatgggtgaaatgtcccgaagcccacctgaccgccataggtggggatgatatcgctgtcgttgccaatgatgctgcggcaaacgaaatccatcgtgacagggccacctttgccatcccttcatcggcttctgccatatcagcatctttgacaactgaaggttgttctcatgcaatggcacttctgtcttgtcgcgttccgcttgatcctactcattcggagagtatgtggcgacgaggagcctgggcagaaatcgacttgtcacatcaaggcaatactacttttgccaatttagcctggatcaccgagagattgccccctcttgatggcttaaaaaactgggcccatattgccgacgatgtatccgagcatgtttgcgagagatgcgccccaagaccacctaagattcactggataaaaagggaaggcaaggctaacaaaaagggaaacaagacgaagagtaccatcattgcgttcgaagataagctagaagctgggcagtatgagcatgctctcaagcatcggccctcgccctttgttgtccagcttaggctcgatcaggatataggttcgttccgtatcggtctcaacattgtttcccttgcccatcgtgcactctcgcgactcccgcctaccacgtccgaacataaaatcagtctttcttggcgtctcaccccggggcatgtgaccgaaagcccacaacctcgccgcgtctttatcctgccaagcaataaacaagatcctgaaaactctcaacccgaagcattcaaattaccccttcgtaaagaacagttgcgatccttgtggtggatgctagagcaagaaaaggcaacggggaaaacccacacgtttgttgaagaagagatttctgagtccttgcttcctgctgtagggtggagagcagagggtaaggctgagaggcccgtcatggtccgcggaggtgtcattgcggatcaggtcggttacggcaaaacagtcatctccatcgctctggtggcgcagaccttgtcccttcctgcgcctgagcctgcaacccccggcttgattgacctgaaagcgacgcttattgttgtgcctggtcatcttagtaaacaatggcccaacgagatagcgcgatttaccggcagcatgttcaaggtcattgtcatccaggggatgaaagatttacaggagaagactatcgctgaacttggcaaagctgatatcatcgtcatggcgtcagaaattttcgagtctgacgtctattggtctcgactcgagtacctctctgcccagcctcgagaatggctccatgacacccagggtggaagatttttctgtgacaggcttgacgcagctatggaaagtcttgtgtcgcagacgaaaatcttgaaagaaaaaggcagcgaggcggcgatgagggctatggaggataagaagaaaagtttggtagataatgtcgggtctaagaaggaagtacacactgccgttaacttcggcaagagaatgaaaggacaagcgtatagagacaaacatgatagtgattccaaagcgaaacccattaccaaggaggagcttgagcgatgggaggcttctgaagatgaggatgatgacgagaatagcaagacttacatccctatacccaaatttcacagcttcacgggctcagaatcaattttcagtgcttcggtcaaaaaggattacaagttgttacccaatccggttctccatatgtttcgattccgtcgagtcattgcagatgaattcacttatcttcagaaaaagagccttgcagccgttttacgcctttcttcttcataccgatggattctgtctggcactccgcctgtcagcgattttgcagcaatcagaagtatcgcaacattcatgggtatacaccttggtgtggaggatgatggcgagggcgatgtccagtatcagaaggctcgcgcaaaagaccagacccaagccgagaagttccatgcttttagggaggttcattcacgagcctggcataatcgacgagatgaactggcacaggagttcttaaacgtttttgtgcgccagaatatcgcggagattgaggatatacccactgtcgagcatatccatactttcaaactgccagcttcagaaggtgctgtatacctcgaactagagcatcatctccaagctttggaaatgcaagcacgcaaggagacgaagtttaaaaatgtcagtcaaggtgatcgcaacgctaggcttgaagaggccttgtccgattcgaaaacggcagaagaagccttactcaaacgatgctgtcatttcactcttgatttgtctgataagactcaggacgcaaagtctgcccaagaagcctgtgatcacattaccagcgctcgagctaggcaattactcgcttgtcaagaagatctttctcggtcagtcaatcaggcaatagctttgcatggatggattaagaagaaggggggatttagcaagaatgatgacgaacgtcaaccatttgctgagtggattgcattctcctccaacatctcgaaacatcaaggagacatcgaagctgcacgcatactactcaaggtgatagagaagtgtggcgtaaaggacggcaatatcccgccttccccttcagataaacagtctccgtcaatagccagtggtgccaaaatggatgacgtcaaatggcagctccgagagcagacccatcttcttcgtaaacttgtcaaggaattggtggcgagagtcagatcattacgcttctttgaggtagtcaggaaaatccagaaaggcaagagcgatgcccaaattgtacttgaatcttcggaatgtggacacaagccatccaccaatccagatatcgaaatggctattctgtcttgttgtggtcatgtcgcgtgtcataagtgcatgcgtaaggcagccgcttctcaacgatgtgttaagtccggagaatgccaagcggcagtgcgaccgaccaacatagtcaaggtaagctccctgggcgttgaaggagagctttcctcagggcggtatggtgccaaactagagcatctagtcaacctcatccattctatccccaaaaatgagcgggtgttagtattccttcaatgggaagacctcgcaggcaaggtctctgaagctctgtctgcaggcagaattccacacgtcacgctatccggttctgccaagtcacgagcaaatacccttgaccgtttccagtccacaaatgccgatagtgcacgagtactgcttctcaagatgaatgatgcaagtgccgctgggtctaatctgaccactgccaaccatgctgtcttcctcggccccttattcaccaattctttattcaactatcgtgccgtagaaacacaagccatcggtcgagtgcgaaggtacggacaacagaaaaaagttcatattcatcggctgttggccctcgacaccattgacatgaccattttcaacgcgaggaggactgagctcaaagaaaagacagattgggaagaaataccccaagaggagtacaaaggaagaggctcatcaattagcatgacaaacgagaaaaggacgccaacattgacagtcaagagtaatccattcaaacgcagttctagctgggcgttagcatcatctttcagaagtaagaaacgatcgatggaggcaagggatgctgaaggtgtaagcgacgatgatgagaattcagaactttcggatatcata

SEQ ID NO: 3 - protein, 2300 aa, Cryptococcus tepidarius
MAVTQSSTRSNRTPRRAAAACVSYVDISDEEMNEDDMILVKKRKRAVNIAAMFRSQVKSQAEASSSRPLRHSTQPQRFGSSTPEKALAKESRLLNESSNQPSITSMFSWQSKAQMILPSSTPNSQSTRSQQTAASTPKKGETQSNAKAFGSGVKVQLRASISSKKSKVQSDDDDDFAASESDDDEDFESENEEEEKSEEEEYEEEDDYDEKPKGKKAKTSVTPMTNKKSSGGWGANKIKGPKKAPVKLGNFKTGVKPLMTKDGKAVVKSANMNEDLPPIADVEEMFDHLTSRIPDIVKVVQRLNGRKLRVATMCSGTESPLLALKMIAKAIERQHGLSFEFDHVFSCEIEPFKQAYIERNFAPPILFRDVTELGRKQAHTAYGSIVKVPGDVDILIAGTSCVDYSGLNNEKQDIDANGESGRTFRGMLQWVNKHRPPIVILENVCSAPWDKIVEYFGQVDYDANYCRLDTKEYYIPHTRTRVYLFATPAHKGDVSHLPEKWVSTLSDLKRPWSASFEAFLLPSDDPNIHRARLDLAAARFQNDGTQRKPTDWNRCESRHARARQEELLGVLRPLTSWKEAGVCKGLDWTWNDWLLSQTERVVDLLDISTLRMAAQNDVDSGYKACIWNVSQNVDRQGTGSRTALAPCLTPNMIPWVTIRGGPVTGREALSLQGIPVRELLLTSENEDQLADLAGNAMTSTVVGASMLSALVLAVDKLAPGEGLESGTTKAEDDGEAKKHHIVGEEYLEEHELDLAKVTSANLVEIMDLAERSSRHCQCEGQSGTVTPIDECTLCGYRACKTCGGRPDHAYSPCRHVRVEPREFEKQFKELLPMRVNIAGVTKASLERVRKRAEAIGKGSVVENDWNLWSEALLEGVTDAEFRFRYLKRQNVWTAVYEAPHATLELVLRSTPEWLLTIKSPSSEAVNSRLRTLLLHPVARMRFNLTSQDVLCGPWELCVPSKQSFDILITGEGELVPTWHASIGLQGKHQGTMRWSELRISVPPEVDMALDRKVSGRYPLLPRCGQAMASLHKKQPTQEDEGLSPLFFFLDPDRVGESSDDTYVFTTSIERLEYGTERAIIAKLESKWRESDKIESKVKLNVRGGWVVCDEVHLTAVGGSDIAIVDCINDAHRDTATYSFPRDIPAKLTSDCSQSLALLSVQVPLDSSHAETMWRKDDWGEIDLQHEGNTTFANLAWITERLPSLDRFKEWSELSAVDTSGSPCSRCSPLPPTIHWVRQAGNLNSKGHKTKATIIAFEDRQEASRYEHALKNRPSAFTVQLRLDNQVGSLRIGLNIASLAHRAFSRLPASQGGVKLSWRLTQDKIADLPRPPREFILPSNKRDPEYAHPKSFLLPLRKEQLRSLWWMLRQEAAEGKTHTFVEEEISEAMLPSLGWRAEGKAERPVMVRGGVIADQVGYGKTIISLALVAETKDDPAPEPAPAGLIDLKATLIVVPGHLSKQWPSEITRFTGKLFKTIVIQSMRELNALNIADLSKADIIVMASEVLDSDAYWERFEYLSAQPEDWLSDQQGGRFFADRLNTAMAALRDQTRLLMNDPEEAVENMKTLHERAREDAAKKKKELKVVNFGKRLKGAAYRDKYDADQTLKKSKSAGTSKSKMWEVSEDEEEDEVEEDVAPPKLVFRKASGKDSYSSSSVRANYKLLSAPVMHMFRFRRVIADEFTYLEKKPLNALLKLSSCYKWVLSGTPPVGDFPAIRSIASFMGLHLGVEDDGEGSKSTQIARTKELTQAEKFHAFREMHSSAWHNRRDDLAQLFLNTFVRQNIAEIDEIPTVEHVHEVKLPAAEGAVYLELEHHLQALEMQARKETKFKNVTQGDRNARLEEALTDSTTAEEALLKRSCHFTLDLNDRKRDAKSAKEACSFIAGKRRVQLMGCEDDLVNTINLAVGLHAFVRFNNGWTNAERQPFKEWVELSFDETKHQGDIEAARRLIAILQNRCGFLDGIPNAPADSRGIKKLAKAKPAEVENMKWELREQTHLLRRLSKELVARVRSLRFFEVVRKLQLNGPEAKEILESSVCDHRPSTHPDVEMAVLSCCGHVACHNCMIQAADNQRCPSPSTCHAAIRHTNIVKVKTLGVEGELSSGRYGAKLQRLVDLIKAIPKRERVLVFLQWEDLAHKVSEALTDGGISHLTLSGSVKQRANTLDSFQSDTASSRVLLLKMQDASAAGSNLTTANHAIFLGPLFVDTLHRYRQAETQAIGRIRRYGQLKHVHIHRLLALDTIDMTIYQNRTRELQSKSDYIPIPIEVYVPRGKPSSRSSEGSAQSVMEVDGMIDVDRPSSPPA

SEQ ID NO: 4 - DNA, 6900 bp, Cryptococcus tepidarius
  source 1..6900: mol_type = genomic DNA
atggcagtcactcagagctcgactcgttccaataggacccctcgcagagccgctgctgcttgtgtcagctacgtcgatatctcagacgaggaaatgaatgaagacgacatgatcttggttaaaaagcgaaaacgggccgtcaatatcgcggcgatgttccgcagtcaagtcaagtcccaagccgaagcgtcgtcttcaaggcctttgaggcattctactcaacctcagcgctttggttcctcaactccagagaaagctttggcgaaagagtcaaggttattgaatgaatcctccaaccagcccagtataacctccatgttctcttggcaatccaaagcgcagatgattttaccatcatcaactccaaatagtcaatcaacgcgttctcagcaaacagctgcgtcaacaccgaaaaaaggtgaaacacaaagtaatgccaaggcgttcgggtctggagtaaaggttcaattgagggcatcaatatccagcaaaaaatccaaggtgcaatctgatgatgatgatgactttgcggcaagtgaatcggatgacgatgaagattttgaatcagaaaatgaagaggaggagaaaagcgaggaggaagaatatgaggaagaagatgactatgatgagaaacccaaaggaaaaaaagcaaagacatcagtgacgcctatgaccaacaagaaatcatcaggaggttggggtgcgaacaaaatcaaaggccccaaaaaagcaccagtgaagcttggcaatttcaaaaccggtgttaagcctttaatgaccaaagatggaaaggcagttgtcaaaagcgccaatatgaacgaggatctcccacctattgctgatgtcgaggagatgttcgatcaccttacatccaggataccagatatcgtcaaagtggttcaacgtctcaacggtcgtaaacttcgtgtcgcgacgatgtgctctggtactgagtcgcctctattggcattaaaaatgattgccaaggctatcgaacggcagcacggtttatcgtttgagttcgatcacgttttctcatgcgagattgaacctttcaaacaagcttacatcgaacgtaattttgctcctcccattcttttccgtgatgttactgaactcggacgcaagcaagcgcatactgcttatggctcaattgtcaaagttcctggtgatgttgatattcttatcgctggtacatcatgtgtagactattctggcctgaacaacgagaaacaagatatcgatgccaatggtgaatcaggtcgaacatttcgtggtatgcttcaatgggtgaacaaacatcgcccgcccattgtcattctggaaaatgtatgtagtgcgccttgggacaaaatagtggagtactttggacaagttgattacgacgcaaattactgcaggcttgacactaaggagtattacattcctcacactcgaactcgtgtctaccttttcgccactccagcccataaaggcgacgtgtctcatctacccgaaaagtgggtttcgactttgtccgacctcaaacgtccatggtcggcgtcatttgaggcatttcttttgccttctgacgatcctaacattcatcgagctcgactcgaccttgctgctgctcgattccaaaacgacgggacacaaaggaaaccaacagactggaatcgttgcgaatctcgacatgctcgagcccgtcaagaagaattgttgggcgtgctccgccctcttacgtcatggaaagaggcaggtgtatgtaaagggttggattggacatggaatgactggcttctctcccaaacagagcgagttgtagatctgttggatatctcaacattgcgcatggctgcccaaaatgacgtcgactccggatacaaagcttgcatttggaacgtcagtcaaaacgttgatcgacaaggtaccggaagtcgaactgcattggcgccttgtttgacacccaatatgatcccgtgggtaacgattcgaggtggtcccgtcacaggtcgagaggctttgtcgcttcagggtataccggtccgtgaactgctccttaccagcgagaatgaggatcagcttgcagatctcgctggtaatgccatgacctcgacagtcgtcggcgcgtcaatgctttcagcactagtgctggctgtggacaagctcgcaccaggtgagggtctcgagagtggaacgaccaaagctgaggacgatggtgaggcaaagaagcatcatatcgtaggcgaagaataccttgaagaacatgagcttgatctcgccaaagtcactagtgccaacctggttgagattatggatctggctgagcgtagctctcgtcattgtcagtgcgaaggtcagtcaggcaccgttacgcccatcgacgaatgtacattgtgcggctatcgagcttgtaaaacttgtggtggccgccctgatcatgcctattctccttgcagacatgtccgcgttgagccgagggagtttgagaagcagttcaaagagcttttaccgatgcgtgtgaatattgctggagtgactaaggcatcgctagaaagggttcgtaaacgtgcggaagctattgggaagggttcagtggtcgaaaacgattggaatctttggagcgaggcgctcttggagggtgtaaccgacgcagaattcagattcagatatttgaaacgtcaaaatgtttggactgcggtttacgaagcaccacatgcgacattggagcttgtgctacgctccactcccgaatggcttttgactatcaaatcaccttcgtctgaagctgtgaatagtcgccttcgtactctgcttcttcaccccgtcgccagaatgcgattcaatttgactagccaggacgtactttgtggcccctgggaactttgtgttccttccaaacagtctttcgacatattaatcactggcgaaggcgaactcgtcccaacctggcatgcgtcgatcggtcttcagggcaaacatcaggggacgatgcgttggtctgaactcagaatctcggttcctcctgaagtcgatatggctctcgatcgtaaggtgtcaggtcgatatccccttctccctcgatgtggtcaagccatggcatctctacacaagaaacaaccgacacaggaggatgagggtctatcaccactattcttcttcctcgacccagatcgcgtgggtgagtcgtccgacgatacctatgtattcaccacatcaatcgaacgtcttgagtacggaactgaacgtgcgattatcgcgaagctagagtccaagtggcgcgagtctgacaaaatcgagagcaaagtcaagctcaacgtccgcggtggatgggtagtgtgtgacgaagtgcacctaacggctgtgggaggtagtgatatcgccattgtggattgtatcaatgacgcccatcgagacacagccacatactcattccctcgggatatccctgcaaaattgacatcggattgtagtcaatcattggctctcttgtcggtccaagttcctttggactcttctcatgctgaaactatgtggcggaaagatgattggggtgagattgatcttcagcacgaagggaacacgacgtttgccaatcttgcttggatcactgaacgtcttccttcgcttgaccggttcaaagaatggagcgagctctcagcggtggatacctccggttcgccctgttctcgttgctcgcctctcccgccaacgattcattgggtcaggcaagcgggaaatcttaattccaaaggccacaaaaccaaggcgaccatcatcgcctttgaggatcggcaagaagctagtcgctatgagcatgcacttaaaaatcgtccttcagcgttcactgttcagctacgcctcgacaaccaggttgggtctttgcgaattggcttaaacattgcctctctggcgcatcgtgcattttctcgtttgcccgcttctcagggcggagtcaaactctcttggcgtcttacacaggacaaaattgccgacctccctcgaccacctcgggagtttatcttgcctagcaacaaacgagaccccgaatatgctcatccaaagtcgttcctcttaccgcttcgtaaagagcagcttcgctccctttggtggatgttgcgtcaggaggcagccgagggcaaaactcacacatttgttgaggaggaaatctcagaggccatgttgccatctcttggatggcgagcggaaggtaaagcagagcgaccagtcatggtgcgtggaggtgtgatcgccgatcaagtcggttacggaaagactattatctctctcgctctggtcgctgagacaaaagatgatcccgctccagaacccgctccagctggtttgattgacctcaaagccacgctcatcgttgttcctggccacttgagtaaacaatggccgagtgagatcacccgattcacgggaaagctgtttaagacgattgtcatccaaagtatgcgagaactcaacgctctcaacattgcagatctttccaaagccgacattatcgttatggcctcggaggtgctcgattcagatgcgtattgggagagattcgaatatctttcagctcagccagaggactggttgtccgatcaacaaggtggacgatttttcgctgatcgcttgaacactgctatggcggcgttgcgcgatcagacgaggttgctcatgaatgacccggaggaagcagtagagaacatgaaaaccctgcacgagcgagctcgggaagacgcagcaaaaaagaaaaaggaactcaaggtcgtcaactttgggaaacgcctaaagggtgccgcatatcgagacaaatatgacgctgatcagaccttaaagaaatccaaatcagctggcacctcaaagagcaagatgtgggaggtatctgaagatgaagaagaagacgaagtggaggaggatgttgctcctccaaagctagtctttcgcaaagcgtctggtaaagactcgtattccagcagttcggtccgtgcaaactacaagcttcttagcgcacccgtcatgcacatgtttagattccgacgggttattgccgatgaatttacgtatctcgaaaagaagcccttgaatgctcttctcaagctctcttcttgctacaaatgggtactttccggaactccacccgtgggagacttcccagcgatccgcagcatcgcttccttcatgggacttcatctcggtgtagaggacgacggcgaaggcagcaaatcgacccagattgcccgcacaaaagaactcacacaagccgaaaagttccacgctttccgtgaaatgcattcatcggcttggcataaccgtcgcgatgaccttgcgcaattgttcttgaacacgttcgtccgtcaaaatatcgcagagattgatgagatcccaacagttgagcatgttcatgaagtcaagcttcctgccgctgaaggtgccgtttacctcgagcttgagcaccaccttcaagcactcgagatgcaagcacgtaaagaaaccaaattcaaaaacgtcacgcaaggtgaccgaaacgcgcgtttggaagaggccttgaccgattcgacaactgctgaagaggctcttctcaagcgaagttgtcatttcacactcgacctgaatgatcgaaagcgagatgcaaagtcagccaaggaagcgtgtagtttcattgcaggaaaacgtagggtccaattgatgggctgcgaagacgacttggtgaatactatcaaccttgctgttggccttcatgcttttgttcgtttcaacaatggctggactaatgcagaacggcaaccgtttaaagagtgggtggagctctcatttgatgagaccaaacatcagggcgacatcgaggccgcacgtcgattgattgctatcctccagaacagatgtggcttcttagacggtatcccaaacgcacctgctgattctcgtggcattaaaaagttggcaaaggcaaaaccggccgaagtagagaatatgaaatgggagttgcgagagcagactcatttacttcgacgattatccaaggaactcgttgcccgtgttaggtctcttcgattctttgaagtagtgcgtaaacttcaattgaatggtcccgaagccaaagaaatccttgagtcttcagtttgcgaccatcgtccatcgacacatccagacgtcgaaatggccgtactttcatgctgcggtcacgttgcttgtcataattgtatgattcaagctgccgacaaccagcgttgtccatcaccctcaacttgtcacgctgccattcgacacaccaatatagtcaaagtcaagacgctgggtgtcgagggcgaactctcgtctggtcgatatggagcaaaacttcaacgcctcgttgatttgatcaaggcaattccaaagagagaacgtgttttggtctttttgcaatgggaagatctcgcccacaaagtctcggaagctctgactgacggtggtatttcccacctcacgctcagcggcagtgtcaagcaacgagccaacactctggattctttccaatccgataccgcatcgtcgcgcgttctcttgctcaaaatgcaggacgcatcagctgctggatccaacttgacaactgccaatcacgccatcttcttgggacctttgtttgtcgatacacttcatcgataccgacaagcagaaactcaggcgatcggtcgaatccgtcgttatggtcaattgaaacatgttcatattcaccgtctcttggctcttgacacgattgatatgacaatttaccagaacaggacgagagaattacaatccaaatcagactatatcccgatccctattgaggtgtacgtccccagaggcaagccgtcctccagatcgagtgagggatcagcccagtccgttatggaagttgatggtatgattgacgttgatagaccatcgagtcctcctgca

SEQ ID NO: 5 - DNA, 140 bp, synthetic construct
  source 1..140: mol_type = other DNA
ccggtaggggcccattatagccgaattcggcgcgccaggtggcacttttcggggaaatgtgcgcggaacccctatttgtttatttttctaaatacattcaaatatgtatccgctcatgagacaataaccctgataaatgc

SEQ ID NO: 6 - DNA, 71 bp, synthetic construct
  source 1..71: mol_type = other DNA
  modified_base 60: mod_base = m5c
gcgaaattaatacgactcactatagggagccggtaggggcccattatagccgaattcggcgcgccaggtgg

SEQ ID NO: 7 - DNA, 61 bp, synthetic construct
  source 1..61: mol_type = other DNA
caaaaaacccctcaagacccgtttaggcatttatcagggttattgtctcatgagcggatac

SEQ ID NO: 8 - DNA, 29 bp, synthetic construct
  source 1..29: mol_type = other DNA
  misc_feature 1: note = Cy3 is present at the 5_prime end
gcgaaattaatacgactcactatagggag

SEQ ID NO: 9 - DNA, 29 bp, synthetic construct
  source 1..29: mol_type = other DNA
  misc_feature 1: note = Atto647 is present at the 5_prime end
gcgaaattaatacgactcactatagggag

SEQ ID NO: 10 - DNA, 26 bp, synthetic construct
  source 1..26: mol_type = other DNA
  misc_feature 1: note = Cy3 is present at the 5_prime end
caaaaaacccctcaagacccgtttag

SEQ ID NO: 11 - DNA, 26 bp, synthetic construct
  source 1..26: mol_type = other DNA
  misc_feature 1: note = Atto647 is present at the 5_prime end
caaaaaacccctcaagacccgtttag

SEQ ID NO: 12 - DNA, 104 bp, synthetic construct
  source 1..104: mol_type = other DNA
  modified_base 39: mod_base = m5c
  modified_base 45: mod_base = m5c
  modified_base 55: mod_base = m5c
gggcggcgacctcgcgggttttcgctatttatgaaaatcgtttccggtttaaggcgtttgtcataacttaatgtttttccgttcttcttcgtcgacgggctccc

SEQ ID NO: 13 - DNA, 104 bp, synthetic construct
  source 1..104: mol_type = other DNA
  misc_feature 1: note = biotin group is attached to the 5_prime end
gggagcccgtcgacgaagaagaacggaaaaacattaagttatgacaaacgccttaaaccggaaacgattttcataaatagcgaaaacccgcgaggtcgccgccc

SEQ ID NO: 14 - protein, 2387 aa, Cryptococcus neoformans
MTTALTFGGGLFKDNTKFDIDMRGTADGAVNGGNIPNSQSQKRKRASPSPEIESEEDGDDWYEIDYIADSRVIRRKGRQILQYLIHWAGYAVHERTWEDEDGIGGEDCALVQEFYRKNPGKPRLSPSSVRKEVKLARMVEVVITTRRIDGKSRAASSTDQPSPHRLGITSPQANNIGGEDPNPSLTRRPVRSTVSEIAKRPTSKKVHPNKKCKASSDDESDFVFEEGEWDEDEDDDNDVDFRSSEDDEDDEQERSAEEPESDEEIIKPAKKTKSSLPKAKLRPKPANLGGFVTGVRPLNQGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKTDWEEIPQEEYKGRGSSISMTNEKRTPTLTVKSNPFKRSSSWALASSFRSKKRSMEARDAEGVSDDDENSELSDIIGGGSHHHHHH

SEQ ID NO: 15-16 - skipped (2 entries)

SEQ ID NO: 17 - DNA, 104 bp, synthetic construct; identical to SEQ ID NO: 13
  source 1..104: mol_type = other DNA
  modified_base 49: mod_base = m5c
  modified_base 59: mod_base = m5c
  modified_base 65: mod_base = m5c
  misc_feature 1: note = biotin group is attached at the 5_prime end

SEQ ID NO: 18 - DNA, 29 bp, Synthetic construct
  source 1..29: mol_type = other DNA
gcgaaattaatacgactcactatagggag

SEQ ID NO: 19 - DNA, 133 bp, Homo sapiens
  source 1..133: mol_type = genomic DNA
  modified_base 33: mod_base = m5c
  modified_base 35: mod_base = m5c
  modified_base 46: mod_base = m5c
  modified_base 48: mod_base = m5c
  modified_base 70: mod_base = m5c
  modified_base 77: mod_base = m5c
  modified_base 85: mod_base = m5c
ccgcctacgtgggggaccctgttaggggcaccngngtagaccctgngngccctcacaggaccctgtgctngttctgngcactgcngcctgggtttccttccttttattgttgtttgtgtttgccaagcgacag

SEQ ID NO: 20 - DNA, 133 bp, Homo sapiens
  source 1..133: mol_type = genomic DNA
ccgcctacgtgggggaccctgttaggggcacccgcgtagaccctgcgcgccctcacaggaccctgtgctcgttctgcgcactgccgcctgggtttccttccttttattgttgtttgtgtttgccaagcgacag

SEQ ID NO: 21 - DNA, 72 bp, Synthetic construct
  source 1..72: mol_type = other DNA
accttcttcgttaccttcttcaccaacaatgaaataatcctgtcgcttggcaaacacaaacaacaataaaag

SEQ ID NO: 22 - DNA, 58 bp, Synthetic construct
  source 1..58: mol_type = other DNA
accacaacggtttccctctagaaataattttgtttaacccgcctacgtgggggaccct

SEQ ID NO: 23 - protein, 2395 aa, synthetic construct
MTTALTFGGGLFKDNTKFDIDMRGTADGAVNGGNIPNSQSQKRKRASPSPEIESEEDGDDWYEIDYIADSRVIRRKGRQILQYLIHWAGYAVHERTWEDEDGIGGEDCALVQEFYRKNPGKPRLSPSSVRKEVKLARMVEVVITTRRIDGKSRAASSTDQPSPHRLGITSPQANNIGGEDPNPSLTRRPVRSTVSEIAKRPTSKKVHPNKKCKASSDDESDFVFEEGEWDEDEDDDNDVDFRSSEDDEDDEQERSAEEPESDEEIIKPAKKTKSSLPKAKLRPKPANLGGFVTGVRPLNQGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKTDWEEIPQEEYKGRGSSISMTNEKRTPTLTVKSNPFKRSSSWALASSFRSKKRSMEARDAEGVSDDDENSELSDIIGGGSDYKDDDDKHHHHHH

SEQ ID NO: 24 - protein, 2339 aa, synthetic construct
MGDDWYEIDYIADSRVIRRKGRQILQYLIHWAGYAVHERTWEDEDGIGGEDCALVQEFYRKNPGKPRLSPSSVRKEVKLARMVEVVITTRRIDGKSRAASSTDQPSPHRLGITSPQANNIGGEDPNPSLTRRPVRSTVSEIAKRPTSKKVHPNKKCKASSDDESDFVFEEGEWDEDEDDDNDVDFRSSEDDEDDEQERSAEEPESDEEIIKPAKKTKSSLPKAKLRPKPANLGGFVTGVRPLNQGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKTDWEEIPQEEYKGRGSSISMTNEKRTPTLTVKSNPFKRSSSWALASSFRSKKRSMEARDAEGVSDDDENSELSDIIGGGSDYKDDDDKHHHHHH

SEQ ID NO: 25 - protein, 2108 aa, synthetic construct
MGGFVTGVRPLNQGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKTDWEEIPQEEYKGRGSSISMTNEKRTPTLTVKSNPFKRSSSWALASSFRSKKRSMEARDAEGVSDDDENSELSDIIGGGSDYKDDDDKHHHHHH

SEQ ID NO: 26 - protein, 2096 aa, synthetic construct
MGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKTDWEEIPQEEYKGRGSSISMTNEKRTPTLTVKSNPFKRSSSWALASSFRSKKRSMEARDAEGVSDDDENSELSDIIGGGSDYKDDDDKHHHHHH

SEQ ID NO: 27 - protein, 2354 aa, synthetic construct
MTTALTFGGGLFKDNTKFDIDMRGTADGAVNGGNIPNSQSQKRKRASPSPEIESEEDGDDWYEIDYIADSRVIRRKGRQILQYLIHWAGYAVHERTWEDEDGIGGEDCALVQEFYRKNPGKPRLSPSSVRKEVKLARMVEVVITTRRIDGKSRAASSTDQPSPHRLGITSPQANNIGGEDPNPSLTRRPVRSTVSEIAKRPTSKKVHPNKKCKASSDDESDFVFEEGEWDEDEDDDNDVDFRSSEDDEDDEQERSAEEPESDEEIIKPAKKTKSSLPKAKLRPKPANLGGFVTGVRPLNQGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKTDWEEIPQEEYKGRGSSISMTNEKRTPTLTVKSNPGGGSDYKDDDDKHHHHHH

SEQ ID NO: 28 - protein, 2298 aa, synthetic construct
MGDDWYEIDYIADSRVIRRKGRQILQYLIHWAGYAVHERTWEDEDGIGGEDCALVQEFYRKNPGKPRLSPSSVRKEVKLARMVEVVITTRRIDGKSRAASSTDQPSPHRLGITSPQANNIGGEDPNPSLTRRPVRSTVSEIAKRPTSKKVHPNKKCKASSDDESDFVFEEGEWDEDEDDDNDVDFRSSEDDEDDEQERSAEEPESDEEIIKPAKKTKSSLPKAKLRPKPANLGGFVTGVRPLNQGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKTDWEEIPQEEYKGRGSSISMTNEKRTPTLTVKSNPGGGSDYKDDDDKHHHHHH

SEQ ID NO: 29 - protein, 2067 aa, synthetic construct
MGGFVTGVRPLNQGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKTDWEEIPQEEYKGRGSSISMTNEKRTPTLTVKSNPGGGSDYKDDDDKHHHHHH

SEQ ID NO: 30 - protein, 2055 aa, synthetic construct
MGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKTDWEEIPQEEYKGRGSSISMTNEKRTPTLTVKSNPGGGSDYKDDDDKHHHHHH

SEQ ID NO: 31 - protein, 2319 aa, synthetic construct
MTTALTFGGGLFKDNTKFDIDMRGTADGAVNGGNIPNSQSQKRKRASPSPEIESEEDGDDWYEIDYIADSRVIRRKGRQILQYLIHWAGYAVHERTWEDEDGIGGEDCALVQEFYRKNPGKPRLSPSSVRKEVKLARMVEVVITTRRIDGKSRAASSTDQPSPHRLGITSPQANNIGGEDPNPSLTRRPVRSTVSEIAKRPTSKKVHPNKKCKASSDDESDFVFEEGEWDEDEDDDNDVDFRSSEDDEDDEQERSAEEPESDEEIIKPAKKTKSSLPKAKLRPKPANLGGFVTGVRPLNQGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKGGGSDYKDDDDKHHHHHH

SEQ ID NO: 32 - protein, 2263 aa, synthetic construct
MGDDWYEIDYIADSRVIRRKGRQILQYLIHWAGYAVHERTWEDEDGIGGEDCALVQEFYRKNPGKPRLSPSSVRKEVKLARMVEVVITTRRIDGKSRAASSTDQPSPHRLGITSPQANNIGGEDPNPSLTRRPVRSTVSEIAKRPTSKKVHPNKKCKASSDDESDFVFEEGEWDEDEDDDNDVDFRSSEDDEDDEQERSAEEPESDEEIIKPAKKTKSSLPKAKLRPKPANLGGFVTGVRPLNQGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKGGGSDYKDDDDKHHHHHH

SEQ ID NO: 33 - protein, 2032 aa, synthetic construct
MGGFVTGVRPLNQGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKGGGSDYKDDDDKHHHHHH

SEQ ID NO: 34 - protein, 2020 aa, synthetic construct
MGLDIKAAVRNMSDDLPPISDIEAMFDHLVSRIPDIVELVRQLNGRKLRVATMCSGTESPLLALNMIAKAIKAQHGLTLAFEHVFSCEIEPFKQAYIERNFTPPILFRDVTELGKKRAHTAYGSMVDVPGDVDILIAGTSCVDYSNLNNVQQDIDANGESGRTFRGMLQWVKKHQPPIVILENVCNAPWDKVVEYFGQIDYDAQYTRLDTKEFYIPHTRTRVYLFATPSSSESDDLPEKWAQTVKDLRRPWSSPFEAFLLHTDDPNIHRARLELASARAQTDGTSRKTTDWNRCESRHQRARQDEALGLLRPLTSWQEAGVCKGLDWTWNDWLLAQTERVVDLLEISTLRMAKDGIDSGFKACIWNVSQNVDRQTGSSKTALAPCLTPNMIPWVTIRGGPVTGREALALQGIPVRELLLTSENEDQLADLAGNAMTTTVVGSAMIAALKVACHKITEGANPEKEAALILEKEAVDDEQVANRIIGEDYLEHHDLDLAKVTKSNLSEILDLACRSSRHCQCEGQSGTAPNILECQECSYRACKSCGGRPEHVYAPCANQRVEPAEFEKRFKGLLPMRVRIAGLTDQCLNAVRKAAEKSNKGSVNDNDWQLWSTALLEGIHDAEFRFRYLKRQSTWTAVYEARRAMLSLVLRNQIPEWRLTIKAPASEPNNSQLRALLLHPVARLQIDIAGQDVLCGPWELCIPSMKTIDIEITGKGELLPSWQASLGLQGPFANTTRWSEVEISLQAEDENTLDRKLSGTYQLLPRCGQAMSSLHKKRPDLSDDGLPQLYFFLDPTRCGESREDRYVFSTSTERLDYGTERPVIARLDSHWREGNEKQRKVKLDVSGAWVKCPEAHLTAIGGDDIAVVANDAAANEIHRDRATFAIPSSASAISASLTTEGCSHAMALLSCRVPLDPTHSESMWRRGAWAEIDLSHQGNTTFANLAWITERLPPLDGLKNWAHIADDVSEHVCERCAPRPPKIHWIKREGKANKKGNKTKSTIIAFEDKLEAGQYEHALKHRPSPFVVQLRLDQDIGSFRIGLNIVSLAHRALSRLPPTTSEHKISLSWRLTPGHVTESPQPRRVFILPSNKQDPENSQPEAFKLPLRKEQLRSLWWMLEQEKATGKTHTFVEEEISESLLPAVGWRAEGKAERPVMVRGGVIADQVGYGKTVISIALVAQTLSLPAPEPATPGLIDLKATLIVVPGHLSKQWPNEIARFTGSMFKVIVIQGMKDLQEKTIAELGKADIIVMASEIFESDVYWSRLEYLSAQPREWLHDTQGGRFFCDRLDAAMESLVSQTKILKEKGSEAAMRAMEDKKKSLVDNVGSKKEVHTAVNFGKRMKGQAYRDKHDSDSKAKPITKEELERWEASEDEDDDENSKTYIPIPKFHSFTGSESIFSASVKKDYKLLPNPVLHMFRFRRVIADEFTYLQKKSLAAVLRLSSSYRWILSGTPPVSDFAAIRSIATFMGIHLGVEDDGEGDVQYQKARAKDQTQAEKFHAFREVHSRAWHNRRDELAQEFLNVFVRQNIAEIEDIPTVEHIHTFKLPASEGAVYLELEHHLQALEMQARKETKFKNVSQGDRNARLEEALSDSKTAEEALLKRCCHFTLDLSDKTQDAKSAQEACDHITSARARQLLACQEDLSRSVNQAIALHGWIKKKGGFSKNDDERQPFAEWIAFSSNISKHQGDIEAARILLKVIEKCGVKDGNIPPSPSDKQSPSIASGAKMDDVKWQLREQTHLLRKLVKELVARVRSLRFFEVVRKIQKGKSDAQIVLESSECGHKPSTNPDIEMAILSCCGHVACHKCMRKAAASQRCVKSGECQAAVRPTNIVKVSSLGVEGELSSGRYGAKLEHLVNLIHSIPKNERVLVFLQWEDLAGKVSEALSAGRIPHVTLSGSAKSRANTLDRFQSTNADSARVLLLKMNDASAAGSNLTTANHAVFLGPLFTNSLFNYRAVETQAIGRVRRYGQQKKVHIHRLLALDTIDMTIFNARRTELKEKGGGSDYKDDDDKHHHHHH

SEQ ID NO: 35 - protein, 17 aa, Synthetic construct
LXVATXCSGTXXPJXAL

SEQ ID NO: 36 - protein, 32 aa, Synthetic construct
HLFSCEIEPFKQXXIXRNXXPXXXIFRDVXEL

SEQ ID NO: 37 - protein, 17 aa, Synthetic construct
DILXAGCSCVDYSNXNX

SEQ ID NO: 38 - protein, 19 aa, Synthetic construct
GESXRTFFAAJXXIXXXRP

SEQ ID NO: 39 - protein, 12 aa, Synthetic construct
JVILENVXXAPW

SEQ ID NO: 40 - protein, 19 aa, Synthetic construct
LDSKXXYLPQTRZRGYLXA

SEQ ID NO: 41 - protein, 47 aa, Synthetic construct
WXXXXXRHXDXRXXXXLXXXXXPXSXKXXXNGXXXXXXXPSXSWXXX

SEQ ID NO: 42 - protein, 15 aa, Synthetic construct
QXXRVXDXXDIXFXX

SEQ ID NO: 43 - protein, 29 aa, Synthetic construct
DLXYKTXXJDXSQNVDRXXXXXXXXXXXX

SEQ ID NO: 44 - protein, 13 aa, Synthetic construct
GXXGCITPSGXPX

SEQ ID NO: 45 - protein, 44 aa, Synthetic construct
EXLALQGLPXDXLXXSTEXQXQLXDLAGNAMTXTVVGAXTLAAL

SEQ ID NO: 46 - protein, 45 aa, Synthetic construct
LRPXQXXXVXWMLZREXXXXXFXXXEIEEEVXXXLXXRXXGKAEX

SEQ ID NO: 47 - protein, 21 aa, Synthetic construct
RGGVXAHEXGYGKTVVXLALX

SEQ ID NO: 48 - protein, 22 aa, Synthetic construct
XATLXVVPXHIXXQWXXEXXKF

SEQ ID NO: 49 - protein, 36 aa, Synthetic construct
FARXVXDEXXYXXXXXJXXXVXXXXAXXKWLLSGTP

SEQ ID NO: 50 - protein, 10 aa, Synthetic construct
PGLPXXXXGP

SEQ ID NO: 51 - protein, 17 aa, Synthetic construct
XLLLBXXXETXAGSNLT

SEQ ID NO: 52 - protein, 34 aa, Synthetic construct
AXHVIFXXPXXXXXXXXXXXTXXQAXGRCXRXGQ

SEQ ID NO: 53 - protein, 17 aa, Synthetic construct
LRVVTMCSGTESPLLAL

SEQ ID NO: 54 - protein, 31 aa, Synthetic construct
HLFSAEIVPFKQAYIERNFHPPYLFRDVSEL

SEQ ID NO: 55 - protein, 17 aa, Synthetic construct
DIVIAGFSCVDFSALNN

SEQ ID NO: 56 - protein, 19 aa, Synthetic construct
GESGVTFYGIRSYIEKYRP

SEQ ID NO: 57 - protein, 12 aa, Synthetic construct
LIILENVKNAPW

SEQ ID NO: 58 - protein, 19 aa, Synthetic construct
IDTKTYYLPQTRERGYALC

SEQ ID NO: 59 - protein, 42 aa, Synthetic construct
WSRYQARHQSYRLDNKLGYKRPMTRFQDNGTCKMPDFVWQEW

SEQ ID NO: 60 - protein, 15 aa, Synthetic construct
QPERIWDTLDMNFLR

SEQ ID NO: 61 - protein, 22 aa, Synthetic construct
DLNFKERYLDLSQGIDREIDGR

SEQ ID NO: 62 - protein, 13 aa, Synthetic construct
GIMGCITPCGIPY

SEQ ID NO: 63 - protein, 44 aa, Synthetic construct
EALSLQGLPLDRLLLTKESQRELQDLAGNAMSSTVVGAATLSAL

SEQ ID NO: 64 - protein, 46 aa, Synthetic construct
LRPEQLRSLSWMIEQEAEEPVPFEEEEVEEALLPFMMWRAEAKVTH

SEQ ID NO: 65 - protein, 21 aa, Synthetic construct
RGGVLADEVGYGKTALILGLI

SEQ ID NO: 66 - protein, 22 aa, Synthetic construct
KATLVVVPKMMVTQWKEEISKF

SEQ ID NO: 67 - protein, 35 aa, Synthetic construct
FNRLVIDEFTYVSERKLGPVLNLKARSVWVLSGTP

SEQ ID NO: 68 - protein, 8 aa, Synthetic construct
NPSTRLAN

SEQ ID NO: 69 - protein, 17 aa, Synthetic construct
VLILNMGSESAAGINLQ

SEQ ID NO: 70 - protein, 34 aa, Synthetic construct
ANHVIFLHPFLAKTQYDYESTMTQAIGRARRFGQ

SEQ ID NO: 71 - protein, 17 aa, Synthetic construct
LRVATMCSGTESPLLAL

SEQ ID NO: 72 - protein, 31 aa, Synthetic construct
HLFSAEIDPFKQSYIQRNFSPDIIFRDVNEL

SEQ ID NO: 73 - protein, 17 aa, Synthetic construct
DLLVVGFSCVDFSNLNV

SEQ ID NO: 74 - protein, 19 aa, Synthetic construct
GESGHTFFAVLRYMERCRP

SEQ ID NO: 75 - protein, 12 aa, Synthetic construct
LAVLENVSLAPW

SEQ ID NO: 76 - protein, 19 aa, Synthetic construct
VDTKNYYLPQTRERGYMLC

SEQ ID NO: 77 - protein, 42 aa, Synthetic construct
WTRYKARHMAYRMEEGLGNKRPLTKWQDNGTCQMPDFYWHDW

SEQ ID NO: 78 - protein, 15 aa, Synthetic construct
QTERVWDTLDVNYLR

SEQ ID NO: 79 - protein, 22 aa, Synthetic construct
DINYKTRVVDLSQGLDRELDHR

SEQ ID NO: 80 - protein, 13 aa, Synthetic construct
GVAGCLTPRGLHF

SEQ ID NO: 81 - protein, 44 aa, Synthetic construct
EALALQGIPIDRLLLGSDSQRALYDLAGNAMSSTVVGAAILSAL

SEQ ID NO: 82 - protein, 46 aa, Synthetic construct
LRPEQLRSLTWMVSQEADDVPPFEEEEVVEALLPVINWRAEGRAST

SEQ ID NO: 83 - protein, 21 aa, Synthetic construct
KGGILGDDVGYGKTAISLGLI

SEQ ID NO: 84 - protein, 22 aa, Synthetic construct
KATLIIVPSHLVDQWAREITKF

SEQ ID NO: 85 - protein, 35 aa, Synthetic construct
FNRLIIDEFTYSKARNYASALAIPARKKWILSGTP

SEQ ID NO: 86 - protein, 8 aa, Synthetic construct
RDNERLKA

SEQ ID NO: 87 - protein, 17 aa, Synthetic construct
VLILTLGDVTASGLNLQ

SEQ ID NO: 88 - protein, 34 aa, Synthetic construct
ANHIIFYSPLVARSQYDYESGMAQAIGRCRRYGQ

SEQ ID NO: 89 - protein, 17 aa, Synthetic construct
LRVATVCSGTDAPIFAL

SEQ ID NO: 90 - protein, 32 aa, Synthetic construct
HVFSCEIEPFKQGFIRRNLPQCSLIFRDIVEL

SEQ ID NO: 91 - protein, 18 aa, Synthetic construct
DIFFAGCSCVDYSNMNTN

SEQ ID NO: 92 - protein, 19 aa, Synthetic construct
GESARTFFAAVKLIAERRP

SEQ ID NO: 93 - protein, 12 aa, Synthetic construct
IVILENVSGAPW

SEQ ID NO: 94 - protein, 19 aa, Synthetic construct
LDSKNFYLPQTRQRGYLVA

SEQ ID NO: 95 - protein, 47 aa, Synthetic construct
WSLSSLRHIDARRRANLRHDDNPFSMKAMRGGQLIHMRPPDHSWRPV

SEQ ID NO: 96 - protein, 15 aa, Synthetic construct
AGPRILDLMDIVFAV

SEQ ID NO: 97 - protein, 25 aa, Synthetic construct
DLGYKTAMIDVSQNVDRNEFCRLGR

SEQ ID NO: 98 - protein, 13 aa, Synthetic construct
GIVGCVTPSGLPI

SEQ ID NO: 99 - protein, 44 aa, Synthetic construct
ETLALQGLPAEELVLSTETQAQQRDLAGNAMSVPVVGAATYAAI

SEQ ID NO: 100 - protein, 45 aa, Synthetic construct
LRPSQRDAVKWMIQRERIPLNFIKREIEEEVVQPLNLRVAGKAEW

SEQ ID NO: 101 - protein, 21 aa, Synthetic construct
RGGVVAHEIGYGKTVVTLALV

SEQ ID NO: 102 - protein, 22 aa, Synthetic construct
SATLVIVPRHITDQWAKEIDKF

SEQ ID NO: 103 - protein, 38 aa, Synthetic construct
FARIVWDECSYDDDKDSYIPVFVANAVANSKWLLSGTP

SEQ ID NO: 104 - protein, 10 aa, Synthetic construct
PGLPAITEGP

SEQ ID NO: 105 - protein, 17 aa, Synthetic construct
ILLLDIDNETSAGSNLT

SEQ ID NO: 106 - protein, 34 aa, Synthetic construct
ANHVIFANPFVHHDEEHQSRTVRQARGRCVRIGQ

SEQ ID NO: 107 - protein, 17 aa, Synthetic construct
LNVATLCSGTDAPIFAL

SEQ ID NO: 108 - protein, 32 aa, Synthetic construct
HLFSCEIEPFKQGFIRRNLPPGTLIFRDVVEL

SEQ ID NO: 109 - protein, 17 aa, Synthetic construct
DILFAGCSCVDYSNMNQ

SEQ ID NO: 110 - protein, 19 aa, Synthetic construct
GESARTFFAAIKLITVIRP

SEQ ID NO: 111 - protein, 12 aa, Synthetic construct
LVILENVYNAPW

SEQ ID NO: 112 - protein, 19 aa, Synthetic construct
LDSKDFYLPQTRQRGYLVA

SEQ ID NO: 113 - protein, 47 aa, Synthetic construct
WALCSLRHADVRQRHGIRRDDNPFSLKAVRNSRLIFAAYPSHSWMQF

SEQ ID NO: 114 - protein, 15 aa, Synthetic construct
QVARVVDFMDIAFAA

SEQ ID NO: 115 - protein, 25 aa, Synthetic construct
DLGYKTCMIDVSQNVDRNDLVNGGR

SEQ ID NO: 116 - protein, 13 aa, Synthetic construct
GVVGCITPSGMPI

SEQ ID NO: 117 - protein, 44 aa, Synthetic construct
ETLALQGLPVDDLVVSTESQAQLRDLAGNAMTVTVVGAVTLAAL

SEQ ID NO: 118 - protein, 45 aa, Synthetic construct
LRPSQREAVNWMLQRERVPLDFVKREVEEEVVAPLNLRVLGKAEW

SEQ ID NO: 119 - protein, 21 aa, Synthetic construct
RGGVVAHEVGYGKTVVTLAVC

SEQ ID NO: 120 - protein, 22 aa, Synthetic construct
SATLVVVPKHITAQWESEAAKF

SEQ ID NO: 121 - protein, 36 aa, Synthetic construct
FARVVWDECSYDDDDNIRLFVAHAVANTKWLLSGTP

SEQ ID NO: 122 - protein, 10 aa, Synthetic construct
PGLPAVTKGP

SEQ ID NO: 123 - protein, 17 aa, Synthetic construct
ILLLDMDSETSAGSNLT

SEQ ID NO: 124 - protein, 34 aa, Synthetic construct
ATRVIFASPYVHQDEEHQVRTVRQAKGRCIRTGQ

SEQ ID NO: 125 - protein, 17 aa, Synthetic construct
LNVATLCSGTDAPIFAL

SEQ ID NO: 126 - protein, 32 aa, Synthetic construct
HLFSCEIEPFKQGFIRRNLPQGTLIFRDVVEM

SEQ ID NO: 127 - protein, 17 aa, Synthetic construct
DILFAGCSCVDYSNMNS

SEQ ID NO: 128 - protein, 19 aa, Synthetic construct
GESSRTFFAAVKLISAMRP

SEQ ID NO: 129 - protein, 12 aa, Synthetic construct
AVILENVYGAPW

SEQ ID NO: 130 - protein, 19 aa, Synthetic construct
LDSKDYYLPQTRQRGYLVA

SEQ ID NO: 131 - protein, 47 aa, Synthetic construct
WALSSLRHYDVRHANGVREDDNPFSLKAMRHGRLIFASYPSHSWRRF

SEQ ID NO: 132 - protein, 15 aa, Synthetic construct
QPPRVTDLMDIVFAV

SEQ ID NO: 133 - protein, 25 aa, Synthetic construct
DLGYKTCMIDASQNVDRIHKIDAKN

SEQ ID NO: 134 - protein, 13 aa, Synthetic construct
GIVGCITPSGLPI

SEQ ID NO: 135 - protein, 44 aa, Synthetic construct
ETLSLQGLPVDELVISTETQANLRDLAGNAMTVTVVGAATLALL

SEQ ID NO: 136 - protein, 45 aa, Synthetic construct
LRPSQERAVEWMLQRENAPLDFVKAEIEEEVVRPLNMRVVGKAEW

SEQ ID NO: 137 - protein, 21 aa, Synthetic construct
KGGVVAHEIGYGKTVVMLALI

SEQ ID NO: 138 - protein, 22 aa, Synthetic construct
SATLVVVPRHITDQWAKEATKF

SEQ ID NO: 139 - protein, 36 aa, Synthetic construct
FARVIWDECSYDGNENVTLFVSNAVANAKWLLSGTP

SEQ ID NO: 140 - protein, 10 aa, Synthetic construct
PGLPAVTDGP

SEQ ID NO: 141 - protein, 17 aa, Synthetic construct
ILLLDIDSETSAGSNLT

SEQ ID NO: 142 - protein, 34 aa, Synthetic construct
ANHVIFANPYVHPDEDHRARTVRQAKGRCVRMGQ

SEQ ID NO: 143 - protein, 17 aa, Synthetic construct
LRVVTMCSGTESPLLAL

SEQ ID NO: 144 - protein, 31 aa, Synthetic construct
HLFSAEIVHFKQAYIERNFHPPIIFRDVREL

SEQ ID NO: 145 - protein, 17 aa, Synthetic construct
DILIAGFSCVDFSNLNR

SEQ ID NO: 146 - protein, 19 aa, Synthetic construct
GESGQTFEGILSYVAQHRP

SEQ ID NO: 147 - protein, 12 aa, Synthetic construct
LVVLENIKHAPW

SEQ ID NO: 148 - protein, 19 aa, Synthetic construct
VDTKNYYLPQTRARGYMLC

SEQ ID NO: 149 - protein, 42 aa, Synthetic construct
WSSYRIRHQDYRRTNQLGYRRPVTNFREGGLCRPIDWMDRSW

SEQ ID NO: 150 - protein, 15 aa, Synthetic construct
QPERVLDTIDADFLY

SEQ ID NO: 151 - protein, 22 aa, Synthetic construct
DNRYKLRVHDVSQGVDREADNR

SEQ ID NO: 152 - protein, 13 aa, Synthetic construct
GIVGCLTPCGMPY

SEQ ID NO: 153 - protein, 44 aa, Synthetic construct
ESLGLQGLPTYRLNLTRESQKELQDLAGNAMSSTVVAGAIASAL

SEQ ID NO: 154 - protein, 46 aa, Synthetic construct
LRPDQLRSLAWMLSRESDGMTPFMEEEIDEAFMAPLDWRAEIKIIV

SEQ ID NO: 155 - protein, 21 aa, Synthetic construct
RGGILADHVGFGKTAVVLGLL

SEQ ID NO: 156 - protein, 22 aa, Synthetic construct
KATLIVVPETLFGQWRAEIEKF

SEQ ID NO: 157 - protein, 35 aa, Synthetic construct
FQRMVVDEFTYLDQDKHTLLASIKARSKWLLSGTP

SEQ ID NO: 158 - skipped

SEQ ID NO: 159 - protein, 17 aa, Synthetic construct
ILLLRLGTKNAAGLNLQ

SEQ ID NO: 160 - protein, 34 aa, Synthetic construct
ANHIIFFAPMSVHHPSEYQASVAQAMGRAIRWGQ

SEQ ID NO: 161-162 - skipped (2 entries)

SEQ ID NO: 163 - protein, 17 aa, Synthetic construct
DIFVAGCSCVDFSNLNS

SEQ ID NO: 164 - protein, 19 aa, Synthetic construct
GESSTTFLAALSYIIKVEP

SEQ ID NO: 165 - protein, 12 aa, Synthetic construct
IVILENVDSAPW

SEQ ID NO: 166 - protein, 19 aa, Synthetic construct
VNSRMYYMPQTRQRGYLVA

SEQ ID NO: 167 - protein, 47 aa, Synthetic construct
WGFSQVRHQSTREEAKLSLTARPFSRGIIVRGRMVDMQPPSRSWRSY

SEQ ID NO: 168 - protein, 15 aa, Synthetic construct
QPARVLDFIDIAYLQ

SEQ ID NO: 169 - protein, 24 aa, Synthetic construct
DLRYKTWVLDVSQNVDRSTVFGTN

SEQ ID NO: 170 - protein, 13 aa, Synthetic construct
GSIGCLTPSGQPI

SEQ ID NO: 171 - protein, 44 aa, Synthetic construct
ESLGLQGLPVDDLVLSTETQAQLQDLAGNAMTVTVIGSVILAAL

SEQ ID NO: 172 - protein, 45 aa, Synthetic construct
LRDDQVLSVKWMLGRETCPAPFLEKEIEELVLPRLAARIIGRAIF

SEQ ID NO: 173 - protein, 21 aa, Synthetic construct
RGGVAAHDIGYGKTVVTIALL

SEQ ID NO: 174 - protein, 22 aa, Synthetic construct
KATLIIVPGHISTQWVAEFKKF

SEQ ID NO: 175 - protein, 35 aa, Synthetic construct
FARIVVDEYSYQAPSTLSFVSSSLAFAKWLLSGTP

SEQ ID NO: 176 - protein, 10 aa, Synthetic construct
PGLPSVTEGP

SEQ ID NO: 177 - protein, 17 aa, Synthetic construct
VLMLNPSEVSCAGVNLT

SEQ ID NO: 178 - protein, 34 aa, Synthetic construct
ANHVMFVSPFLEPDPYRRNQGMRQAVGRCVRQGQ

SEQ ID NO: 179 - protein, 17 aa, Synthetic construct
LAVATICSGTDAPIFAL

SEQ ID NO: 180 - protein, 32 aa, Synthetic construct
HVFSCEIEPFKQGFISRNMPPATVIFRDVVEL

SEQ ID NO: 181 - protein, 17 aa, Synthetic construct
DILFAGCSCVDYSNLNH

SEQ ID NO: 182 - protein, 19 aa, Synthetic construct
GESARTFFAAVKLIARTRP

SEQ ID NO: 183 - protein, 12 aa, Synthetic construct
AVILENVLGAPW

SEQ ID NO: 184 - protein, 19 aa, Synthetic construct
LDSKEFYLPQTRQRGYLVA

SEQ ID NO: 185 - protein, 47 aa, Synthetic construct
WNLCSIRHAIVRQKYGLRRDENPVSKKAMRNGRIIFANFPSHSWMRF

SEQ ID NO: 186 - protein, 15 aa, Synthetic construct
QVPRIPDLVDIVFAI

SEQ ID NO: 187 - protein, 25 aa, Synthetic construct
DLGYKTGMIDVSQNVDRTEYTRSGS

SEQ ID NO: 188 - protein, 13 aa, Synthetic construct
GIIGCITPSGLPV

SEQ ID NO: 189 - protein, 44 aa, Synthetic construct
EALALQGLPVDELVISTETQAQLRDLAGNAMTVTVVGAATLALI

SEQ ID NO: 190 - protein, 45 aa, Synthetic construct
LRDSQKEAVKWMLRRELVPLDFIKCEIEEEVVSPLNMRVMGKAEW

SEQ ID NO: 191 - protein, 21 aa, Synthetic construct
RGGVVAHEIGYGKTVVTLALI

SEQ ID NO: 192 - protein, 22 aa, Synthetic construct
SATLVIVPKHITEQWANEAYKF

SEQ ID NO: 193 - protein, 36 aa, Synthetic construct
FARVVWDECSYNDEEKIPLVIANVVANSKWLLSGTP

SEQ ID NO: 194 - protein, 10 aa, Synthetic construct
PGLPPVTKGP

SEQ ID NO: 195-196 - skipped (2 entries)

SEQ ID NO: 197 - protein, 8 aa, Synthetic construct
LNVATLCS

SEQ ID NO: 198 - protein, 32 aa, Synthetic construct; identical to SEQ ID NO: 108

SEQ ID NO: 199 - protein, 17 aa, Synthetic construct
DILFAGCSCVDYSNMNQ

SEQ ID NO: 200 - protein, 19 aa, Synthetic construct
GESARTFFAAIKLITVIRP

SEQ ID NO: 201 - protein, 12 aa, Synthetic construct
LVILENVYNAPW

SEQ ID NO: 202 - protein, 19 aa, Synthetic construct
LDSKEFYLPQTRQRGYLLA

SEQ ID NO: 203 - protein, 47 aa, Synthetic construct
WALCSLRHADVRQKHGIRRDDNPFSLKAMRNSRIIFAAYPSHSWMQF

SEQ ID NO: 204 - protein, 15 aa, Synthetic construct
QVARVVDFMDIAFAT

SEQ ID NO: 205 - protein, 25 aa, Synthetic construct
DLGFKTCMIDVSQNVDRNDLVNGGR

SEQ ID NO: 206 - protein, 13 aa, Synthetic construct
GIVGCITPSGLPI

SEQ ID NO: 207 - protein, 44 aa, Synthetic construct
ETLALQGLPVNDLIVSTETQAQLRDLAGNAMTVTVVGAVTLALL

SEQ ID NO: 208 - protein, 45 aa, Synthetic construct
LRPSQKEAVNWMLQRERVALNFVKREIEEEVVAPLNLRVMGKAEW

SEQ ID NO: 209 - protein, 21 aa, Synthetic construct
RGGVVAHEIGYGKTVVTLAVC

SEQ ID NO: 210 - protein, 22 aa, Synthetic construct
SATLVIVPKHITAQWESEAAKF

SEQ ID NO: 211 - protein, 36 aa, Synthetic construct
FARIVWDECSYDDDSNIRLFVANAVANAKWLLSGTP

SEQ ID NO: 212 - protein, 10 aa, Synthetic construct
PGLPAVTKGP

SEQ ID NO: 213 - protein, 17 aa, Synthetic construct
ILLLDMDSETSAGSNLT

SEQ ID NO: 214 - protein, 34 aa, Synthetic construct
ASRVIFANPYVHGDEEHQVRTVRQARGRCIRTGQ

SEQ ID NO: 215 - protein, 17 aa, Synthetic construct
LNVATLCSGTDAPIFAL

SEQ ID NO: 216 - protein, 32 aa, Synthetic construct
HLFSCEIEPFKQGFIRRNLPHGTVVFRDVIEM

SEQ ID NO: 217 - protein, 17 aa, Synthetic construct
DILFAGCSCVDYSNMNH

SEQ ID NO: 218 - protein, 19 aa, Synthetic construct
SESARTFFAAIKLITILRP

SEQ ID NO: 219 - protein, 12 aa, Synthetic construct
FVILENVYSAPW

SEQ ID NO: 220 - protein, 19 aa, Synthetic construct
LDSKDFYLPQTRQRGYLVA

SEQ ID NO: 221 - protein, 47 aa, Synthetic construct
WALSSLRHADARHQQGLKKDDNPFSLKAMRNNRIIHATYPSHSWRSF

SEQ ID NO: 222 - protein, 15 aa, Synthetic construct
QPARIIDLMDIIFAT

SEQ ID NO: 223 - protein, 25 aa, Synthetic construct
DLGYKTYMIDASQNVDRNTLVISDR

SEQ ID NO: 224 - protein, 13 aa, Synthetic construct
GIVGCITPSGLPV

SEQ ID NO: 225 - protein, 44 aa, Synthetic construct
ETLALQGLPVDEMVLSTETQGQLRDLAGNAMTVTVVGAATLALL

SEQ ID NO: 226 - protein, 45 aa, Synthetic construct
LRPSQKSAVAWMLERERAQLNFVKCEIEEEVVPSLGIRVLGKAEW

SEQ ID NO: 227 - protein, 21 aa, Synthetic construct
RGGIVAHEIGYGKTVVTLAVI

SEQ ID NO: 228 - protein, 22 aa, Synthetic construct
TATLIVVPKHIAHQWEAEAAKF

SEQ ID NO: 229 - protein, 36 aa, Synthetic construct
FARIVWDEYTYDGGDIIHLFVSNAVANAKWLLSGTP

SEQ ID NO: 230 - protein, 10 aa, Synthetic construct
PGLPPITTGP

SEQ ID NO: 231 - protein, 17 aa, Synthetic construct
VILLDMDSETSAGSNLT

SEQ ID NO: 232 - protein, 34 aa, Synthetic construct
ATHLIFASPYVHYDPEHQIRTVQQARGRCIRTGQ

SEQ ID NO: 233 - protein, 17 aa, Synthetic construct
LNVATLCSGTDAPIFAL

SEQ ID NO: 234 - protein, 32 aa, Synthetic construct; identical to SEQ ID NO: 216

SEQ ID NO: 235 - protein, 17 aa, Synthetic construct
DILFAGCSCVDYSNMNH

SEQ ID NO: 236 - protein, 19 aa, Synthetic construct
SESARTFFAAIKLITILRP

SEQ ID NO: 237 - protein, 12 aa, Synthetic construct
FVILENVYSAPW

SEQ ID NO: 238 - protein, 19 aa, Synthetic construct
LDSKDFYLPQTRQRGYLVA

SEQ ID NO: 239 - protein, 47 aa, Synthetic construct; identical to SEQ ID NO: 221

SEQ ID NO: 240 - protein, 15 aa, Synthetic construct
QPARIIDLMDIIFAT

SEQ ID NO: 241 - protein, 25 aa, Synthetic construct
DLGYKTYMIDASQNVDRNTLVISDR

SEQ ID NO: 242 - protein, 13 aa, Synthetic construct
GIVGCITPSGLPV

SEQ ID NO: 243 - protein, 44 aa, Synthetic construct; identical to SEQ ID NO: 225

SEQ ID NO: 244 - protein, 45 aa, Synthetic construct; identical to SEQ ID NO: 226

SEQ ID NO: 245 - protein, 21 aa, Synthetic construct
RGGIVAHEIGYGKTVVTLAVI

SEQ ID NO: 246 - protein, 22 aa, Synthetic construct
TATLIVVPKHIAHQWEAEAAKF

SEQ ID NO: 247 - protein, 36 aa, Synthetic construct; identical to SEQ ID NO: 229

SEQ ID NO: 248 - protein, 10 aa, Synthetic construct
PGLPPITTGP

SEQ ID NO: 249 - protein, 17 aa, Synthetic construct
VILLDMDSETSAGSNLT

SEQ ID NO: 250 - protein, 34 aa, Synthetic construct; identical to SEQ ID NO: 232

SEQ ID NO: 251 - protein, 17 aa, Synthetic construct
LSVGTICSGTDAPIYAL

SEQ ID NO: 252 - protein, 31 aa, Synthetic construct
HHFSAEIEPAKQGFIRRNLDDIVIFRDVIEL

SEQ ID NO: 253 - protein, 17 aa, Synthetic construct
DILFAGCSCVDYSNMNH

SEQ ID NO: 254 - protein, 19 aa, Synthetic construct
SESAMTFFAAIKLITIIRP

SEQ ID NO: 255 - protein, 12 aa, Synthetic construct
FVILENVYSAPW

SEQ ID NO: 256 - protein, 19 aa, Synthetic construct
LDSKDYYLPQTRQRGYLVA

SEQ ID NO: 257 - protein, 47 aa, Synthetic construct
WALSSLRHADARHKNKLRRDDNPFSMKAMRNGKIVNAAYQSHSWMAF

SEQ ID NO: 258 - protein, 15 aa, Synthetic construct
QVARVIDLMDINFAV

SEQ ID NO: 259 - protein, 25 aa, Synthetic construct
DLGYKTYMIDASQNVDRNQLVTSGN

SEQ ID NO: 260 - protein, 13 aa, Synthetic construct
GIVGCITPSGLPV

SEQ ID NO: 261 - protein, 44 aa, Synthetic construct
EALALQGLPVDELVLSTETQSQLRDLAGNAMSLPVVGAATLALL

SEQ ID NO: 262 - protein, 45 aa, Synthetic construct
LRPSQKLAVDWMLLRERASLRFAECEIEEEVVAPLGIRVMGKAEW

SEQ ID NO: 263 - protein, 21 aa, Synthetic construct
RGGVVAHEIGYGKTVVSLALI

SEQ ID NO: 264 - protein, 22 aa, Synthetic construct
SATLIIVPKHISQQWAAEAEKF

SEQ ID NO: 265 - protein, 36 aa, Synthetic construct
FARIIWDEYTYDGGDLIHLFVSNAVANAKWLLSGTP

SEQ ID NO: 266 - protein, 10 aa, Synthetic construct
PGLPPVTAGP

SEQ ID NO: 267 - protein, 17 aa, Synthetic construct
ILLLDMDSETSAGSNLT

SEQ ID NO: 268 - protein, 34 aa, Synthetic construct
ATHIIFANPYVHPDPEHQIRTVSQARGRCIRTGQ

SEQ ID NO: 269 - protein, 17 aa, Synthetic construct
LNVATLCSGTDAPIFAL

SEQ ID NO: 270 - protein, 32 aa, Synthetic construct; identical to SEQ ID NO: 108

SEQ ID NO: 271 - protein, 17 aa, Synthetic construct
DILFAGCSCVDYSNMNQ

SEQ ID NO: 272 - protein, 19 aa, Synthetic construct
GESARTFFAAIKLITVIRP

SEQ ID NO: 273 - protein, 12 aa, Synthetic construct
LVILENVYNAPW

SEQ ID NO: 274 - protein, 19 aa, Synthetic construct
LDSKDFYLPQTRQRGYLVA

SEQ ID NO: 275 - protein, 47 aa, Synthetic construct
WALCSLRHADVRQRHGIRRDDNPFSLKAMRNSRHIFAAYPSHSWMQF

SEQ ID NO: 276 - protein, 15 aa, Synthetic construct
QVARVADFMDIAFAA

SEQ ID NO: 277 - protein, 25 aa, Synthetic construct
DLGYKTCMIDVSQNVDRNDLVNGGR

SEQ ID NO: 278 - protein, 13 aa, Synthetic construct
GVVGCITPSGMPI

SEQ ID NO: 279 - protein, 44 aa, Synthetic construct; identical to SEQ ID NO: 117

SEQ ID NO: 280 - protein, 45 aa, Synthetic construct; identical to SEQ ID NO: 118

SEQ ID NO: 281 - protein, 21 aa, Synthetic construct
RGGVVAHEVGYGKTVVTLAVC

SEQ ID NO: 282 - protein, 22 aa, Synthetic construct
SATLVVVPKHITAQWESEAAKF

SEQ ID NO: 283 - protein, 36 aa, Synthetic construct; identical to SEQ ID NO: 121

SEQ ID NO: 284 - protein, 10 aa, Synthetic construct
PGLPAVTKGP

SEQ ID NO: 285 - protein, 17 aa, Synthetic construct
ILLLDMDSETSAGSNLT

SEQ ID NO: 286 - protein, 34 aa, Synthetic construct; identical to SEQ ID NO: 124

SEQ ID NO: 287 - protein, 17 aa, Synthetic construct
LNVATLCSGTDAPIFAL

SEQ ID NO: 288 - protein, 32 aa, Synthetic construct
HLFSCEIEPFKQGFIRRNLPPGTLIFRDIVEL

SEQ ID NO: 289 - protein, 17 aa, Synthetic construct
DILFAGCSCVDYSAMNQ

SEQ ID NO: 290 - protein, 19 aa, Synthetic construct
GESARTFFAAIKLITVIRP

SEQ ID NO: 291 - protein, 12 aa, Synthetic construct
VVILENVYNAPW

SEQ ID NO: 292 - protein, 19 aa, Synthetic construct
LDSKDFYLPQTRQRGYLVA

SEQ ID NO: 293 - protein, 47 aa, Synthetic construct
WALCSLRHADVRQRHGIRRDDNPFSLKAMRNSRLIFAAYPSHSWMQF

SEQ ID NO: 294 - protein, 15 aa, Synthetic construct
QGARVVDFMDIAFSA

SEQ ID NO: 295 - protein, 25 aa, Synthetic construct
DLGYKTCMIDVSQNVDRNDLVTGGR

SEQ ID NO: 296 - protein, 13 aa, Synthetic construct
GLIGCITPSGMPI

SEQ ID NO: 297 - protein, 44 aa, Synthetic construct; identical to SEQ ID NO: 117

SEQ ID NO: 298 - protein, 45 aa, Synthetic construct
LRPSQREAVNWMLQRERVPLDFVKREIEEEVVSPLNLRVLGKAEW

SEQ ID NO: 299 - protein, 21 aa, Synthetic construct
RGGVVAHEVGYGKTVVTLAVC

SEQ ID NO: 300 - protein, 22 aa, Synthetic construct
SATLVVVPRHITAQWESEAAKF

SEQ ID NO: 301 - protein, 36 aa, Synthetic construct
FARIVWDECSYDDDDNIRLFVAHAVANAKWLLSGTP

SEQ ID NO: 302 - protein, 10 aa, Synthetic construct
PGLPSVTKGP

SEQ ID NO: 303 - protein, 17 aa, Synthetic construct
ILLLDMDSETSAGSNLT

SEQ ID NO: 304 - protein, 34 aa, Synthetic construct
ATRVIFANPYVHQDEEHQARTVRQAKGRCIRTGQ

SEQ ID NO: 305 - protein, 17 aa, Synthetic construct
LNVATLCSGTDAPIFAL

SEQ ID NO: 306 - protein, 32 aa, Synthetic construct; identical to SEQ ID NO: 108

SEQ ID NO: 307 - protein, 17 aa, Synthetic construct
DILFAGCSCVDYSNMNQ

SEQ ID NO: 308 - protein, 19 aa, Synthetic construct
GESARTFFAAIKLITVIRP

SEQ ID NO: 309 - protein, 12 aa, Synthetic construct
LVILENVYNAPW

SEQ ID NO: 310 - protein, 19 aa, Synthetic construct
LDSKDFYLPQTRQRGYLVA

SEQ ID NO: 311 - protein, 48 aa, Synthetic construct
EWALCSLRHADVRQRHGIRGDDNPFSLKTMRNSRLIFAAYPSHSWMQF

SEQ ID NO: 312 - protein, 15 aa, Synthetic construct
QVARVVDFMDIAFAA

SEQ ID NO: 313 - protein, 25 aa, Synthetic construct
DLGYKTCMIDVSQNVDRNDLVNGGR

SEQ ID NO: 314 - protein, 13 aa, Synthetic construct
GVVGCITPSGMPI

SEQ ID NO: 315 - protein, 44 aa, Synthetic construct; identical to SEQ ID NO: 117

SEQ ID NO: 316 - protein, 45 aa, Synthetic construct
LRPSQREAVKWMLQRERVPLDFVKREVEEEVVAPLNLRVLGKAEW

SEQ ID NO: 317 - protein, 21 aa, Synthetic construct
RGGVVAHEVGYGKTVVTLAVC

SEQ ID NO: 318 - protein, 22 aa, Synthetic construct
SATLVVVPKHITAQWESEAAKF

SEQ ID NO: 319 - protein, 36 aa, Synthetic construct
FARVVWDECSYDDDDNIRLFVAHAVANAKWLLSGTP

SEQ ID NO: 320 - protein, 10 aa, Synthetic construct
PGLPAVTKGP

SEQ ID NO: 321 - protein, 17 aa, Synthetic construct
ILLLDMDSETSAGSNLT

SEQ ID NO: 322 - protein, 34 aa, Synthetic construct; identical to SEQ ID NO: 124

SEQ ID NO: 323 - protein, 17 aa, Synthetic construct
LNVATLCSGTDAPIFAL

SEQ ID NO: 324 - protein, 32 aa, Synthetic construct
HLFSCEIEPFKQGFIRRNLPPDTLIFRDIVEL

SEQ ID NO: 325 - protein, 17 aa, Synthetic construct
DILFAGCSCVDYSNMNQ

SEQ ID NO: 326 - protein, 19 aa, Synthetic construct
GESARTFFAAIKLITIIRP

SEQ ID NO: 327 - protein, 12 aa, Synthetic construct
LVILENVYNAPW

SEQ ID NO: 328 - protein, 19 aa, Synthetic construct
LDSKDFYLPQTRQRGYLVA

SEQ ID NO: 329 - protein, 47 aa, Synthetic construct
WALCSLRHADVRQKHGIRRDDNPFSLKAMRNSRLVFAAYPSHSWMEF

SEQ ID NO: 330 - protein, 15 aa, Synthetic construct
QSARVVDFMDIAFSA

SEQ ID NO: 331 - protein, 25 aa, Synthetic construct
DLAYKTCMIDVSQNVDRNDLVNGGK

SEQ ID NO: 332 - protein, 13 aa, Synthetic construct
GLIGCITPSGLPI

SEQ ID NO: 333 - protein, 44 aa, Synthetic construct
ETLALQGLPVDDLVISTESQAQLRDLAGNAMTVTVVGAVTLAAL

SEQ ID NO: 334 - protein, 45 aa, Synthetic construct
LRPSQKEAVNWMIQRERVPLEFVKREIEEEVVAPLNMRVLGKAEW

SEQ ID NO: 335 - protein, 21 aa, Synthetic construct
RGGVVAHEVGYGKTVVTLALC

SEQ ID NO: 336 - protein, 22 aa, Synthetic construct
SATLVVVPRHITAQWESEAAKF

SEQ ID NO: 337 - protein, 37 aa, Synthetic construct
FARIVWDECSYDDDEKNVHLFVAHAVANAKWLLSGTP

SEQ ID NO: 338 - protein, 10 aa, Synthetic construct
PGLPPVTKGP

SEQ ID NO: 339 - protein, 17 aa, Synthetic construct
ILLLDMDEETSAGSNLT

SEQ ID NO: 340 - protein, 34 aa, Synthetic construct; identical to SEQ ID NO: 124

SEQ ID NO: 341 - protein, 17 aa, Synthetic construct
LNVATLCSGTDAPIFAL

SEQ ID NO: 342 - protein, 32 aa, Synthetic construct
HLFSCEIEPFKQGFIRRNLPHGTLIFRDVVEL

SEQ ID NO: 343 - protein, 17 aa, Synthetic construct
DILFAGCSCVDYSNMNQ

SEQ ID NO: 344 - protein, 19 aa, Synthetic construct
GESARTFFAAIKLITALRP

SEQ ID NO: 345 - protein, 12 aa, Synthetic construct
LIILENVAGAPW

SEQ ID NO: 346 - protein, 19 aa, Synthetic construct
LDSKYFYLPQTRQRGYLVA

SEQ ID NO: 347 - protein, 47 aa, Synthetic construct
WSLCQLRHADTRRNKDLRRDDNPFSKKAMRNGRVIFASFPSHSWRPF

SEQ ID NO: 348 - protein, 15 aa, Synthetic construct
QVPRIVDLMDIVVAI

SEQ ID NO: 349 - protein, 25 aa, Synthetic construct
DLAFKTGMVDASQNVDRLPYVVGSG

SEQ ID NO: 350 - protein, 13 aa, Synthetic construct
GIVGCITPNGLPI

SEQ ID NO: 351 - protein, 44 aa, Synthetic construct
ETLALQGLPIDELVISTETQAQLRDLAGNAMSVTVVGAVTLALL

SEQ ID NO: 352 - protein, 45 aa, Synthetic construct
LRASQKDAVNWMLQREQVPLDFVKSEIEEEVVAPLNVRIVGKAEW

SEQ ID NO: 353 - protein, 21 aa, Synthetic construct
RGGVVAHEIGYGKTVVTLALL

SEQ ID NO: 354 - protein, 22 aa, Synthetic construct
SATLVIVPKHITDQWAAEIVKF

SEQ ID NO: 355 - protein, 36 aa, Synthetic construct
FARVVWDECSYDEGGLVPLFVANVVANAKWLLSGTP

SEQ ID NO: 356 - protein, 10 aa, Synthetic construct
PGLPAVTKGP

SEQ ID NO: 357 - protein, 17 aa, Synthetic construct
ILLLDMDSETSAGSNLT

SEQ ID NO: 358 - protein, 34 aa, Synthetic construct
ATHVIFANPYVHNDEEHQARTIRQARGRCIRTGQ

SEQ ID NO: 359 - protein, 17 aa, Synthetic construct
LKVATLCSGTDAPIFAL

SEQ ID NO: 360 - protein, 32 aa, Synthetic construct
HLFSCEIEPEKQGFIRRNLGADTLIFRDVFEL

SEQ ID NO: 361 - protein, 17 aa, Synthetic construct
DILFAGCSCVDYSNMNM

SEQ ID NO: 362 - protein, 19 aa, Synthetic construct
GESARTFFSAIKLVSKLRP

SEQ ID NO: 363 - protein, 12 aa, Synthetic construct
TVMLENVLGAPW

SEQ ID NO: 364 - protein, 19 aa, Synthetic construct
LDTKHFYLPQTRKRGYLIA

SEQ ID NO: 365 - protein, 47 aa, Synthetic construct
WGFSSLRHFDERRRHGLLLDENPFSLKAMRNGRLIYASYPSHSWKRL

SEQ ID NO: 366 - protein, 15 aa, Synthetic construct
LGARVVDSMDILFAA

SEQ ID NO: 367 - protein, 26 aa, Synthetic construct
DIGYKTMLADVSQNIDRSNKFLPVGG

SEQ ID NO: 368 - protein, 13 aa, Synthetic construct
GIIGCITPSGIPV

SEQ ID NO: 369 - protein, 44 aa, Synthetic construct
EALALQGLPADELVISTETQAQLRDLAGNAMTVTVVGAATLVLL

SEQ ID NO: 370 - protein, 45 aa, Synthetic construct
LRPSQRDAVHWMMQRERSPVDFTKVEIEEEVVRPLNMRVMGKAEW

SEQ ID NO: 371 - protein, 21 aa, Synthetic construct
RGGVVAHEIGYGKTVVMLALH

SEQ ID NO: 372 - protein, 22 aa, Synthetic construct
SATLVVVPRHITKQWAKEAARF

SEQ ID NO: 373 - protein, 36 aa, Synthetic construct
FARIIWDECSYDGNSTISLFVTNAIANSKWLISGTP

SEQ ID NO: 374 - protein, 10 aa, Synthetic construct
PGLPSVIKGP

SEQ ID NO: 375 - protein, 17 aa, Synthetic construct
VLLLDIESDTSAGSNLT

SEQ ID NO: 376 - protein, 34 aa, Synthetic construct
ASHVIFANPFVHSDKEFQARTVRQARGRCIRLGQ

SEQ ID NO: 377 - protein, 17 aa, Synthetic construct
LRVVTMCSGTESPLLAL

SEQ ID NO: 378 - protein, 31 aa, Synthetic construct
HLFSAEIVPFKQAYIERNFHPRYLFRDVEQL

SEQ ID NO: 379 - protein, 17 aa, Synthetic construct
DLVIAGFSCVDFSGLNN

SEQ ID NO: 380 - protein, 19 aa, Synthetic construct
GESGGTFWGIIRYAVAYRP

SEQ ID NO: 381 - protein, 12 aa, Synthetic construct
IVVLENIKNAPW

SEQ ID NO: 382 - protein, 19 aa, Synthetic construct
VDTKAYYLPQTRERGYMLC

SEQ ID NO: 383 - protein, 42 aa, Synthetic construct
WKKYQSRHQNYRSTQGLGFERPMSKFQDSGACQTPDFAWQTW

SEQ ID NO: 384 - protein, 15 aa, Synthetic construct
LPERIWDTIDVNFLR

SEQ ID NO: 385 - protein, 22 aa, Synthetic construct
DMNFKERCIELSQGVDREIDSR

SEQ ID NO: 386 - protein, 13 aa, Synthetic construct
GIIGCITPSGMPY

SEQ ID NO: 387 - protein, 44 aa, Synthetic construct
ESLALQGLPLDRLLLTRESSRDLQDLAGNAMSSTVVGAAVLSAL

SEQ ID NO: 388 - protein, 46 aa, Synthetic construct
LRPEQLRSLSWMLEQEKDDIAPFVEEEVEEEILPSMMWRAEGKVVI

SEQ ID NO: 389 - protein, 21 aa, Synthetic construct
RGGVLADDVGYGKTAITLGLI

SEQ ID NO: 390 - protein, 22 aa, Synthetic construct
KATLVLVPQIVLEQWRLEIAKF

SEQ ID NO: 391 - protein, 35 aa, Synthetic construct
FNRIVIDEFTYAGNERLAPLLALRARSKWILSGTP

SEQ ID NO: 392 - protein, 8 aa, Synthetic construct
SQNTRLKV

SEQ ID NO: 393 - protein, 17 aa, Synthetic construct
VVILNLGSETAAGLNLQ

SEQ ID NO: 394 - protein, 34 aa, Synthetic construct
TNHVIFLSPMLAQTQYDYDSSMTQAIGRALRYGQ

SEQ ID NO: 395 - protein, 12 aa, Synthetic construct
MCSGTESPLLAL

SEQ ID NO: 396 - protein, 31 aa, Synthetic construct
HLFSAEIVPFKQAYIERNFHPPYIFRDVREL

SEQ ID NO: 397 - protein, 17 aa, Synthetic construct
DIVIAGFSCVDFSALNN

SEQ ID NO: 398 - protein, 19 aa, Synthetic construct
GESGITFHAILQYIEKYRP

SEQ ID NO: 399 - protein, 12 aa, Synthetic construct
LAVLENVSHAPW

SEQ ID NO: 400 - protein, 19 aa, Synthetic construct
VDSKAYYLPQTRERGYAIC

SEQ ID NO: 401 - protein, 42 aa, Synthetic construct
WTRYQARHQSYRLNNKLGYKRPMTRYQDNGTCKMPDFVWHEW

SEQ ID NO: 402 - protein, 15 aa, Synthetic construct
QPERIWDTLDMNFLR

SEQ ID NO: 403 - protein, 22 aa, Synthetic construct
DLNFKERYLDLSQGIDREQDSR

SEQ ID NO: 404 - protein, 13 aa, Synthetic construct
GIMGCITPCGIPY

SEQ ID NO: 405 - protein, 44 aa, Synthetic construct
EALSLQGLPLDRLLLTKESQRELQDLAGNAMTSTVVGAATLSAL

SEQ ID NO: 406 - protein, 46 aa, Synthetic construct
LRPEQLRSLRWMIQQEADDPMPFEQEEVEEALLPSMMWRAEAKVTA

SEQ ID NO: 407 - protein, 21 aa, Synthetic construct
RGGVLADQVGYGKTALILGLI

SEQ ID NO: 408 - protein, 22 aa, Synthetic construct
KATLIVVPRMMVKQWQSEITKF

SEQ ID NO: 409 - protein, 35 aa, Synthetic construct
FSRLVVDEFTYVDQRKLGPVQKLKSRSVWVLSGTP

SEQ ID NO: 410 - protein, 8 aa, Synthetic construct
MSSARLAS

SEQ ID NO: 411 - protein, 17 aa, Synthetic construct
VLILNMGTESAAGINLQ

SEQ ID NO: 412 - protein, 34 aa, Synthetic construct
ANHVIFLHPFLAKTQYDYESTMTQAIGRSRRFGQ

SEQ ID NO: 413 - protein, 17 aa, Synthetic construct
LRVVTMCSGTESPLLAM

SEQ ID NO: 414 - protein, 31 aa, Synthetic construct
HLFSSEIDPFKQAYIERNFQPPVLFRDVKQL

SEQ ID NO: 415 - protein, 17 aa, Synthetic construct
DILIAGFSCVDFSVLNN

SEQ ID NO: 416 - protein, 19 aa, Synthetic construct
GESGQTFFAIIGYAKLHRP

SEQ ID NO: 417 - protein, 12 aa, Synthetic construct
LVVLENVKNAPW

SEQ ID NO: 418 - protein, 19 aa, Synthetic construct
VDTKHFYLPQTRERGYMVC

SEQ ID NO: 419 - protein, 42 aa, Synthetic construct
WLRYKIRHQDYRREKGLGHKRPLTNYQDGGTCRPPSWTDRHW

SEQ ID NO: 420 - protein, 15 aa, Synthetic construct
QVERVWDTIDMNFLR

SEQ ID NO: 421 - protein, 22 aa, Synthetic construct
DINYKLRIIDLSQGIDREIDGR

SEQ ID NO: 422 - protein, 13 aa, Synthetic construct
GVAGCITPCGMQY

SEQ ID NO: 423 - protein, 44 aa, Synthetic construct
ESLALQGLPLDRLTLTRESSRELQDLAGNAMSSTVVGSAILAAL

SEQ ID NO: 424 - protein, 46 aa, Synthetic construct
LRPEQLRSLHWIRACESDDAPPFREEEVEEAVLGPLEWRVEAKVMB

SEQ ID NO: 425 - protein, 21 aa, Synthetic construct
RGGVLADVVGFGKTALVLGTI

SEQ ID NO: 426 - protein, 22 aa, Synthetic construct
KATLIVVPDILFDQWQSEIKKF

SEQ ID NO: 427 - protein, 35 aa, Synthetic construct
FQRVIIDEFTYLETDKHALLSTIKARSRWLLSGTP

SEQ ID NO: 428 - skipped

SEQ ID NO: 429 - protein, 17 aa, Synthetic construct
VLILMLGSENAAGINLQ

SEQ ID NO: 430 - protein, 34 aa, Synthetic construct
ANHVIFLAEMCAATAYDHESSMTQAIGRALRYGQ

SEQ ID NO: 431 - protein, 17 aa, Synthetic construct
LRVATMCSGTESPLLAL

SEQ ID NO: 432 - protein, 31 aa, Synthetic construct
HVFSCEIEPFKQAYIERNFTPPILFRDVTEL

SEQ ID NO: 433 - protein, 17 aa, Synthetic construct
DILIAGTSCVDYSNLNN

SEQ ID NO: 434 - protein, 19 aa, Synthetic construct
GESGRTFRGMLQWVKKHQP

SEQ ID NO: 435 - protein, 12 aa, Synthetic construct
IVILENVCNAPW

SEQ ID NO: 436 - protein, 19 aa, Synthetic construct
LDTKEFYIPHTRTRVYLFA

SEQ ID NO: 437 - protein, 42 aa, Synthetic construct
WNRCESRHQRARQDEALGLLRPLTSWQEAGVCKGLDWTWNDW

SEQ ID NO: 438 - protein, 15 aa, Synthetic construct
QTERVVDLLEISTLR

SEQ ID NO: 439 - protein, 21 aa, Synthetic construct
DSGFKACIWNVSQNVDRQTGS

SEQ ID NO: 440 - protein, 13 aa, Synthetic construct
ALAPCLTPNMIPW

SEQ ID NO: 441 - protein, 44 aa, Synthetic construct
EALALQGIPVRELLLTSENEDQLADLAGNAMTTTVVGSAMIAAL

SEQ ID NO: 442 - protein, 48 aa, Synthetic construct
LRKEQLRSLWWMLEQEKATGKTHTFVEEEISESLLPAVGWRAEGKAER

SEQ ID NO: 443 - protein, 21 aa, Synthetic construct
RGGVIADQVGYGKTVISIALV

SEQ ID NO: 444 - protein, 22 aa, Synthetic construct
KATLIVVPGHLSKQWPNEIARF

SEQ ID NO: 445 - protein, 35 aa, Synthetic construct
FRRVIADEFTYLQKKSLAAVLRLSSSYRWILSGTP

SEQ ID NO: 446 - protein, 7 aa, Synthetic construct
DVQYQKA

SEQ ID NO: 447 - protein, 17 aa, Synthetic construct
VLLLKMNDASAAGSNLT

SEQ ID NO: 448 - protein, 34 aa, Synthetic construct
ANHAVFLGPLFTNSLFNYRAVETQAIGRVRRYGQ

SEQ ID NO: 449 - protein, 17 aa, Synthetic construct
LRVATMCSGTESPLLAL

SEQ ID NO: 450 - protein, 31 aa, Synthetic construct
HVFSCEIEPFKQAYIERNFAPPILFRDVTEL

SEQ ID NO: 451 - protein, 17 aa, Synthetic construct
DILIAGTSCVDYSGLNN

SEQ ID NO: 452 - protein, 19 aa, Synthetic construct
GESGRTFRGMLQWVNKHRP

SEQ ID NO: 453 - protein, 12 aa, Synthetic construct
IVILENVCSAPW

SEQ ID NO: 454 - protein, 19 aa, Synthetic construct
LDTKEYYIPHTRTRVYLFA

SEQ ID NO: 455 - protein, 42 aa, Synthetic construct
WNRCESRHARARQEELLGVLRPLTSWKEAGVCKGLDWTWNDW

SEQ ID NO: 456 - protein, 15 aa, Synthetic construct
QTERVVDLLDISTLR

SEQ ID NO: 457 - protein, 21 aa, Synthetic construct
DSGYKACIWNVSQNVDRQGTG

SEQ ID NO: 458 - protein, 13 aa, Synthetic construct
ALAPCLTPNMIPW

SEQ ID NO: 459 - protein, 44 aa, Synthetic construct
EALSLQGIPVRELLLTSENEDQLADLAGNAMTSTVVGASMLSAL

SEQ ID NO: 460 - protein, 48 aa, Synthetic construct
LRKEQLRSLWWMLRQEAAEGKTHTFVEEEISEAMLPSLGWRAEGKAER

SEQ ID NO: 461 - protein, 21 aa, Synthetic construct
RGGVIADQVGYGKTIISLALV

SEQ ID NO: 462 - protein, 22 aa, Synthetic construct
KATLIVVPGHLSKQWPSEITRF

SEQ ID NO: 463 - protein, 35 aa, Synthetic construct
FRRVIADEFTYLEKKPLNALLKLSSCYKWVLSGTP

SEQ ID NO: 464 - protein, 7 aa, Synthetic construct
SKSTQIA

SEQ ID NO: 465 - protein, 17 aa, Synthetic construct
VLLLKMQDASAAGSNLT

SEQ ID NO: 466 - protein, 34 aa, Synthetic construct
ANHAIFLGPLFVDTLHRYRQAETQAIGRIRRYGQ

(19) United States
(12) Patent Application Publication
(10) Pub. No.: US 2026/0015677 A1
(43) Pub. Date: Jan. 15, 2026

(54) METHYLATION METHOD

(71) Applicant: Syndex Bio Ltd, Cambridge (GB)

(72) Inventors: Olivier Lan-Chow-Wing, Cambridge (GB); Christopher Thomas Hodson, Cambridge (GB); Gary Mark Carre-Skinner, Newmarket (GB); Geoffrey Paul Smith, Cambridge (GB)

(21) Appl. No.: 19/330,044

(22) Filed: Sep. 16, 2025

Related U.S. Application Data
(63) Continuation of application No. PCT/GB24/52101, filed on Aug. 8, 2024.

(30) Foreign Application Priority Data
Aug. 8, 2023 (GB) 2312147.8

(51) Int. Cl. C12Q 1/6886; C12Q 1/6858

(52) U.S. Cl. CPC C12Q 1/6886; C12Q 1/6858; C12Q 2600/154

(57) ABSTRACT

The invention relates to methods of amplifying methylated DNA, particularly to methods using a methyl transferase which has activity on hemimethylated DNA, but which essentially lacks de novo methylation activity. The invention further relates to methyl transferases having such activity, and products comprising such methyl transferases, as well as applications of said methods and methyl transferase.

### CROSS-REFERENCE TO RELATED APPLICATIONS

This application is a continuation of International Patent Application No. PCT/GB2024/052101, filed Aug. 8, 2024, which claims priority to United Kingdom Application GB 2312147.8, filed on Aug. 8, 2023, the entire contents of each of which is hereby incorporated by reference.

### SEQUENCE LISTING

The instant application contains a Sequence Listing which has been submitted electronically in XML format and is hereby incorporated by reference in its entirety. Said XML copy, created on Aug. 8, 2024, is named P75713WO.xml and is 462, 141 bytes in size.

### FIELD OF THE INVENTION

The invention relates to methods of amplifying methylated DNA, particularly to methods using a methyl transferase which has activity on hemimethylated DNA, but which essentially lacks de novo methylation activity. The invention further relates to methyl transferases having such activity, and products comprising such methyl transferases, as well as applications of said methods and methyl transferases.

### BACKGROUND TO THE INVENTION

Epigenetic factors cause heritable changes to cells without making any changes to the DNA sequence of the cell's genome. Such factors can result in changes in gene activity or function. Although virtually all cells in an organism contain the same genetic information, epigenetic mechanisms enable differential gene expression by different cell types.

DNA methylation is a major epigenetic factor influencing gene activities, regulating gene expression by recruiting proteins involved in gene repression, or by inhibiting the binding of transcription factor(s) to DNA. DNA methylation involves the addition of methyl groups to DNA. Two DNA bases, cytosine and adenosine can be methylated. In particular, cytosine can be methylated at the fifth carbon to form 5-methylcytosine (5 mC) and/or the fourth position of the cytosine pyrimidine ring to form N4-methylcytosine (N4-mC). Adenine can be methylated at the N6 position to form N6-methyladenine (N6-mA). In mammals, DNA methylation primarily occurs on cytosine residues, particularly in the context of prolonged cytosine-guanine dinucleotide (CpG) repeats referred to as CpG islands, with 50-90% of CpG sites being methylated genome-wide.

DNA methylation involves the transfer of a methyl group onto specific positions within the cytosine or adenine rings, e.g. the fifth carbon of a cytosine to form 5-methylcytosine (5 mC). The methylation reaction is catalysed by a family of enzymes called DNA methyltransferases (DNMTs). At least four differently active DNA methyltransferases have been identified in mammals. They are named DNMT1, two isoforms transcribed from the DNMT3a gene: DNMT3a1 and DNMT3a2, and DNMT3b. Recently, another enzyme DNMT3c has been discovered specifically expressed in the male germline in the mouse. DNMT3 is a family of DNA methyltransferases that could methylate hemimethylated and unmethylated CpG at the same rate. The architecture of DNMT3 enzymes is similar to that of DNMT1, with a regulatory region attached to a catalytic domain. DNMT3a1, DNMT3a2 and DNMT3b can mediate methylation of CpG sites in gene promoters, resulting in gene repression. These DNA methyltransferases can also methylate CpG sites within the coding regions of genes, where such methylation can increase gene transcription. Work with DNMT3a1 showed it preferentially localized to CpG islands bivalently marked by H3K4 me3 (a transcription promoting mark) and H3K27 me3 (a transcription repressive mark), coinciding with the promoters of many transcription factors.

Epigenetic evolution occurs over million-year timescales in Cryptococcus neoformans and is mediated by DNMT5, the first maintenance type cytosine methyltransferase identified in the fungal or protist kingdoms, the first dependent on adenosine triphosphate (ATP), and the most hemimethyl DNA-specific enzyme known. These features are reflected in DNMT5s unique enzyme mechanism and a unique domain architecture and structure, with less than 5% of the protein bearing any similarity to DNMT1.

In the 5 mC reaction, a methyl group is transferred from a S-adenyl methionine (SAM), which acts as a methyl group donor, by a DNMT to the C5 position of a cytosine to form 5 mC.

DNA methylation is an important epigenetic factor in numerous cellular processes, including embryonic development, for silencing retroviral elements, regulating tissue-specific gene expression, genomic imprinting, preserving chromosomal stability and X chromosome inactivation. DNA methylation in different genomic regions may exert different influences on gene activities based on the underlying genetic sequence. Dysregulation of DNA methylation, particularly, 5 mC, can cause abnormal gene expression, potentially affecting cancer risk, progression and/or treatment response. 5-hydroxymethylcytosine (5hmC or hmC) can be an intermediate in the cell's active DNA demethylation pathway with tissue-specific distribution affecting gene expression and carcinogenesis.

In view of the important role of DNA methylation in controlling gene expression, identifying, characterising and studying the methylation pattern of DNA can provide valuable insights into epigenetic gene regulation, and have potential clinical implications. DNA methylation patterns are tissue-specific, and so can be a useful diagnostic tool, for example in cancer diagnosis. In particular, the methylation pattern of cell-free DNA (cfDNA) provides information about cell death activity within an individual, and can be useful in diagnosing and/or prognosing a range of pathological conditions, including sepsis and cancer.

However, there is currently no good way to amplify DNA whilst preserving the original methylation pattern in the amplified strands. There are many issues associated with conventional methods and reagents. For example, DNMT1 and DNMT3 do not have the necessary specificity for hemimethylated dsDNA, such that the de novo activity of DNMT1 and DNMT3 is too high. As a result, the methylation pattern is not maintained between cycles of replication.

Rolling circle amplification (RCA) and DNMTs are not compatible, because the rates of extension (via RCA) and methylation are not balanced; RCA will produce many more strands of DNA than can be methylated by a DNMT, because the rate of methylation by DNMT is typically orders of magnitude slower than the rate of extension/polymerisation by a DNA polymerase. Similar effects will also be seen for other methods where the rates of DNA extension and DNA methylation are not balanced. As a result, the amplified product will contain many more strands of unmethylated DNA compared with methylated DNA. Samples with limited amounts of DNA, such as cfDNA, can also be problematic, because the limited amount of sample material may mean that the amount of DNA in the sample is lower than the amount required for analytical or diagnostic testing. Furthermore, due to the limited quantities of cell-free DNA (cfDNA) present in plasma, it may be necessary to collect multiple tubes of blood to obtain sufficient material for accurate analysis. Drawing large volumes of blood can be uncomfortable and inconvenient for patients, potentially discouraging them from undergoing necessary tests. Furthermore, the need for multiple samples can introduce variability and potential inaccuracies in the results, as each sample may contain different concentrations of cfDNA. This variability can compromise the overall accuracy and reliability of the analysis, making it less consistent and potentially affecting the diagnostic outcome. This same issue can also cause problems should there be a requirement for the retesting of samples through analytical or diagnostic tests.

Furthermore, conventional methods for amplifying methylated DNA can be problematic for primer design, where typically the sample is processed through a step involving hydrolytic deamination of non-methylated cytosines to uracils, prior to amplification. Consequently, primers for amplification via conventional techniques must be designed using a predominantly three-base code, (because the large number of unmethylated cytosine residues will be converted to uracil), and also take into account the ambiguity of whether a given cytosine is methylated or not. The challenge posed by the three-base code issue also significantly affects the design of primers used for the enrichment of specific genomic loci, such as those targeted in genetic screening and diagnostics. Again, enrichment primers must be designed taking into account the methylation state of the genomic loci of interest. Methylation can alter the DNA sequence's accessibility and recognition by the primers, which can cause challenges in the placement and specificity of enrichment primers. These factors thereby impact on the sensitivity and specificity of the amplification, and generates complexity in the placement of amplification primers in a given DNA sequence.

Therefore, it is an object of the present invention to provide a means of amplifying methylated DNA, in which strand extension and methylation are synchronised such that symmetrical methylation of the synthesised (daughter) strands can be achieved. Such a method would be technically very beneficial, as it would facilitate epigenetic analysis of samples containing very little starting DNA, and also would allow for parallel detection of hmC and mC.

### SUMMARY OF THE INVENTION

The inventors have devised a method of amplifying DNA in which methylation of cytosine residues in the parent strand (referred to interchangeably herein as the template strand) are accurately copied to the daughter strand. In particular, the present inventors have developed a method of amplifying methylated DNA, particularly double-stranded DNA (dsDNA) using a methyltransferase (MT) enzyme which accurately copies methylation from a parent strand to the daughter strands, with minimal de novo methylation. This is achieved by use of a MT which is orders of magnitude more active on hemimethylated DNA compared with catalysing de novo methylation. In particular, the inventors have surprisingly demonstrated that enzymes comprising both methyltransferase activity and ATPase activity and comprising a methyltransferase domain and an ATPase domain can be used in recursive methods to copy methylated cytosine residues in hemimethylated DNA. In particular, the inventors have demonstrated this with a DNMT, specifically DNMT5, which comprises both methyltransferase activity and ATP hydrolysis (ATPase) activity. This ATP hydrolysis activity drives substrate specificity of the MT, such that the MT is many orders of magnitude more active on hemimethylated DNA compared with catalysing de novo methylation. The inventors have shown that an MT specific for hemimethylated DNA, such as DNMT5, can be used in a recursive method to copy methylated cytosine residues in hemimethylated DNA. This allows for small amounts of template DNA in a sample to be amplified for further analysis, whilst faithfully retaining the methylation pattern of the template DNA. Given the differences in structure and properties (such as different co-factor requirements) between enzymes comprising both methyltransferase activity and ATPase activity (such as DNMT5) and conventional methyltransferase such as DNMT1, it is surprising that enzymes comprising both methyltransferase activity and ATPase activity have utility in such methods.

Accordingly the present invention provides a method of amplifying methylated DNA, comprising or consisting of the following steps: (a) using a DNA polymerase to amplify one or more target sequence within at least one DNA strand to form a hemimethylated double-stranded (dsDNA) substrate; (b) contacting the hemimethylated dsDNA substrate with a DNA methyltransferase (DNMT), thereby producing a symmetrically-methylated dsDNA product; wherein: (i) a cycle comprising steps (a)-(b) is repeated at least once; and (ii) the DNMT has at least 100-fold greater activity for hemimethylated DNA compared with unmethylated DNA.

The methylated DNA may be dsDNA or single-stranded DNA (ssDNA), preferably dsDNA.

Step (a) of said method may comprise hybridising a primer to the at least one DNA strand. Optionally the methylated DNA is dsDNA and step (a) comprises hybridising a first primer to one strand of the dsDNA and hybridising a second primer to the other strand of the dsDNA, wherein the first and second primers are different.

Step (a) of said method may comprise denaturation of the DNA to allow primer hybridisation. Optionally said denaturation of the DNA is carried out by: (i) thermal denaturation (e.g. by thermal cycling); or (ii) chemical denaturation.

The method may comprise a step of ligating a first primer to the at least one DNA strand prior to step (a). Optionally: (i) the first primer is a linear primer, a hairpin primer or a forked adapter; and/or (ii) step (a) comprises hybridising a second primer to the first primer, wherein the first and second primers are different.

At least one of the first primer sequence and the second primer sequence may comprise a recognition sequence for a DNA modifying enzyme, particularly a DNA cleaving enzyme. Alternatively or in addition, the first primer sequence and second primer sequence together may comprise a double-stranded recognition sequence for a DNA modifying enzyme, particularly a DNA cleaving enzyme, and wherein in step (b) of the method the DNA modifying enzyme, particularly a DNA cleaving enzyme generates a nick in (i) the hemimethylated dsDNA substrate or (ii) the symmetrically-methylated dsDNA product to generate a substrate for the DNA polymerase. The DNA modifying enzyme, particularly the DNA cleaving enzyme, may be: (i) a type IIS restriction enzyme; or (ii) a Cas protein complex.

Any of the one or more primers used in a method of the invention, such as the first primer and/or second primer may comprise a tag and/or a barcode sequence.

In a method of the invention: (i) DNMT activity may not present during step (a); (ii) DNMT activity may be removed from the symmetrically-methylated dsDNA product produced in step (b) before starting a new cycle of steps comprising steps (a) and (b); (iii) DNA polymerase activity may not present during step (b); (iv) DNA polymerase activity may be removed from the hemimethylated dsDNA substrate before starting step (b); (v) DNA modifying enzyme activity may not present during step (a); and/or (vi) DNA modifying enzyme activity (DNA cleaving enzyme activity) may be removed in step (b) before starting a new cycle of steps comprising steps (a) and (b).

The DNMT may be immobilised, optionally permanently immobilised. In such methods, optionally the cycle comprising steps (a) and (b) is carried out two to 35 times, optionally two to 20 times, two to ten times, two to seven times, or two to six times.

Any one or more primers used in a method of the invention, such as the first primer and/or second primer and/or the DNA (e.g. the at least one DNA strand) may be immobilised, wherein optionally: (i) the cycle comprising steps (a) and (b) is carried out two to 40 times, such as two to 10 times, 10 to 40 times, 20 to 40 times, 30 to 40 times; and/or (ii) the DNMT is in solution. Any one or more primers used in a method of the invention, such as the first primer and/or second primer and/or the DNA (e.g. the at least one DNA strand) may be immobilised throughout steps (a)-(b) and between cycles, and preferably said primers and/or DNA are immobilised on the surface of a flow cell or chip. Alternatively, any one or more primers used in a method of the invention, such as the first primer and/or second primer and/or the DNA (e.g. the at least one DNA strand) may be captured and released between cycles, and preferably said primers and/or DNA are captured on the surface of a flow cell or chip, or on the surface of beads.

The DNMT may be added for step (b) and/or removed after step (b), optionally using flow/injection techniques.

In a method of the invention: (i) hybridisation and/or ligation may be carried out in the same reaction vessel as the DNA polymerase reaction and step (b) may be carried out in a different reaction vessel; (ii) hybridisation and/or ligation, the DNA polymerase reaction and step (b) may each be carried out in a separate reaction vessel; (iii) hybridisation and/or ligation, the DNA polymerase reaction and step (b) may be carried out in the same reaction vessel.

The DNMT may be labelled, optionally wherein a label is present at the N- and/or C-terminal of DNMT, preferably at the C-terminal. Alternatively or in addition, any one or more primers used in a method of the invention, such as the first primer and/or second primer and/or the DNA (e.g. the at least one DNA strand) may be labelled, optionally wherein a label is present at the 5′ and/or 3′ end of said primers and/or DNA, preferably at the 5′ end.

The DNA polymerase used in step (a) may retain at least partial activity: (i) in the presence of ATP and/or S-adenosyl methionine (SAM); and/or (ii) in methyltransferase buffer.

The DNA polymerase may be: (i) a high-fidelity DNA polymerase; and/or (ii) a strand displacing DNA polymerase.

A method of the invention may comprise a step of detecting methylcytosine (mC), particularly 5 mC, following a final step (b); wherein optionally: (i) the mC are detected using an array, nanopores, next-generation sequencing and/or restriction digest analysis; (ii) the symmetrically-methylated dsDNA product is divided into two or more samples, each which is analysed using a different technique; and/or (iii) the original strands of the DNA and the copied strands produced by the method are analysed separately, or the original strands of the DNA and the copied strands produced by each cycle of the method are analysed separately.

A method of the invention may comprise oxidising one or more mC in the symmetrically-methylated dsDNA product produced in step (b) to hydroxymethylcytosine (hmC); wherein optionally: (i) a TET enzyme is used to oxidise the one or more mC; and/or (ii) the oxidisation is carried out prior to detection of mC, wherein preferably detection can differentiate between C, mC and hmC.

Each step (b) of the method may be carried out at a temperature of between about 10° C. to about 70° C., preferably at a temperature of between about 10° C. to about 60° C., more preferably at a temperature of between about 10° C. to about 40° C., still more preferably at a temperature of between about 20° C. to about 37° C., most preferably at a temperature of about 25° C.

Step (b) of the method may comprise (i) a single addition of SAM; or (ii) two or more, preferably two, additions of SAM. In some embodiments, step (b) of the method may comprises a single addition of SAM.

Steps (a) and (b) of the method may be carried out in the same reaction buffer, which optionally may be replenished and/or supplemented with one or more different components during any of steps (a) and/or (b). Alternatively, step (a) and/or (b) may be carried out in a buffer optimised for that step.

The DNMT may comprise or consist of both methyltransferase activity and ATP hydrolysis (ATPase) activity. Alternatively or in addition, the DNMT may be a DNMT5, wherein optionally said DNMT5 comprises or consists of an amino acid sequence having at least 60% sequence identity with SEQ ID NO: 1 or 3.

A method of the invention may further comprise a step of copying at least one single-stranded DNA (ssDNA) methylated strand to produce dsDNA, wherein the resulting dsDNA is used in step (a).

One or more of the resulting mC produced by a method of the invention may be labelled by the use of SAM analogues, wherein optionally the SAM analogues used to label the mC add a protecting group to the one or more mC.

The invention further provides a method of detecting methylation in DNA comprising carrying out a method of any one of amplifying methylated DNA according to the invention and detecting mC and/or hmC in the symmetrically-methylated dsDNA product produced by a final step (b).

The invention also provides a method of generating a library of methylated DNA, said method comprising carrying out a method of any one of amplifying methylated DNA according to the invention and separating the strands of the symmetrically-methylated dsDNA product produced by a final step (b) to form a single-stranded DNA library.

The invention further provides a DNMT permanently immobilised on an inert support, wherein optionally: (i) said DNMT is a DNMT5 as defined herein; and/or said support is beads, optionally magnetic beads.

The invention also provides a kit comprising DNMT which is (i) a DNMT5 as defined herein and/or (ii) permanently immobilised on a solid support optionally as defined herein; and optionally one or more of: (i) SAM; (ii) ATP; (iii) a DNA polymerase; (iv) one or more buffer; and (v) control DNA; wherein optionally the kit further comprises instructions for use.

Embodiments of the invention include the following:

Embodiment 1: A method of amplifying a target double-stranded DNA (dsDNA) containing symmetrically methylated nucleotides to produce an amplified methylated dsDNA product, that includes the steps of:
(a) copying at least one methylated single-stranded DNA (ssDNA) using a polymerase and at least one target specific synthetic DNA oligonucleotide primer to produce a hemi-methylated dsDNA substrate;
(b) contacting the hemi-methylated dsDNA substrate with an enzyme comprising both methyltransferase activity and ATPase activity wherein the enzyme comprises a methyltransferase domain and an ATPase domain, to produce a symmetrically-methylated dsDNA product; and
(c) removing or inactivating the enzyme from step (b);
wherein an amplification cycle comprising steps (a)-(c) is repeated at least once.

Embodiment 2: The method in embodiment 1 in which steps (a)-(b) are conducted in a cation-containing buffer.

Embodiment 3: A method of amplifying a target double-stranded DNA (dsDNA) containing symmetrically methylated nucleotides to produce an amplified methylated dsDNA product, comprising:
(a) copying at least one methylated single-stranded DNA (ssDNA), using a polymerase and at least one target specific synthetic DNA oligonucleotide primer to produce a hemimethylated dsDNA substrate;
(b) contacting the hemimethylated dsDNA substrate with an enzyme comprising both methyltransferase activity and ATPase activity wherein the enzyme comprises a methyltransferase domain and an ATPase domain, to produce a symmetrically-methylated dsDNA product; and;
wherein:
(i) an amplification cycle comprising steps (a)-(b) is repeated at least once; and
(ii) steps (a)-(b) are conducted in a cation-containing buffer.

Embodiment 4: The method in Embodiment 3, which following step (b) further comprises:
(c) removing or inactivating the enzyme.

Embodiment 5: The method of any of the preceding embodiment wherein the methyl transferase domain in the enzyme comprises a sequence having at least 90% sequence identity to a sequence motif selected from the group consisting of: SEQ ID NOs: 35 to 45.

Embodiment 6: The method of embodiment 5 wherein the methyl transferase domain in the enzyme comprises a sequence having at least 90% sequence identity to a sequence motif selected from at least one of: an MT1 motif having a sequence selected from the group consisting of SEQ ID NOs: 53, 71, 89, 107, 125, 143, 179, 197, 215, 233, 251, 269, 287, 305, 323, 341, 359, 37, 395, 413, 431 and 449; an MT23 motif having a sequence selected from the group consisting of SEQ ID NOs: 54, 72, 90, 108, 126, 144, 180, 198, 216, 234, 252, 270, 288, 306, 324, 342, 360, 378, 396, 414, 432 and 450; an MT4 motif having a sequence selected from the group consisting of SEQ ID NOs: 55, 73, 91, 109, 127, 145, 163, 181, 199, 217, 235, 253, 271, 289, 307, 325, 343, 361, 379, 397, 415, 433 and 451; an MT5 motif having a sequence selected from the group consisting of SEQ ID NOs: 56, 74, 92, 110, 128, 146, 164, 182, 200, 218, 236, 254, 272, 290, 308, 326, 344, 362, 380, 398, 416, 434, and 452; an MT6 motif having a sequence selected from the group consisting of SEQ ID NOs: 57, 75, 93, 111, 129, 147, 165, 183, 201, 219, 237, 255, 273, 291, 309, 327, 345, 363, 381, 399, 417, 435, and 453; an MT7 motif having a sequence selected from the group consisting of SEQ ID NOs: 58, 76, 94, 112, 130, 148, 166, 184, 202, 220, 238, 256, 274, 292, 310, 328, 346, 364, 382, 400, 418, 436, and 454; an MT7B motif having a sequence selected from the group consisting of SEQ ID NOs: 59, 77, 95, 113, 131, 149, 167, 185, 203, 221, 239, 257, 275, 293, 311, 329, 347, 365, 383, 401, 419, 437, and 455; an MT8 motif having a sequence selected from the group consisting of SEQ ID NOs: 60, 78, 96, 114, 132, 150, 168, 186, 204, 222, 240, 258, 276, 294, 312, 330, 348, 366, 384, 402, 420, 438, and 456; an MT8B motif having a sequence selected from the group consisting of SEQ ID NOs: 61, 79, 97, 115, 133, 151, 169, 187, 205, 223, 241, 259, 277, 295, 313, 331, 349, 367, 385, 403, 421, 439, and 457; an MT9 motif having a sequence selected from the group consisting of SEQ ID NOs: 62, 80, 98, 116, 134, 152, 170, 188, 206, 224, 242, 260, 278, 296, 314, 332, 350, 368, 386, 404, 422, 440, and 458; and/or MT10 motif having a sequence selected from the group consisting of SEQ ID NOs: 63, 81, 99, 117, 135, 153, 171, 189, 207, 225, 243, 261, 279, 297, 315, 333, 351, 369, 387, 405, 423, 441, and 459.

Embodiment 7: The method of any proceeding embodiment, wherein the ATPase domain in the enzyme comprises a sequence having at least 90% sequence identity to a sequence motif selected from the group consisting of: SEQ ID NOs: 46 to 52.

Embodiment 8: The method of embodiment 7 wherein the ATPase domain in the enzyme comprises a sequence having at least 90% sequence identity to a sequence motif selected from the group consisting of: a ATP11B motif having a sequence selected from the group consisting of SEQ ID NOs: 64, 82, 100, 118, 136, 154, 172, 190, 208, 226, 244, 262, 280, 298, 316, 334, 352, 370, 388, 406, 424, 442, and 460; a ATP12 motif having a sequence selected from the group consisting of SEQ ID NOs: 65, 83, 101, 119, 137, 155, 173, 191, 209, 227, 245, 263, 281, 299, 317, 335, 353, 371, 389, 407, 425, 443, and 461; a ATP13 motif having a sequence selected from the group consisting of SEQ ID NOs: 66, 84, 102, 120, 138, 156, 174, 192, 210, 228, 246, 264, 282, 300, 318, 336, 354, 372, 390, 408, 426, 444, and 462; a ATP14 motif having a sequence selected from the group consisting of SEQ ID NOs: 67, 85, 103, 121, 139, 157, 175, 193, 211, 229, 247, 265, 283, 301, 319, 337, 355, 373, 391, 409, 427, 445, and 463; a ATP14B motif having a sequence selected from the group consisting of SEQ ID NOs: 68, 86, 104, 122, 140, 176, 194, 212, 230, 248, 266, 284, 302, 320, 338, 356, 374, 392, 410, 446, and 464; a ATP15 motif having a sequence selected from the group consisting of SEQ ID NOs: 69, 87, 105, 123, 141, 159, 177, 213, 231, 249, 267, 285, 303, 321, 339, 357, 375, 393, 411, 429, 447, and 465; and/or a ATP15B motif having a sequence selected from the group consisting of SEQ ID NOs: 70, 88, 106, 124, 142, 160, 178, 214, 232, 250, 268, 286, 304, 322, 340, 358, 376, 394, 412, 430, 448, and 466.

Embodiment 9: The method of any one of the preceding embodiments, wherein:
(i) the enzyme in step (b) is thermostable;
(ii) the enzyme in step (b) is a C-terminal truncated and/or an N-terminal truncated DNMT;
(iii) the enzyme in step (b) has a label and/or a tag, the tag and/or label preferably located at the N- and/or C-terminal of the enzyme, preferably at the C-terminal; wherein the tag is optionally selected from the group consisting of a His-tag, biotin, CBD, MBP, FLAG, V5, myc, HA, strep-tag or SNAP-tag; and/or the label is a detectable label selected from the group consisting of an antibody, a fluorescent label, a dye, a SAM analogue and a chemiluminescent label;
and/or
(iv) the enzyme in step (b) is immobilized on a solid substrate such as the surface of a flow cell or chip, or on beads.

Embodiment 10: The method of embodiment 9, wherein the enzyme with methyltransferase activity and ATPase activity is a DNMT5 or variant thereof which:
(i) comprises or consists of an amino acid sequence having at least 60% sequence identity with SEQ ID NO: 1 or 3; or
(ii) comprises or consists of an amino acid sequence of any one of SEQ ID. Nos 23-34, particularly any one of SEQ ID NOs: 25 to 34, more, particularly any one of SEQ ID NOs: 28 to 34.

Embodiment 11: A method of amplifying methylated DNA to produce an amplified dsDNA product, comprising:
(a) copying at least one methylated single-stranded DNA (ssDNA) to produce hemimethylated dsDNA substrate, using a polymerase and at least one target specific synthetic DNA oligonucleotide primer;
(b) contacting the hemimethylated dsDNA substrate with a DNA methyltransferase (DNMT), thereby producing a symmetrically-methylated dsDNA product;
wherein:
(i) an amplification cycle comprising steps (a)-(b) is repeated at least once; and
(ii) the DNMT has at least 100-fold greater activity for hemimethylated DNA compared with unmethylated DNA.

Embodiment 12: The method of embodiment 11, which following step (b) further comprises:
(c) removing or inactivating the DNMT.

Embodiment 13: The method of embodiments 11 or 12, wherein steps (a)-(b) are conducted in a cation-containing buffer.

Embodiment 14: The method of any of embodiments 11-13, wherein:
(i) the DNMT in step (b) is thermostable;
(ii) the DNMT in step (b) is a C-terminal truncated and/or N-terminal truncated DNMT;
(iii) the DNMT in step (b) has a label and/or a tag, the tag and/or label preferably located at the N- and/or C-terminal of the enzyme, preferably at the C-terminal; wherein the tag is optionally selected from the group consisting of a His-tag, biotin, CBD, MBP, FLAG, V5, myc, HA or SNAP-tag; and/or the label is a detectable label selected from the group consisting of an antibody, a fluorescent label, a dye, a SAM analogue and a chemiluminescent label; and/or
(iv) the DNMT in step (b) is immobilized on a solid substrate such as the surface of a flow cell or chip, or on beads.

Embodiment 15: The method of any of embodiments 11 to 14, wherein the DNMT comprises a methyl transferase domain comprising a sequence having at least 90% sequence identity to a sequence motif selected from the group consisting of: SEQ ID NOs: 35 to 45.

Embodiment 16: The method of embodiment 15, wherein the methyl transferase domain in the DNMT comprises a sequence having at least 90% sequence identity to a sequence motif selected from at least one of: an MT1 motif having a sequence selected from the group consisting of SEQ ID NOs: 53, 71, 89, 107, 125, 143, 179, 197, 215, 233, 251, 269, 287, 305, 323, 341, 359, 37, 395, 413, 431 and 449; an MT23 motif having a sequence selected from the group consisting of SEQ ID NOs: 54, 72, 90, 108, 126, 144, 180, 198, 216, 234, 252, 270, 288, 306, 324, 342, 360, 378, 396, 414, 432 and 450; an MT4 motif having a sequence selected from the group consisting of SEQ ID NOs: 55, 73, 91, 109, 127, 145, 163, 181, 199, 217, 235, 253, 271, 289, 307, 325, 343, 361, 379, 397, 415, 433 and 451; an MT5 motif having a sequence selected from the group consisting of SEQ ID NOs: 56, 74, 92, 110, 128, 146, 164, 182, 200, 218, 236, 254, 272, 290, 308, 326, 344, 362, 380, 398, 416, 434, and 452; an MT6 motif having a sequence selected from the group consisting of SEQ ID NOs: 57, 75, 93, 111, 129, 147, 165, 183, 201, 219, 237, 255, 273, 291, 309, 327, 345, 363, 381, 399, 417, 435, and 453; an MT7 motif having a sequence selected from the group consisting of SEQ ID NOs: 58, 76, 94, 112, 130, 148, 166, 184, 202, 220, 238, 256, 274, 292, 310, 328, 346, 364, 382, 400, 418, 436, and 454; an MT7B motif having a sequence selected from the group consisting of SEQ ID NOs: 59, 77, 95, 113, 131, 149, 167, 185, 203, 221, 239, 257, 275, 293, 311, 329, 347, 365, 383, 401, 419, 437, and 455; an MT8 motif having a sequence selected from the group consisting of SEQ ID NOs: 60, 78, 96, 114, 132, 150, 168, 186, 204, 222, 240, 258, 276, 294, 312, 330, 348, 366, 384, 402, 420, 438, and 456; an MT8B motif having a sequence selected from the group consisting of SEQ ID NOs: 61, 79, 97, 115, 133, 151, 169, 187, 205, 223, 241, 259, 277, 295, 313, 331, 349, 367, 385, 403, 421, 439, and 457; an MT9 motif having a sequence selected from the group consisting of SEQ ID NOs: 62, 80, 98, 116, 134, 152, 170, 188, 206, 224, 242, 260, 278, 296, 314, 332, 350, 368, 386, 404, 422, 440, and 458; and/or MT10 motif having a sequence selected from the group consisting of SEQ ID NOs: 63, 81, 99, 117, 135, 153, 171, 189, 207, 225, 243, 261, 279, 297, 315, 333, 351, 369, 387, 405, 423, 441, and 459.

Embodiment 17: The method of any of embodiments 11 to 16, wherein the DNMT comprises an ATPase domain comprising a sequence having at least 90% sequence identity to a sequence motif selected from the group consisting of: SEQ ID NOs: 46 to 52.

Embodiment 18: The method of embodiment 17, wherein the ATPase domain in the DNMT comprises a sequence having at least 90% sequence identity to a sequence motif selected from the group consisting of: a ATP11B motif having a sequence selected from the group consisting of SEQ ID NOs: 64, 82, 100, 118, 136, 154, 172, 190, 208, 226, 244, 262, 280, 298, 316, 334, 352, 370, 388, 406, 424, 442, and 460; a ATP12 motif having a sequence selected from the group consisting of SEQ ID NOs: 65, 83, 101, 119, 137, 155, 173, 191, 209, 227, 245, 263, 281, 299, 317, 335, 353, 371, 389, 407, 425, 443, and 461; a ATP13 motif having a sequence selected from the group consisting of SEQ ID NOs: 66, 84, 102, 120, 138, 156, 174, 192, 210, 228, 246, 264, 282, 300, 318, 336, 354, 372, 390, 408, 426, 444, and 462; a ATP14 motif having a sequence selected from the group consisting of SEQ ID NOs: 67, 85, 103, 121, 139, 157, 175, 193, 211, 229, 247, 265, 283, 301, 319, 337, 355, 373, 391, 409, 427, 445, and 463; a ATP14B motif having a sequence selected from the group consisting of SEQ ID NOs: 68, 86, 104, 122, 140, 176, 194, 212, 230, 248, 266, 284, 302, 320, 338, 356, 374, 392, 410, 446, and 464; a ATP15 motif having a sequence selected from the group consisting of SEQ ID NOs: 69, 87, 105, 123, 141, 159, 177, 213, 231, 249, 267, 285, 303, 321, 339, 357, 375, 393, 411, 429, 447, and 465; and/or a ATP15B motif having a sequence selected from the group consisting of SEQ ID NOs: 70, 88, 106, 124, 142, 160, 178, 214, 232, 250, 268, 286, 304, 322, 340, 358, 376, 394, 412, 430, 448, and 466.

Embodiment 19: The method of any of embodiments 11 to 18, wherein the DNMT is a DNMT5 which:
(i) comprises or consists of an amino acid sequence having at least 60% sequence identity with SEQ ID NO: 1 or 3; or
(ii) comprises or consists of an amino acid sequence of any one of SEQ ID NOs: 23 to 34, particularly any one of SEQ ID NOs: 28 to 34.

Embodiment 20: The method of any one of the preceding embodiments, wherein step (a) comprises denaturation of the DNA to allow primer hybridisation;
wherein optionally the denaturation of the DNA is carried out by:
(i) thermal denaturation; or
(ii) chemical denaturation.

Embodiment 21: The method of any one of the previous embodiments, which comprises a step of ligating a first primer to the one strand of DNA prior to step (a);
wherein optionally:
(i) the first primer is a linear primer, a hairpin primer or a forked adapter; and/or
(ii) step (a) comprises hybridising a second primer to the first primer, wherein the first and second primers are different.

Embodiment 22: The method of embodiment 21, wherein at least one of the first primer sequence and the second primer sequence comprises a recognition sequence for a DNA cleaving enzyme, or wherein the first primer sequence and second primer sequence together comprise a double-stranded recognition sequence for a DNA cleaving enzyme, and wherein in step (b) the DNA cleaving enzyme generates a nick in (i) the hemimethylated dsDNA substrate or (ii) the symmetrically-methylated dsDNA product to generate a substrate for the DNA polymerase.

Embodiment 23: The method of any one of the preceding embodiments, wherein the DNA polymerase used in step (a) retains at least partial activity:
(i) in the presence of ATP, S-adenosyl methionine (SAM) and/or Mg²⁺ cations; and/or
(ii) in methyltransferase buffer.

Embodiment 24: The method of any one of the preceding embodiments, wherein the DNA polymerase is:
(i) a high-fidelity DNA polymerase;
(ii) a strand displacing DNA polymerase; and/or
(iii) Q5® DNA polymerase, Pfusion® DNA polymerase, Pfu DNA polymerase, Klenow DNA polymerase, Taq DNA polymerase, KAPA HiFi DNA polymerase, phi29 DNA polymerase, Bst DNA polymerase, or Bsu DNA polymerase.

Embodiment 25: The method any one of the preceding embodiments, wherein:
(i) DNA polymerase activity is not present during step (b);
(ii) DNA polymerase activity is removed from the hemimethylated dsDNA substrate before starting step (b).

Embodiment 26: The method of any one of the preceding embodiments, carrying out the amplification cycle comprising steps (a)-(b) or (a)-(c) between two to 40 times, optionally between 10 to 40 times, or between 20 to 40 times.

Embodiment 27: The method of any one of the preceding embodiments, wherein a primer and/or dsDNA are immobilised, or in solution; wherein optionally immobilisation is on the surface of a flow cell or chip, or on beads.

Embodiment 28: The method of any one of the preceding embodiments, wherein:
(i) hybridisation and/or ligation, the DNA polymerase reaction and step (b) are carried out in the same reaction vessel;
(ii) hybridisation and/or ligation is carried out in the same reaction vessel as the DNA polymerase reaction and step (b) is carried out in a different reaction vessel; or
(iii) hybridisation and/or ligation, the DNA polymerase reaction and step (b) are each carried out in a separate reaction vessel;

Embodiment 29: The method of any one of the preceding embodiments, which comprises:
(i) carrying out steps (a) and (b) in the same reaction buffer, and optionally replenishing and/or supplementing the reaction buffer with one or more different components during any of steps (a) and/or (b); or
(ii) carrying out step (a) and/or (b) in a buffer optimised for that step.

Embodiment 30: The method of any one of the preceding embodiments, wherein step (b) comprises (i) a single addition of SAM; or (ii) two or more, preferably two, additions of SAM.

Embodiment 31: The method of any one of the preceding embodiments, further comprising carrying out step (b) at a temperature of between about 10° C. to about 70° C., preferably at a temperature of between about 10° C. to about 60° C., more preferably at a temperature of between about 10° C. to about 40° C., still more preferably at a temperature of between about 20° C. to about 37° C., most preferably at a temperature of about 25° C.

Embodiment 32: The method of any one of the preceding embodiments, which further comprises a step of copying at least one single-stranded DNA (ssDNA) methylated strand to produce dsDNA, wherein the resulting dsDNA is used in step (a).

Embodiment 33: The method of any one of the preceding embodiments, wherein:
(i) the primer, first primer or second primer comprises a tag and/or a barcode sequence, optionally at the 5′ and/or 3′ end; and/or
(ii) the one strand of dsDNA comprising the target sequence is labelled, optionally at the 5′ and/or 3′ end.

Embodiment 34: The method according to embodiment 33, wherein:
(i) the tag is selected from the group consisting of a His-tag, biotin, CBD, MBP, strep tag or SNAP-tag; and/or
(ii) the label is a detectable label selected from the group consisting of an antibody, a fluorescent label, a dye, a SAM analogue and a chemiluminescent label.

Embodiment 35: The method of any one of the preceding embodiments, comprising a step of detecting methylcytosine (mC) following a final step (b) or (c);
optionally further comprising:
(i) detecting the mC using an array, nanopores, next-generation sequencing and/or restriction digest analysis;
(ii) dividing the symmetrically-methylated dsDNA product into two or more samples, and detecting mC and in each sample using a different technique; and/or
(iii) detecting separately, the mC in the original strands of the DNA and the copied strands, or the mC of the original strands of the DNA and the copied strands produced by each cycle of the method.

Embodiment 36: The method of any one of the preceding embodiments, comprising oxidising one or more mC in the symmetrically-methylated dsDNA product produced in step (b) to hydroxymethylcytosine (hmC);
optionally further comprising:
(i) oxidising the one or more mC using a TET enzyme; wherein the oxidisation is carried out prior to detection of mC; and
(ii) differentiating between C, mC and hmC.

Embodiment 37: A method of detecting methylation in DNA comprising carrying out a method of any of the preceding embodiments and detecting mC and/or hmC in the symmetrically-methylated dsDNA product.

Embodiment 38: A method of generating a library of methylated DNA, the method comprising carrying out a method of any one of the preceding embodiments and optionally separating the strands of the symmetrically-methylated dsDNA product produced by a final step (b) to form a single-stranded DNA library.

Embodiment 39: A method of determining the presence of a methylated biomarker in a biological fluid, the method comprising amplifying methylated double-stranded DNA (dsDNA) from the biological fluid to produce an amplified dsDNA product using a method of any one of the preceding embodiments, wherein methylation of the biomarker is associated with a medical condition.

Embodiment 40: A method of diagnosing a medical condition comprising determining the presence of a methylated biomarker in a biological fluid, the method comprising amplifying methylated double-stranded DNA (dsDNA) from the biological fluid to produce an amplified dsDNA product using a method of any one of the preceding embodiments wherein methylation of the biomarker is associated with the medical condition.

Embodiment 41: The method of any of embodiments 39 or 40 wherein the methylated biomarker is diagnostic for a cancer in the biological fluid of an animal.

Embodiment 42: The method according to embodiment 41, further comprising, administering an effective dose of a therapeutic agent or using surgical means for treating the cancer.

Embodiment 43: The method according to embodiment 42, wherein the therapeutic agent is selected from immunotherapy, chemotherapy, and radiation.

Embodiment 44: The method of any of the preceding embodiments wherein step (a) and step (b) are carried out in the same reaction vessel.

Embodiment 45: The method of any one of embodiment 39 to 44 wherein the biological fluid is selected from blood, plasma, saliva, serum, sputum, urine, cerebrospinal fluid, cells, a cellular extract, sweat or synovial fluid.

Embodiment 46: An enzyme comprising both methyltransferase activity and ATPase activity immobilised on an inert support such as beads, particularly magnetic beads, wherein the enzyme is as defined in any of embodiments 5 to 10 or 14 to 19, and optionally:
(i) the enzyme is labelled or tagged, optionally wherein a label is present at the N- and/or C-terminal of the enzyme, preferably at the C-terminal end;
(ii) the enzyme is combined with one or more of SAM; ATP; dATP; a DNA polymerase; a ligase; Mg²⁺ cations; a proteinase, such as proteinase K; and an oligonucleotide primer in a mixture; and/or
(iii) the enzyme and one or more of SAM, ATP, dATP, a DNA polymerase, a ligase, Mg²⁺ cations, a proteinase and an oligonucleotide primer may be separately lyophilized.

Embodiment 47: A DNMT5 truncated at the C-terminus and/or N-terminus, which comprises both methyltransferase activity and ATPase activity, which optionally:
(i) comprises or consists of an amino acid sequence of any one of SEQ ID NOs: 25 to 34, particularly any one of SEQ ID NOs: 28 to 34;
(ii) is labelled, optionally wherein a label is present at the N- and/or C-terminal of the enzyme, preferably at the C-terminal; and/or
(iii) is tagged optionally wherein the tag is present at the N- and/or C-terminal of the enzyme, preferably at the C-terminal.

Embodiment 48: A kit comprising an enzyme comprising both methyltransferase activity and ATPase activity wherein the enzyme comprises a methyltransferase domain and an ATP hydrolysis domain, wherein optionally:
(i) the enzyme is defined according to embodiments in embodiments 5 to 10, 14 to 19, 46 or 47;
(ii) the kit further comprising SAM; ATP; dATP; a DNA polymerase; a DNA cleaving enzyme; one or more buffers; one or more oligonucleotide primers; Mg²⁺ cations; a proteinase, such as proteinase K; and/or control DNA in a mixture or separately wherein any of the SAM, ATP, dATP, DNA polymerase, DNA cleaving enzyme, primers, Mg²⁺ cations, proteinase, and control DNA may be separately or together lyophilized and/or
(i) instructions for use.

### BRIEF DESCRIPTION OF THE DRAWINGS

FIG. 1: Schematic of DNA extension (a) and methylation (b) and hybridisation, extension and methylation (dotted lines, H-E-M) steps in a method of the invention.

FIG. 2: (A) Schematic of the reaction; (B) Experimental data from an anti-hmC ELISA (C) Quantification of the ELISA normalised against the background signal.

FIG. 3: Development of an Ascl enzyme assay to test the activity of DNMT5 on hemimethylated DNA. (A) Schematic illustration of the assay used in this study. (B) Exemplary experimental data demonstrating the activity of DNMT5 on hemimethylated dsDNA. Lane 1. Untreated hemimethylated dsDNA substrate. Lane 2. Ascl digestion of hemimethylated dsDNA substrate. Lane 3. Treatment of hemimethylated dsDNA substrate with DNMT5 in the absence of Ascl digestion. Lane 4. Treatment of hemimethylated dsDNA substrate with DNMT5 and subsequent Ascl digestion.

FIG. 4: Two H-E-M cycles in which a copied methyl cytosine can act as a template for further copying from one DNA strand to another. (A) Schematic illustration of the experiment. (B) Schematic representation of the expected data; (C) Experimental data from the experiment outlined in (A) and (B). Labels above the gel images indicate the presence (+) or absence (−) of Ascl, EcoRI and DNMT5 enzymes in steps (ii) and (iv). DNA labelled with Cy3 and Atto647 are shown in the top and bottom panels respectively.

FIG. 5: Two cycles of H-E-M PCR starting with a methylated dsDNA template. (A) Schematic illustration of the experiment. (B) Schematic representation of the expected data. Expected migration patterns of DNA in the first (top panel) and second (bottom panel) H-E-M cycles are shown. (C) Experimental results on samples prepared as outlined in (A) and (B) in which the dsDNA was treated in the presence (+) or absence (−) of DNMT5 and Ascl. The top image shows data from imaging the dsDNA for the presence of the Atto647 fluorescent label and the bottom shows data from imaging the dsDNA for the presence of the Cy3 fluorescent label.

FIG. 6: Activity of DNMT5 immobilised on beads. Results of an ELISA in which hemimethylated DNA was incubated with His-tagged DNMT5 immobilised on a bead (top panel), or with beads alone (bottom panel). The DNA strand that was originally unmethylated was then captured on a streptavidin microtitre plate and the presence of methyl cytosine detected with an anti-mC antibody.

FIG. 7: Activity of DNMT5 in a cation-containing buffer compared with activity of DNMT5 in a buffer in which EDTA has been added to chelate cations, was investigated. Lane 1: control sample containing ATP, SAM, MgSO₄ and DNMT5, Lane 2: as lane 1 with 50 mM EDTA, Lane 3: control sample not treated with Ascl.

FIG. 8: Optimisation of Mg²⁺in the buffer for a one-pot reaction. Primer extension with Taq DNA polymerase in the presence (+) or absence (−) of ATP, SAM or MgSO₄ (Mg²⁺). 3% agarose gel visualised using the Cy3 label on the reverse primer.

FIG. 9: N- and C-terminal truncated forms of C. neoformans DNTM5 methylate template DNA. NeoFLAGHis and 11 other variants were assay for enzymatic activity on hemimethylated DNA with the Ascl restriction enzyme. A schematic of the truncated proteins are indicated with the grey arrow (top) with numbers representing the residue numbers relative to SEQ NO: 1. The gel is migrating from left to right as indicated by the black arrow. The negative and positive controls for Ascl digestion are shown.

FIG. 10: 5 cycles of H-E-M successfully amplifies a methylated human DNA template sequence and preserves the original methylation pattern. Non-methylated (non-meth) or methylated (Meth) DNA underwent 5 cycles of H-E-M with DNMT5. The DNA was treated (Y) or not treated (N) with the restriction enzyme BstUI. 3% agarose gel visualised using SYBR safe stain.

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only, and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

The headings provided herein are not limitations of the various aspects or embodiments of this disclosure.

As used herein, the term “capable of when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. “About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of′ and/or “consisting essentially of” such features.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

A “fragment” of a polypeptide typically comprises at least 50%, at least 60%, at least 70%, at least 80%, at least 90%, at least 95%, at least 97% or more of the original polypeptide.

A “truncation” of a polypeptide is shorter in length than the full-length polypeptide from which it is derived, by removal of one or more amino acid at the N-terminus and/or C-terminus. Typically a truncation of a polypeptide retains the function or activity of the full-length polypeptide. Thus, a “truncation” of an enzyme comprising both methyltransferase activity and ATP hydrolysis (ATPase) activity (e.g. a DNMT5) may refer to an enzyme which is shorter in length than the full-length enzyme from which it is derived, by removal of one or more amino acid at the N-terminus and/or C-terminus. An enzyme with methyltransferase activity and ATPase activity which has amino acids removed from the N-terminus may be referred to as an N-terminal truncation. An enzyme with methyltransferase activity and ATPase activity which has amino acids removed from the C-terminus may be referred to as a C-terminal truncation. An enzyme with methyltransferase activity and ATPase activity with amino acids removed from the N-terminus and the C-terminus may be referred to as an N- and C-terminal truncation. A truncated enzyme with methyltransferase activity and ATPase activity according to the invention typically retains both methyltransferase activity and ATPase activity. Accordingly, a truncated enzyme according to the invention typically retains an intact methyltransferase domain and an intact ATPase domain, as described herein.

A “variant” amino acid sequence has substantial homology or substantial similarity to a reference amino acid sequence (or a fragment thereof). A amino acid sequence or fragment thereof is “substantially homologous” (or “substantially identical”) to a reference sequence if, when optimally aligned (with appropriate amino acid insertions or deletions) with the other amino acid there is amino acid sequence identity in at least about 70%, 75%, 80%, 85%, 90%, 91%, 92%, 93%, 94%, 95%, 96%, 97%, 98%, 99% or more % of the amino acids. Methods for homology determination of amino acid sequences are known in the art. Typically a variant polypeptide of the invention retains the function or activity of the full-length polypeptide.

A variant polypeptide may be one in which amino acid residues from one species are substituted for the corresponding residue in another species, either at the conserved or non-conserved positions. Variants of DNMTs disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of DNMTs can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, lan H. et al Data Mining: Practical Machine Learning Tools and Techniques with Java Implementations. Morgan Kaufmann; (Oct. 11, 1999), ISBN: 1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of a DNMT can be derived from empirical and theoretical models (for example, analysis of likely contact residues or calculated physicochemical property) of the DNMT sequence, functional and three-dimensional structures and these properties can be considered individually and in combination.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a DNMT of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other examples of nucleic acid molecules are RNA, including siRNA, shRNA, and antisense oligonucleotides.

Unless otherwise indicated, any nucleic acid sequences are written left to right in 5′ to 3′ orientation.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

In view of the degeneracy of the genetic code, considerable sequence variation is possible among the polynucleotides of the present invention. Degenerate codons encompassing all possible codons for a given amino acid are set forth below:

| Amino | | Degenerate |
|---|---|---|
| Acid | Codons | Codon |
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85%, 90%, 91%, 92%, 93%, 94%, 95%, 96%, 97%, 98%, 99% or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). Typically, a fragment as defined herein retains the same function as the full-length polynucleotide.

When applied to a nucleic acid sequence, the term “isolated” denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment. In the context of the invention, an isolated nucleic acid is one which has been separated from one or more of the reagents used in its production according to methods of the invention; one which has been separated from the other nucleic acid sequences synthesised in the same iteration of the method and/or one which has been separated from the one or more units on which it was synthesised.

As used herein, the term “hemimethylated DNA” is used to refer to dsDNA in which only one of the two DNA strands is methylated. A hemimethylated site is a single CpG that is methylated on one strand, but not on the other.

The term “de novo methylation” as used herein refers to the addition of a methyl group to unmethylated DNA.

As used herein, the term “symmetrical-methylation” describes the situation where, if the cytosine in a CpG dinucleotides is methylated on one strand of a dsDNA molecule, the corresponding residue on the complementary strand is also methylated. In a symmetrically-methylated dsDNA product of the invention, all or substantially all (e.g. all but one, all but two, all but three, all but four, all but five, all but six, all but seven, all but eight, all but nine, all but ten) of the CpG dinucleotides are symmetrically-methylated. For the avoidance of doubt, the term “symmetrically-methylated dsDNA product” as used herein does not mean that the primary base sequence of the DNA is symmetrical, or that the methylation pattern across any given DNA strand is symmetrical.

The term “target region”, “target sequence” or “target” are used herein to refer to a region of at least one DNA strand which is to be amplified and the methylation pattern copied using a method of the invention. A “target region” may be of any size, e.g. depending on a particular gene or sequence of interest, and/or the intended application of the resulting amplified product. By way of non-limiting example, a target region may be from 10 base pairs (bp) to 10,000 bp of the polynucleotide substate (e.g. dsDNA), i.e. the target region may be from 10 bp to 10,000 bp in length, such as from 100 bp to 1,000 bp, 100 bp to 500 bp, 100 bp to 400 bp, 100 bp to 350 bp, 100 bp to 300 bp, 100 bp to 250 bp, 100 bp to 200 bp, 100 bp to 150 bp, 150 bp to 1,000 bp, 150 bp to 500 bp, 150 bp to 400 bp, 150 bp to 350 bp, 150 bp to 300 bp, 150 bp to 250 bp, 150 bp to 200 bp. The target region may be the entire length of the at least one DNA strand.

As used herein, the term “extension,” when used in reference to a primer is intended to include processes wherein one or more nucleotides are added to the primer (e.g. via polymerase activity) or wherein one or more oligonucleotides are added to the primer (e.g. via ligase activity).

As used herein, the term “flow cell” is intended to mean a chamber having a surface across which one or more fluid reagents can be flowed. Generally, a flow cell will have an ingress opening and an egress opening to facilitate flow of fluid. Examples of flowcells and related fluidic systems and detection platforms that can be readily used in the methods of the present disclosure are described, for example, in Bentley et al, Nature 456:53-59 (2008), WO 04/018497; U.S. Pat. No. 7,057,026; WO 91/06678; WO 07/123744; U.S. Pat. Nos. 7,329,492; 7,211,414; 7,315,019; 7,405,281, and US 2008/0108082, each of which is incorporated herein by reference.

The term “thermoresistant” is used herein to refer to a DNMT, particularly a DNMT5, which retains its secondary and/or tertiary structure at elevated temperatures, such as above about 25° C. Thermoresistance may or may not be associated with retained function at elevated temperatures. Thus, a DNMT may be thermoresistant and thermofunctional, or thermoresistant but not thermofunctional.

The term “thermofunctional” is used herein to refer to a DNMT, particularly a DNMT5, which retains its enzymatic activity at elevated temperatures, such as above about 25° C. Typically if a DNMT is thermofunctional, it will also be thermoresistant.

The term “thermostable” is used herein to refer to a DNMT, particularly a DNMT5, which is thermoresistant, thermofunctional, or both thermoresistant and thermofunctional.

The terms “DNMT activity” and “DNMT5 activity” are used herein to refer to the ability of DNMT or DNMT5 respectively to methylate cytosine residues to produce 5 mC. As described herein, the activity of a DNMT or DNMT5 of the invention is specific to hemimethylated polynucleotide (e.g. dsDNA) substrates. Appropriate methods for determining the activity of a DNMT or DNMT5 of interest are known in the art. Examples of suitable techniques are described and exemplified herein.

The terms “DNMT specificity” and “DNMT5 specificity” are used herein to refer to the ability of DNMT or DNMT5 respectively to methylate cytosine residues to produce 5 mC in a hemimethylated polynucleotide (e.g. dsDNA) substrate, compared with de novo methylation. Appropriate methods for determining the specificity of a DNMT or DNMT5 of interest are known in the art. Examples of suitable techniques are described and exemplified herein.

As used herein, a reference to step (a) of a method of the invention encompasses all substeps within step (a), e.g. steps (a1), (a2), (a3), etc., unless expressly stated to the contrary. Similarly, a reference to step (b) of a method of the invention encompasses all substeps within step (a), e.g. steps (b1), (b2), (b3), etc., unless expressly stated to the contrary. References to “a method comprising steps (a)-(b)” or “a cycle comprising steps (a)-(b)” encompasses all substeps within steps (a) and/or (b), preferably all substeps within both steps (a) and (b) (e.g. steps (a1), (a2), (a3), (b1), (b2), (b3), etc.), unless expressly stated to the contrary.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more.

As used herein, “reduction” or “inhibition” encompasses a complete inhibition or reduction as compared to a reference level. “Complete inhibition” is a 100% inhibition (i.e. abrogation) as compared to a reference level.

The terms “increased”, “increase”, “enhance”, or “activate” are all used herein to mean an increase by a statically significant amount. The terms “increased”, “increase”, “enhance”, or “activate” can mean an increase of at least 25%, at least 50% as compared to a reference level, for example an increase of at least about 50%, or at least about 75%, or at least about 80%, or at least about 90%, at least about 95%, or at least about 98%, or at least about 99%, or at least about 100%, or at least about 250% or more compared with a reference level, or at least about a 1.5-fold, or at least about a 2-fold, or at least about a 2.5-fold, or at least about a 3-fold, or at least about a 4-fold, or at least about a 5-fold or at least about a 10-fold increase, or any increase between 1.5-fold and 10-fold or greater as compared to a reference level.

A label may be used for the detection of a component used in a method of the invention (e.g. the polymerase, enzyme with methyltransferase activity and ATPase activity, the target DNA, etc.). A tag may be used for the isolation or removal of a component used in a method of the invention (e.g. the polymerase, enzyme with methyltransferase activity and ATPase activity, the target DNA, etc.). Some tags may be used as labels and some labels may be used as tags (e.g. His-tag, biotin, etc.). Non-limiting examples of labels and tags that may be used include ALFA-tags, AviTag, C-tag, CapTag, polyarginine or polyglutamate tags, FLAG-tag, His-tag, Myc-tag, SpyTag, SdyTag, HUH-tag, MBP-tag, biotin and digoxigenin (DIG). An exemplary C-terminally-labelled DNMT5 is given in SEQ ID NO: 14. When the DNMT is labelled and/or tagged, the DNMT may be labelled and/or tagged at the N- and/or C-terminus, preferably at the C-terminus. In some preferred embodiments, the DNMT is labelled and/or tagged with biotin or a His-tag. Wherein the primers and/or polynucleotide substrate (e.g. DNA) are labelled and/or tagged, the label and/or tag may be present at the 5′ or 3′ end of the primer(s) and/or polynucleotide substrate (e.g. DNA), preferably at the 5′ end. In some preferred embodiments, the primers and/or polynucleotide substrate (e.g. DNA) are labelled and/or tagged with biotin or DIG. When one or more first primers and one or more second primer are used, each of the first and/or second primer may be labelled and/or tagged. The label and/or tag for the one or more first primers and/or one or more second primer may be selected independently. The same label and/or tag may be used for one or more of the first primers and/or for one or more of the second primers.

As used herein, the term “solid support” refers to a solid material to which a component of the invention (e.g. the polymerase, enzyme with methyltransferase activity and ATPase activity, the target DNA, etc.). may be immobilised). A solid support may be made of any appropriate material, e.g. treptavidin magnetic beads, silica or agarose. Typically, the solid support itself is made of an inert substance, meaning that the material of the support has no effect or involvement on the method of the invention. The solid support may be of any suitable form, e.g. beads, a microplate well, a chamber in a flow cell or chip, a membrane, etc. The component may be immobilised to the support using any appropriate technique, examples of which are known in the art, including using immobilising enzymes, or chemical cross-linking reagents.

As used herein, the term, “polymerase” refers to an enzyme that catalyses the formation of DNA polynucleotides from nucleoside triphosphates in a 5′-3′ direction. Addition of nucleoside triphosphates may be templated or untemplated, in the context of the present disclosure, addition of nucleoside triphosphates is templated. Polymerases include DNA polymerases classified into families A, B, C, D, X, Y and RT. Examples of polymerases include Q5® polymerases, Taq polymerases, Bst polymerases, Bsu polyerases, phi29 polymerases, T4 DNA polymerase, T7 DNA polymerase, DNA pol I, Therminator™, Klenow, Vent® polymerases, Deep Vent® polymerases, terminal transferase, 9°N polymerase.

As used herein, the term “adapter” refers to a sequence that is joined to or can be joined to another molecule (e.g., ligated or copied onto via primer extension). An adapter can be DNA or RNA, or a mixture of the two. An adapter may be 15 to 100 bases, e.g., 50 to 70 bases, although adapters outside of this range are envisioned. In a library of polynucleotide molecules that contain an adapter (e.g., a 3′ or 5′ adapter, the adapter sequence used is not present in the DNA sequences under examination (i.e., the sequence in between the adapters). For example, if the library of polynucleotide molecules contains sequences derived from mammalian genomic DNA, cDNA or RNA, then the sequences of the adapters are not present in the mammalian genome under study. In many cases, the 5′ and 3′ adapters are of a different sequence and are not complementary. In many cases, an adapter will not contain a contiguous sequence of at least 8, 10 or 12 nucleotides that is found in the DNA under examination. Adapters may be designed to serve a specific purpose. For example, adapters may be designed for use in sequencing applications. Sequencing adapters may comprise, for example, an oligo-(dT) overhang, a barcode sequence, an overhang (other than oligo-(dT)) to anneal to another adapter, a site for anchoring a motor protein, and a sequence to bind to tethering oligos with affinity to polymer membrane for guiding a DNA or RNA fragment (on which it resides) to the vicinity of a nanopore, and combinations thereof.

The term “adapter” “adapter-containing” refers to either a nucleic acid that has been ligated to an adapter, or to a nucleic acid to which an adapter has been added by primer extension. In some embodiments, the adapters of a library of nucleic acid molecules may be made by ligating oligonucleotides to the 5′ and 3′ ends of the molecules (or specific sequences of the same) in an initial nucleic acid sample, e.g., DNA or genomic DNA, cDNA.

As used herein, the term “adapter”, “buffer” or “buffering agent” refers to an agent that, when in solution or in contact with a solution, contributes to our causes such solution to resist changes in pH upon addition of acid(s) or alkali(s) to the solution. Examples of suitable non-naturally occurring buffering agents that may be used include, for example, any of Tris, HEPES, TAPS, MOPS, tricine, and MES.

Any appropriate nucleotide sequence may be used as a barcode (also referred to in the art as a unique molecular identifier or UMI). A barcode may be present in a primer or adapter as described herein. A barcode may comprise or consist of a unique random oligonucleotide sequence (e.g. 12-14 nucleotides in length). A barcode may be used to uniquely tag individual DNA strands, allowing for barcoded strands to be individually identified.

As used herein, a “primer” or “oligonucleotide primer” refers to a short single-stranded nucleic acid (DNA or RNA) that is complementary to a target sequence, which allow DNA polymerase to copy the target sequence by the addition of nucleotides to the 3′-end of the primer sequence. A primer of the invention may be of any appropriate length as described herein, e.g. between about 5-30 bases. The term “primer” encompasses random primers, bump primers, exonuclease-resistant primers, chemically-modified primers, custom sequence primers, or combinations thereof. In particular, primers used in the method of the invention may be pyrollo C modified oligonucleotide primers. A primer of the invention is typically specific to a target sequence. Typically a primer according to the disclosure is synthetic.

As used herein, the term “sample” refers to a sample of biological materials (cells, tissue, fluid, etc.). Said material may be obtained from a eukaryotic or prokaryotic organism. Typically, the sample is from a eukaryotic multicellular organism, such as an animal or plant. The sample may be from a mammal, particularly a human. The mammal, particularly a human may be an adult or a juvenile, male or female. Thus a sample may be obtained from an individual, and particularly in the case of a diagnostic application, from an individual suspected of having or at risk of having a medical condition. The sample may be any suitable biological material, for example blood, plasma, saliva, serum, sputum, urine, cerebrospinal fluid, sweat, synovial fluid, cells, a cellular extract, a tissue sample, a tissue biopsy, a stool sample and the like. Typically the sample is a biological fluid, such as blood, plasma, saliva, serum, sputum, urine, cerebrospinal fluid, sweat, synovial fluid, preferably a blood sample. The precise biological sample that is taken from the individual may vary, but the sampling preferably is minimally invasive and is easily performed by conventional techniques. The sample may be a whole blood sample, a purified peripheral blood leukocyte sample or a cell type sorted leukocyte sample, such as a sample of the individual's neutrophils.

Amplification as used herein refers to at least the manufacture of at least one copy of a single strand of the target double stranded DNA. Amplification may include multiple copies of complementary single strand DNA to create multiple copies of dsDNA with the limit of copy number being only the amount of dsDNA needed to detect the presence and/or location of nucleotide modifications in polynucleotides. Unless stated otherwise, amplification as used in the claims refers to amplification of DNA containing methylated cytosine particularly methyl cytosine in CpG wherein the methylated cytosine is preserved in the copies of the original DNA. The amplification of methylated or hemimethylated DNA may be performed on target nucleic acid that originated as RNA such that a reverse transcription reaction occurred prior to formation of ds DNA in which the methylation marks were preserved. Any appropriate means of amplification may be used, including PCR (including droplet PCT, quantitative PCR, etc.), bridge amplification, hybrid capture amplification, linked linear amplification and multiple displacement amplification (MDA).

The term “pharmaceutically acceptable” as used herein means approved by a regulatory agency of the Federal or a state government, or listed in the U.S. Pharmacopeia, European Pharmacopeia or other generally recognized pharmacopeia.

The publications discussed herein are provided solely for their disclosure prior to the filing date of the present application. Nothing herein is to be construed as an admission that such publications constitute prior art to the claims appended hereto.

Disclosure related to the various methods of the invention are intended to be applied equally to other methods, therapeutic uses or methods, the data storage medium or device, the computer program product, and vice versa.

### Methyltransferases

DNA methylation is catalysed by one of a family of enzymes known as DNA methyltransferases (DNMTs or DNA MTs) which transfer a methyl group from a methyl group donor, typically a S-adenyl methionine (SAM) or SAM analogue, to DNA.

There are three classes of DNMTs, defined by their specific methylation activity. The m6A class of DNMTs add a methyl group to the N6 position of adenine to produce N6-methyladenine (m6A). The m4C class of DNMTs add a methyl group to the N4 position of cytosine to produce N4-methylcytosine (m4C). The 5 mC class of DNMTs add a methyl group to the C5 position of cytosine to produce 5-methylcytosine (5 mC). The invention is particularly concerned with 5 mC DNMTs. The invention particularly relates to methylation of the C5 position of cytosine to produce 5 mC. Any and all references herein to DNA methylation preferably relate to the production of 5 mC. Any reference herein to methylcytosine or mC preferably relates to 5 mC.

Further, the invention allows for the faithfully copying of methyl groups present within one or more target sequence on at least one parent DNA strand (also referred to interchangeably herein as the template strand or the DNA substrate) to the amplified DNA product or daughter strand(s), such that the methylation pattern of the DNA substrate is accurately conserved in the amplified product or daughter strand(s). Thus, a method of the invention can produce a symmetrically-methylated dsDNA product. In other words, cytosine residues which are methylated within the DNA substrate will be methylated at the corresponding position in the amplified product, and unmethylated cytosine residues within the DNA substrate will be unmethylated at the corresponding position in the amplified product. Unmethylated cytosine residues within the DNA substrate will not be methylated at the corresponding position in the amplified product.

Accordingly, a DNMT used in the present invention typically has activity on and/or specificity for hemimethylated DNA that is significantly greater than its activity on and/or specificity for unmethylated DNA. The activity and/or specificity of a DNMT of the invention for hemimethylated DNA may be greater than 50-fold more, such as at least 60-fold, at least 70-fold, at least 80-fold, at least 90-fold, at least 100-fold, at least 150-fold greater, or more, than its activity on and/or specificity for unmethylated DNA. Preferably, a DNMT of the invention has at least 80-fold, at least 90-fold, at least 100-fold, at least 150-fold greater, or more, more preferably at least 100-fold or more, at least 120-fold, at least 130-fold, at least 140-fold, at least 150-fold, at least 200-fold greater, or more, than its activity on and/or specificity for unmethylated DNA. A DNMT of the invention may have activity and/or specificity for hemimethylated DNA that is at least 100-fold greater than its activity on and/or specificity for unmethylated DNA. This specificity for hemimethylated DNA is typically measured globally across the one or more target region and/or at least one DNA strand.

The ability of a DNMT to add a methyl group to an unmethylated DNA substrate is referred to as de novo methylation. In view of its specificity for hemimethylated DNA, a DNMT of the invention typically has decreased de novo methylation activity. Thus, a DNMT used in the present invention typically has decreased de novo methylation activity compared with its activity on and/or specificity for hemimethylated DNA. The de novo methylation activity of a DNMT of the invention may be more than 50-fold less, such as at least 60-fold, at least 70-fold, at least 80-fold, at least 90-fold, at least 100-fold, at least 150-fold lower, or less, than its activity on and/or specificity for hemimethylated DNA. Preferably, a DNMT of the invention has de novo methylation activity that is at least 80-fold, at least 90-fold, at least 100-fold, at least 150-fold lower, or less, more preferably at least 100-fold lower, at least 120-fold lower, at least 130-fold lower, at least 140-fold lower, at least 150-fold lower, at least 200-fold lower, or less, than its activity on and/or specificity for hemimethylated DNA. A DNMT of the invention may have de novo methylation activity that is at least 100-fold lower than its activity on and/or specificity for hemimethylated DNA. The specificity for hemimethylated DNA and/or decreased de novo methylation is typically measured globally across the one or more target region and/or at least one DNA strand.

The de novo methylation activity of a DNMT of the invention may be less than 10%, less than 9%, less than 8%, less than 7%, less than 6%, less than 5%, less than 4%, less than 3%, less than 2%, less than 1.5%, less than 1%, less than 0.9%, less than 0.8%, less than 0.7%, less than 0.6%, less than 0.5% or lower, of its activity on hemimethylated DNA. Preferably, a DNMT of the invention has de novo methylation activity that is less than 1.5%, less than 1%, or lower, of its activity on hemimethylated DNA. The specificity for hemimethylated DNA and/or decreased de novo methylation is typically measured globally across the one or more target region and/or at least one DNA strand.

DNMT1 has previously been shown in vitro to have a 30- to 40-fold preference, and up to 50-fold preference for hemimethylated DNA compared with unmethylated DNA, and a 15-fold preference for methylated CG sites. Whilst this specificity is the consensus in the art, there are isolated disclosures which attribute a lower level of de novo methylation activity to DNMT1. For example, US2020/0063213, and U.S. Pat. No. 11,130,991, both of which are incorporated herein by reference, disclose methods of amplification that utilise DNMT1 and specify a lower level of de novo methylation by DNMT1 than is generally accepted in the art. However, even these isolated publications described de novo methylation at a level which is disadvantageous in practice, particularly in applications using cfDNA where the amount of sample DNA is low. For example, US2020/0063213 reports de novo methylation activity of DNMT1 of at least 1.7%. Although this percentage appears low, when considering 1.7% of a genome, which millions if not billions of bases, this seemingly small percentage has the potential to equate to thousands to millions of de novo methylations, having a significant detrimental effect on the methylation analysis. Whilst other isolated publications identify the de novo activity of DNMT1 as potentially problematic (e.g. U.S. Pat. No. 7,449,297, which is incorporated by reference), this problem remains unresolved, with no practical solutions to this problem provided. Therefore, for the avoidance of doubt, in preferred embodiments of the invention, the DNMT is not DNMT1 or a variant thereof which retains the de novo methylation activity of DNMT1.

A DNMT used in the present invention may have no activity, de minimis activity or background activity on and/or specificity for unmethylated DNA. In other words, a DNMT used in the present invention may have essentially no or de minimis de novo methylation activity (e.g. less than 1% de novo methylation activity).

Typically, a DNMT used in the present invention has activity on and/or specificity for hemimethylated DNA that is significantly greater than its activity on and/or specificity for unmethylated DNA, as defined herein.

To achieve this faithfully copying of the methylation pattern a DNMT used in the present invention typically possesses both methyltransferase activity and ATP hydrolysis (ATPase) activity. ATP hydrolysis by the DNMT enables the enzyme to specifically recognise hemimethylated DNA substrates. Therefore, a DNMT according to the invention may comprise both a MT domain and an ATPase domain. The ATPase domain may be from the Snf2-superfamily of proteins, or be an Snf2-like ATPase domain. As described herein, one such example of a DNMT with both ATPase activity and MT activity is DNMT5.

Thus, the invention relates to the use of enzymes comprising both methyltransferase activity and ATP hydrolysis (ATPase) activity to amplify target dsDNA containing symmetrically methylated nucleotides. Typically, a DNMT of the invention comprises both methyltransferase activity and ATPase activity, such that the term “DNMT” and “an enzyme comprising both methyltransferase activity and ATP hydrolysis (ATPase) activity wherein the enzyme comprises a methyltransferase domain and an ATPase domain” may be used interchangeably. As such, any and all disclosure herein in relation to DNMTs (and particularly DNMT5) applies equally and without reservation to enzymes comprising both methyltransferase activity and ATP hydrolysis (ATPase) activity and which comprise a methyltransferase domain and an ATPase domain. By way of non-limiting example, the disclosure in relation to thermostability of DNMTs of the invention applies equally to said enzymes. Accordingly, the invention relates to the use of thermostable enzymes comprising both methyltransferase activity and ATPase activity and which comprise a methyltransferase domain and an ATPase domain in the methods described herein. By way of further non-limiting example, disclosure herein in relation to tagging and/or labelling of DNMTs (e.g. DNMT5s) applies equally to said enzymes. Accordingly, the invention relates to the use of enzymes comprising both methyltransferase activity and ATPase activity and which comprise a methyltransferase domain and an ATPase domain which are tagged and/or labelled. By way of yet further non-limiting example, disclosure herein in relation to truncations of DNMTs (e.g. DNMT5) applies equally to said enzymes. Accordingly, the invention relates to the use of truncated enzymes comprising both methyltransferase activity and ATPase activity and which comprise a methyltransferase domain and an ATPase domain in the methods described herein.

The invention also encompasses DNMT with increased activity on and/or specificity for hemimethylated DNA compared with its activity on and/or specificity for unmethylated DNA; and/or decreased de novo methylation activity by virtue of a mechanism other than through ATPase activity.

The activity on and/or specificity of a DNMT for (i) hemimethylated DNA and/or (ii) unmethylated DNA may be determined using any appropriate technique, examples of which are known in the art. By way of non-limiting example, a suitable DNA methyltransferase assay is described in Catania et al. (2020, Cell 180, 263-277), which is herein incorporated by reference in its entirety, although for the avoidance of doubt, the focus of Catania et al. is on the evolutionary persistence of DNA methylation in fungi, and is entirely silent regarding potential applications of this DNMT.

Any DNMT with the above functional properties, e.g. ATP hydrolysis activity, increased activity on and/or specificity for hemimethylated DNA and/or decreased de novo methylation activity compared with its activity on and/or specificity for hemimethylated DNA may be used according to the invention.

Fragments and/or variants of DNMTs with the above functional properties are also encompassed within the present invention, provided said fragments and/or variants at least partially retain these properties. A fragment of a DNMT may comprise or consist of at least 200, at least 300, at least 400, at least 500, at least 750, at least 1000, at least 1500, at least 2000 or more amino acids from a full-length DNMT sequence. Alternatively, or in addition, a DNMT fragment may comprise or consist of at least 50%, at least 60%, at least 70%, at least 80%, at least 90%, at least 95%, at least 97% or more of the original DNMT.

A DNMT fragment according to the invention may be a continuous or a discontinuous fragment. A discontinuous DNMT fragment is one which is lacking at least one region of continuous amino acids from within the full length DNMT protein, such that the discontinuous fragment has at least one gap or break in the full length DNMT sequence. Thus, a discontinuous DNMT fragment of the invention comprises at least two regions, at least three regions, at least four regions, at least five regions, at least six regions, at least seven regions, at least eight regions, at least nine regions, at least ten regions, or more regions of continuous amino acid sequence from the full length DNMT protein which are separated in the full length DNMT protein, but which form a single polypeptide in the discontinuous fragment. A discontinuous fragment of a DNMT may comprise a methyltransferase domain of said DNMT and/or the ATPase domain of said DNMT.

A DNMT of the invention may be a truncated DNMT, such as those exemplified herein. Typically, a truncated DNMT will comprise the MT domain and an ATPase domain. Thus, a DNMT5 fragment of the invention may be truncated at the N- or C-terminus of the protein, provided that the MT domain and/or ATPase domain (preferably both) remain functional. Preferably, a truncated DNMT of the invention may be truncated at the N- or C-terminus of the protein, provided that the amino acid sequences of the MT domain and/or ATPase domain (preferably both) are not themselves truncated. Thus, a truncated DNMT of the invention typically comprises both methyltransferase activity and ATPase activity.

Variants of DNMTs may comprise or consist of an amino acid sequence having at least 50%, at least 60%, at least 70%, at least 80%, at least 90%, at least 95%, at least 97% or more of the original DNMT amino acid sequence.

According to the invention, variants of DNMT also include structural variants. A structural variant may have sequence identity with a DNMT as defined herein, or may have lower sequence identity, but have key structural domains, motifs and/or residues in common with said DNMT. For example, a structural variant of a DNMT as defined herein may comprise a Rossmann fold or a Rossmannoid fold, as described herein in connection with DNMT5 structural variants. Alternatively or in addition, a structural variant of a DNMT as defined herein may comprise a Walker motif, as described herein in connection with DNMT5 structural variants, which has ATPase activity. Structural DNMT variants that contain a Rossmann fold and/or Walker motifs can be identified through protein structure-based comparisons, using tools such as DALI (Holm L, Laiho A, Toronen P, Salgado M (2023) DALI shines a light on remote homologs: one hundred discoveries. Protein Science 23, e4519), MADOKA (Deng, L., Zhong, G., Liu, C. et al. MADOKA: an ultra-fast approach for large-scale protein structure similarity searching. BMC Bioinformatics 20 (Suppl 19), 662 (2019)) and is an ongoing area of high research activity.

The methyltransferase domains and ATPase domains of enzymes comprising both methyltransferase activity and ATPase activity wherein the enzyme comprises a methyltransferase domain and an ATPase domain (i.e. DNMTs according to the invention) comprise sequence motifs.

The methyltransferase domain of an enzyme (DNMT) of the invention may comprise one or more (e.g. any 2, 3, 4, 5, 6, 7, 8, 9 or all 10) of the methyltransferase (MT) sequence motifs MT1, MT23, MT4, MT5, MT6, MT7, MT7B, MT8, MT8B, MT9 or MT10, or any combination thereof. The MT motifs (and combinations thereof) can be selected independently. Further, the MT motif nomenclature is not intended to limit the specific MT motifs to a particular location within an enzyme of the invention. By way of non-limiting example, a first enzyme may comprise MT1, MT23 and MT7 in the N- to C-terminal direction, with or without intervening MT motifs. By way of a further non-limiting example, a second enzyme may comprise MT4, MT23, MT6 and MT7 in the N- to C-terminal direction, with or without intervening MT motifs.

The MT1 motif may comprise or consist of a sequence having at least 80%, at least 85%, at least 90%, at least 95%, at least 96%, at least 97%, at least 98%, at least 99% or more sequence identity to the consensus sequence of SEQ ID NO 35. The MT1 motif may comprise or consist of a sequence having at least 90% sequence identity to the consensus sequence of SEQ ID NO 35.

Alternatively or in addition, the MT23 motif may comprise or consist of a sequence having at least 80%, at least 85%, at least 90%, at least 95%, at least 96%, at least 97%, at least 98%, at least 99% or more sequence identity to the consensus sequence of SEQ ID NO 36. The MT23 motif may comprise or consist of a sequence having at least 90% sequence identity to the consensus sequence of SEQ ID NO 36.

Further alternatively or in addition, the MT4 motif may comprise or consist of a sequence having at least 80%, at least 85%, at least 90%, at least 95%, at least 96%, at least 97%, at least 98%, at least 99% or more sequence identity to the consensus sequence of SEQ ID NO 37. The MT4 motif may comprise or consist of a sequence having at least 90% sequence identity to the consensus sequence of SEQ ID NO 37.

Further alternatively or in addition, the MT5 motif may comprise or consist of a sequence having at least 80%, at least 85%, at least 90%, at least 95%, at least 96%, at least 97%, at least 98%, at least 99% or more sequence identity to the consensus sequence of SEQ ID NO 38. The MT5 motif may comprise or consist of a sequence having at least 90% sequence identity to the consensus sequence of SEQ ID NO 38.

Further alternatively or in addition, the MT6 motif may comprise or consist of a sequence having at least 80%, at least 85%, at least 90%, at least 95%, at least 96%, at least 97%, at least 98%, at least 99% or more sequence identity to the consensus sequence of SEQ ID NO 39. The MT6 motif may comprise or consist of a sequence having at least 90% sequence identity to the consensus sequence of SEQ ID NO 39.

Further alternatively or in addition, the MT7 motif may comprise or consist of a sequence having at least 80%, at least 85%, at least 90%, at least 95%, at least 96%, at least 97%, at least 98%, at least 99% or more sequence identity to the consensus sequence of SEQ ID NO 40. The MT7 motif may comprise or consist of a sequence having at least 90% sequence identity to the consensus sequence of SEQ ID NO 40.

Further alternatively or in addition, the MT7B motif may comprise or consist of a sequence having at least 80%, at least 85%, at least 90%, at least 95%, at least 96%, at least 97%, at least 98%, at least 99% or more sequence identity to the consensus sequence of SEQ ID NO 41. The MT7B motif may comprise or consist of a sequence having at least 90% sequence identity to the consensus sequence of SEQ ID NO 41.

Further alternatively or in addition, the MT8 motif may comprise or consist of a sequence having at least 80%, at least 85%, at least 90%, at least 95%, at least 96%, at least 97%, at least 98%, at least 99% or more sequence identity to the consensus sequence of SEQ ID NO 42. The MT8 motif may comprise or consist of a sequence having at least 90% sequence identity to the consensus sequence of SEQ ID NO 42.

Further alternatively or in addition, the MT8B motif may comprise or consist of a sequence having at least 80%, at least 85%, at least 90%, at least 95%, at least 96%, at least 97%, at least 98%, at least 99% or more sequence identity to the consensus sequence of SEQ ID NO 43. The MT8B motif may comprise or consist of a sequence having at least 90% sequence identity to the consensus sequence of SEQ ID NO 43.

Further alternatively or in addition, the MT9 motif may comprise or consist of a sequence having at least 80%, at least 85%, at least 90%, at least 95%, at least 96%, at least 97%, at least 98%, at least 99% or more sequence identity to the consensus sequence of SEQ ID NO 44. The MT9 motif may comprise or consist of a sequence having at least 90% sequence identity to the consensus sequence of SEQ ID NO 44.

Further alternatively or in addition, the MT10 motif may comprise or consist of a sequence having at least 80%, at least 85%, at least 90%, at least 95%, at least 96%, at least 97%, at least 98%, at least 99% or more sequence identity to the consensus sequence of SEQ ID NO 45. The MT10 motif may comprise or consist of a sequence having at least 90% sequence identity to the consensus sequence of SEQ ID NO 45.

In particular, the MT1 motif may comprise or consist of a sequence having at least 80%, at least 85%, at least 90%, at least 95%, at least 96%, at least 97%, at least 98%, at least 99% or more sequence identity to any one of SEQ ID NOs 53, 71, 89, 107, 125, 143, 179, 197, 215, 233, 251, 269, 287, 305, 323, 341, 359, 37, 395, 413, 431 and 449. The MT1 motif may comprise or consist of a sequence having at least 90% sequence identity to any one of SEQ ID NOs 53, 71, 89, 107, 125, 143, 179, 197, 215, 233, 251, 269, 287, 305, 323, 341, 359, 37, 395, 413, 431 and 449.

Alternatively or in addition, the MT23 motif may comprise or consist of a sequence having at least 80%, at least 85%, at least 90%, at least 95%, at least 96%, at least 97%, at least 98%, at least 99% or more sequence identity to any one of SEQ ID NOs 54, 72, 90, 108, 126, 144, 180, 198, 216, 234, 252, 270, 288, 306, 324, 342, 360, 378, 396, 414, 432 and 450. The MT23 motif may comprise or consist of a sequence having at least 90% sequence identity to any one of SEQ ID NOs: 54, 72, 90, 108, 126, 144, 180, 198, 216, 234, 252, 270, 288, 306, 324, 342, 360, 378, 396, 414, 432 and 450.

Further alternatively or in addition, the MT4 motif may comprise or consist of a sequence having at least 80%, at least 85%, at least 90%, at least 95%, at least 96%, at least 97%, at least 98%, at least 99% or more sequence identity to any one of SEQ ID NOs: 55, 73, 91, 109, 127, 145, 163, 181, 199, 217, 235, 253, 271, 289, 307, 325, 343, 361, 379, 397, 415, 433 and 451. The MT4 motif may comprise or consist of a sequence having at least 90% sequence identity to any one of SEQ ID NOs: 55, 73, 91, 109, 127, 145, 163, 181, 199, 217, 235, 253, 271, 289, 307, 325, 343, 361, 379, 397, 415, 433 and 451.

Further alternatively or in addition, the MT5 motif may comprise or consist of a sequence having at least 80%, at least 85%, at least 90%, at least 95%, at least 96%, at least 97%, at least 98%, at least 99% or more sequence identity to any one of SEQ ID NOs: 56, 74, 92, 110, 128, 146, 164, 182, 200, 218, 236, 254, 272, 290, 308, 326, 344, 362, 380, 398, 416, 434, and 452. The MT5 motif may comprise or consist of a sequence having at least 90% sequence identity to any one of SEQ ID NOs: 56, 74, 92, 110, 128, 146, 164, 182, 200, 218, 236, 254, 272, 290, 308, 326, 344, 362, 380, 398, 416, 434, and 452.

Further alternatively or in addition, the MT6 motif may comprise or consist of a sequence having at least 80%, at least 85%, at least 90%, at least 95%, at least 96%, at least 97%, at least 98%, at least 99% or more sequence identity to any one of SEQ ID NOs: 57, 75, 93, 111, 129, 147, 165, 183, 201, 219, 237, 255, 273, 291, 309, 327, 345, 363, 381, 399, 417, 435, and 453. The MT6 motif may comprise or consist of a sequence having at least 90% sequence identity to any one of SEQ ID NOs: 57, 75, 93, 111, 129, 147, 165, 183, 201, 219, 237, 255, 273, 291, 309, 327, 345, 363, 381, 399, 417, 435, and 453.

Further alternatively or in addition, the MT7 motif may comprise or consist of a sequence having at least 80%, at least 85%, at least 90%, at least 95%, at least 96%, at least 97%, at least 98%, at least 99% or more sequence identity to any one of SEQ ID NOs: 58, 76, 94, 112, 130, 148, 166, 184, 202, 220, 238, 256, 274, 292, 310, 328, 346, 364, 382, 400, 418, 436, and 454. The MT7 motif may comprise or consist of a sequence having at least 90% sequence identity to any one of SEQ ID NOs: 58, 76, 94, 112, 130, 148, 166, 184, 202, 220, 238, 256, 274, 292, 310, 328, 346, 364, 382, 400, 418, 436, and 454.

Further alternatively or in addition, the MT7B motif may comprise or consist of a sequence having at least 80%, at least 85%, at least 90%, at least 95%, at least 96%, at least 97%, at least 98%, at least 99% or more sequence identity to any one of SEQ ID NOs: 59, 77, 95, 113, 131, 149, 167, 185, 203, 221, 239, 257, 275, 293, 311, 329, 347, 365, 383, 401, 419, 437, and 455. The MT7B motif may comprise or consist of a sequence having at least 90% sequence identity to any one of SEQ ID NOs: 59, 77, 95, 113, 131, 149, 167, 185, 203, 221, 239, 257, 275, 293, 311, 329, 347, 365, 383, 401, 419, 437, and 455.

Further alternatively or in addition, the MT8 motif may comprise or consist of a sequence having at least 80%, at least 85%, at least 90%, at least 95%, at least 96%, at least 97%, at least 98%, at least 99% or more sequence identity to any one of SEQ ID NOs: 60, 78, 96, 114, 132, 150, 168, 186, 204, 222, 240, 258, 276, 294, 312, 330, 348, 366, 384, 402, 420, 438, and 456. The MT8 motif may comprise or consist of a sequence having at least 90% sequence identity to any one of SEQ ID NOs: 60, 78, 96, 114, 132, 150, 168, 186, 204, 222, 240, 258, 276, 294, 312, 330, 348, 366, 384, 402, 420, 438, and 456.

Further alternatively or in addition, the MT8B motif may comprise or consist of a sequence having at least 80%, at least 85%, at least 90%, at least 95%, at least 96%, at least 97%, at least 98%, at least 99% or more sequence identity to any one of SEQ ID NO: 61, 79, 97, 115, 133, 151, 169, 187, 205, 223, 241, 259, 277, 295, 313, 331, 349, 367, 385, 403, 421, 439, and 457. The MT8B motif may comprise or consist of a sequence having at least 90% sequence identity to any one of SEQ ID NOs: 61, 79, 97, 115, 133, 151, 169, 187, 205, 223, 241, 259, 277, 295, 313, 331, 349, 367, 385, 403, 421, 439, and 457.

Further alternatively or in addition, the MT9 motif may comprise or consist of a sequence having at least 80%, at least 85%, at least 90%, at least 95%, at least 96%, at least 97%, at least 98%, at least 99% or more sequence identity to any one of SEQ ID NOs: 62, 80, 98, 116, 134, 152, 170, 188, 206, 224, 242, 260, 278, 296, 314, 332, 350, 368, 386, 404, 422, 440, and 458. The MT9 motif may comprise or consist of a sequence having at least 90% sequence identity to any one of SEQ ID NOs: 62, 80, 98, 116, 134, 152, 170, 188, 206, 224, 242, 260, 278, 296, 314, 332, 350, 368, 386, 404, 422, 440, and 458.

Further alternatively or in addition, the MT10 motif may comprise or consist of a sequence having at least 80%, at least 85%, at least 90%, at least 95%, at least 96%, at least 97%, at least 98%, at least 99% or more sequence identity to any one of SEQ ID NOs: 63, 81, 99, 117, 135, 153, 171, 189, 207, 225, 243, 261, 279, 297, 315, 333, 351, 369, 387, 405, 423, 441, and 459. The MT10 motif may comprise or consist of a sequence having at least 90% sequence identity to any one of SEQ ID NOs: 63, 81, 99, 117, 135, 153, 171, 189, 207, 225, 243, 261, 279, 297, 315, 333, 351, 369, 387, 405, 423, 441, and 459.

The ATPase domain of an enzyme (DNMT) of the invention may comprise one or more (e.g. any 2, 3, 4, 5, 6 or all 7) of the ATPase domain sequence motifs (ATP motifs) ATP11B, ATP12, ATP13, ATP14, ATP14B, ATP15 or ATP15B, or any combination thereof. The ATP motifs (and combinations thereof) can be selected independently. Further, the ATP motif nomenclature is not intended to limit the specific ATP motifs to a particular location within an enzyme of the invention. By way of non-limiting example, a first enzyme may comprise ATP11B, ATP13 and ATP15B in the N- to C-terminal direction, with or without intervening ATP motifs. By way of a further non-limiting example, a second enzyme may comprise ATP11B, ATP14B, ATP13, ATP12 and ATP15B in the N- to C-terminal direction, with or without intervening ATP motifs.

The ATP11B motif may comprise or consist of a sequence having at least 80%, at least 85%, at least 90%, at least 95%, at least 96%, at least 97%, at least 98%, at least 99% or more sequence identity to the consensus sequence of SEQ ID NO 46. The ATP11B motif may comprise or consist of a sequence having at least 90% sequence identity to the consensus sequence of SEQ ID NO 46.

Alternatively or in addition, the ATP12 motif may comprise or consist of a sequence having at least 80%, at least 85%, at least 90%, at least 95%, at least 96%, at least 97%, at least 98%, at least 99% or more sequence identity to the consensus sequence of SEQ ID NO 47. The ATP12 motif may comprise or consist of a sequence having at least 90% sequence identity to the consensus sequence of SEQ ID NO 47.

Further alternatively or in addition, the ATP13 motif may comprise or consist of a sequence having at least 80%, at least 85%, at least 90%, at least 95%, at least 96%, at least 97%, at least 98%, at least 99% or more sequence identity to the consensus sequence of SEQ ID NO 48. The ATP13 motif may comprise or consist of a sequence having at least 90% sequence identity to the consensus sequence of SEQ ID NO 48.

Further alternatively or in addition, the ATP14 motif may comprise or consist of a sequence having at least 80%, at least 85%, at least 90%, at least 95%, at least 96%, at least 97%, at least 98%, at least 99% or more sequence identity to the consensus sequence of SEQ ID NO 49. The ATP14 motif may comprise or consist of a sequence having at least 90% sequence identity to the consensus sequence of SEQ ID NO 49.

Further alternatively or in addition, the ATP14B motif may comprise or consist of a sequence having at least 80%, at least 85%, at least 90%, at least 95%, at least 96%, at least 97%, at least 98%, at least 99% or more sequence identity to the consensus sequence of SEQ ID NO 50. The ATP14B motif may comprise or consist of a sequence having at least 90% sequence identity to the consensus sequence of SEQ ID NO 51.

Further alternatively or in addition, the ATP15 motif may comprise or consist of a sequence having at least 80%, at least 85%, at least 90%, at least 95%, at least 96%, at least 97%, at least 98%, at least 99% or more sequence identity to the consensus sequence of SEQ ID NO 51. The ATP15 motif may comprise or consist of a sequence having at least 90% sequence identity to the consensus sequence of SEQ ID NO 51.

Further alternatively or in addition, the ATP15B motif may comprise or consist of a sequence having at least 80%, at least 85%, at least 90%, at least 95%, at least 96%, at least 97%, at least 98%, at least 99% or more sequence identity to the consensus sequence of SEQ ID NO 52. The ATP15B motif may comprise or consist of a sequence having at least 90% sequence identity to the consensus sequence of SEQ ID NO 52.

In particular, the ATP11B motif may comprise or consist of a sequence having at least 80%, at least 85%, at least 90%, at least 95%, at least 96%, at least 97%, at least 98%, at least 99% or more sequence identity to any one of SEQ ID NOs: 64, 82, 100, 118, 136, 154, 172, 190, 208, 226, 244, 262, 280, 298, 316, 334, 352, 370, 388, 406, 424, 442, and 460. The ATP11B motif may comprise or consist of a sequence having at least 90% sequence identity to any one of SEQ ID NOs: 64, 82, 100, 118, 136, 154, 172, 190, 208, 226, 244, 262, 280, 298, 316, 334, 352, 370, 388, 406, 424, 442, and 460.

Alternatively or in addition, the ATP12 motif may comprise or consist of a sequence having at least 80%, at least 85%, at least 90%, at least 95%, at least 96%, at least 97%, at least 98%, at least 99% or more sequence identity to any one of SEQ ID NOs: 65, 83, 101, 119, 137, 155, 173, 191, 209, 227, 245, 263, 281, 299, 317, 335, 353, 371, 389, 407, 425, 443, and 461. The ATP12 motif may comprise or consist of a sequence having at least 90% sequence identity to any one of SEQ ID NOs: 65, 83, 101, 119, 137, 155, 173, 191, 209, 227, 245, 263, 281, 299, 317, 335, 353, 371, 389, 407, 425, 443, and 461.

Further alternatively or in addition, the ATP13 motif may comprise or consist of a sequence having at least 80%, at least 85%, at least 90%, at least 95%, at least 96%, at least 97%, at least 98%, at least 99% or more sequence identity to any one of SEQ ID NOs: 66, 84, 102, 120, 138, 156, 174, 192, 210, 228, 246, 264, 282, 300, 318, 336, 354, 372, 390, 408, 426, 444, and 462. The ATP13 motif may comprise or consist of a sequence having at least 90% sequence identity to any one of SEQ ID NOs: 66, 84, 102, 120, 138, 156, 174, 192, 210, 228, 246, 264, 282, 300, 318, 336, 354, 372, 390, 408, 426, 444, and 462.

Further alternatively or in addition, the ATP14 motif may comprise or consist of a sequence having at least 80%, at least 85%, at least 90%, at least 95%, at least 96%, at least 97%, at least 98%, at least 99% or more sequence identity to any one of SEQ ID NOs: 67, 85, 103, 121, 139, 157, 175, 193, 211, 229, 247, 265, 283, 301, 319, 337, 355, 373, 391, 409, 427, 445, and 463. The ATP14 motif may comprise or consist of a sequence having at least 90% sequence identity to any one of SEQ ID NOs: 67, 85, 103, 121, 139, 157, 175, 193, 211, 229, 247, 265, 283, 301, 319, 337, 355, 373, 391, 409, 427, 445, and 463.

Further alternatively or in addition, the ATP14B motif may comprise or consist of a sequence having at least 80%, at least 85%, at least 90%, at least 95%, at least 96%, at least 97%, at least 98%, at least 99% or more sequence identity to any one of SEQ ID NOs: 68, 86, 104, 122, 140, 176, 194, 212, 230, 248, 266, 284, 302, 320, 338, 356, 374, 392, 410, 446, and 464. The ATP14B motif may comprise or consist of a sequence having at least 90% sequence identity to any one of SEQ ID NOs: 68, 86, 104, 122, 140, 176, 194, 212, 230, 248, 266, 284, 302, 320, 338, 356, 374, 392, 410, 446, and 464.

Further alternatively or in addition, the ATP15 motif may comprise or consist of a sequence having at least 80%, at least 85%, at least 90%, at least 95%, at least 96%, at least 97%, at least 98%, at least 99% or more sequence identity to any one of SEQ ID NOs: 69, 87, 105, 123, 141, 159, 177, 213, 231, 249, 267, 285, 303, 321, 339, 357, 375, 393, 411, 429, 447, and 465. The ATP15 motif may comprise or consist of a sequence having at least 90% sequence identity to any one of SEQ ID NOs: 69, 87, 105, 123, 141, 159, 177, 213, 231, 249, 267, 285, 303, 321, 339, 357, 375, 393, 411, 429, 447, and 465.

Further alternatively or in addition, the ATP15B motif may comprise or consist of a sequence having at least 80%, at least 85%, at least 90%, at least 95%, at least 96%, at least 97%, at least 98%, at least 99% or more sequence identity to any one of SEQ ID NOs: 70, 88, 106, 124, 142, 160, 178, 214, 232, 250, 268, 286, 304, 322, 340, 358, 376, 394, 412, 430, 448, and 466. The ATP15B motif may comprise or consist of a sequence having at least 90% sequence identity to any one of SEQ ID NOs: 70, 88, 106, 124, 142, 160, 178, 214, 232, 250, 268, 286, 304, 322, 340, 358, 376, 394, 412, 430, 448, and 466.

An enzyme (DNMT) of the invention (or its methyltransferase domain) may therefore comprise two or more, three or more, four or more, five or more, six or more, seven or more, eight or more, nine or more or all 10 of the MT motifs described herein. Alternatively or in addition, an enzyme (DNMT) of the invention (or its ATPase domain) may therefore comprise two or more, three or more, four or more, five or more, six or more, or all seven ATP motifs as described herein. The MT motifs and ATP motifs may be selected independently. Thus, by way of non-limiting example, an enzyme (DNMT) of the invention may comprise two or more MT motifs as described herein and/or two or more ATP motifs as described herein. By way of further non-limiting example, an enzyme (DNMT) of the invention may comprise four or more MT motifs as described herein and/or two or more ATP motifs as described herein.

In a combination of MT motifs and/or ATP motifs as described above, the individual MT and/or ATP motifs (in any combination) may optionally be from the same enzyme.

Typically a variant or fragment of DNMT of the invention will retain at least partial activity compared with the wild-type DNMT amino acid sequence from which it is derived. Partial activity may be defined at least at least 50%, at least 60%, at least 70%, at least 80%, at least 90%, at least 95%, at least 97% or more of the activity of the wild-type DNMT. A variant or fragment of DNMT of the invention may retain the full activity of the wild-type DNMT, or essentially full activity (e.g. at least 90%, at least 95%, at least 97% or more, up to 100% of the activity).

The invention also encompasses the use of chimeric DNMTs. A chimeric DNMT may comprise amino acid sequences from two or more (e.g. from two, three, four, five or more) DNMTs. A chimeric DNMT may contain domains from two or more (e.g. from two, three, four, five or more) DNMTs. By way of non-limiting example, a chimeric DNMT may comprise a methyltransferase domain from a first DNMT, and an ATPase domain from a second DNMT, wherein the first and second DNMT are different. By way of a further non-limiting example, a chimeric DNMT may comprise a methyltransferase domain from an DNMT5, and an ATPase domain from a second DNMT, which is not a DNMT5.

In some preferred embodiments, the DNTM5 of the invention is DNMT5, or a functional fragment or variant thereof, as described in further detail below. For the avoidance of doubt, any and all disclosure herein in relation to DNMT, whether in the context of methods, products or applications of the invention applies equally and without reservation to the use of DNMT5, with embodiments relating to DNMT5 being preferred.

A DNMT may be a thermostable (i.e. thermoresistant and/or thermofunctional) DNMT. A thermostable (i.e. thermoresistant and/or thermofunctional) DNMT of the invention may be thermofunctional at a temperature of up to 30° C., up to 35° C., up to 37° C., up to 40° C., up to 50° C., up to 60° C., up to 70° C., or more. A DNMT used in the invention may be thermofunctional between 25 to 60° C.

Alternatively or in addition, a thermostable (i.e. thermoresistant and/or thermofunctional) DNMT of the invention may be thermoresistant at a temperature of up to 50° C., up to 55° C., up to 60° C., up to 65° C., up to 70° C., up to 80° C., up to 90° C., up to 95° C., up to 98° C. or more. Thermostable DNMT may be isolated from thermoresistant organisms. A DNMT used in the invention may be thermoresistant up to 94° C., preferably up to 95° C. or up to 98° C.

In some embodiments, a DNMT is thermoresistant up to 94° C., up to 95° C. or up to 98° C., and thermofunctional between 25 to 60° C.

A DNMT of the invention may be produced by any suitable means, non-limiting examples of which are known in the art and which are within the routine practice of one of ordinary skill in the art. For example, a DNMT of the invention may be produced by an in vitro transcription/translation (IVTT) system, by culture of cells (particularly bacterial cells) which express DNMT, particularly cells which endogenously express said DNMT, or by recombinant expression. Thus, a DNMT may be used in the methods of the present invention in any appropriate form, e.g. as a crude preparation (e.g. cell lysate) or in an essentially pure form (e.g. recombinant DNMT). Essentially pure DNMT may comprise less than 10%, less than 9%, less than 8%, less than 7%, less than 6%, less than 5%, less than 4%, less than 3%, less than 2%, less than 1% or lower, down to 0% protein constituents other than said DNMT.

### DNMT5

DNMT5 is a member of the DNMT family which has a high level of activity on and/or specificity for hemimethylated DNA, as described above in relation the wider DNMT family.

DNMT5 typically comprises both a MT domain and an ATPase domain. The ATPase domain may be from the Snf2-superfamily of proteins, or be an Snf2-like ATPase domain.

An exemplary, but non-limiting amino acid sequence of DNMT5 is SEQ ID NO: 1.

In some preferred embodiments, the DNMT5 may be a thermostable (i.e. thermoresistant and/or thermofunctional) DNMT5. For example, a DNMT5 may be more thermostable (i.e. thermoresistant and/or thermofunctional) than the DNMT5 of SEQ ID NO: 1. A thermostable (i.e. thermoresistant and/or thermofunctional) DNMT5 of the invention may be thermofunctional at a temperature of up to 30° C., up to 35° C., up to 37° C., up to 40° C., up to 50° C., up to 60° C., up to 70° C. or more. A DNMT5 used in the invention may be thermofunctional between 25 to 60° C.

Alternatively or in addition, a thermostable (i.e. thermoresistant and/or thermofunctional) DNMT5 of the invention may be thermoresistant at a temperature of up to 50° C., up to 55° C., up to 60° C., up to 65° C., up to 70° C., up to 80° C., up to 90° C., up to 95° C., up to 98° C. or more. A DNMT5 used in the invention may be thermoresistant up to 94° C., preferably up to 95° C. or up to 98° C.

An exemplary, but non-limiting amino acid sequence of a thermostable DNMT5 is SEQ ID NO: 3. This DNTM5 was originally isolated from Takashimella tepidaria formerly known as Cryptococcus tepidarius. Other thermostable DNMT5 variants may be isolated from other thermoresistant organisms and used in the present invention.

In some embodiments, a DNMT5 is thermoresistant up to 94° C., up to 95° C. or up to 98° C., and thermofunctional between 25 to 60° C.

Fragments of DNMT5, including fragments of the DNMT5 of SEQ ID NO: 1 or 3 or variants thereof, are also encompassed within the present invention. A fragment of DNMT5 may comprise or consist of at least 200, at least 300, at least 400, at least 500, at least 750, at least 1000, at least 1500, at least 2000 or more amino acids from a full-length DNMT5 sequence, such as the DNMT5 of SEQ ID NO: 1 or 3. Alternatively or in addition, a DNMT5 fragment may comprise or consist of at least 50%, at least 60%, at least 70%, at least 80%, at least 90%, at least 95%, at least 97% or more of the original DNMT5.

A DNMT5 fragment according to the invention may be a continuous or a discontinuous DNMT5 fragment. A discontinuous DNMT5 fragment is one which is lacking at least one region of continuous amino acids from within the full length DNMT5 protein, such that the discontinuous fragment has at least one gap or break in the full length DNMT5 sequence. Thus, a discontinuous DNMT5 fragment of the invention comprises at least two regions, at least three regions, at least four regions, at least five regions, at least six regions, at least seven regions, at least eight regions, at least nine regions, at least ten regions, or more regions of continuous amino acid sequence from the full length DNMT5 protein which are separated in the full length DNMT5 protein, but which form a single polypeptide in the discontinuous fragment. A discontinuous fragment of a DNMT5 may comprise a methyltransferase domain of said DNMT5 and/or the ATPase domain of said DNMT5.

Typically, a DNMT5 fragment of the invention will comprise the MT domain and an ATPase domain.

Thus, a DNMT5 of the invention may be a truncated DNMT5, such as those exemplified herein. Typically, a truncated DNMT5 will comprise the MT domain and an ATPase domain. Thus, a DNMT5 fragment of the invention may be truncated at the N- and/or C-terminus of the protein, provided that the MT domain and/or ATPase domain (preferably both) remain functional. Preferably, a DNMT5 fragment of the invention may be truncated at the N- and/or C-terminus of the protein, provided that the amino acid sequences of the MT domain and/or ATPase domain (preferably both) are not themselves truncated. Thus, a truncated DNMT5 of the invention typically comprises both methyltransferase activity and ATPase activity. By way of non-limiting example, a truncated DNMT5 of the invention may comprise or consist of any one of SEQ ID NOs: 25 to 34, particularly SEQ ID NOs: 28 to 34. Variants of said truncated DNMT5s (as defined herein) are also encompassed.

Variants of DNMT5, including variants of the DNMT5 of SEQ ID NO: 1 or 3, or fragments thereof, are also encompassed within the present invention. A variant of DNMT5 may comprise or consist of an amino acid sequence having at least 50%, at least 60%, at least 70%, at least 80%, at least 90%, at least 95%, at least 97% identity or more of the original DNMT5, such as SEQ ID NO: 1 or 3. In some preferred embodiments, a DNMT of the invention, particularly a DNMT5 of the invention will have at least 60% identity with the DNMT5 of SEQ ID NO: 3, more preferably at least 80%, at least 90%, at least 95%, at least 97% identity or more with the DNMT5 of SEQ ID NO: 3. Preferably, a variant of DNMT5 of the invention will comprise one or more conserved amino acid residues which make contact with hemimethylated DNA in DNMT5 from C. neoformans (SEQ ID NO: 1), such as N447, G465, K510, T519, R592, C593, R596, T636, R638, S667, Q668, N669, R672, Q673 and C684, and based on structural and multiple sequence alignments, their equivalent amino acid residues in other DNMT5 variants. SEQ ID NO: 23 as described herein is a non-limiting example of a variant DNMT5 of the invention.

According to the invention, variants of DNMT5, also include structural variants. A structural variant may have sequence identity with a DNMT5 as defined herein (e.g. the DNMT5 of SEQ ID NO: 1 or 3), or may have lower sequence identity, but have key structural domains, motifs and/or residues in common with said DNMT5. By way of non-limiting example a structural variant of DNMT5 will typically comprise both an ATPase domain (e.g. a SNF2 ATPase domain) and a methyltransferase domain. A structural variant of a DNMT5 as defined herein (e.g. the DNMT5 of SEQ ID NO: 1 or 3) may comprise a Rossmann fold or a Rossmannoid fold. The Rossmann fold is a tertiary fold found in proteins that bind nucleotides, such as enzyme cofactors FAD, NAD⁺, and NADP⁺. This fold is composed of alternating beta strands and alpha helical segments where the beta strands are hydrogen bonded to each other forming an extended beta sheet and the alpha helices surround both faces of the sheet to produce a three-layered sandwich. The classical Rossmann fold contains six beta strands whereas Rossmann-like folds, also referred to as Rossmannoid folds, contain only five strands. The initial beta-alpha-beta (bab) fold is the most conserved segment of the Rossmann fold. Rossmann folds are described further in Hanukoglu (“Proteopedia: Rossmann fold: A beta-alpha-beta fold at dinucleotide binding sites”. Biochemistry and Molecular Biology Education. (2015) 43 (3): 206-9, which is herein incorporated by reference). It has been shown that the nucleoside cofactor-binding Rossmann enzymes, particularly the nicotinamide adenine dinucleotide (NAD)-, flavin adenine dinucleotide (FAD)-, and SAM-binding MTases enzymes, share common binding motifs. Thus a structural variant of DNMT5 comprising a Rossmann or Rossmannoid fold will possess MT activity.

Alternatively or in addition, a structural variant of a DNMT5 as defined herein (e.g. the DNMT5 of SEQ ID NO: 1 or 3) may comprise a Walker motif, which is a protein tertiary structure with a highly conserved three-dimensional structure which binds ATP. One important family of ATPases are the Nucleic acid-dependent ATPases such as helicases, Swi2/SNF2 proteins, and PhoH. The ATPase activity of Swi2/SNF2 proteins are stimulated by DNA and typically remodel the way DNA is packaged on chromatin. A structural variant of the invention may therefore comprise a Walker motif from any such ATPase.

Structural DNMT5 variants that contain a Rossmann fold and/or Walker motifs can be identified through protein structure-based comparisons, using tools such as DALI (Holm L, Laiho A, Toronen P, Salgado M (2023) DALI shines a light on remote homologs: one hundred discoveries. Protein Science 23, e4519), MADOKA (Deng, L., Zhong, G., Liu, C. et al. MADOKA: an ultra-fast approach for large-scale protein structure similarity searching. BMC Bioinformatics 20 (Suppl 19), 662 (2019)) and is an ongoing area of high research activity.

A DNMT5 of the invention may comprise one or more (e.g. any 2, 3, 4, 5, 6, 7, 8, 9 or all 10) of the sequence motifs MT1, MT23, MT4, MT5, MT6, MT7, MT7B, MT8, MT8B, MT9 or MT10 described herein. Alternatively or in addition, a DNMT5 of the invention may comprise one or more (e.g. any 2, 3, 4, 5, 6, or all 7) of the sequence motifs ATP11B, ATP12, ATP13, ATP14, ATP14B, ATP15 or ATP15B described herein. Thus, for the avoidance of doubt, any reference herein to an enzyme of the invention comprising one or more of said MT domain motifs and/or ATPase domain motifs applies equally and without reservation to DNMT5.

Typically a variant, truncation or fragment of DNMT5 of the invention will retain at least partial activity compared with the wild-type DNMT5 amino acid sequence from which it is derived. Partial activity may be defined at least at least 50%, at least 60%, at least 70%, at least 80%, at least 90%, at least 95%, at least 97% or more of the activity of the wild-type DNMT5. A variant, truncation or fragment of DNMT5 of the invention may retain the full activity of the wild-type DMNT5, or essentially full activity (e.g. at least 90%, at least 95%, at least 97% or more, up to 100% of the activity).

The invention also encompasses the use of chimeric DNMT5s. A chimeric DNMT5 may comprise amino acid sequences from two or more (e.g. from two, three, four, five or more) DNMT5s. A chimeric DNMT5 may contain domains from two or more (e.g. from two, three, four, five or more) DNMT5s, or from one or more DNMT5 and one or more DNMT which is not a DNMT5. By way of non-limiting example, a chimeric DNMT5 may comprise a methyltransferase domain from a first DNMT5, and an ATPase domain from a second DNMT5, wherein the first and second DNMT5 are different. By way of a further non-limiting example, a chimeric DNMT5 may comprise a methyltransferase domain from a first DNMT5, and an ATPase domain from a second DNMT5, and a third domain from a third DNMT which is not a DNMT5.

The activity of DNMT5 on unmethylated vs hemimethylated DNA may be essentially zero or insignificant compared to background. Thus, a DNMT5 used in the present invention may have no activity, de minimis activity or background activity on and/or specificity for unmethylated DNA. In other words, a DNMT5 used in the present invention may have essentially no or de minimis de novo methylation activity (e.g. less than 1% de novo methylation activity).

Typically, a DNMT used in the present invention has activity on and/or specificity for hemimethylated DNA that is significantly greater than its activity on and/or specificity for unmethylated DNA, as defined herein.

DNMT5 typically has activity on and/or specificity for hemimethylated DNA that is significantly greater than its activity on and/or specificity for unmethylated DNA. The activity and/or specificity of a DNMT5 of the invention for hemimethylated DNA may be greater than 50-fold more, such as at least 60-fold, at least 70-fold, at least 80-fold, at least 90-fold, at least 100-fold, at least 150-fold greater, or more, than its activity on and/or specificity for unmethylated DNA. Preferably, a DNMT5 of the invention has at least 80-fold, at least 90-fold, at least 100-fold, at least 150-fold greater, or more, more preferably at least 100-fold, at least 120-fold, at least 130-fold, at least 140-fold, at least 150-fold, at least 200-fold greater, or more, than its activity on and/or specificity for unmethylated DNA. A DNMT5 of the invention may have activity and/or specificity for hemimethylated DNA that is at least 100-fold greater than its activity on and/or specificity for unmethylated DNA. The specificity for hemimethylated DNA is typically measured globally across the one or more target region and/or at least one DNA strand.

In view of its specificity for hemimethylated DNA, a DNMT5 of the invention typically has decreased de novo methylation activity. Thus, a DNMT5 used in the present invention typically has decreased de novo methylation activity compared with its activity on and/or specificity for hemimethylated DNA. The de novo methylation activity of a DNMT5 of the invention may be more than 50-fold less, such as at least 60-fold, at least 70-fold, at least 80-fold, at least 90-fold, at least 100-fold, at least 150-fold lower, or less, than its activity on and/or specificity for hemimethylated DNA. Preferably, a DNMT5 of the invention has de novo methylation activity that is at least 80-fold, at least 90-fold, at least 100-fold, at least 150-fold lower, or less, more preferably at least 100-fold lower, at least 120-fold lower, at least 130-fold lower, at least 140-fold lower, at least 150-fold lower, at least 200-fold lower, or less, than its activity on and/or specificity for hemimethylated DNA. A DNMT5 of the invention may have de novo methylation activity that is at least 100-fold lower than its activity on and/or specificity for hemimethylated DNA. The specificity for hemimethylated DNA and/or decreased de novo methylation is typically measured globally across the one or more target region and/or at least one DNA strand.

The de novo methylation activity of a DNMT5 of the invention may be less than 10%, less than 9%, less than 8%, less than 7%, less than 6%, less than 5%, less than 4%, less than 3%, less than 2%, less than 1.5%, less than 1%, less than 0.9%, less than 0.8%, less than 0.7%, less than 0.6%, less than 0.5% or lower, of its activity on hemimethylated DNA. Preferably, a DNMT5 of the invention has de novo methylation activity that is less than 1.5%, less than 1%, or lower, of its activity on hemimethylated DNA. The specificity for hemimethylated DNA and/or decreased de novo methylation is typically measured globally across the one or more target region and/or at least one DNA strand.

A DNMT5 of the invention may be produced by any suitable means, non-limiting examples of which are known in the art and which are within the routine practice of one of ordinary skill in the art. For example, a DNMT5 of the invention may be produced by an IVTT system, by culture of cells (e.g. bacterial, yeast, mammalian, insect or plant cells, particularly bacterial cells) which express DNMT5, particularly cells which endogenously express said DNMT5, or by recombinant expression. Thus, a DNMT5 may be used in the methods of the present invention in any appropriate form, e.g. as a crude preparation (e.g. cell lysate) or in an essentially pure form (e.g. recombinant DNMT5). Essentially pure DNMT5 may comprise less than 10%, less than 9%, less than 8%, less than 7%, less than 6%, less than 5%, less than 4%, less than 3%, less than 2%, less than 1% or lower, down to 0% protein constituents other than said DNMT5.

### Amplification Methods

The invention provides a method for generating a double-stranded polynucleotide from a template (also known as a substrate), wherein the original methylation pattern of the substrate is accurately copied to the nascent double-stranded polynucleotide. The method allows for the polynucleotide substrate to be amplified, including its methylation pattern. Thus, the invention potentially enables the epigenetic analysis of samples containing very little starting DNA, such as samples containing cfDNA.

In particular, the invention provides a method of amplifying methylated DNA, said method comprising or consisting of the following steps: (a) using a DNA polymerase to amplify one or more target sequence within at least one DNA strand to form a hemimethylated double-stranded (dsDNA) substrate; and (b) contacting the hemimethylated dsDNA substrate with a DNA methyltransferase (DNMT), thereby producing a symmetrically-methylated dsDNA product; wherein: (i) a cycle comprising steps (a)-(b) is repeated at least once; and (ii) the DNMT has at least 50-fold greater activity and/or specificity for hemimethylated DNA compared with unmethylated DNA, preferably at least 100-fold greater activity and/or specificity for hemimethylated DNA compared with unmethylated DNA.

The methylated DNA to be amplified may be dsDNA or single-stranded DNA (ssDNA). Preferably the methylated DNA to be amplified is dsDNA.

The invention provides a method of amplifying a target double-stranded DNA (dsDNA) containing symmetrically methylated nucleotides to produce an amplified methylated dsDNA product, comprising: (a) copying at least one methylated single-stranded DNA (ssDNA) to produce hemimethylated dsDNA substrate, using a polymerase and at least one target specific synthetic DNA oligonucleotide primer; (b) contacting the hemimethylated dsDNA substrate with an enzyme comprising both methyltransferase activity and ATP hydrolysis (ATPase) activity, thereby producing a symmetrically-methylated dsDNA product; and (c) removing or inactivating the enzyme comprising both methyltransferase activity and ATP hydrolysis activity; wherein an amplification cycle comprising steps (a)-(c) is repeated at least once. Typically steps (a)-(b) are conducted in a cation-containing buffer, as the presence of a cation (particularly Mg²⁺) is required for the functioning of the enzyme comprising both methyltransferase activity and ATPase activity.

The invention also provides a method of amplifying a target double-stranded DNA (dsDNA) containing symmetrically methylated nucleotides to produce an amplified methylated dsDNA product, comprising: (a) copying at least one methylated single-stranded DNA (ssDNA) to produce hemimethylated dsDNA substrate, using a polymerase and at least one target specific synthetic DNA oligonucleotide primer; (b) contacting the hemimethylated dsDNA substrate with an enzyme comprising both methyltransferase activity and ATPase activity, thereby producing a symmetrically-methylated dsDNA product; and wherein: (i) an amplification cycle comprising steps (a)-(b) is repeated at least once; and (ii) steps (a)-(b) are conducted in a cation-containing buffer. After step (b), said method may further comprise removing or inactivating the enzyme comprising both methyltransferase activity and ATPase activity. This may be referred to as step (c).

The invention provides a method of amplifying methylated DNA to produce an amplified dsDNA product, comprising: (a) copying at least one methylated single-stranded DNA (ssDNA) to produce hemimethylated dsDNA substrate, using a polymerase and at least one target specific synthetic DNA oligonucleotide primer; (b) contacting the hemimethylated dsDNA substrate with a DNA methyltransferase (DNMT), thereby producing a symmetrically-methylated dsDNA product; wherein: (i) an amplification cycle comprising steps (a)-(b) is repeated at least once; and (ii) the DNMT has at least 100-fold greater activity for hemimethylated DNA compared with unmethylated DNA. After step (b), said method may further comprise removing or inactivating the DNMT. This may be referred to as step (c).

The method may involve the hybridisation and/or ligation of one or more primer to at least one DNA strand.

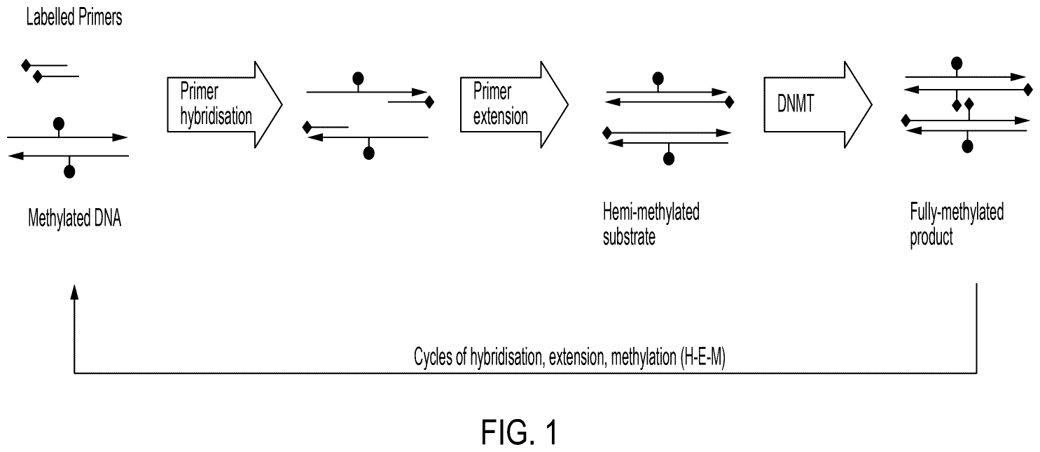

In some embodiments, the method may comprise or consist of cycles of (a1) Hybridisation of primers to the polynucleotide substrate; (a2) Extension of the primers to produce a hemimethylated substrate (wherein the original at least one polynucleotide (i.e. template polynucleotide strand) is methylated and the nascent polynucleotide (i.e. daughter strand(s) is unmethylated); and (b) Methylation of the nascent polynucleotide strands by a DNMT as described herein to produce a symmetrically-methylated double-stranded polynucleotide product. A single cycle of steps (a1), (a2) and (b) can be described as a hybridisation-extension-methylation (H-E-M) cycle. By way of non-limiting example, the invention provides a method of generating or amplifying methylated DNA, said method comprising or consisting of the following steps: (a1) hybridising a primer to one or more target region of at least one strand of DNA; (a2) extending the primers using a DNA polymerase to form a hemimethylated dsDNA substrate; (b) contacting the hemimethylated dsDNA substrate with a DNMT as described herein, thereby producing a symmetrically-methylated dsDNA product. This method is illustrated in the schematic in FIG. 1.

Step (a) of a method of the invention may comprise hybridising a primer to the at least one DNA strand, or a target sequence thereof. Where the at least one DNA strand comprises multiple target regions, one or more primers (e.g. two, three, four, five, six, seven, eight, nine, ten, 20, 50 or more primers) may be used. Each primer may have a unique sequence, or there may be overlap between one or more of the primer sequences. References herein to “a primer” encompass one or more primers unless expressly stated to the contrary. Following hybridisation, the primer(s) can be extended to produce one or more daughter strands comprising or consisting of complementary sequence to the target region(s).

In embodiments wherein the methylated DNA is dsDNA, step (a) may comprise hybridising a first primer to one strand of the dsDNA and hybridising a second primer to the other strand of the dsDNA, wherein the first and second primer are different (i.e. have different sequences). Wherein the methylated dsDNA comprises multiple target regions of interest, then a first primer and a second primer may be used for each target region. Whilst the first primer and second primer for a given target region will be different, the first primer and/or second primer for a given target region may be the same as or overlap with the first primer and/or second primer for one or more different target region. References herein to “a first primer” encompass one or more first primers unless expressly stated to the contrary. Similarly, references herein to “a second primer” encompass one or more second primers unless expressly stated to the contrary. Following hybridisation, the primer(s) can be extended to produce one or more daughter strands comprising or consisting of complementary sequence to the target region(s).

Preferably, the method uses a DNMT5 as described herein. Thus, in particularly preferred embodiments, the invention provides a method of generating or amplifying methylated DNA (e.g. dsDNA), said method comprising or consisting of the following steps: (a) using a DNA polymerase to amplify one or more target sequence within at least one DNA strand to form a hemimethylated double-stranded (dsDNA) substrate; and (b) contacting the hemimethylated dsDNA substrate with a DNMT5, thereby producing a symmetrically-methylated dsDNA product; wherein: (i) a cycle comprising steps (a)-(b) is repeated at least once; and (ii) the DNMT5 has at least 50-fold greater activity and/or specificity, preferably at least 100-fold greater activity and/or specificity, for hemimethylated DNA compared with unmethylated DNA. Again, step (a) of said method may comprise hybridising a primer (or one or more primers in the case of multiple target sequences) to the at least one DNA strand, or a target sequence thereof, as described above. In embodiments wherein the methylated DNA is dsDNA, step (a) of said method may comprise hybridising a first primer to one strand of the dsDNA and hybridising a second primer to the other strand of the dsDNA, wherein the first and second primer are different (i.e. have different sequences). Step (b) of said method then typically comprises or consists of contacting the hemimethylated dsDNA substrate with a DNMT5 as described herein, thereby producing a symmetrically-methylated dsDNA product.

A method of the invention may comprise a step of ligating or transposing a first primer to the at least one polynucleotide (e.g. DNA) strand prior to step (a). This ligation or transposition step typically is not part of the cycle of steps comprising steps (a)-(b). Thus, the ligation step is typically carried out once in a method of the invention. Any suitable transposase may be used to transpose a first primer. Any suitable ligase may be used to ligate a first primer. For the avoidance of doubt, any reference herein to using a ligase to ligating a first primer is equally and unreservedly applicable to using a transposase to transpose a first primer, unless expressly stated to the contrary.

Where the at least one DNA strand comprises multiple target regions, one or more primers (e.g. two, three, four, five, six, seven, eight, nine, ten, 20, 50 or more primers) may be ligated, one or more for each target region. Each primer may have a unique sequence, or there may be overlap between one or more of the primer sequences. References herein to “a primer” encompass one or more primers unless expressly stated to the contrary. Optionally step (a) may comprise hybridising a second primer to the first primer, wherein the first and second primer are different (i.e. have different sequences). Wherein the methylated dsDNA comprises multiple target regions of interest, then a first primer and a second primer may be used for each target region. Whilst the first primer and second primer for a given target region will be different, the first primer and/or second primer for a given target region may be the same as or overlap with the first primer and/or second primer for one or more different target region. References herein to “a first primer” encompass one or more first primers unless expressly stated to the contrary. Similarly, references herein to “a second primer” encompass one or more second primers unless expressly stated to the contrary. Following hybridisation, of the second primer(s), the second primer can be extended to produce one or more daughter strands comprising or consisting of complementary sequence to the target region(s).

Preferably, the method uses a DNMT5 as described herein. Thus, in particularly preferred embodiments, the invention provides a method of generating or amplifying methylated DNA (e.g. dsDNA), said method comprising or consisting of the following steps: ligating a first primer to the at least one DNA strand prior to step (a), (a) using a DNA polymerase to amplify one or more target sequence within at least one DNA strand to form a hemimethylated double-stranded (dsDNA) substrate; and (b) contacting the hemimethylated dsDNA substrate with a DNMT5, thereby producing a symmetrically-methylated dsDNA product; wherein: (i) a cycle comprising steps (a)-(b) is repeated at least once; and (ii) the DNMT5 has at least 50-fold greater activity and/or specificity, preferably at least 100-fold greater activity and/or specificity, for hemimethylated DNA compared with unmethylated DNA. The ligation step prior to step (a) of said method may comprise ligating a primer (or one or more primers in the case of multiple target sequences) to the at least one DNA strand, or a target sequence thereof, as described above. Optionally, step (a) of said method may comprise hybridising a second primer to the first primer, wherein the first and second primer are different (i.e. have different sequences). Step (b) of said method then typically comprises or consists of contacting the hemimethylated dsDNA substrate with a DNMT5 as described herein, thereby producing a symmetrically-methylated dsDNA product.

A cycle comprising steps (a)-(b) may result in linear amplification of the polynucleotide (e.g. DNA) substrate, or exponential amplification of the polynucleotide (e.g. DNA) substrate. By way of example, the primers may be suitable for standard PCR, enabling exponential amplification. Alternatively, the primers may be suitable for linked linear amplification (LLA), such that nonreplicable elements in the LLA primers renders the primer extension products (i.e. the nascent daughter strands) unusable as templates for further amplification, leading to linear accumulation of products. Linear amplification may be achieved using a single primer for a given target region within a polynucleotide substrate (e.g. DNA) or a given polynucleotide substrate (e.g. DNA).

A cycle comprising steps (a)-(b) can be repeated. By way of non-limiting example, the cycle of steps (a)-(b) may be carried out between 2-50 times, between 2-40 times, between 2-35 times, between 2-30 times, between 2-25 times, between 2-20 times, between 2-20 times, between 2-10 times, between 2-9 times, between 2-8 times, between 2-7 times, between 2-6 times, or between 2-5 times, between 10-50 times, between 10-40 times, between 20-50 times, or between 20-40 times. By way of further non-limiting example, the cycle of steps (a)-(b) may be carried out between 50 times, 45 times, 40 times, 39 times, 38 times, 37 times, 36 times, 35 times, 34 times, 33 times, 32 times, 31 times, 30 times, 29 times, 28 times, 27 times, 26 times, 25 times, 24 times, 23 times, 22 times, 21 times, 20 times, 19 times, 18 times, 17 times, 16 times, 15 times, 14 times, 13 times, 12 times, 11 times, 10 times, 9 times, 8 times, 7 times, 6 times, 5 times, 4 times, 3 times or 2 times. In other words, the cycle of steps (a)-(b) may be repeated any number of times as listed above.

The number of repeats of a cycles comprising steps (a)-(b) may be determined by one of ordinary skill in the art, without undue burden. The number of repeats of a cycle comprising steps (a)-(b) may be selected to amplify the polynucleotide substrate to produce the desired amount of product. The amount of product may be dependent on its intended use.

Alternatively or in addition, the number of repeats of the cycle comprising steps (a)-(b) may be selected based on the configuration of the method used (different configurations are discussed in more detail herein). By way of non-limiting example, when the DNMT5 is immobilised, then the number of cycles comprising steps (a)-(b) may be selected from between 2-40 times, between 2-35 times, between 2-20 times, between 2-10 times, between 2-9 times, between 2-8 times, between 2-7 times, between 2-6 times, or between 2-5 times, preferably between 2-10 times, more preferably between 2-7 times, even more preferably between 2-6 times. By way of further non-limiting example, when the DNMT5 is immobilised, then the number of cycles comprising steps (a)-(b) may be selected from 35 times, 34 times, 33 times, 32 times, 31 times, 30 times, 29 times, 28 times, 27 times, 26 times, 25 times, 24 times, 23 times, 22 times, 21 times, 20 times, 19 times, 18 times, 17 times, 16 times, 15 times, 14 times, 13 times, 12 times, 11 times, 10 times, 9 times, 8 times, 7 times, 6 times, 5 times, 4 times, 3 times or 2 times, preferably from 10 times, 9 times, 8 times, 7 times, 6 times, 5 times, 4 times, 3 times or 2 times. By way of further non-limiting example, when the DNMT5 is in solution, then the number of cycles of steps (a)-(b) may be selected from between 2-20 times, between 2-50 times, between 2-40 times, between 2-35 times, between 2-30 times, between 10-50 times, between 10-40 times, between 10-30 times, between 20-50 times, or between 20-40 times, preferably between 10-50 times, more preferably between 10-40 times, even more preferably between 20-40 times.

Steps (a) and (b) may be consecutive steps. By “consecutive step” is meant any step that comes after a first step, is excluded. On the other hand, and unless stated otherwise, “consecutive steps” do not exclude buffer exchange and/or dilution steps, for the purpose of modifying buffer conditions (i.e. concentration of salt; pH) of said compositions. Alternatively or additionally, unless stated otherwise, “consecutive steps” do not exclude steps to clean or purify the products of any one of steps (a)-(b) before the next step or cycle is carried out.

Alternatively, steps (a) and (b) may be non-consecutive steps. By “non-consecutive step” is meant a step that comes after a first step, is present.

A method of the invention may therefore include one or more additional step in addition to steps (a) and (b). By way of non-limiting example, a buffer exchange step, dilution/concentration step, and/or a clean-up step may be included between steps (a) and (b) and/or between step (b) of one cycle and the start of step (a) of the next cycle.

DNMT activity may be present throughout a complete cycle comprising steps (a)-(b). DNMT activity may be present throughout multiple repetitions of a cycle comprising steps (a)-(b), wherein the number of cycles is as defined above. DNMT activity may be present throughout the whole method, i.e. throughout all cycles comprising steps (a)-(b) conducted in the method. When DNMT activity is present throughout one or more cycle comprising steps (a)-(b), preferably the DNMT is a thermostable DNMT, as described herein. The use of a thermostable DNMT may facilitate its inclusion throughout the one or more cycle comprising steps (a)-(b), as the thermostable DNMT may remain structurally intact and, preferably functionally active, at the temperature required for primer extension by an appropriate polymerase.

Alternatively, the DNMT activity may not be present in step (a). The DNMT activity may be introduced for step (b), and may be removed from the symmetrically-methylated polynucleotide (e.g. DNA) product produced in step (b), (i) before starting a new cycle comprising steps (a)-(b), and/or (ii) at the end of the method. Removal of the DNMT activity may be defined as removal of sufficient DNMT activity to significantly reduce the methylation within a reaction vessel. Thus, removal of the DNMT activity may encompass removal of at least 70%, at least 80%, at least 85%, at least 90%, at least 85%, at least 96%, at least 97%, at least 98%, at least 99% or more of the DNMT activity, up to completed (100%) removal of the DNMT activity.

Thus, a method of the invention preferably comprises a step of removing or inactivating the DNMT after producing the symmetrically-methylated dsDNA product. In other words, a method of the invention preferably comprises a step of removing or inactivating the DNMT after step (b). This may be referred to as step (c). Removal of the DNMT is advantageous, as active DNMT can reduce amplification of a dsDNA template by a DNA polymerase. Therefore, in one-pot reactions as described herein (i.e, wherein the method steps take place in a single reaction vessel), removal or inactivation of DNMT may be preferred.

“DNMT activity” requires the presence of a functionally active DNMT, i.e. the DNMT must be capable of methylating the hemimethylated polynucleotide (e.g. DNA) substrate and have specificity for hemimethylated DNA compared with unmethylated DNA as described herein. DNMT activity may be removed by physically removing the DNMT protein from the reaction or reaction chamber. Physical removal of the DNMT (and hence DNMT activity) may be by any appropriate means. By way of non-limiting example, as described herein, the DNMT may be immobilised on beads, which are then removed from a reaction vessel, e.g. using centrifugation, or, if magnetic beads are used, a magnetic field. By way of further non-limiting example, if the DNMT is in solution and the primer(s) and/or polynucleotide substrate (e.g. DNA) are immobilised, then the DNMT activity may be removed by flow of the DNMT-containing solution away from the immobilised primer(s) and/or polynucleotide substrate (e.g. DNA), or by removal of the immobilised primers and/or polynucleotide substrate (e.g. DNA) from the DNMT-containing solution. Alternatively, DNMT activity may be removed functionally, such that the DNMT protein remains present in the reaction or reaction chamber, but is non-functional. Functional removal of DNMT activity may be by any appropriate means. By way of non-limiting example, DNMT activity may be removed by denaturing the DNMT, optionally by enzymatic, chemical or thermal denaturation, or by a reversible reaction with a DNMT antagonist. Thus, functional removal of DNMT activity may be referred to interchangeably as inactivating the DNMT. Non-limiting examples of enzymes which can be used to remove DNMT activity include proteinase K, elastase, aminopeptidase, pepsin, carboxypeptidase, trypsin, subtilisin and chymotrypsin.

The same DNMT activity may be removed and reintroduced in each cycle comprising steps (a)-(b). In other words, the DNMT activity may be introduced for a first step (b), be removed from the symmetrically-methylated polynucleotide (e.g. DNA) product produced in this first step (b), before starting a new (second) cycle comprising steps (a)-(b), and the same DNMT reintroduced for the second step (b) and so on. Alternatively, fresh DNMT may be introduced in each step (b). In other words, the DNMT may be replaced with fresh DNMT each cycle. By way of example, a first DNMT aliquot may be introduced for a first step (b), the first DNMT aliquot may be removed from the symmetrically-methylated polynucleotide (e.g. DNA) product produced in this first step (b), before starting a new (second) cycle comprising steps (a)-(b), and a fresh (second) aliquot of DNMT reintroduced for the second step (b) and so on. Alternatively, the same DNMT may be removed and reintroduced in each cycle of steps (a)-(b), and may optionally be supplemented with additional DNMT for one or more cycle. In other words, a first aliquot of DNMT may be introduced for a first step (b), the first aliquot of DNMT may be removed from the symmetrically-methylated polynucleotide (e.g. DNA) product produced in this first step (b), before starting a new (second) cycle comprising steps (a)-(b), and the first aliquot of DNMT may be reintroduced for the second step (b), supplemented with a second aliquot of DNMT and so on.

Wherein DNMT activity is removed by enzymatic, chemical or thermal denaturation, removal of the DNMT activity may require introduction of said DNMT in each cycle comprising steps (a)-(b). Typically, in such aspects, the DNMT may be replaced with fresh DNMT each cycle. The fresh DNMT activity may be added at any appropriate point in a cycle.

Polymerase activity may not be present in step (b). The polymerase may be introduced for step (a), and may be removed from the hemimethylated substrate (e.g. dsDNA) produced in step (a), before starting step (b). Removal of the polymerase activity may be defined as removal of sufficient polymerase activity to significantly reduce primer extension within a reaction vessel. Thus, removal of the polymerase activity may encompass removal of at least 70%, at least 80%, at least 85%, at least 90%, at least 85%, at least 96%, at least 97%, at least 98%, at least 99% or more of the polymerase activity, up to completed (100%) removal of the polymerase activity. “Polymerase activity” requires the presence of a functionally active polymerase, i.e. the polymerase must be capable of producing or extending a DNA strand as described herein. Polymerase activity may be removed by physically removing the polymerase protein from the reaction or reaction chamber. Physical removal of the polymerase (and hence polymerase activity) may be by any appropriate means. By way of non-limiting example, as described herein, the polymerase may be removed by proteinaseK, or by other enzymatic (e.g. using elastase, aminopeptidase, pepsin, carboxypeptidase, trypsin, subtilisin and chymotrypsin) or non-enzymatic methods prior to the introduction of DNMT and the start of step (b). Alternatively, polymerase activity may be removed functionally, such that the polymerase protein remains present in the reaction or reaction chamber, but is non-functional. Functional removal of polymerase activity may be by any appropriate means. By way of non-limiting example, polymerase activity may be removed by denaturing the polymerase, optionally by enzymatic, chemical or thermal denaturation, or by a reversible reaction with a polymerase antagonist.

Wherein polymerase activity is removed by enzymatic, chemical or thermal denaturation, removal of the polymerase activity may require introduction of polymerase in each cycle comprising steps (a)-(b). The same polymerase activity may be removed and reintroduced in each cycle comprising steps (a)-(b). In other words, the polymerase activity may be introduced for a first step (a) and be removed from the symmetrically-methylated polynucleotide (e.g. DNA) product produced in this first step (b), before starting a new (second) cycle comprising steps (a)-(b), and the same polymerase reintroduced for the second step (a) and so on. Alternatively, fresh polymerase may be introduced in each step (a). In other words, the polymerase may be replaced with fresh polymerase each cycle. By way of example, a first polymerase aliquot may be introduced for a first step (a), the first polymerase aliquot may be removed from the symmetrically-methylated polynucleotide (e.g. DNA) product produced in the first step (b), before starting a new (second) cycle comprising steps (a)-(b), and a fresh (second) aliquot of polymerase reintroduced for the second step (a) and so on. Alternatively, the same polymerase may be removed and reintroduced in each cycle of steps (a)-(b), and may optionally be supplemented with additional polymerase for one or more cycle. In other words, a first aliquot of polymerase may be introduced for a first step (a), the first aliquot of polymerase may be removed either from the hemimethylated double-stranded polynucleotide (e.g. dsDNA) substrate produced in the first step (a) or from the symmetrically-methylated polynucleotide (e.g. DNA) product produced in the first step (b), before starting a new (second) cycle comprising steps (a)-(b), and the first aliquot of polymerase may be reintroduced for the second step (a), supplemented with a second aliquot of polymerase and so on. Typically, the polymerase may be replaced with fresh polymerase each cycle. The fresh polymerase activity may be added at any appropriate point in a cycle.

Wherein DNMT activity and/or polymerase activity is removed by the addition of an agent, e.g. an enzyme, chemical, following removal of the DNMT activity and/or polymerase activity it is typically necessary to remove the activity of the agent such that DNMT and/or polymerase activity can be present for the next cycle comprising steps (a)-(b). In other words, to prevent fresh DNMT activity and/or polymerase activity also being removed by the agent, it is typically necessary to remove the activity of the agent after it has removed the DNMT activity and/or polymerase activity. The activity of the agent can be removed by any appropriate means.

Step (a) and optionally the ligation step prior to step (a) (when present) may be carried out in a first reaction vessel and step (b) carried out in a second reaction vessel. In other words, steps (a) and optionally the ligation step prior to step (a) (when present) may be carried out in the same reaction vessel and step (b) carried out in a different reaction vessel. Alternatively, each of the ligation step prior to step (a) (when present), step (a) and step (b) may be carried out in a separate reaction vessel. In other words, step (a) may be carried out in a first reaction vessel, step (b) in a second reaction vessel and, when present, the ligation step prior to step (a) may be carried out in a third reaction vessel. When steps (a) and (b) are carried out in the same reaction vessel, a method may be described as a “one-pot” reaction. Further alternatively, steps (a) and (b) may all be carried out in the same reaction vessel. When present, the ligation step prior to step (a) may also be carried out in this same reaction vessel. The number of reaction vessels used for steps (a)-(b) and the ligation step prior to step (a) (when present) may be selected based on the configuration of the method used (different configurations are discussed in more detail herein). Each reaction vessel may independently be selected from any suitable vessel type, e.g. a tube, microplate well or chamber in a flow chip. Suitable examples of reaction vessels are known in the art and can be selected by one of skill in the art without undue burden.

The hybridisation and/or ligation may be carried out in the same reaction vessel as the polymerase reaction (i.e. primer extension) and step (b) carried out in a second reaction vessel. In other words, hybridisation and/or ligation and the polymerase reaction (i.e. primer extension) may be carried out in the same reaction vessel and step (b) carried out in a different reaction vessel. Alternatively, each of the hybridisation and/or ligation and the polymerase reaction (i.e. primer extension) and step (b) may be carried out in a separate reaction vessel. For example, hybridisation and/or ligation may be carried out in a first reaction vessel, the polymerase reaction (i.e. primer extension) may be carried out in a second reaction vessel, step (b) in a third reaction vessel. Further alternatively, the hybridisation and/or ligation, the polymerase reaction (i.e. primer extension) and step (b) may all be carried out in the same reaction vessel. The number of reaction vessels used for the hybridisation and/or ligation, the polymerase reaction (i.e. primer extension) and step (b) may be selected based on the configuration of the method used (different configurations are discussed in more detail herein). Each reaction vessel may independently be selected from any suitable vessel type, e.g. a tube, microplate well or chamber in a flow chip. Suitable examples of reaction vessels are known in the art and can be selected by one of skill in the art without undue burden.

The DNMT, primer(s) and/or polynucleotide substrate (e.g. DNA) may be labelled. Whether each of these components is labelled is independent of whether each/any of the others are labelled. By way of example, the DNMT may be labelled, but the primer(s) may not be labelled, the primers may be labelled but the DNMT is not labelled, or both the DNMT and the primer(s) are labelled. Non-limiting examples of labels that may be used include ALFA-tags, AviTag, C-tag, CapTag, polyarginine or polyglutamate tags, FLAG-tag, His-tag, Myc-tag, SpyTag, a chitin binding domain (CBD)-tag, FLAG, V5, myc, HA, strep-tag, SNAP-tag, SdyTag, HUH-tag, MBP-tag, biotin and digoxigenin (DIG). An exemplary C-terminally-labelled DNMT5 is given in SEQ ID NO: 14. When the DNMT is labelled, the DNMT may be labelled at the N- and/or C-terminus, preferably at the C-terminus. In some preferred embodiments, the DNMT is labelled with biotin or a His-tag. Wherein the primers and/or polynucleotide substrate (e.g. DNA) are labelled, the label may be present at the 5′ or 3′ end of the primer(s) and/or polynucleotide substrate (e.g. DNA), preferably at the 5′ end. In some preferred embodiments, the primers and/or polynucleotide substrate (e.g. DNA) are labelled with biotin or DIG. When one or more first primers and one or more second primer are used, each of the first and/or second primer may be labelled. The label for the one or more first primers and/or one or more second primer may be selected independently. The same label may be used for one or more of the first primers and/or for one or more of the second primers.

The DNMT may be immobilised or in solution. Independently, the primer(s) and/or polynucleotide substrate (e.g. DNA) may be immobilised or in solution. By way of non-limiting example: (i) the DNMT and the primer(s) (e.g. one or more first primer) may be immobilised; (ii) the DNMT and the polynucleotide substrate (e.g. DNA) may be immobilised; (iii) the DNMT, the primer(s) (e.g. one or more first primer) and the polynucleotide substrate (e.g. DNA) may be immobilised; (iv) the DNMT and the primer(s) (e.g. one or more first primer) may be solution; (v) the DNMT and the polynucleotide substrate (e.g. DNA) may be solution; (vi) the DNMT, the primer(s) (e.g. one or more first primer) and the polynucleotide substrate (e.g. DNA) may be solution; (vii) the DNMT may be immobilised and the primer(s) (e.g. one or more first primer) may be in solution; (viii) the DNMT may be immobilised and the polynucleotide substrate (e.g. DNA) may be in solution; (ix) the DNMT may be immobilised and the primer(s) (e.g. one or more first primer) and polynucleotide substrate (e.g. DNA) may be in solution; (x) the DNMT may be in solution and the primer(s) (e.g. one or more first primer) may be immobilised; (xi) the DNMT may be in solution and the polynucleotide substrate (e.g. DNA) may be immobilised; or (xii) the DNMT may be in solution and the primer(s) (e.g. one or more first primer) and polynucleotide substrate (e.g. DNA) may be immobilised. Whether the DNMT, primer(s) (e.g. one or more first primer) and/or the polynucleotide substrate (e.g. DNA) is immobilised or in solution may be selected based on the configuration of the method used (different configurations are discussed in more detail herein).

Immobilisation means that the DNMT, primer(s) (e.g. one or more first primer) and/or polynucleotide substrate (e.g. DNA) is immobilised to a solid support. The solid support may be made of any appropriate material, e.g. streptavidin magnetic beads, silica or agarose. Typically the solid support itself is made of an inert substance, meaning that the material of the support has no effect or involvement on the method of the invention. The solid support may be of any suitable form, e.g. beads, a microplate well, a chamber in a flow cell or chip, a membrane, etc. Suitable examples of such solid supports are known in the art, and the selection of such a support is within the routine skill of one of ordinary skill in the art.

The DNMT, primer(s) (e.g. one or more first primer) and/or polynucleotide substrate (e.g. DNA) may be permanently immobilised. By “permanently immobilised”, it is meant that the immobilised component, whether DNMT, primer(s) (e.g. one or more first primer) and/or polynucleotide substrate (e.g. DNA), will remain immobilised unless and until an active step is taken to release the immobilised component, wherein said step requires one or reagent and/or condition not present within steps (a)-(b) of the claimed method. Typically permanent immobilisation means that the immobilised component will remain immobilised the throughout at least the relevant step of the method of the invention, preferably for one complete cycle comprising steps (a)-(b), and more preferably throughout the entire run of the method. This can have numerous technical and commercial advantages. By way of non-limiting example, the activity of non-thermostable DNMT may potentially be reduced, or even the enzyme denatured at the temperatures required for primer hybridisation and/or extension in step (a). Therefore, immobilisation of the DNMT may facilitate its removal at the end of step (b), before a new cycle comprising steps (a)-(b) begins, allowing for the DNMT to be reused in multiple cycles comprising steps (a)-(b) and reducing costs.

Permanent immobilisation may be carried out using any appropriate technique, and encompasses both covalent and non-covalent means. Again, suitable techniques are known in the art.

By way of non-limiting example, in embodiments where in the DNMT is immobilised, the DNMT may be tagged (as described herein) and the tag used to capture the component using an appropriate binding partner on the solid support. By way of non-limiting example, the DNMT may comprise a His-tag, allowing it to be immobilised on protein A beads or other protein A coated solid surfaces. By way of further non-limiting example, the DNMT may comprise a biotin tag, allowing it to be immobilised on streptavidin beads or other streptavidin coated solid surfaces. Other examples of suitable tags include strep-tag, SNAP-tag, FLAG, V5, myc, HA, CBD, and MBP, as described herein. In some preferred embodiments, the DNMT is immobilised to magnetic beads, particularly streptavidin or protein A coated magnetic beads.

Alternatively or in addition, by way of further non-limiting example, in embodiments where in the primer(s) (e.g. one or more first primer) and/or polynucleotide substrate (e.g. DNA) is immobilised, the primer(s) (e.g. one or more first primer) and/or polynucleotide substrate (e.g. DNA) may be tagged (as described herein) and the tag used to capture the component using an appropriate binding partner on the solid support. By way of non-limiting example, the primer(s) (e.g. one or more first primer) and/or polynucleotide substrate (e.g. DNA) may be tagged with biotin or DIG. Other examples of suitable tags include strep-tag, SNAP-tag, CBD, FLAG, V5, myc, HA and MBP, as described herein. The polynucleotide (e.g. DNA) may be captured based on its size, for example using commercially available systems such as SPRI/AMPure (from Beckman).

In embodiments wherein the primer(s) (e.g. one or more first primer) and/or polynucleotide substrate (e.g. DNA) are immobilised, the primer(s) (e.g. one or more first primer) and/or polynucleotide substrate (e.g. DNA) may be immobilised throughout steps (a)-(b) and between cycles comprising steps (a)-(b). In such embodiments, the primer(s) (e.g. one or more first primer) and/or polynucleotide substrate (e.g. DNA) may be immobilised on any type of support and by any means as described herein. Preferably the primer(s) (e.g. one or more first primer) and/or polynucleotide substrate (e.g. DNA) are immobilised on the surface of a flow cell or chip and/or within a chamber on a flow cell or chip. Alternatively, the primer(s) (e.g. one or more first primer) and/or polynucleotide substrate (e.g. DNA) may be captured and released between cycles comprising steps (a)-(b). In such embodiments, the primer(s) (e.g. one or more first primer) and/or polynucleotide substrate (e.g. DNA) may be immobilised on any type of support and by any means as described herein. Preferably, the primer(s) (e.g. one or more first primer) and/or polynucleotide substrate (e.g. DNA) may be immobilised on the surface of a flow cell or chip and/or within a chamber on a flow cell or chip, or on the surface of beads. In any of these embodiments, the DNMT may preferably be in solution, and may be (i) added after step (a) for step (b) to proceed; and/or (ii) removed after step (b). The DNMT may be contacted and/or removed from the primer(s) (e.g. one or more first primer) and/or polynucleotide substrate (e.g. DNA) using flow/injection techniques, examples of which are known in the art.

Step (a)—DNA Amplification

Step (a) of a method of the invention amplifies one or more target region of interest within at least one DNA strand to form a hemimethylated dsDNA substrate. The at least one DNA strand, which is referred to interchangeably herein as the polynucleotide substrate of the method may be a ssDNA, or may be one strand of a dsDNA. Preferably the at least one DNA strand is one strand of a dsDNA. In other words, step (a) of a method of the invention comprises or consists of copying at least one methylated single-stranded DNA (ssDNA) (a target region) to produce hemimethylated dsDNA substrate, using a polymerase and at least one target specific synthetic DNA oligonucleotide primer.

Amplification of the at least one DNA strand may be carried out using any appropriate technique, provided that amplification can be controlled such that the rates of extension by the DNA polymerase can be controlled such that methylation (in step (b) is balanced with the amplification. In this way, essentially all (e.g. at least 80%, at least 90%, at least 91%, at least 92%, at least 93%, at least 94%, at least 95%, at least 96%, at least 97%, at least 98%, at least 99% or more, up to 100%) of the hemimethylated dsDNA substrates produced in step (a) are methylated in step (b) to form a symmetrically-methylated dsDNA product.

Step (a) may comprise hybridising one or more primer to at least one DNA strand (i.e. the ssDNA), as described herein. Also as described herein, when the methylated DNA is dsDNA, step (a) may comprise hybridising a first primer to one strand of the dsDNA and hybridising a second primer to the other strand of the dsDNA, wherein the first and second primer are different (i.e. have different sequences). This hybridisation may be carried out using any appropriate reagents or combinations thereof, at suitable concentrations and/or any appropriate conditions (e.g. temperature and/or pH). Suitable reagents and/or reaction conditions can readily be determined by one of ordinary skill in the art.

Step (a) may comprise denaturation of the DNA to allow primer hybridisation to occur. Typically denaturisation is required when the at least one DNA strand is one strand of a dsDNA (i.e. the ssDNA), or in a second or subsequent cycle comprising steps (a)-(b), wherein the amplification in step (a) uses the symmetrically-methylated dsDNA product produced in step (b) of the previous cycle.

Thus, step (a) may comprise both denaturation of the polynucleotide substate (e.g. DNA) and hybridisation of one or more primers to the polynucleotide substate (e.g. DNA). A reference to hybridising one or more primers in step (a) therefore may encompass the denaturation of the polynucleotide substate (e.g. DNA) in addition to the hybridisation per se.

As described herein, wherein each cycle may comprise the addition of fresh polymerase activity to facilitate amplification of the one or more target region within at the least one DNA strand (ssDNA). The fresh polymerase activity may be added at any appropriate point in a cycle. By way of non-limiting example, fresh polymerase may be added before or at the start of step (a) of a cycle comprising steps (a)-(b). In particular, this may be required in “one-pot” reactions in which hybridisation and/or ligation, the polymerase reaction (i.e. primer extension) and step (b) may be carried out in the same reaction vessel, as presence of DNMT activity can reduce amplification efficiency.

When the polynucleotide substrate is double-stranded, e.g. dsDNA, the two strands of the polynucleotide substrate must be denatured to allow primer hybridisation to occur. Suitable techniques and conditions for substrate denaturation and primer hybridisation are routine in the art. By way of non-limiting example, denaturation of the polynucleotide substate (e.g. dsDNA) in step (a) may be carried out by: (i) thermal denaturation (also known as thermal cycling); or (ii) chemical denaturation.

For thermal cycling, the temperature of the polynucleotide substate (e.g. dsDNA) is increased to separate the two polynucleotide strands. The denaturation of the double-stranded polynucleotide substrate (e.g. dsDNA) may be carried out at any appropriate temperature, such as a temperature of between about 90° C. to about 100° C., between about 90° C. to about 98° C., between about 90° C. to about 96° C., between about 92° C. to about 100° C., between about 92° C. to about 98° C., between about 92° C. to about 96° C., between about 94° C. to about 100° C., between about 94° C. to about 98° C., between about 94° C. to about 96° C., preferably at a temperature of between about 92° C. to about 98° C., more preferably at a temperature of between about 94° C. to about 98° C. The denaturation of the double-stranded polynucleotide substrate (e.g. dsDNA) may be carried out at 95° C., 92° C., 93° C., 94° C., 96° C., 97° C. or 98° C., preferably at 95° C. or 94° C. A non-limiting example of thermal denaturation may comprise heating the polynucleotide substate (e.g. dsDNA) to 95° C. for up to 4 minutes, typically 95° C. for 30 seconds.

For chemical denaturation, a non-limiting exemplary protocol comprises contacting the polynucleotide substate (e.g. dsDNA) with NaOH (e.g. 1 μl of 1N NaOH), followed by neutralisation with HCl (e.g. 1 μl of 1M HCl).

Following denaturation, primer hybridisation may occur. Hybridisation may be carried out at any appropriate temperature, such as a temperature of between about 50° C. to about 80° C., between about 50° C. to about 75° C., between about 50° C. to about 72° C., between about 50° C. to about 70° C., between about 55° C. to about 80° C., between about 55° C. to about 75° C., between about 55° C. to about 72° C., or between about 55° C. to about 70° C., between about 50° C. to about 65° C., or between about 50° C. to about 60° C., preferably at a temperature of between about 55° C. to about 72° C., more preferably at a temperature of between about 55° C. to about 70° C.

Lower temperatures may be used to limit background (non-specific) amplification. For example, where a method is used to amplify a specific region of DNA (rather than using common primers to amplify a library), it may be desirable to limit background amplification, such that lower hybridisation temperatures may be used.

Alternatively, rather than thermal cycling requiring different temperatures for each of denaturation, hybridisation and primer extension, a method of the invention may cycle between two temperatures, a “low” temperature at which hybridisation and primer extension can occur, and a “high” temperature at which denaturation may occur.

As described herein, a method of the invention may comprise a step of ligating one or more first primer to the at least one DNA strand prior to step (a). Also as described herein, step (a) may further comprise hybridising a second primer to the first primer, wherein the first and second primer are different. This ligation and optional hybridisation may be carried out using any appropriate reagents or combinations thereof, at suitable concentrations and/or any appropriate conditions (e.g. temperature and/or pH). Suitable reagents and/or reaction conditions can readily be determined by one of ordinary skill in the art.

As for the primers used in the methods of the invention, these may be suitable for standard PCR or for LLA. It is within the routine practice of one of ordinary skill in the art to design suitable primers for a target sequence of interest. Thus, a primer of the invention may be of any appropriate length, e.g. between about 5-30 bases, between about 10-30 bases, between about 15-30 bases, between about 16-30 bases, between about 17-30 bases, between about 18-30 bases, between about 19-30 bases, between about 20-30 bases, between about 15-25 bases, between about 16-25 bases, between about 17-25 bases, between about 18-25 bases, between about 19-25 bases, between about 20-25 bases, between about 15-24 bases, between about 16-24 bases, between about 17-24 bases, between about 18-24 bases, or between about 19-24 bases. By way of example, standard PCR primers are typically between about 18-30 bases. By way of further example, random hexamer nucleotides may be used, e.g. random hexamers (i.e. a mixture of all possible combinations of six nucleotide sequences) can be used to facilitate amplification of the entire pool of nucleotides within a sample.

A primer of the invention may be of any appropriate G/C content, e.g. between about 40-60% G/C content.

Other standard features of primer design may be incorporated into primers according to the invention. By way of non-limiting example, a primer may have 1 or 2 G/C pairs at the 5′ and/or 3′ end, preferably 1 or 2 G/C pairs at both the 5′ and 3′ ends. By way of further limiting example, a primer may have a melting temperature (Tm) of between about 50-75° C., such as between about 50-70° C., between about 50-65° C., between about 50-60° C., between about 60-75° C. or between about 65-75° C. By way of further non-limiting example, primer pairs may have a Tm within 7° C., preferably 5° C. of each other.

Significantly, and as described above, as amplification occurs before methylation in methods of the invention, primers accordingly to the present invention may use four-base code (A, C, G and T), whereas conventional methods involve the hydrolytic deamination of non-methylated cytosines to uracils prior to amplification, requiring amplification primers to use only a three-base code (A, G and T).

Primers of the invention may comprise or consist of one or more (e.g. one, two, three, four, five, six, seven, eight, nine, 10, 11, 12, 13, 14, 15 or more, up to the whole length of the primer) modified nucleotides, as described herein. For example, primers may include one or more nucleotide which is modified to resist deamination. Thus, the invention encompasses the use of primers comprising protected cytosine groups.

A single primer (forward or reverse) may be used in a method of the invention for each target site within a polynucleotide substrate (e.g. DNA) or within a given polynucleotide substrate (e.g. DNA). Alternatively, primer pairs (e.g. forward and reverse) may be used in a method of the invention for each target site within a polynucleotide substrate (e.g. DNA) or a given polynucleotide substrate (e.g. DNA). Unless expressly stated to the contrary, reference herein to primers for use in methods of the invention encompasses both the use of a single primer and of primer pairs.

A primer of the invention, particularly in embodiments where one or more primer is ligated to the at least one DNA strand, may be a forked or Y-shaped adapter. As used herein, the terms “forked adapter” and “Y-shaped adapter” are used interchangeably and refer to a double stranded nucleic acid having a first end wherein the two strands are annealed to each other and a second end wherein the two strands are not annealed to each other. Examples of forked or Y-shaped adapters are described, for example, in U.S. Pat. No. 7,741,463, which is incorporated herein by reference.

A primer of the invention, particularly in embodiments where one or more primer is ligated to the at least one DNA strand, may be a linear primer, a hairpin primer or hairpin adapter. A hairpin primer may including a 5′ overhanging sequence portion (which may be generated by designing said hairpin to have regions of internal self-complementarity to encompass the 3′ end but with additional nucleotides at the 5′end which do not have complements on the 3′ strand) Alternatively, a hairpin primer may be “blunt-ended” such that the region of complementarity enables the formation of the intramolecular duplex including each of the 5′ and 3′ ends of the primer sequence, each nucleotide thus having a complementary nucleotide in the other arm of the intermolecular duplex.

A primer of the invention may comprise a tag or label, as described herein. Alternatively or in addition, a primer of the invention may comprise a barcode. A barcode may comprise or consist of a unique random oligonucleotide sequence (e.g. 12-14 nucleotides in length). A barcode may be used to uniquely tag individual DNA strands, allowing for barcoded strands to be individually identified. The use of barcodes for identification can be used to reduce sequencing errors introduced during next generation sequencing (NGS) library construction.

A primer of the invention, particularly one or more first primer for ligation to the at least one strand of DNA and/or one or more second primer for hybridising to said first primer may comprise a recognition sequence for a DNA modifying enzyme. Typically said DNA modifying enzyme is a DNA cleaving enzyme. Any reference herein to a DNA modifying enzyme applies equally and without reservation to DNA cleaving enzymes unless expresses stated to the contrary. Alternatively, a pair of such first primer and second primers may together comprise a double-stranded recognition sequence for a DNA modifying enzyme (e.g. a DNA cleaving enzyme). Said DNA modifying enzyme (DNA cleaving enzyme) is typically capable of creating a single-strand nick in a dsDNA. Suitable DNA modifying enzymes (e.g. DNA cleaving enzymes) are known in the art, and the selection of a suitable DNA modifying enzyme (e.g. DNA cleaving enzyme), and the corresponding recognition sequence is within the routine practice of one of ordinary skill in the art. By way of non-limiting examples, suitable DNA modifying enzymes (DNA cleaving enzymes) include type IIS restriction enzymes and/or Cas/Cas protein complexes. Type IIS restriction enzymes recognize asymmetric DNA sequences and cleave outside of their recognition sequence.

One or more primer may be hybridised and/or ligated to the 5′ end of the polynucleotide substrate and/or the 3′ end of the polynucleotide substrate.

The amplification of one or more target sequence in step (a) requires a DNA polymerase, which catalyses the synthesis of DNA. This amplification typically comprises the extension of one or more primer. Said primer may be a primer that has been hybridised to the at least one DNA strand (ssDNA), a primer that has been ligated to the at least one DNA strand, or a second primer that has hybridised to a first primer which is ligated to the at least one DNA strand.

Any suitable polymerase may be used. When the polynucleotide substate is DNA, a DNA polymerase is typically used. As described herein, amplification of the at least one DNA strand, and hence primer extension may be carried out using any appropriate technique, provided that amplification can be controlled such that the rates of extension by the DNA polymerase can be controlled such that methylation in step (b) is balanced with the amplification. By way of non-limiting example extension may be by PCR, strand displacement amplification or linked linear amplification (LLA).

Primer extension (i.e. the DNA polymerase reaction) produces a hemimethylated polynucleotide (e.g. DNA), wherein the nascent daughter polynucleotide strands produced by primer extension are unmethylated, but the strands of the polynucleotide substrate (e.g. DNA) retain their methylation.

Primer extension (i.e. the DNA polymerase reaction) may comprise generating a nascent polynucleotide strand comprising or consisting of from 10 base pairs (bp) to 10,000 bp of the polynucleotide substate (e.g. DNA), i.e. the amplicon is from 10 bp to 10,000 bp in length, such as from 100 bp to 1,000 bp, 100 bp to 500 bp, 100 bp to 400 bp, 100 bp to 350 bp, 100 bp to 300 bp, 100 bp to 250 bp, 100 bp to 200 bp, 100 bp to 150 bp, 150 bp to 1,000 bp, 150 bp to 500 bp, 150 bp to 400 bp, 150 bp to 350 bp, 150 bp to 300 bp, 150 bp to 250 bp, 150 bp to 200 bp. Preferably the amplicon is from 100 bp to 350 bp. More preferably the amplicon is from 150 bp to 350 bp.

As discussed in more detail herein in the context of preferred configurations, the invention provides embodiments in which primer extension (i.e. the DNA polymerase reaction) and step (b) are carried out in the same reaction vessel and/or in the same reaction buffer.

The polymerase used in the primer extension (i.e. the DNA polymerase reaction) of step (a) may retain at least partial activity in the presence of ATP and/or S-adenosyl methionine (SAM); and/or in methyltransferase buffer. Preferably the polymerase used in step (b) will retain at least 50%, at least 60%, at least 70%, at least 80%, at least 90%, at least 95%, at least 99% or more of its activity in the presence of ATP and/or S-adenosyl methionine (SAM); and/or in methyltransferase buffer. The activity of the polymerase may be measured using standard techniques, such as qPCR, or by using electrophoresis to assess the quantity of polynucleotide product after a specific number of cycles comprising steps (a)-(b).

Wherein the polymerase is a DNA polymerase, preferably the DNA polymerase is (i) a high-fidelity DNA polymerase; (ii) a strand-displacing polymerase; and/or (iii) thermostable. A DNA polymerase may be a high-fidelity and strand-displacing DNA polymerase; a high-fidelity and thermostable DNA polymerase; a strand-displacing polymerase and thermostable DNA polymerase; or a high-fidelity, strand-displacing and thermostable DNA polymerase. A high-fidelity DNA polymerase is one which has an error rate that is at least 10-times lower than Taq (which itself has an error rate of 2 mismatches per 3 KB). High-fidelity DNA polymerase are well-known in the art and commercially available. Non-limiting examples of high-fidelity DNA polymerases which may be used according to the invention include Q5® and Q5UR (both from New England BioLabs) and Phusion™ (from Thermofisher). Other non-limiting examples of DNA polymerases include Bst, Taq, phi29 DNA polymerase, Bsu and Klenow. The origin of the DNA polymerase is not particularly limited, e.g. the polymerase may be bacterial (e.g. Taq, Klenow or Bst), or the polymerase may be archaeal (e.g. Pfu, Q5).

Alternatively or in addition, the polymerase may be a thermostable (i.e. thermoresistant and/or thermofunctional) polymerase. A thermostable (i.e. thermoresistant and/or thermofunctional) polymerase of the invention may be thermofunctional at a temperature of up to 72° C., up to 75° C., up to 80° C., up to 85° C., up to 90° C., up to 94° C., up to 95° C., up to 98° C. or more. Alternatively or in addition, a thermostable (i.e. thermoresistant and/or thermofunctional) polymerase of the invention may be thermoresistant at a temperature of up to up to 72° C., up to 75° C., up to 80° C., up to 85° C., up to 90° C., up to 94° C., up to 95° C., up to 98° C. or more.

Alternatively or in addition, a DNA polymerase may be a strand-displacing DNA polymerase, which displace downstream DNA encountered during synthesis. Strand-displacing polymerases are well-known in the art and commercially available. Non-limiting examples of strand displacing DNA polymerases which may be used according to the invention include phi29 (from New England BioLabs). In addition, primer extension (i.e. the DNA polymerase reaction) in step (a) may be carried out using any other appropriate reagents or combinations thereof, at suitable concentrations and/or any appropriate conditions (e.g. temperature and/or pH).

By way of non-limiting example, primer extension (i.e. the DNA polymerase reaction) may be carried out at any appropriate temperature, such as a temperature of between about 20° C. to about 80° C., between about 25° C. to about 80° C., between about 30° C. to about 80° C., between about 37° C. to about 80° C., between about 40° C. to about 80° C., 50° C. to about 80° C., between about 50° C. to about 75° C., between about 50° C. to about 73° C., between about 50° C. to about 72° C., between about 60° C. to about 80° C., between about 60° C. to about 75° C., between about 60° C. to about 73° C., between about 60° C. to about 72° C., between about 65° C. to about 80° C., between about 65° C. to about 75° C., between about 65° C. to about 73° C., between about 65° C. to about 72° C., between about 68° C. to about 80° C., between about 68° C. to about 75° C., between about 68° C. to about 73° C., between about 68° C. to about 72° C., preferably at a temperature of between about 65° C. to about 75° C., more preferably at a temperature of between about 68° C. to about 72° C.

Step (b)—Methylation

The hemimethylated polynucleotide (e.g. dsDNA) produced in step (a) is used as the substrate for methylation by a DNMT in step (b). Thus, step (a), and particularly the primer extension (i.e. the DNA polymerase reaction) can be considered to produce a hemimethylated polynucleotide (e.g. dsDNA) substrate for step (b).

Step (b) can be carried out using any enzyme with methyltransferase activity and ATPase activity (i.e. any DNMT) as described herein, particularly a DNMT5 as described herein.

Step (b) requires the presence of a methyl donor group. This methyl donor group is used as the source of the methyl group which is added to the hemimethylated polynucleotide substrate (e.g. hemimethylated dsDNA) by the DNMT of the invention. Typically the methyl donor group is S-Adenosyl methionine (SAM), or a SAM analogue. Appropriate SAM analogues for use according to the invention are known in the art, such as carboxy SAM (CxSAM), CLICKable SAM, and 2-ynyl-SAM (bSAM). One or more modification (e.g. mutation) may be made to a DNMT (e.g. DNMT5) of the invention to facilitate use of a given SAM analogue as the methyl donor group used in step (b). For the avoidance of doubt, any reference herein to SAM encompasses SAM analogues unless explicitly stated otherwise.

As described herein, the SAM (or an appropriate SAM analogue) may be present in the initial buffer used for step (b) and/or may be added to the buffer during step (b) to facilitate the methylation reaction.

Depending on the precise reaction time and/or condition for step (b), then more than one addition of SAM (or an appropriate SAM analogue) and/or other reagents or methylation cofactors (e.g. Zn²⁺) may be required to enable production of a symmetrically-methylated polynucleotide (e.g. dsDNA) product. By way of non-limiting example, when step (b) is carried out for a long time (e.g. up to 10 minutes, up to 5 minutes, up to 4 minutes, up to 3 minutes, up to 2 minutes) and/or at elevated temperatures (e.g. up to 90° C., up to 94° C., up to 95° C., up to 98° C.), at least two, at least three or more, preferably two, additions of SAM (or an appropriate SAM analogue) and/or other reagents or methylation cofactors (e.g. Zn²⁺) may be required. The first addition of SAM (or an appropriate SAM analogue) may be in the initial buffer used for step (b), with one or more further addition of SAM (or an appropriate SAM analogue) during step (b). Alternatively, the first addition of SAM (or an appropriate SAM analogue) and the one or more further addition of SAM (or an appropriate SAM analogue) may each be added during step (b). Alternatively, a single addition of SAM (or an appropriate SAM analogue) and/or other reagents or methylation cofactors (e.g. Zn²⁺) may be made in step (b). By way of non-limiting example, a single addition of SAM (or an appropriate SAM analogue) and/or other reagents or methylation cofactors (e.g. Zn²⁺) may be added when step (b) is carried out at a temperature of up to 94° C. and/or for up to two minutes.

In addition, step (b) may be carried out using any other appropriate reagents or combinations thereof, at suitable concentrations and/or any appropriate conditions (e.g. temperature and/or pH).

By way of non-limiting example, step (b) may be carried out at any appropriate temperature, such as a temperature of between about 10° C. to about 60° C., between about 10° C. to about 50° C., between about 10° C. to about 40° C., between about 10° C. to about 37° C., between about 10° C. to about 30° C., between about 10° C. to about 25° C., between about 20° C. to about 60° C., between about 20° C. to about 50° C., between about 20° C. to about 40° C., between about 20° C. to about 37° C., between about 20° C. to about 30° C., or between about 20° C. to about 25° C., preferably at a temperature of between about 20° C. to about 40° C., more preferably at a temperature of between about 20° C. to about 37° C., still more preferably at a temperature of between about 25° C. to about 37° C., still more preferably at a temperature of about 25° C. Step (b) may be carried out at any appropriate temperature, such as about 25° C., about 30° C. or about 37° C.

In embodiments where one or more primer of the invention comprises a recognition sequence for a DNA modifying enzyme (i.e. DNA cleaving enzyme as described herein), step (b) may comprise using said DNA modifying enzyme (DNA cleaving enzyme) to generate a single-strand nick in a dsDNA. Nicking may occur before or after methylation occurs in step (b). In other words, the DNA modifying enzyme (DNA cleaving enzyme) may generate a nick in the hemimethylated dsDNA substrate and/or in the symmetrically-methylated dsDNA product. This nicking generates a substrate for the DNA polymerase in step (a) of the next cycle.

DNA modifying enzyme (DNA cleaving enzyme) activity is typically not present at the same time as the DNA polymerase activity. Having both the DNA modifying enzyme (DNA cleaving enzyme) and the DNA polymerase active at the same time will lead to the rate of the amplification of step (a) outpacing the methylation of step (b), such that not all of the hemimethylated DNA substrate produced in step (a) will be methylated in step (b).

Accordingly, DNA modifying enzyme (DNA cleaving enzyme) activity is typically not present during the primer extension (i.e. the DNA polymerase reaction). In other words, typically, DNA modifying enzyme (DNA cleaving enzyme) activity is not present during step (a). Alternatively, DNA modifying enzyme (DNA cleaving enzyme) activity may be present during the primer extension (i.e. the DNA polymerase reaction). The DNA modifying enzyme (DNA cleaving enzyme) activity may be introduced before or during step (b), and may be removed (i) following nicking of the hemimethylated dsDNA substrate or the symmetrically-methylated dsDNA product produced in step (b), (ii) before starting a new cycle comprising steps (a)-(b), and/or (iii) at the end of the method. Removal of the DNA modifying enzyme (DNA cleaving enzyme) activity may be defined as removal of sufficient DNA modifying enzyme (DNA cleaving enzyme) activity to significantly reduce the nicking reaction within a reaction vessel. Thus, removal of the DNA modifying enzyme (DNA cleaving enzyme) activity may encompass removal of at least 70%, at least 80%, at least 85%, at least 90%, at least 85%, at least 96%, at least 97%, at least 98%, at least 99% or more of the DNA modifying enzyme activity, up to completed (100%) removal of the DNA modifying enzyme activity.

“DNA modifying enzyme (DNA cleaving enzyme) activity” requires the presence of a functionally active DNA modifying enzyme (DNA cleaving enzyme), i.e. the DNA modifying enzyme must be capable of generating a single-strand nick in the hemimethylated dsDNA substrate and/or the symmetrically-methylated ds DNA product. DNA modifying enzyme (DNA cleaving enzyme) activity may be removed by physically removing the DNA modifying enzyme (DNA cleaving enzyme) protein from the reaction or reaction chamber. Physical removal of the DNA modifying enzyme (DNA cleaving enzyme) (and hence DNA modifying enzyme (DNA cleaving enzyme) activity) may be by any appropriate means. By way of non-limiting example, the DNA modifying enzyme (DNA cleaving enzyme) may be immobilised on beads, which are then removed from a reaction vessel, e.g. using centrifugation, or, if magnetic beads are used, a magnetic field. By way of further non-limiting example, if the DNA modifying enzyme (DNA cleaving enzyme) is in solution and the primer(s) and/or polynucleotide substrate (e.g. DNA) are immobilised, then the DNA modifying enzyme (DNA cleaving enzyme) activity may be removed by flow of the DNA modifying enzyme (DNA cleaving enzyme)-containing solution away from the immobilised primer(s) and/or polynucleotide substrate (e.g. DNA), or by removal of the immobilised primers and/or polynucleotide substrate (e.g. DNA) from the DNA modifying enzyme (DNA cleaving enzyme)-containing solution. Alternatively, DNA modifying enzyme (DNA cleaving enzyme) activity may be removed functionally, such that the DNA modifying enzyme (DNA cleaving enzyme) protein remains present in the reaction or reaction chamber, but is non-functional. Functional removal of DNA modifying enzyme (DNA cleaving enzyme) activity may be by any appropriate means. By way of non-limiting example, DNA modifying enzyme (DNA cleaving enzyme) activity may be removed by denaturing the DNA modifying enzyme (DNA cleaving enzyme), optionally by chemical or thermal denaturation, or by a reversible reaction with a DNA modifying enzyme antagonist.

The same DNA modifying enzyme (DNA cleaving enzyme) activity may be removed and reintroduced before/during each step (b) of a cycle comprising steps (a)-(b). In other words, the DNA modifying enzyme (DNA cleaving enzyme) activity may be introduced for a first step (b), be removed from the hemimethylated dsDNA substrate or the symmetrically-methylated dsDNA following nicking, before starting a new (second) cycle comprising steps (a)-(b), and the same DNA modifying enzyme (DNA cleaving enzyme) reintroduced for the second step (b) and so on. Alternatively, fresh DNA modifying enzyme (DNA cleaving enzyme) may be introduced in each step (b). In other words, the DNA modifying enzyme (DNA cleaving enzyme) may be replaced with fresh DNA modifying enzyme (DNA cleaving enzyme) each cycle. By way of example, a first DNA modifying enzyme (DNA cleaving enzyme) aliquot may be introduced for a first step (b), the first DNA modifying enzyme (DNA cleaving enzyme) aliquot may be removed from the hemimethylated dsDNA substrate or symmetrically-methylated dsDNA product after nicking, before starting a new (second) cycle comprising steps (a)-(b), and a fresh (second) aliquot of DNA modifying enzyme (DNA cleaving enzyme) reintroduced for the second step (b) and so on. Alternatively, the same DNA modifying enzyme (DNA cleaving enzyme) may be removed and reintroduced in each cycle of steps (a)-(b), and may optionally be supplemented with additional DNA modifying enzyme (DNA cleaving enzyme) for one or more cycle. In other words, a first aliquot of DNA modifying enzyme (DNA cleaving enzyme) may be introduced for a first step (b), the first aliquot of DNA modifying enzyme (DNA cleaving enzyme) may be removed from the hemimethylated dsDNA substrate or symmetrically-methylated dsDNA product following nicking, before starting a new (second) cycle comprising steps (a)-(b), and the first aliquot of DNA modifying enzyme (DNA cleaving enzyme) may be reintroduced for the second step (b), supplemented with a second aliquot of DNA modifying enzyme (DNA cleaving enzyme) and so on.

### Additional Steps

A method of the invention may comprise one or more further step in addition to steps (a) and (b) and any optional buffer exchange/dilution or concentration steps. One or more additional step may be in relation to processing and/or analysing the symmetrically-methylated polynucleotide (e.g. dsDNA) product produced in step (b). Alternatively, one or more additional step may be in relation to preparing a polynucleotide substrate (e.g. DNA) for use in step (a).

The method may further comprise one or more steps of capturing a methylated single-stranded polynucleotide (e.g. ssDNA) and/or copying said methylated single-stranded polynucleotide (e.g. ssDNA) to produce a double-stranded polynucleotide (e.g. dsDNA). Said one or more steps are typically carried out before step (a) in a method of the invention, and the resulting double-stranded polynucleotide (e.g. dsDNA) used as the substrate in step (a) of the method.

Alternatively or in addition, the method may further comprise one or more steps of detecting methylcytosine (mC) in the symmetrically-methylated polynucleotide (e.g. dsDNA). As used herein, detection of mC may comprise or consist of detecting, locating and/or quantifying mC within either or both strands of the symmetrically-methylated polynucleotide (e.g. dsDNA).

Thus, a method of the invention may further comprise one or more steps of detecting mC following step (b). Typically said one or more steps of detecting mC is carried out following the final step (b) in the final repetition of the cycle comprising steps (a)-(b). By way of non-limiting example, the method may comprise 6 complete cycles each comprising steps (a)-(b), with one or more step of detecting mC carried out after step (b) of the sixth cycle (i.e. after the sixth repetition of step (b)).

Alternatively or in addition, the method may further comprise one or more step to oxidise one or more mC in the symmetrically-methylated polynucleotide (e.g. dsDNA) produced in step (b). Oxidation converts mC to hydroxymethylcytosine (hmC), 5-Carboxylcytosine (caC) or 5-Formylcytosine (fC). Oxidation of mC to hmC, caC or fC may facilitate detection of methylated cytosine residues, and hence epigenetic analysis.

Oxidation of mC to hmC may be carried out using any appropriate means. By way of non-limiting example, a Ten-eleven translocation (TET) enzyme may be used to oxidise mC to hmC, caC or fC. Oxidation of mC may be carried out before detection of mC, simultaneously with detection of mC or after detection of mC. Preferably, oxidation of mC is carried out before detection of mC.

Any appropriate means may be used to detect mC and/or hmC, including both enzymatic and non-enzymatic methods. By way of non-limiting example, immunoprecipitation, endonuclease digestion, sodium bisulfite treatment (including whole genome bisulfite sequencing), TET-assisted pyridine borane sequencing (TAPS), enzymatic-methyl sequencing (Em-seq), or direct read-out of DNA methylation (e.g. using nanopore sequencing) may be used. Suitable protocols are known in the art, and are within the routine practice of one of ordinary skill in the art. For example, Chapter 16 of Jeltsch and Jurkowska (2022) DNA Methyltransferases—Role and Function, 2^{nd} Ed, Switzerland: Springer, which is herein incorporated by reference in its entirety. Other suitable methods include the single-enzyme methylation sequencing described in Vaisvila et al., 2023 (bioRxiv 2023.06.29.547047; doi: https://doi.org/10.1101/2023.06.29.547047) and the direct enzymatic sequencing technique described in Wang et al. (Nat Chem Biol 2023, https://doi.org/10.1038/s41589-023-01318-1), both of which are herein incorporated by reference in their entirety. Preferably, the method of detection can differentiate between C (unmethylated), mC and hmC. In some preferred embodiments, the mC are detected using an array, nanopores, next-generation sequencing and/or restriction enzyme digest.

The two strands of the symmetrically-methylated polynucleotide (e.g. dsDNA) product produced in step (b) may be processed and/or analysed together, or in an identical manner. Alternatively, two strands of the symmetrically-methylated polynucleotide (e.g. dsDNA) product produced in step (b) may be separated and the original strands processed in a different manner to the daughter strands (i.e. the copied strands produced in step (a) of the method). Thus, the identity of the modified cytosines in the original strand and the copied strands may be determined separately. As hydroxymethylation is not copied from the original strand(s) to the copied strand(s), in the absence of an active step of oxidation, hmC will be present only in the original strand(s) and not the copied strand(s). The hmC can then be isolated using appropriate techniques and analysed separately. Appropriate techniques for isolating and/or analysing hmC are well known in the art. For example, enzymatic-methylation sequencing (EM-Seq) uses TET and β-glucosyltransferase (BGT) enzymes before enzymatic deamination to achieve a readout akin to bisulfite merging 5 mC and 5hmC. Other methods include APOBEC-coupled epigenetic sequencing (ACE-Seq), which uses a DNA deaminase, APOBEC3A (A3A), to selectively deaminate unmodified Cs and mCs, while leaving protected hmCs unconverted; and immunoprecipitation with an antibody specific for hmC. Other suitable techniques are described, for example in Chapter 16.9 of Jeltsch and Jurkowska (2022) DNA Methyltransferases—Role and Function, 2^{nd} Ed, Switzerland: Springer, which is herein incorporated by reference in its entirety. The remaining strand(s), i.e. the copied strand(s) may then be analysed, e.g. for mC using any appropriate techniques, such as those described herein (e.g. in the preceding paragraph).

The symmetrically-methylated polynucleotide (e.g. dsDNA) product produced in in a step (b) or following a final step (b) may be divided into two or more samples (e.g. two, three, four, five, six, seven, eight, nine, ten or more samples). These two or more samples may be analysed using the same or different techniques. By way of non-limiting example, if the symmetrically-methylated polynucleotide (e.g. dsDNA) product is divided into two samples, each sample may be analysed using a different technique. By way of further non-limiting example, if the symmetrically-methylated polynucleotide (e.g. dsDNA) product is divided into three samples, two of the samples may be analysed by the same technique and the remaining three samples may each be analysed using a different technique, which is different to the technique used to analyse the first two samples. The different techniques may be different techniques for detecting mC.

Alternatively or in addition, step (b) of the method may involve the use of a SAM analogue which adds a protecting group at the same time as the methyl group is transferred. In this way, the resulting mC may be protected. This may be useful, for example, by allowing deamidation of unmethylated cytosine residues (e.g. by APOBEC3A (A3A)), whilst leaving the protected mC. Thus, the use of SAM analogues comprising a protecting group may facilitate downstream sequencing. For example, a modified CxSAM can be used to incorporate a carboxymethyl group instead of a methyl group at the C5 position of cytosine, and the resulting 5cxmC base is resistant to deamination by bacterial deaminase enzymes (Wang et al., Nature Chemical Biology, 2023). Thus deamination of target DNA will preserve the cytosine base pairing with guanine for 5cxmC bases, and not be deaminated to uracil by these enzymes. These modifications have utility in the decoding of the DNA sequence and the identification of different cytosine bases in the target DNA.

### Buffers

A separate reaction buffer may be used for any of primer hybridisation, primer ligation, primer extension and/or methylation. In other words: (i) primer hybridisation and/or primer ligation, primer extension, and step (b) may each be carried out in a different reaction buffer; (ii) primer hybridisation and/or primer ligation and primer extension may be carried out in the same reaction buffer and step (b) may be carried out in a different reaction buffer; (iii) primer hybridisation and/or primer ligation and step (b) may be carried out in the same reaction buffer and primer extension may be carried out in a different reaction buffer; or (iv) primer extension and step (b) may be carried out in the same reaction buffer, and primer hybridisation and/or primer ligation may be carried out in a different reaction buffer. Preferably, if at least two different reaction buffers are used, primer hybridisation and/or primer ligation and primer extension are carried out in the same reaction buffer and step (b) is carried out in a different reaction buffer. Wherein a different buffer is used for any of primer hybridisation, primer ligation, primer extension and/or step (b), said buffer may be optimised for that specific step.

Steps (a)-(b) may each be carried out in a different reaction buffer; or steps (a) and (b) may be carried out in the same reaction buffer Wherein different buffers are used for steps (a) and (b), said buffer may be optimised for that specific step.

Wherein the same reaction buffer is used for any of primer hybridisation and/or primer ligation, primer extension and/or step (b), the reaction buffer may be replenished before commencement of primer hybridisation and/or primer ligation, primer extension and/or step (b), during primer hybridisation and/or primer ligation, primer extension and/or step (b), and/or before a new cycle comprising steps (a)-(b) is begun. Alternatively or in addition, wherein the same reaction buffer is used for any of primer hybridisation and/or primer ligation, primer extension and/or step (b), particularly for all of primer hybridisation and/or primer ligation, primer extension and/or step (b), the reaction buffer may be supplemented with one or more different components before commencement of primer hybridisation and/or primer ligation, primer extension and/or step (b), during primer hybridisation and/or primer ligation, primer extension and/or step (b), and/or before a new cycle comprising steps (a)-(b) is begun.

Wherein the same reaction buffer is used for steps (a) and (b), the reaction buffer may be replenished before commencement of any step, during any step, and/or before a new cycle comprising steps (a)-(b) is begun. Alternatively or in addition, wherein the same reaction buffer is used for steps (a) and (b), the reaction buffer may be supplemented with one or more different components before commencement of any step, during any step, and/or before a new cycle comprising steps (a)-(b) is begun.

Wherein the same reaction buffer is used for one or more of primer hybridisation and/or primer ligation, primer extension and/or step (b) (e.g. for primer hybridisation and primer extension, for primer hybridisation and step (b), for primer extension and step (b), for primer ligation and primer extension, for primer ligation and step (b), for primer hybridisation, primer ligation and primer extension, or for primer hybridisation, primer ligation and step (b)) then preferably (i) the polymerase; (ii) the DNMT; or (iii) the polymerase and the DNMT each have at least partial activity. If present, a DNA modifying enzyme (DNA cleaving enzyme) may also have at least partial activity. Partial activity may be defined at least at least 50%, at least 60%, at least 70%, at least 80%, at least 90%, at least 95%, at least 97% or more of the activity of the wild-type enzyme (polymerase, DNMT and/or DNA modifying enzyme (DNA cleaving enzyme), respectively).

Wherein the same reaction buffer is used for steps (a) and (b), then preferably (i) the polymerase; (ii) the DNMT; or (iii) the polymerase and the DNMT each have at least partial activity. If present, a DNA modifying enzyme (DNA cleaving enzyme) may also have at least partial activity. Partial activity may be defined at least at least 50%, at least 60%, at least 70%, at least 80%, at least 90%, at least 95%, at least 97% or more of the activity of the wild-type enzyme (polymerase, DNMT and/or DNA modifying enzyme (DNA cleaving enzyme), respectively).

Standard reaction buffers and components thereof for use with DNA polymerases, DNMTs and/or DNA modifying enzymes (DNA cleaving enzymes) are known in the art and can be readily selected by one of ordinary skill in the art. By way of non-limiting example, some typical buffers are listed below for reference. The concentrations of each component stated below are the final reaction concentrations, and typically buffers would be made as concentrated stock solutions to be diluted with water prior to use.

Buffer M: 50 mM Tris-HCl pH 8, 25 mM NaCl, 10% Glycerol, 2 mM DTT, 1 mM ATP, 1 mM MgCl2 and 1.6 mM SAM.

NEBuffer 2 (New England Biolabs catalog number B7002) diluted to a 1× working concentration: 50 mM NaCl, 10 mM Tris-HCl, 10 mM MgCl2, 100 microg/ml Recombinant Albumin, 160 microM SAM, PH 7.9@25° C.

Cutsmart (New England Biolabs catalog number B6004S) diluted to a 1× working concentration: 50 mM Potassium Acetate, 20 mM Tris-acetate, 10 mM Magnesium Acetate, 100 microg/ml Recombinant Albumin, 160 microM SAM, PH 7.9@25° C.

These exemplary buffers are optimised for DNMT, i.e. they may be referred to as methyltransferase buffers, but may also be used for primer hybridisation and/or ligation, and/or primer extension. In other words, any one of these buffers may be used in step (a) as well as step (b), if appropriately supplemented with the necessary reagents for primer hybridisation and/or ligation, and/or primer extension, as appropriate.

As described herein, a DNMT (e.g. DNMT5) of the invention has ATPase activity. Cations, particularly magnesium ions (Mg²⁺) are a cofactor for ATP hydrolysis to ADP by this domain and hence are a requirement for methylation by the DNMT. Cations, particularly magnesium ions (Mg²⁺), are also a cofactor for DNA polymerase activity. Therefore, for a one-pot reaction of the invention to work optimally, the concentration of cations, particularly Mg²⁺has to support both the DNMT and DNA polymerase. This is in contrast to other commonly used DNMTs, such as DNMT1, which can function in the absence of Mg²⁺, such that published methylation protocols using such DNMTs can involve chelation of any Mg²⁺within the reaction mixture to increase DNA methylation.

Accordingly, a reaction buffer for use according to the present invention may be supplemented with cations, particularly Mg²⁺. In particular, steps (a) and (b) may be conducted in a cation-containing buffer. A method of the invention may exclude the addition of a chelating agent (e.g. EDTA) in any of the steps thereof.

The concentration of cations, particularly Mg²⁺in the reaction buffer may be increased compared with the concentration of cations, particularly Mg²⁺in a standard buffer used for DNA polymerase reactions. Alternatively, or in addition, the concentration of cations, particularly Mg²⁺in the reaction buffer may be increased compared with the concentration of cations, particularly Mg²⁺in a standard buffer used for DNMT reactions, particularly compared with the concentration of Mg²⁺in a buffer used for a standalone methylation reactions (i.e. not linked to a DNA polymerase reaction) using the same DNMT. In particular, the concentration of cations, particularly Mg²⁺in the reaction buffer may need to be increased wherein the same reaction buffer is used for any of primer hybridisation and/or primer ligation, primer extension and/or step (b), and even more particularly when the method of the invention is a one-pot reaction in which primer hybridisation and/or primer ligation, primer extension and step (b) are carried out in the same reaction vessel.

Whilst Mg²⁺is preferred, other cations may be used as a replacement for Mg²⁺, such as Mn²⁺. Therefore, the disclosure herein about supplementing reaction buffers with Mg²⁺applies equally and without reservation to other suitable cations, such as Mn²⁺.

The concentration of cations, particularly Mg²⁺in the reaction buffer may be increased by the addition of any appropriate cationic salt. In the case of magnesium, any appropriate magnesium salt, such as MgSO₄, MgCl₂, MgCO₃ or MgO. In some preferred embodiments, MgSO₄ is used.

The concentration of cations, particularly Mg²⁺in the reaction buffer required for optimal DNMT and polymerase activity will depend on numerous factors, including the concentration of ATP present and the specific polymerase use. A person skilled in the art will appreciate that for optimal activity, an excess of cations, particularly Mg²⁺is required compared with the concentration of ATP. It will be within the routine practice of one of ordinary skill in the art to select a suitable concentration of cations, particularly Mg²⁺to provide the required excess, without any undue burden.

Thus, wherein the same reaction buffer is used for any of primer hybridisation and/or primer ligation, primer extension and/or step (b), the reaction buffer may be replenished before commencement of primer hybridisation and/or primer ligation, primer extension and/or step (b), during primer hybridisation and/or primer ligation, primer extension and/or step (b) and/or before a new cycle comprising steps (a)-(b) is begun, such that the cations, particularly Mg²⁺ concentration in the reaction buffer is sufficient to support both DNMT activity and polymerase activity.

Alternatively or in addition, wherein the same reaction buffer is used for any of primer hybridisation and/or primer ligation, primer extension and/or step (b), particularly for all of primer hybridisation and/or primer ligation, primer extension and/or step (b), the reaction buffer may be supplemented with cations, particularly Mg²⁺before commencement of primer hybridisation and/or primer ligation, primer extension and/or step (b), during primer hybridisation and/or primer ligation, primer extension and/or step (b), and/or before a new cycle comprising steps (a)-(b) is begun, such that the cations, particularly Mg²⁺ concentration in the reaction buffer is sufficient to support both DNMT activity and polymerase activity.

Wherein the same reaction buffer is used for steps (a) and (b), the reaction buffer may be replenished before commencement of any step, during any step, and/or before a new cycle comprising steps (a)-(b) is begun, such that the cations, particularly Mg²⁺ concentration in the reaction buffer is sufficient to support both DNMT activity and polymerase activity. Alternatively, or in addition, wherein the same reaction buffer is used for steps (a) and (b), the reaction buffer may be supplemented with cations, particularly Mg²⁺before commencement of any step, during any step, and/or before a new cycle comprising steps (a)-(b) is begun, such that the cations, particularly Mg²⁺ concentration in the reaction buffer is sufficient to support both DNMT activity and polymerase activity.

Accordingly, a reaction buffer for use according to the present invention may be supplemented with ATP and/or dATP.

As described herein, a DNMT of the invention requires a methyl group donor, typically a SAM or SAM analogue in order to methylate DNA. Accordingly, a reaction buffer for use according to the present invention may be supplemented with SAM and/or a SAM analogue.

Wherein a separate reaction buffer is used for step (b), said buffer may be optimised for that specific step to have a concentration of SAM and/or SAM analogue that supports methylation.

Alternatively, wherein the same reaction buffer is used for any of primer hybridisation and/or primer ligation, primer extension and/or step (b), the reaction buffer may be replenished before commencement of primer hybridisation and/or primer ligation, primer extension and/or step (b), during primer hybridisation and/or primer ligation, primer extension and/or step (b) and/or before a new cycle comprising steps (a)-(b) is begun, such that the concentration of SAM and/or SAM analogue in the reaction buffer is sufficient to support DNMT activity.

Alternatively or in addition, wherein the same reaction buffer is used for any of primer hybridisation and/or primer ligation, primer extension and/or step (b), particularly for all of primer hybridisation and/or primer ligation, primer extension and/or step (b), the reaction buffer may be supplemented with SAM and/or SAM analogue before commencement of primer hybridisation and/or primer ligation, primer extension and/or step (b), during primer hybridisation and/or primer ligation, primer extension and/or step (b), and/or before a new cycle comprising steps (a)-(b) is begun, such that the concentration of SAM and/or SAM analogue in the reaction buffer is sufficient to support DNMT activity.

Wherein the same reaction buffer is used for steps (a) and (b), the reaction buffer may be replenished before commencement of any step, during any step, and/or before a new cycle comprising steps (a)-(b) is begun, such that the concentration of SAM and/or SAM analogue in the reaction buffer is sufficient to support DNMT activity. Alternatively or in addition, wherein the same reaction buffer is used for steps (a) and (b), the reaction buffer may be supplemented with SAM and/or SAM analogue before commencement of any step, during any step, and/or before a new cycle comprising steps (a)-(b) is begun, such that the concentration of SAM and/or SAM analogue in the reaction buffer is sufficient to support DNMT activity.

### Exemplary Method Configurations

As described herein, the present inventors have devised a method of amplifying DNA in which methylation of cytosine residues in the template strand are accurately copied to the daughter strand. This new foundational technique can be applied to multiple different method configurations, all of which rely on the same underpinning technical concept and so are encompassed by the present invention. By way of exemplification, some preferred, but non-limiting examples of method configurations falling within the present invention are described herein.

In some embodiments, the DNMT may be immobilised, e.g. on beads, and the one or more primer and/or polynucleotide substrate (e.g. DNA) are in solution. Step (a) is carried out in one reaction vessel, containing the one or more primer, polynucleotide substrate (e.g. DNA), polymerase and other amplification reagents. Following the completion of step (a), the hemimethylated polynucleotide product (e.g. dsDNA) produced in step (a) is transferred to a second reaction vessel comprising the immobilised DNMT and reagents required for methylation. Following completion of step (b), the symmetrically-methylated polynucleotide product (e.g. dsDNA) is isolated from the immobilised DNMT. If one or more further cycle comprising steps (a)-(b) is required, then the isolated symmetrically-methylated polynucleotide product (e.g. dsDNA) is returned to the first reaction vessel for step (a) of the next cycle to begin.

These configurations may be described as “two-pot” reactions, due to the use of two reaction vessels, and/or “ping-pong” reactions, as the polynucleotide is moved back and forth between two reaction vessels. Such configurations can keep the polymerase and DNMT separate, preventing any interference between the enzymes or the conditions and/or reagents required for each to function. These configurations also allow for optimisation of reagents and/or conditions required for each step. Thus, in some embodiments, the invention comprises a method of amplifying a methylated double-stranded polynucleotide (e.g. dsDNA), method comprising or consisting of the following steps: (a) using a DNA polymerase to amplify one or more target region within at least one DNA strand in a first reaction vessel to form a hemimethylated polynucleotide (e.g. dsDNA) substrate; (b1) transferring the hemimethylated polynucleotide (e.g. dsDNA) substrate to a second reaction vessel; and (b2) contacting the hemimethylated polynucleotide (e.g. dsDNA) substrate with a DNMT (e.g. DNMT5) in the second reaction vessel, thereby producing a symmetrically-methylated polynucleotide (e.g. dsDNA) product. Said method may comprise repeating a cycle of steps (a), (b1) and (b2) to produce a desired amount of the fully-methylated polynucleotide (e.g. dsDNA) product. The number of cycle repeats may be as described herein. Said method may optionally further include a step of ligating one or more first primer to the at least one DNA strand prior to step (a), said ligation step typically not forming part of the cycle of steps, but instead being carried out only once at the start of the method. Amplification in step (a) may be by any appropriate means. By way of non-limiting example, the invention comprises a method of amplifying a methylated double-stranded polynucleotide (e.g. dsDNA), method comprising or consisting of the following steps: (a) hybridising a primer to each strand of the double-stranded polynucleotide (e.g. dsDNA) in a first reaction vessel and extending the primers in the first reaction vessel using a (DNA) polymerase to form a hemimethylated polynucleotide (e.g. dsDNA) substrate; (b1) transferring the hemimethylated polynucleotide (e.g. dsDNA) substrate to a second reaction vessel; and (b2) contacting the hemimethylated polynucleotide (e.g. dsDNA) substrate with a DNMT (e.g. DNMT5) in the second reaction vessel, thereby producing a symmetrically-methylated polynucleotide (e.g. dsDNA) product. Said method may comprise repeating a cycle of steps (a), (b1) and (b2) to produce a desired amount of the symmetrically-methylated polynucleotide (e.g. dsDNA) product. The number of cycle repeats may be as described herein.

In alternative embodiments, this “two-pot” configuration could be conducted without immobilising the DNMT. Here, DNMT activity could be removed either by functionally removing the DNMT activity, or by physically removing the DNMT protein (i.e. removing or inactivating the DNMT), both of which are described in more detail here. Otherwise, this configuration could remain unchanged. Thus, step (a) may be carried out in one reaction vessel, containing the one or more primer, polynucleotide substrate (e.g. DNA), polymerase and other amplification reagents. Following the completion of step (a), the hemimethylated polynucleotide product (e.g. dsDNA) produced in step (a) is transferred to a second reaction vessel comprising the DNMT and reagents required for methylation. Following completion of step (b), the symmetrically-methylated polynucleotide product (e.g. dsDNA) is isolated from the DNMT. If one or more further cycle comprising steps (a)-(b) is required, then the isolated symmetrically-methylated polynucleotide product (e.g. dsDNA) is returned to the first reaction vessel for step (a) of the next cycle to begin. Thus, in some embodiments, the invention comprises a method of amplifying a methylated double-stranded polynucleotide (e.g. dsDNA), method comprising or consisting of the following steps: (a) using a DNA polymerase to amplify one or more target region within at least one DNA strand in a first reaction vessel to form a hemimethylated polynucleotide (e.g. dsDNA) substrate; (b1) transferring the hemimethylated polynucleotide (e.g. dsDNA) substrate to a second reaction vessel; and (b2) contacting the hemimethylated polynucleotide (e.g. dsDNA) substrate with a DNMT (e.g. DNMT5) in the second reaction vessel, thereby producing a symmetrically-methylated polynucleotide (e.g. dsDNA) product. Said method may comprise repeating a cycle of steps (a), (b1) and (b2) to produce a desired amount of the fully-methylated polynucleotide (e.g. dsDNA) product. The number of cycle repeats may be as described herein. Said method may optionally further include a step of ligating one or more first primer to the at least one DNA strand prior to step (a), said ligation step typically not forming part of the cycle of steps, but instead being carried out only once at the start of the method. Amplification in step (a) may be by any appropriate means. By way of non-limiting example, the invention comprises a method of amplifying a methylated double-stranded polynucleotide (e.g. dsDNA), method comprising or consisting of the following steps: (a) hybridising a primer to each strand of the double-stranded polynucleotide (e.g. dsDNA) in a first reaction vessel and extending the primers in the first reaction vessel using a (DNA) polymerase to form a hemimethylated polynucleotide (e.g. dsDNA) substrate; (b1) transferring the hemimethylated polynucleotide (e.g. dsDNA) substrate to a second reaction vessel; and (b2) contacting the hemimethylated polynucleotide (e.g. dsDNA) substrate with a DNMT (e.g. DNMT5) in the second reaction vessel, thereby producing a symmetrically-methylated polynucleotide (e.g. dsDNA) product. Said method may comprise repeating a cycle of steps (a), (b1) and (b2) to produce a desired amount of the symmetrically-methylated polynucleotide (e.g. dsDNA) product. The number of cycle repeats may be as described herein.

In some embodiments, the DNMT may be immobilised, e.g. on beads, and one or more primer and/or polynucleotide substrate (e.g. DNA) are in solution. Primer hybridisation and/or primer ligation, primer extension and step (b) may each be carried out in a separate reaction vessel. Thus, a first reaction vessel may comprise a polynucleotide substrate (e.g. dsDNA) and one or more primer; following hybridisation and/or ligation of one or more primer to the polynucleotide substrate (e.g. dsDNA), the hybridised and/or ligated primers/polynucleotides may be added to a second reaction vessel comprising a polymerase and other amplification reagents. Following primer extension, the hemimethylated polynucleotide product (e.g. dsDNA) is transferred to a third reaction vessel comprising the immobilised DNMT and reagents required for methylation (in step (b)). Following the completion of methylation, the symmetrically-methylated polynucleotide product (e.g. dsDNA) is isolated from the immobilised DNMT. If one or more further cycle comprising steps (a)-(b) is required, then the isolated symmetrically-methylated polynucleotide product (e.g. dsDNA) is returned to the first reaction vessel for the hybridisation of the next cycle to begin.

These configurations may be described as “three-pot” reactions, due to the use of three reaction vessels. Again, such configurations can keep the polymerase and DNMT separate, preventing any interference between the enzymes or the conditions and/or reagents required for each to function. These configurations also allow for optimisation of reagents and/or conditions required for each step. Thus, in some embodiments, the invention comprises a method of amplifying a methylated double-stranded polynucleotide (e.g. dsDNA), method comprising or consisting of the following steps: (a1) hybridising one or more primer to at least one strand of DNA in a first reaction vessel; (a2) transferring the hybridised primers/polynucleotides (e.g. dsDNA) to a second reaction vessel; (a3) using a DNA polymerase to amplify one or more target region within at least one DNA strand in a first reaction vessel to form a hemimethylated polynucleotide (e.g. dsDNA) substrate in a second reaction vessel; (b1) transferring the hemimethylated polynucleotide (e.g. dsDNA) substrate to a second reaction vessel; and (b2) contacting the hemimethylated polynucleotide (e.g. dsDNA) substrate with a DNMT (e.g. DNMT5) in the second reaction vessel, thereby producing a symmetrically-methylated polynucleotide (e.g. dsDNA) product. Said method may comprise repeating a cycle comprising steps (a1), (a2), (a3), (b1) and (b2) to produce a desired amount of the symmetrically-methylated polynucleotide (e.g. dsDNA) product. The number of cycle repeats may be as described herein.

In alternative embodiments, this “three-pot” configuration could be conducted without immobilising the DNMT. Here, DNMT activity could be removed either by functionally removing the DNMT activity, or by physically removing the DNMT protein (i.e. removing or inactivating the DNMT), both of which are described in more detail here. Otherwise, this configuration could remain unchanged. Thus, primer hybridisation and/or primer ligation, primer extension and step (b) may each be carried out in a separate reaction vessel. Thus, a first reaction vessel may comprise a polynucleotide substrate (e.g. dsDNA) and one or more primer; following hybridisation and/or ligation of one or more primer to the polynucleotide substrate (e.g. dsDNA), the hybridised and/or ligated primers/polynucleotides may be added to a second reaction vessel comprising a polymerase and other amplification reagents. Following primer extension, the hemimethylated polynucleotide product (e.g. dsDNA) is transferred to a third reaction vessel comprising the DNMT and reagents required for methylation (in step (b)). Following the completion of methylation, the symmetrically-methylated polynucleotide product (e.g. dsDNA) is isolated from the DNMT. If one or more further cycle comprising steps (a)-(b) is required, then the isolated symmetrically-methylated polynucleotide product (e.g. dsDNA) is returned to the first reaction vessel for the hybridisation of the next cycle to begin. By way of non-limiting example, the invention comprises a method of amplifying a methylated double-stranded polynucleotide (e.g. dsDNA), method comprising or consisting of the following steps: (a1) hybridising one or more primer to at least one strand of DNA in a first reaction vessel; (a2) transferring the hybridised primers/polynucleotides (e.g. dsDNA) to a second reaction vessel; (a3) using a DNA polymerase to amplify one or more target region within at least one DNA strand in a first reaction vessel to form a hemimethylated polynucleotide (e.g. dsDNA) substrate in a second reaction vessel; (b1) transferring the hemimethylated polynucleotide (e.g. dsDNA) substrate to a second reaction vessel; and (b2) contacting the hemimethylated polynucleotide (e.g. dsDNA) substrate with a DNMT (e.g. DNMT5) in the second reaction vessel, thereby producing a symmetrically-methylated polynucleotide (e.g. dsDNA) product. Said method may comprise repeating a cycle comprising steps (a1), (a2), (a3), (b1) and (b2) to produce a desired amount of the symmetrically-methylated polynucleotide (e.g. dsDNA) product. The number of cycle repeats may be as described herein.

In some embodiments, the DNMT, primers and/or polynucleotide substrate (e.g. DNA) are in solution. Steps (a) and (b) are carried out in a single reaction vessel, containing the one or more primers, polynucleotide substrate (e.g. DNA), polymerase and other amplification reagents, as well as the DNMT and the reagents required for methylation. Steps (a)-(b) occur simultaneously within the reaction vessel. If multiple cycles comprising steps (a)-(b) are required, then these will occur within the reaction vessel. The reaction can be stopped after the desired number of cycles, after a certain period of time has elapsed and/or after sufficient symmetrically-methylated polynucleotide product (e.g. dsDNA) has been performed. Said method may optionally further include a step of ligating one or more first primer to the at least one DNA strand prior to step (a), said ligation step typically not forming part of the cycle of steps, but instead being carried out only once at the start of the method. These configurations may be described as “one-pot” reactions, due to the use of a single reaction vessel.

Thus, in some embodiments, the invention comprises a method of amplifying a methylated polynucleotide (e.g. DNA), method comprising or consisting of the following steps: (a) using a DNA polymerase to amplify one or more target region within at least one DNA strand in a first reaction vessel to form a hemimethylated polynucleotide (e.g. dsDNA) substrate; and (b) contacting the hemimethylated polynucleotide (e.g. dsDNA) substrate with a DNMT (e.g. DNMT5) in the same reaction vessel, thereby producing a symmetrically-methylated polynucleotide (e.g. dsDNA) product. Said method may comprise repeating a cycle comprising steps (a) and (b) to produce a desired amount of the symmetrically-methylated polynucleotide (e.g. dsDNA) product. The number of cycle repeats may be as described herein. Said method may optionally further include a step of ligating one or more first primer to the at least one DNA strand prior to step (a), said ligation step typically not forming part of the cycle of steps, but instead being carried out only once at the start of the method. Amplification in step (a) may be by any appropriate means. By way of non-limiting example, the invention provides a method of amplifying a methylated double-stranded polynucleotide (e.g. dsDNA), method comprising or consisting of the following steps: (a) hybridising a primer to each strand of the double-stranded polynucleotide (e.g. dsDNA) in a first reaction vessel, extending the primers in the first reaction vessel using a (DNA) polymerase to form a hemimethylated polynucleotide (e.g. dsDNA) substrate; and (b) contacting the hemimethylated polynucleotide (e.g. dsDNA) substrate with a DNMT (e.g. DNMT5) in the first reaction vessel, thereby producing a symmetrically-methylated polynucleotide (e.g. dsDNA) product. Said method may comprise repeating a cycle comprising steps (a) and (b) to produce a desired amount of the symmetrically-methylated polynucleotide (e.g. dsDNA) product. The number of cycle repeats may be as described herein. A one-pot method of the invention may be carried out at a constant temperature (e.g. between about 30° C. to about 40° C., preferably about 37° C.). Alternatively, a one-pot method of the invention may be carried out at an elevated temperature (e.g. between about 40° C. to about 70° C., preferably about 55° C.) using a thermostable DNMT (as described herein) and/or a thermostable polymerase. In such configurations the polymerase and DNMT may be present throughout. Alternatively, the polymerase may be removed prior to addition of the DNMT and/or the DNMT may be removed to allow step (a) to repeat. Further alternatively, the one-pot method may comprise changing the temperature of the first reaction vessel, such that the primer hybridisation and/or primer ligation, DNA polymerase reaction and methylation reaction take place at different temperatures. Exemplary temperatures for these different reactions are described in detail herein. By way of non-limiting example, the first reaction vessel may be heated (e.g. to 94° C. or 98° C.) to facilitate the DNA polymerase reaction. By way of further non-limiting example, the first reaction vessel may be at a lower temperature (e.g. 25° C., 37° C. or 50° C.) for the methylation reaction compared with the temperature for the DNA polymerase reaction.

In alternative embodiments, this “one-pot” configuration may comprise addition of DNMT activity to the reaction vessel (e.g. before or at the start of step (b)) and/or removal of the DNMT activity from the reaction vessel (e.g. at the end of step (b) or before the start of step (a) of the next cycle). Here, DNMT activity could be removed either by functionally removing the DNMT activity, or by physically removing the DNMT protein (i.e. removing or inactivating the DNMT), both of which are described in more detail here. Alternatively or in addition, this “one-pot” configuration may comprise addition of the one or more primers, polynucleotide substrate (e.g. DNA), DNA polymerase activity and/or reagents for the DNA polymerase reaction to the reaction vessel (e.g. before or at the start of step (a)) and/or removal of the one or more primers, polynucleotide substrate (e.g. DNA), DNA polymerase activity and/or reagents for the DNA polymerase reaction from the reaction vessel (e.g. at the end of step (a) or before the start of step (b)). Here, DNA polymerase activity could be removed either by functionally removing the DNA polymerase activity, or by physically removing the DNA polymerase protein, both of which are described in more detail here. Thus, steps (a) and (b) may be carried out in a single reaction vessel, containing the one or more primers, polynucleotide substrate (e.g. DNA), and other reagents for the DNA polymerase reaction. The DNA polymerase and/or the reagents required for the DNA polymerase reaction may be added before/at the start of each step (a), and/or removed at the end of step (a) or before the start of step (b). Said method may optionally further include a step of ligating one or more first primer to the at least one DNA strand prior to step (a), said ligation step typically not forming part of the cycle of steps, but instead being carried out only once at the start of the method.

By way of non-limiting example, the invention comprises a method of amplifying a methylated polynucleotide (e.g. DNA), method comprising or consisting of the following steps: (a) using a DNA polymerase to amplify one or more target region within at least one DNA strand in a first reaction vessel to form a hemimethylated polynucleotide (e.g. dsDNA) substrate; and (b) contacting the hemimethylated polynucleotide (e.g. dsDNA) substrate with a DNMT (e.g. DNMT5) in the same reaction vessel, thereby producing a symmetrically-methylated polynucleotide (e.g. dsDNA) product. Said method may comprise repeating a cycle comprising steps (a) and (b) to produce a desired amount of the symmetrically-methylated polynucleotide (e.g. dsDNA) product. The DNMT activity may be added to the reaction vessel (e.g. before or at the start of step (b)) and/or the DNMT activity may be removed from the reaction vessel (e.g. at the end of step (b) or before the start of step (a) of the next cycle). Alternatively or in addition DNA polymerase activity may be added before/at the start of each step (a), and/or removed at the end of step (a) or before the start of step (b). The number of cycle repeats may be as described herein. Said method may optionally further include a step of ligating one or more first primer to the at least one DNA strand prior to step (a), said ligation step typically not forming part of the cycle of steps, but instead being carried out only once at the start of the method. Amplification in step (a) may be by any appropriate means. By way of non-limiting example, the invention provides a method of amplifying a methylated double-stranded polynucleotide (e.g. dsDNA), method comprising or consisting of the following steps: (a) hybridising a primer to each strand of the double-stranded polynucleotide (e.g. dsDNA) in a first reaction vessel, extending the primers in the first reaction vessel using a (DNA) polymerase to form a hemimethylated polynucleotide (e.g. dsDNA) substrate; (b1) introducing DNMT (e.g. DNMT5) activity to the first reaction vessel and contacting the hemimethylated polynucleotide (e.g. dsDNA) substrate with said DNMT5 (e.g. DNMT5) activity in the first reaction vessel, thereby producing a symmetrically-methylated polynucleotide (e.g. dsDNA) product; and (b2) removing the DNMT (e.g. DNMT5) activity from the first reaction vessel. Said method may comprise repeating a cycle comprising steps (a), (b1) and (b2) to produce a desired amount of the symmetrically-methylated polynucleotide (e.g. dsDNA) product. The number of cycle repeats may be as described herein. A one-pot method of the invention may be carried out at a constant temperature (e.g. between about 30° C. to about 40° C., preferably about 37° C.). Alternatively, a one-pot method of the invention may be carried out at an elevated temperature (e.g. between about 40° C. to about 70° C., preferably about 55° C.) using a thermostable DNMT (as described herein) and/or a thermostable polymerase. Further alternatively, the one-pot method may comprise changing the temperature of the first reaction vessel, such that the primer hybridisation and/or primer ligation, DNA polymerase reaction and methylation reaction take place at different temperatures. Exemplary temperatures for these different reactions are described in detail herein. By way of non-limiting example, the first reaction vessel may be heated (e.g. to 94° C. or 98° C.) to facilitate the DNA polymerase reaction. By way of further non-limiting example, the first reaction vessel may be at a lower temperature (e.g. 25° C., 37° C. or 50° C.) for the methylation reaction compared with the temperature for the DNA polymerase reaction.

In some embodiments, the DNMT, primers and/or polynucleotide substrate (e.g. DNA) are in solution. Steps (a) and (b) are carried out in a single reaction vessel, containing the one or more primers, polynucleotide substrate (e.g. DNA), polymerase and other amplification reagents, as well as the DNMT and the reagents required for methylation. Said method includes a step of ligating one or more first primer to the at least one DNA strand prior to step (a), wherein said one or more first primer sequence comprises a recognition sequence for a DNA modifying enzyme (DNA cleaving enzyme). Said ligation step typically not forming part of the cycle of steps, but instead being carried out only once at the start of the method. In step (a), amplification of the one or more target region occurs via extension of the one or more first primer to produce corresponding daughter strand(s) and hence a hemimethylated polynucleotide (e.g. dsDNA) substrate. Following amplification, in step (b) a DNA modifying enzyme (DNA cleaving enzyme) which recognises the recognition sequence generates a nick in the hemimethylated polynucleotide (e.g. dsDNA) substrate. In step (b) nicking may occur first (step (b1), followed by methylation (step (b2). Alternatively, in step (b) the methylation may occur first (step (b1), followed by nicking (step (b2)). These configurations may be described as “one-pot” reactions, due to the use of a single reaction vessel. Thus, in some embodiments, the invention comprises a method of amplifying a methylated polynucleotide (e.g. DNA), method comprising or consisting of the following steps: ligating one or more first primer to the at least one DNA strand, wherein said one or more first primer sequence comprises a recognition sequence for a DNA modifying enzyme (DNA cleaving enzyme); step (a) using a DNA polymerase to amplify one or more target region within at least one DNA strand in a first reaction vessel to form a hemimethylated polynucleotide (e.g. dsDNA) substrate; and step (b) contacting the hemimethylated polynucleotide (e.g. dsDNA) substrate with a DNMT (e.g. DNMT5) and a DNA modifying enzyme (DNA cleaving enzyme) which recognises the DNA modifying recognition sequence and generates a nick in the hemimethylated polynucleotide (e.g. dsDNA) substrate (wherein nicking and methylation can occur in either order) in the same reaction vessel, thereby producing a symmetrically-methylated polynucleotide (e.g. dsDNA) product. Said method may comprise repeating a cycle comprising steps (a) and (b) to produce a desired amount of the symmetrically-methylated polynucleotide (e.g. dsDNA) product. The number of cycle repeats may be as described herein. The number of cycle repeats may be as described herein. A one-pot method of the invention may be carried out at a constant temperature (e.g. between about 30° C. to about 40° C., preferably about 37° C.). Alternatively, a one-pot method of the invention may be carried out at an elevated temperature (e.g. between about 40° C. to about 70° C., preferably about 55° C.) using a thermostable DNMT (as described herein) and/or a thermostable polymerase. In such configurations the polymerase and DNMT may be present throughout. Alternatively, DNMT activity and/or the DNA modifying enzyme (DNA cleaving enzyme) activity may be added to the reaction vessel (e.g. before or at the start of step (b)) and/or the DNMT activity and/or the DNA modifying enzyme (DNA cleaving enzyme) activity may be removed from the reaction vessel (e.g. at the end of step (b) or before the start of step (a) of the next cycle). Alternatively or in addition DNA polymerase activity may be added before/at the start of each step (a), and/or removed at the end of step (a) or before the start of step (b). Further alternatively, the one-pot method may comprise changing the temperature of the first reaction vessel, such that the primer hybridisation and/or primer ligation, DNA polymerase reaction and methylation reaction take place at different temperatures. Exemplary temperatures for these different reactions are described in detail herein. By way of non-limiting example, the first reaction vessel may be heated (e.g. to 94° C. or 98° C.) to facilitate the DNA polymerase reaction. By way of further non-limiting example, the first reaction vessel may be at a lower temperature (e.g. 25° C., 37° C. or 50° C.) for the methylation reaction compared with the temperature for the DNA polymerase reaction.

In some embodiments, the DNMT, primers and/or polynucleotide substrate (e.g. DNA) are in solution. Steps (a) and (b) are carried out in a single reaction vessel, containing the one or more primers, polynucleotide substrate (e.g. DNA), polymerase and other amplification reagents, as well as the DNMT and the reagents required for methylation. Said method includes a step of ligating one or more first primer to the at least one DNA strand prior to step (a). Said ligation step typically not forming part of the cycle of steps, but instead being carried out only once at the start of the method. Step (a) comprises addition of one or more second primer which hybridises to the one or more first primer, wherein said one or more second primer sequence comprises a recognition sequence for a DNA modifying enzyme (DNA cleaving enzyme). Amplification of the one or more target region occurs via extension of the one or more second primer to produce corresponding daughter strand(s) and hence a hemimethylated polynucleotide (e.g. dsDNA) substrate. Following amplification, in step (b) a DNA modifying enzyme (DNA cleaving enzyme) which recognises the recognition sequence in the one or more second primer sequence generates a nick in the hemimethylated polynucleotide (e.g. dsDNA) substrate. In step (b) nicking may occur first (step (b1), followed by methylation (step (b2). Alternatively, in step (b) the methylation may occur first (step (b1), followed by nicking (step (b2)). These configurations may be described as “one-pot” reactions, due to the use of a single reaction vessel. Thus, in some embodiments, the invention comprises a method of amplifying a methylated polynucleotide (e.g. DNA), method comprising or consisting of the following steps: ligating one or more first primer to the at least one DNA strand; step (a1) hybridising one or more second primer to said one or more first primer, wherein the one or more second primer sequence comprises a recognition sequence for a DNA modifying enzyme (DNA cleaving enzyme), and step (a2) using a DNA polymerase to amplify one or more target region within at least one DNA strand in a first reaction vessel to form a hemimethylated polynucleotide (e.g. dsDNA) substrate; and step (b) contacting the hemimethylated polynucleotide (e.g. dsDNA) substrate with a DNMT (e.g. DNMT5) and a DNA modifying enzyme (DNA cleaving enzyme) which recognises the DNA modifying (DNA cleaving) recognition sequence and generates a nick in the hemimethylated polynucleotide (e.g. dsDNA) substrate (wherein nicking and methylation can occur in either order) in the same reaction vessel, thereby producing a symmetrically-methylated polynucleotide (e.g. dsDNA) product. Said method may comprise repeating a cycle comprising steps (a) and step (b) to produce a desired amount of the symmetrically-methylated polynucleotide (e.g. dsDNA) product. The number of cycle repeats may be as described herein. A one-pot method of the invention may be carried out at a constant temperature (e.g. between about 30° C. to about 40° C., preferably about 37° C.). Alternatively, a one-pot method of the invention may be carried out at an elevated temperature (e.g. between about 40° C. to about 70° C., preferably about 55° C.) using a thermostable DNMT (as described herein) and/or a thermostable polymerase.

In such configurations the polymerase and DNMT may be present throughout. Alternatively, DNMT activity and/or the DNA modifying enzyme (DNA cleaving enzyme) activity may be added to the reaction vessel (e.g. before or at the start of step (b)) and/or the DNMT activity and/or the DNA modifying enzyme (DNA cleaving enzyme) activity may be removed from the reaction vessel (e.g. at the end of step (b) or before the start of step (a) of the next cycle). Alternatively or in addition DNA polymerase activity may be added before/at the start of each step (a), and/or removed at the end of step (a) or before the start of step (b). Further alternatively, the one-pot method may comprise changing the temperature of the first reaction vessel, such that the primer hybridisation and/or primer ligation, DNA polymerase reaction and methylation reaction take place at different temperatures. Exemplary temperatures for these different reactions are described in detail herein. By way of non-limiting example, the first reaction vessel may be heated (e.g. to 94° C. or 98° C.) to facilitate the DNA polymerase reaction. By way of further non-limiting example, the first reaction vessel may be at a lower temperature (e.g. 25° C., 37° C. or 50° C.) for the methylation reaction compared with the temperature for the DNA polymerase reaction.

In some embodiments, the DNMT, primers and/or polynucleotide substrate (e.g. DNA) are in solution. Steps (a) and (b) are carried out in a single reaction vessel, containing the one or more primers, polynucleotide substrate (e.g. DNA), polymerase and other amplification reagents, as well as the DNMT and the reagents required for methylation. Said method includes a step of ligating one or more first primer to the at least one DNA strand prior to step (a). Said ligation step typically not forming part of the cycle of steps, but instead being carried out only once at the start of the method. Step (a) comprises addition of one or more second primer which hybridises to the one or more first primer, wherein the one or more first primer sequence and one or more second primer sequence together comprise a double-stranded recognition sequence for a DNA modifying enzyme (DNA cleaving enzyme). Amplification of the one or more target region occurs via extension of the one or more second primer to produce corresponding daughter strand(s) and hence a hemimethylated polynucleotide (e.g. dsDNA) substrate. Following amplification, in step (b) a DNA modifying enzyme (DNA cleaving enzyme) which recognises the recognition sequence generates a nick in the hemimethylated polynucleotide (e.g. dsDNA) substrate. In step (b) the nicking may occur first (step (b1), followed by methylation (step (b2). Alternatively, in step (b) the methylation may occur first (step (b1), followed by nicking (step (b2)). These configurations may be described as “one-pot” reactions, due to the use of a single reaction vessel. Thus, in some embodiments, the invention comprises a method of amplifying a methylated polynucleotide (e.g. DNA), method comprising or consisting of the following steps: ligating one or more first primer to the at least one DNA strand; step (a1) hybridising one or more second primer to said one or more first primer, wherein the one or more first primer sequence and the one or more second primer together comprise a double-stranded recognition sequence for a DNA modifying enzyme (DNA cleaving enzyme), and step (a2) using a DNA polymerase to amplify one or more target region within at least one DNA strand in a first reaction vessel to form a hemimethylated polynucleotide (e.g. dsDNA) substrate; and (b) contacting the hemimethylated polynucleotide (e.g. dsDNA) substrate with a DNMT (e.g. DNMT5) and a DNA modifying enzyme (DNA cleaving enzyme) which recognises the DNA modifying (DNA cleaving) recognition sequence and generates a nick in the hemimethylated polynucleotide (e.g. dsDNA) substrate (wherein nicking and methylation can occur in either order) in the same reaction vessel, thereby producing a symmetrically-methylated polynucleotide (e.g. dsDNA) product. Said method may comprise repeating a cycle comprising steps (a) and step (b) to produce a desired amount of the symmetrically-methylated polynucleotide (e.g. dsDNA) product. The number of cycle repeats may be as described herein. A one-pot method of the invention may be carried out at a constant temperature (e.g. between about 30° C. to about 40° C., preferably about 37° C.). Alternatively, a one-pot method of the invention may be carried out at an elevated temperature (e.g. between about 40° C. to about 70° C., preferably about 55° C.) using a thermostable DNMT (as described herein) and/or a thermostable polymerase. In such configurations the polymerase and DNMT may be present throughout. Alternatively, DNMT activity and/or the DNA modifying enzyme (DNA cleaving enzyme) activity may be added to the reaction vessel (e.g. before or at the start of step (b)) and/or the DNMT activity and/or the DNA modifying enzyme (DNA cleaving enzyme) activity may be removed from the reaction vessel (e.g. at the end of step (b) or before the start of step (a) of the next cycle). Alternatively or in addition DNA polymerase activity may be added before/at the start of each step (a), and/or removed at the end of step (a) or before the start of step (b). Further alternatively, the one-pot method may comprise changing the temperature of the first reaction vessel, such that the primer hybridisation and/or primer ligation, DNA polymerase reaction and methylation reaction take place at different temperatures. Exemplary temperatures for these different reactions are described in detail herein. By way of non-limiting example, the first reaction vessel may be heated (e.g. to 94° C. or 98° C.) to facilitate the DNA polymerase reaction. By way of further non-limiting example, the first reaction vessel may be at a lower temperature (e.g. 25° C., 37° C. or 50° C.) for the methylation reaction compared with the temperature for the DNA polymerase reaction.

In some embodiments, the DNMT is in solution, and the one or more primer and/or polynucleotide substrate (e.g. DNA) are immobilised, e.g. on a flow cell or chip. Steps (a) and (b) may be carried out within the flow cell or chip. One or more primer may hybridise to their target polynucleotide within an immobilised polynucleotide library, resulting in the production of primers hybridised to a methylated double-stranded polynucleotide (e.g. dsDNA) in step (a). The reagents necessary for primer hybridisation and extension (e.g. the polymerase and other amplification reagents) can be flowed over the immobilised primers hybridised to a methylated double-stranded polynucleotide (e.g. dsDNA) in step (a), resulting in the production of an immobilised hemimethylated polynucleotide (e.g. dsDNA) substrate. The DNMT can then be flowed over the immobilised hemimethylated polynucleotide (e.g. dsDNA) substrate in step (b), resulting in the production of a symmetrically-methylated polynucleotide (e.g. dsDNA) product. Following completion of step (b), the symmetrically-methylated polynucleotide product (e.g. dsDNA) may be isolated, or may be analysed in situ. If one or more further cycle comprising steps (a)-(b) is required, the process can be repeated. Said method may optionally further include a step of ligating one or more first primer to the at least one DNA strand prior to step (a), said ligation step typically not forming part of the cycle of steps, but instead being carried out only once at the start of the method. These configurations may be described as “clustering” reactions, as the specific polynucleotide template will cluster on the surface of the flow cell or chip. Thus, in some embodiments, the invention comprises a method of amplifying a methylated polynucleotide (e.g. DNA), said method comprising or consisting of the following steps: (a) hybridising a primer to each strand of the double-stranded polynucleotide (e.g. dsDNA) immobilised on a surface, e.g. the surface of a flow cell or chip, extending the primers using a (DNA) polymerase to form a hemimethylated polynucleotide (e.g. dsDNA) substrate; and (b) contacting the hemimethylated polynucleotide (e.g. dsDNA) substrate with a DNMT (e.g. DNMT5) by flowing the DNMT in solution over the surface, thereby producing a symmetrically-methylated polynucleotide (e.g. dsDNA) product. Said method may comprise repeating a cycle comprising steps (a) and (b) to produce a desired amount of the symmetrically-methylated polynucleotide (e.g. dsDNA) product. The number of cycle repeats may be as described herein.

In some embodiments, the DNMT is in solution, and one or more primer and/or polynucleotide substrate (e.g. dsDNA) are labelled to allow for capture and release. Steps (a) and (b) may be carried out in the same reaction vessel. Thus, the invention provides a method of amplifying a methylated polynucleotide (e.g. DNA), comprising or consisting of the following steps: (a) using a DNA polymerase to amplify one or more target region within at least one DNA strand in a first reaction vessel to form a hemimethylated polynucleotide (e.g. dsDNA) substrate; and (b) contacting the hemimethylated polynucleotide (e.g. dsDNA) substrate with a DNMT (e.g. DNMT5) in the first reaction vessel, thereby producing a symmetrically-methylated polynucleotide (e.g. dsDNA) product; wherein steps (a) and (b) are carried out in the same reaction vessel, and following amplification (in step (a)), DNMT and the reagents required for methylation (in step (b)) may be added in solution to the reaction vessel containing the hemimethylated polynucleotide product (e.g. dsDNA). Following the methylation (in step (b)), the labelled one or more primers and/or polynucleotide substrate (e.g. dsDNA) can be captured using an appropriate capture method, and the solution comprising the DNMT and any other residual reagents may be removed. If one or more further cycle comprising steps (a)-(b) is required, then the one or more labelled primers and/or polynucleotide substrate (e.g. dsDNA) can then be released and fresh reaction buffer added for step (a) of the next cycle to begin. The number of cycle repeats may be as described herein. Said method may optionally further include a step of ligating one or more first primer to the at least one DNA strand prior to step (a), said ligation step typically not forming part of the cycle of steps, but instead being carried out only once at the start of the method. By way of non-limiting example, the invention comprises a method of amplifying a methylated double-stranded polynucleotide (e.g. dsDNA), said method comprising or consisting of the following steps: (a1) hybridising a primer to each strand of the double-stranded polynucleotide (e.g. dsDNA), wherein the primer and/or each strand of the double-stranded polynucleotide (e.g. dsDNA) are labelled; (a2) extending the primers using a (DNA) polymerase to form a hemimethylated polynucleotide (e.g. dsDNA) substrate; (b1) contacting the hemimethylated polynucleotide (e.g. dsDNA) substrate with a DNMT (e.g. DNMT5) in solution, thereby producing a symmetrically-methylated polynucleotide (e.g. dsDNA) product; (b2) capturing the labelled primers and/or symmetrically-methylated polynucleotide (e.g. dsDNA) product; (b3) removing the DNMT and releasing the captured labelled primers and/or symmetrically-methylated polynucleotide (e.g. dsDNA) product. Said method may comprise repeating a cycle of steps (a1), (a2) (b1), (b2) and (b3) to produce a desired amount of the symmetrically-methylated polynucleotide (e.g. dsDNA) product. The number of cycle repeats may be as described herein.

The reaction vessel may comprise a polynucleotide substrate (e.g. dsDNA) and one or more primer. The polymerase and other amplification reagents may also be present in the reaction vessel, or may be added following hybridisation of the one or more primer to the polynucleotide substrate (e.g. dsDNA) (in step (a1)). Following primer extension (in step (a2)), DNMT and the reagents required for methylation (in step (b)).may be added in solution to the reaction vessel containing the hemimethylated polynucleotide product (e.g. dsDNA). Following the methylation (in step (b)), the labelled primers and/or polynucleotide substrate (e.g. dsDNA) can be captured using an appropriate capture method, and the solution comprising the DNMT and any other residual reagents may be removed. If one or more further cycle comprising steps (a)-(b) is required, then the labelled primers and/or polynucleotide substrate (e.g. dsDNA) can then be released and fresh reaction buffer added for step (a) of the next cycle to begin. The number of cycle repeats may be as described herein. Said method may optionally further include a step of ligating one or more first primer to the at least one DNA strand prior to step (a1), said ligation step typically not forming part of the cycle of steps, but instead being carried out only once at the start of the method. By way of non-limiting example, the invention comprises a method of amplifying a methylated double-stranded polynucleotide (e.g. dsDNA), method comprising or consisting of the following steps: (a) hybridising a primer to each strand of the double-stranded polynucleotide (e.g. dsDNA), wherein the primer and/or each strand of the double-stranded polynucleotide (e.g. dsDNA) are labelled, extending the primers using a (DNA) polymerase to form a hemimethylated polynucleotide (e.g. dsDNA) substrate; (b1) contacting the hemimethylated polynucleotide (e.g. dsDNA) substrate with a DNMT (e.g. DNMT5) in solution, thereby producing a symmetrically-methylated polynucleotide (e.g. dsDNA) product; (b2) capturing the labelled primers and/or symmetrically-methylated polynucleotide (e.g. dsDNA) product; (b3) removing the DNMT and releasing the captured labelled primers and/or symmetrically-methylated polynucleotide (e.g. dsDNA) product. Said method may comprise repeating a cycle of steps (a1), (a2) (b1), (b2) and (b3) to produce a desired amount of the symmetrically-methylated polynucleotide (e.g. dsDNA) product. The number of cycle repeats may be as described herein. The different zones or chambers of the microfluidic chip may be at a constant temperature (e.g. between about 30° C. to about 40° C., preferably about 37° C.). Alternatively, the different zones or chambers of the microfluidic chip may be at different temperatures, such that the primer hybridisation and/or primer ligation,

DNA polymerase reaction and methylation reaction take place at different temperatures. Exemplary temperatures for these different reactions are described in detail herein. By way of non-limiting example, the second zone or chamber may be heated (e.g. to 94° C. or 98° C.) to facilitate the DNA polymerase reaction. By way of further non-limiting example, the third zone or chamber may be at a lower temperature (e.g. 25° C., 37° C. or 50° C.) for the methylation reaction compared with the temperature of the second zone or chamber for the DNA polymerase reaction.

In some embodiments, the method is carried out in a microfluidic chip, wherein the DNMT and polymerase are immobilised in different chambers or zones of the chip, and the one or more primer and/or polynucleotide substrate (e.g. DNA) are in solution. Steps (a) and (b) may be carried out within the chip. Denaturation and primer hybridisation may occur in a first zone or chamber of the chip (in step (a1)). The primers hybridised to the polynucleotide substrate (e.g. dsDNA) may then be moved (e.g. by fluid flow or injection) to a second zone or chamber of the chip, wherein the polymerase is immobilised. The reagents necessary for primer extension (e.g. the polymerase and other amplification reagents) can be added to this second zone or chamber of the chip, resulting in the production of a hemimethylated polynucleotide (e.g. dsDNA) substrate (in step (a2)). The hemimethylated polynucleotide (e.g. dsDNA) substrate (and optionally the one or more primers) can then be moved to a third zone or chamber of the chip (e.g. by fluid flow or injection), wherein the DNMT is immobilised. The reagents necessary for methylation can be added to this third zone or chamber of the chip, resulting in the production of a symmetrically-methylated polynucleotide (e.g. dsDNA) product (in step (b)). If one or more further cycle comprising steps (a1), (a2) and (b) is required, the process can be repeated. Said method may optionally further include a step of ligating one or more first primer to the at least one DNA strand prior to step (a1), said ligation step typically not forming part of the cycle of steps, but instead being carried out only once at the start of the method. By way of non-limiting example, the invention comprises a method of amplifying a methylated double-stranded polynucleotide (e.g. dsDNA), method comprising or consisting of the following steps: (a1) hybridising a primer to each strand of the double-stranded polynucleotide (e.g. dsDNA) in solution within a first zone or chamber of a microfluidic chip; (a2) extending the primers using a (DNA) polymerase immobilised to the surface of a second zone or chamber of the microfluidic chip to form a hemimethylated polynucleotide (e.g. dsDNA) substrate; and (b) contacting the hemimethylated polynucleotide (e.g. dsDNA) substrate with a DNMT (e.g. DNMT5) immobilised to the surface of a third zone or chamber of the microfluidic chip, thereby producing a symmetrically-methylated polynucleotide (e.g. dsDNA) product. Said method may comprise repeating a cycle comprising steps (a1) (a2) and (b) to produce a desired amount of the symmetrically-methylated polynucleotide (e.g. dsDNA) product. The number of cycle repeats may be as described herein.

In some embodiments, the method is carried out in a microfluidic chip, wherein the DNMT and polymerase are immobilised in different chambers or zones of the chip, and the one or more primer and/or polynucleotide substrate (e.g. DNA) are in solution. Steps (a) and (b) may be carried out within the chip. In step (a1), denaturation and primer hybridisation may occur in a first zone or chamber of the chip, where the polymerase is immobilised. The reagents necessary for primer extension (e.g. other amplification reagents) can be added to this first zone or chamber of the chip, resulting in the production of a hemimethylated polynucleotide (e.g. dsDNA) substrate (in step (a2)). The hemimethylated polynucleotide (e.g. dsDNA) substrate (and optionally the one or more primers) can then be moved to a second zone or chamber of the chip (e.g. by fluid flow or injection), wherein the DNMT is immobilised.

The reagents necessary for methylation can be added to this second zone or chamber of the chip, resulting in the production of a symmetrically-methylated polynucleotide (e.g. dsDNA) product (in step (b)). If one or more further cycle comprising steps (a1), (a2) and (b) is required, the process can be repeated. Said method may optionally further include a step of ligating one or more first primer to the at least one DNA strand prior to step (a1), said ligation step typically not forming part of the cycle of steps, but instead being carried out only once at the start of the method. By way of non-limiting example, the invention comprises a method of amplifying a methylated double-stranded polynucleotide (e.g. dsDNA), method comprising or consisting of the following steps: (a1) hybridising a primer to each strand of the double-stranded polynucleotide (e.g. dsDNA) in solution within a first zone or chamber of a microfluidic chip; (a2) extending the primers using a (DNA) polymerase immobilised to the surface of said first zone or chamber of the microfluidic chip to form a hemimethylated polynucleotide (e.g. dsDNA) substrate; and (b) contacting the hemimethylated polynucleotide (e.g. dsDNA) substrate with a DNMT (e.g. DNMT5) immobilised to the surface of a second zone or chamber of the microfluidic chip, thereby producing a symmetrically-methylated polynucleotide (e.g. dsDNA) product. Said method may comprise repeating a cycle comprising steps (a1), (a2) and (b) to produce a desired amount of the symmetrically-methylated polynucleotide (e.g. dsDNA) product. The number of cycle repeats may be as described herein. The different zones or chambers of the microfluidic chip may be at a constant temperature (e.g. between about 30° C. to about 40° C., preferably about 37° C.). Alternatively, the different zones or chambers of the microfluidic chip may be at different temperatures, such that the primer hybridisation and/or primer ligation, DNA polymerase reaction and methylation reaction take place at different temperatures. Exemplary temperatures for these different reactions are described in detail herein. By way of non-limiting example, the first zone or chamber may be heated (e.g. to 94° C. or 98° C.) to facilitate the DNA polymerase reaction. By way of further non-limiting example, the second zone or chamber may be at a lower temperature (e.g. 25° C., 37° C. or 50° C.) for the methylation reaction compared with the temperature in the first zone or chamber for the DNA polymerase reaction.

Wherein a method of the invention is carried out using a flow chip or cell (also referred to interchangeably herein as a microfluidic chip), the chip may be a sequencing chip. In other words, it may be possible to detect and/or analyse the presence of mC and/or hmC in situ within the chip used for the method of the invention.

In some particularly preferred embodiments, steps (a) and (b) are carried out in a single reaction vessel, containing the one or more primers, polynucleotide substrate (e.g. dsDNA), polymerase and other amplification reagents, as well as the DNMT and the reagents required for methylation. Steps (a)-(b) occur simultaneously within the reaction vessel. If multiple cycles comprising steps (a)-(b) are required, then these will occur within the reaction vessel.

The reaction can be stopped after the desired number of cycles, after a certain period of time has elapsed and/or after sufficient symmetrically-methylated polynucleotide product (e.g. dsDNA) has been performed. Typically, the DNMT, primers and/or polynucleotide substrate (e.g. DNA) are in solution. Said method may optionally further include a step of ligating one or more first primer to the at least one DNA strand prior to step (a), said ligation step typically not forming part of the cycle of steps, but instead being carried out only once at the start of the method. Alternatively or in addition, these embodiments may comprise addition of the one or more primers, polynucleotide substrate (e.g. dsDNA), DNA polymerase activity and/or reagents for the DNA polymerase reaction to the reaction vessel (e.g. before or at the start of step (a)) and/or removal of the one or more primers, polynucleotide substrate (e.g. DNA), DNA polymerase activity and/or reagents for the DNA polymerase reaction from the reaction vessel (e.g. at the end of step (a) or before the start of step (b)).

These configurations may be described as “one-pot” reactions, due to the use of a single reaction vessel. The present inventors are the first to provide a system in which DNA amplification and methylation can occur (i) in a single reaction vessel (ii) using a common buffer and (iii) without needing to purify the amplified DNA before methylation occurs. As such, these “one-pot” reactions are particularly advantageous over the prior art, and are therefore particularly preferred embodiments of the invention. Accordingly, any and all disclosure herein relating to methods of the invention applies particularly and without reservation to these “one-pot” reactions, unless expressly stated otherwise.

### Applications of the Amplification Method

As described herein, conventional methods for amplifying DNA, such as PCR do not copy methylation from the parent strand (referred to interchangeably herein as the template or substrate strand) to the daughter strand, meaning that this important epigenetic information is lost. The present invention provides a valuable alternative to techniques such as PCR for applications where it is important and/or desirable to retain the methylation patent of the parent DNA strand. This is because, as exemplified herein, the methods of the invention are able to amplify DNA whilst accurately copying the methylation pattern from a parent strand to the daughter strands, with minimal de novo methylation. This allows for small amounts of template DNA in a sample to be amplified for further analysis, whilst faithfully retaining the methylation pattern of the template DNA.

The methods of the invention have potential utility in a number of applications, particularly in the context of next generation sequencing (NGS) and/or epigenetic analysis. These applications are also encompassed by the present invention.

Methylated DNA is an important biomarker and prognostic assessment indicator. As such, DNA methylation can be used as a prophylactic, prognostic, diagnostic and therapeutic marker for a range of diseases including cancer, but also other diseases such as type 1 diabetes.

Accurate copies of methylated DNA serves as a powerful diagnostic tool. For instance, certain cancers can be identified by their unique methylation signatures, even in early stages when other diagnostic methods might fail. Similarly, the detection of aberrant methylation patterns can aid in diagnosing diseases such as type 1 diabetes, where autoimmune-related methylation changes can be indicative of disease presence. For instance, the present invention provides methods for the analysis of methylated cancer biomarker, human SEPT9, an important biomarker for colorectal cancer. As exemplified herein, human SEPT9 has been successfully amplified and protected from digest using a method of the invention, demonstrating the potential of the invention for diagnostic application. Whilst human SEPT9 was used herein for proof of concept, it will be appreciated that method of the invention can amplify any suitable marker. For example, suitable markers may include cancer methylation markers (MGMT (e.g., O-6-methylguanine-DNA methyltransferase), BRCA1 (Breast Cancer 1), GSTP1 (Glutathione S-transferase Pi 1), RASSF1A (Ras Association Domain Family Member 1) and CDKN2A/p16 (Cyclin-Dependent Kinase Inhibitor 2A), type 1 diabetes markers (e.g., GAD2 (Glutamate Decarboxylase 2), neurodegenerative disease markers (APP (Amyloid Precursor Protein) and PSEN1 (Presenilin 1), cardiovascular disease markers (e.g., LINE-1 (Long Interspersed Nuclear Element-1) and autoimmune disease markers (e.g., TGAL (Integrin Subunit Alpha L) and CD40LG (CD40 Ligand).

Methylation markers also guide therapeutic decisions. In oncology, the presence or absence of methylation in specific genes can determine the suitability of particular treatments, such as demethylating agents. Monitoring changes in methylation patterns during treatment provides feedback on therapeutic efficacy and helps adjust treatment strategies for better outcomes.

Methylation analysis may help to monitor residual disease after an intervention (e.g. surgery), or during on-going treatment. Further, monitoring methylation patterns may also be valuable for monitoring the effectiveness of therapeutic entities in treating disease. In particular, cell-free DNA (cfDNA) is a marker of cell death, tissue injury, and inflammation. DNA methylation deconvolution may be used to determine the source of cfDNA, opening up further possibilities for the use of cfDNA as a clinical biomarker. Determining the methylation pattern of cfDNA in maternal-fetal medicine may be particularly beneficial for fetal genetic testing and placental disorders. Other potential applications include paediatric medicine. Methylation patterns change during aging, opening up the possibility of determining and monitoring the biological age of tissues and the reversal of the aging process. The methods of the invention may be of particular utility in such applications.

DNA from any appropriate sample may be used in a method/application of the invention. Examples of suitable sample materials are described herein. Preferably the sample is a biological fluid, such as a blood, plasma, saliva, serum, sputum, urine, cerebrospinal fluid, sweat or synovial fluid. So-called liquid biopsies, particularly blood, plasma or serum samples may be of particular use in the invention.

The invention provides a method of detecting methylation in a polynucleotide (e.g. dsDNA) substrate comprising carrying out a method of amplifying a methylated polynucleotide (e.g. dsDNA) according to the invention, and detecting mC and/or hmC in the symmetrically-methylated polynucleotide (e.g. dsDNA) product produced by a final step (b). As described herein, the methylation status of the original strands of the polynucleotide (e.g. dsDNA) substrate may be analysed separately from the strands copied in the amplification method. By way of non-limiting example, the original strands may be analysed for the presence of hmC, and the copied strands may be analysed for the presence of mC, as described herein.

The invention also provides a method of generating a methylated DNA library, said method comprising carrying out a method of amplifying a methylated polynucleotide (e.g. dsDNA) according to the invention and separating the strands of the symmetrically-methylated dsDNA product produced by a final step (b) to form a single-stranded DNA library.

The methods of the invention are particularly useful in the analysis of cfDNA. It is well-established that the methylation patterns of cfDNA can be used as a biomarker for numerous diseases, as well as in cancer diagnostics. By way of non-limiting example, the invention provides a method for determining the presence of a methylated DNA biomarker, said method comprising: (a) carrying out a method of amplifying methylated polynucleotide (e.g. dsDNA) according to the invention; (b) detecting methylation of the polynucleotide (e.g. dsDNA) to determine the methylation pattern of the polynucleotide (e.g. dsDNA); and (c) comparing the methylation pattern with known methylation biomarkers to determine whether or not a methylation biomarker of interest is present within the methylated polynucleotide (e.g. dsDNA). Preferably the methylated polynucleotide (e.g. dsDNA) dsDNA, more preferably cfDNA. In said method any appropriate means may be used to detect methylation within the polynucleotide (e.g. dsDNA). Non-limiting examples of such techniques are described herein.

The methods of the present invention also have potential utility in any application where the amplification and/or analysis of methylated DNA may be of interest. By way of further non-limiting examples, in amplifying and/or analysing DNA in clinical samples such as stool samples, tissue samples, samples of body fluids for the purposes of diagnosis, prognosis or other uses, such as forensic analysis. Non-limiting examples of sample types are described herein.

Thus, the invention provides a method of determining the presence of a methylated biomarker in a biological fluid, the method comprising amplifying methylated double-stranded DNA (dsDNA) from the biological fluid to produce an amplified dsDNA product using a method as described herein, wherein methylation of the biomarker is associated with a medical condition.

The invention also provides a method of diagnosing a medical condition comprising determining the presence of a methylated biomarker in a biological fluid, the method comprising amplifying methylated double-stranded DNA (dsDNA) from the biological fluid to produce an amplified dsDNA product using a method of any one of claims 1 to 30, wherein methylation of the biomarker is associated with the medical condition.

The methylated biomarker of such a method may be associated with, i.e. diagnostic for a cancer. In such instances, the biological fluid is typically that of an animal, preferably a mammal, still more preferably a human.

Such a method may further comprise administering an effective dose of a therapeutic agent or using surgical means for treating the cancer. The appropriate therapeutic agent and doses thereof may be selected by a clinician using routine skill. Non-limiting examples of suitable therapeutic agents selected from immunotherapy, chemotherapy and radiation.

The “one-pot” methods of the invention are particularly suited for such methods of detection and diagnosis, as the simplified one-pot format lends itself for use in clinical settings. Thus, methods of determining the presence of a methylated biomarker or methods of diagnosing a medical condition according to the invention may comprise carrying out step (a) and step (b) in the same reaction vessel. The biological fluid may be selected from blood, plasma, saliva, serum, sputum, urine, cerebrospinal fluid, cells, a cellular extract, sweat or synovial fluid.

### DNA Storage

DNA data storage is a newly emerging way of storing digital data at very high densities, and much lower long-term costs of traditional data storage methods. It has been mooted for many years, first discussed in the late 1950s and early 1960s by Feynman (RP (29 Dec. 1959). “There's Plenty of Room at the Bottom”. Annual meeting of the American Physical Society. California Institute of Technology), Neiman (1964). “Some fundamental issues of microminiaturization” (PDF). Radiotekhnika (in Russian) (1): 3-12)) and Wiener (1964. “Interview: machines smarter than men?”. U.S. News & World Report. 56:84-86). Techniques and methods have developed over the proceeding decades, with increasingly complex demonstrations of data stored into DNA. Notably, in 2019 the entire English language version of Wikipedia was encoded into DNA by the company CATALOG. Whilst DNA data storage has focused primarily on encoding the data directly into the bases of the DNA double-helix, there have also been examples of using epigenetic marks, such as methylated cytosine to store digital data. The methods of the present application have the potential to be applied to the storing of digital data using mC.

In particular, the invention encompasses the application of the methods described herein to DNA data storage. In DNA data storage where data is stored as mC in the DNA (either as an alternative to storage within the DNA helix, or in addition thereto), the methods of the invention have potential utility to faithfully copy these mC groups. Thus, the invention provides the use of a method described herein for DNA data storage. The invention further provides a method of DNA data storage comprising the amplification of methylated polynucleotides (e.g. dsDNA) according to the present invention.

Whilst the Examples herein demonstrate recursive copying of mC from a biological source of DNA, the underlying principles apply equally to enable copying of mC for DNA data storage. Copying of mC using the methods of the invention has the potential to be significantly more faithful than the methods using PCR methods to encode data that is stored directly within the base-sequence of the DNA. As described herein, DNMT according to the invention typically use energy in the form of ATP (via an ATPase domain), to ensure exquisitely faithful copying of the methylated cytosine, with the potential to yield error free copies of the data. Even the most accurate PCR reactions are known to introduce errors into the copied DNA at the rate of approximately between 1 in 1000 to 1 in 1 million errors per base. Due to the faithful nature of the DNMT copying of mC, the copying of data described by this invention could be essentially error-free.

When methods of the invention are used for data storage, the SAM analogues used as methyl donors may add a protecting group at the same time as the methyl group, as described herein. This is potentially advantageous, as it increases the stability of the resulting mC, reducing the likelihood and/or rate of degradation of the DNA, making it more suitable for long-term storage.

### Synchronised DNA Amplification and Methylation

As described and exemplified herein, the present inventors are the first to describe a system in which DNA amplification and methylation can occur (i) at the same time, (ii) in a single reaction vessel, (iii) using a common buffer, and (iv) without needing to purify the amplified DNA before methylation occurs. As such, entirely new approach has potential utility beyond the methods using H-E-M cycles as described herein.

Accordingly, the present invention provides a method for performing DNA amplification and methylation in a single reaction vessel. Typically, in these methods, DNA amplification and methylation occurs at the same time. Thus, these methods may be described as synchronised DNA amplification and methylation methods.

Typically, a common reaction buffer is used throughout the method, and/or no purification of the amplified DNA is required before methylation can occur.

In particular, the buffer may be selected such that a polymerase (e.g. DNA polymerase) and DNMT (e.g. DNMT5) are both at least partially active in said buffer. Partial activity of the polymerase (e.g. DNA polymerase) and DNMT (e.g. DNMT5) may be as defined herein.

As described herein, typically a DNMT (e.g. a DNMT5) requires (i) SAM or a SAM analogue and (ii) ATP or dATP, to be active. Therefore, a buffer for use in said method typically comprises (i) SAM or a SAM analogue and (ii) ATP or dATP.

Also as described herein, typically the DNMT (e.g. DNMT5) and polymerase (e.g. DNA polymerase) require cations, particularly Mg²⁺to be active.

Therefore, the buffer for use according to the present invention is typically supplemented with cations, particularly Mg²⁺, as described herein.

Accordingly, the present invention provides a method for performing DNA amplification and methylation in a single reaction vessel, wherein a common (i.e. single) reaction buffer is used throughout, which buffer comprises (i) SAM or a SAM analogue, (ii) ATP or dATP and (iii) cations, particularly Mg²⁺.

Any appropriate buffer may be used, provided it is supplemented with (i) SAM or a SAM analogue, (ii) ATP or dATP and (iii) Mg²⁺as described herein. Suitable examples of buffers are described herein and also known in the art, and may be selected by one of ordinary skill without undue burden. By way of non-limiting example, suitable buffers include Tris-based buffers and HEPES buffers.

As described herein, said method may occur with DNA amplification and methylation taking place simultaneously within the reaction vessel, without the need to (i) purify the amplified DNA prior to methylation; and/or (ii) purify the methylated DNA prior to amplification.

Accordingly, a method of the invention may take place without any liquid transfers of substrate into or out of the reaction vessel, or transfer of substrate into a second reaction vessel.

A method of the invention may comprise the addition of reagents to the reaction vessel. For example, one or more addition of: (i) SAM or a SAM analogue, (ii) ATP or dATP; and/or (iii) cations, particularly Mg²⁺; may be made to the reaction vessel during the course of the method.

Alternatively, or in addition, the DNMT and/or DNA polymerase may be removed or inactivated during the course of the method.

One-pot methods of the invention comprising H-E-M cycles are preferred aspects of the synchronised DNA amplification and methylation methods of the invention.

### Ascl Assay

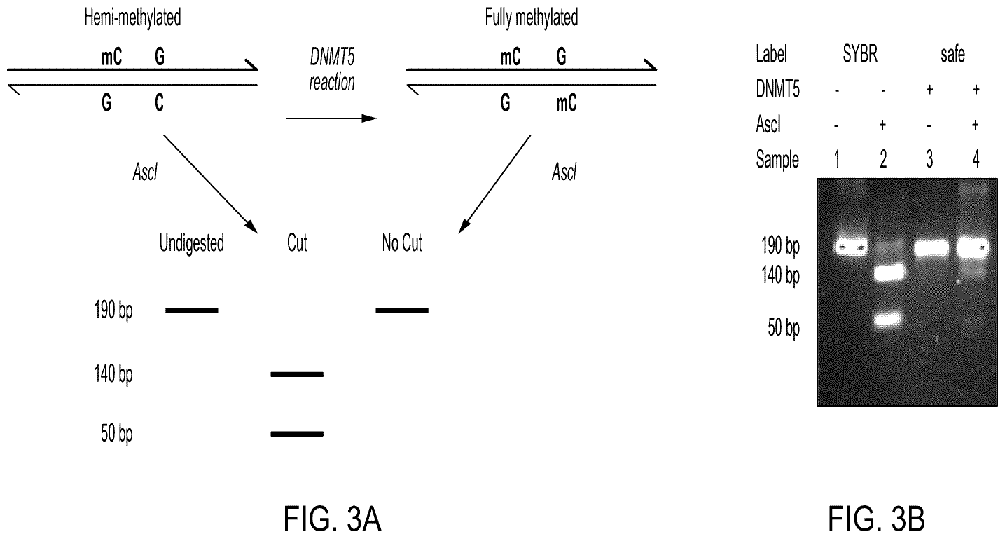

As exemplified herein, the inventors have also developed an assay which allows for methylation on the first and second strand of a symmetrically-methylated dsDNA product to be differentiated. A schematic of such an assay is shown in FIG. 3A.

Thus, the invention provides a method of differentiating between mC on the first and second strand of a symmetrically-methylated dsDNA product. Said method typically comprises or consists of (i) introducing a first restriction enzyme recognition site into a dsDNA, wherein said site can be cleaved by a corresponding first restriction enzyme when said site is hemimethylated, but not when said site is symmetrically-methylated; (ii) carrying out a method of amplifying methylated DNA; (iii) contacting the amplified methylated DNA with the first restriction enzyme; and (iv) visualising the digestion of the methylated DNA by the first restriction enzyme; wherein digestion of the methylated DNA indicates that step (ii) did not produce a symmetrically-methylated dsDNA product, and reduced or no digestion of the methylated DNA indicates that step (ii) produced a symmetrically-methylated dsDNA product.

Any restriction enzyme that cleaves hemimethylated DNA but not symmetrically-methylated DNA could be used herein. By way of a non-limiting example, a first restriction enzyme is Ascl. An exemplary Ascl restriction enzyme recognition site is 5′-GG|CGCGCC-3′ (SEQ ID NO: 15, where the | mark indicates the site of cleavage on dsDNA).

Step (i) of said method may comprise introducing a second restriction enzyme recognition site into the dsDNA, which second site is cleavable by a second restriction enzyme which is different to the first restriction enzyme. Typically said second site can be cleaved irrespective of its methylation status, i.e. it can be cleaved by the second restriction enzyme when said site is hemimethylated or symmetrically-methylated. Inclusion of a second site and use of a second restriction enzyme can be used to confirm that amplification has been successful (e.g. if the second site is comprised within a primer which is extended in step (ii), but not in the original dsDNA substrate), allowing the use of the first site and first restriction enzyme to confirm methylation.

Preferably, the second restriction enzyme is EcoR1. An exemplary EcoR1 restriction enzyme recognition site is 5′-G|AATTC-3′ (SEQ ID NO: 16, where the | mark indicates the site of cleavage on dsDNA).

### DNMT Products and Kits

The invention also provides products for use in the methods and applications of the invention.

In particular, the invention provides a DNMT5 truncated at the C-terminus and/or N-terminus, which comprises both methyltransferase activity and ATPase activity. The truncated DNMT5 may comprise or consist of an amino acid sequence of any one of SEQ ID NOs: 28 or 30 to 34, preferably any one of SEQ ID NOs: 25 to 34, particularly any one of SEQ ID NOS: 28 to 34. Alternatively or in addition, said truncated DNMT5 may be labelled and/or tagged, as described herein.

The invention provides an enzyme comprising both methyltransferase activity and ATPase activity immobilised on an inert support. The enzyme comprising both methyltransferase activity and ATPase activity may be as defined herein. In particular, the enzyme comprising both methyltransferase activity and ATPase activity may comprise or consist of an amino acid sequence of any one of SEQ ID NOs: 23 to 34, typically an amino acid sequence of any one of SEQ ID NOs: 28 or 30 to 34, preferably any one of SEQ ID NOS: 25 to 34, particularly any one of SEQ ID NOs: 28 to 34. Alternatively or in addition, said enzyme comprising both methyltransferase activity and ATPase activity may be labelled and/or tagged, as described herein.

The enzyme comprising both methyltransferase activity and ATPase activity (e.g. truncated DNMT5) may be combined with one or more of SAM; ATP, dATP; a DNA polymerase; Mg²⁺ cations; a proteinase, such as proteinase K, a ligase an oligonucleotide primer, any of which may be in lyophilised form.

The inert support may be as described herein, optionally the support is beads, such as magnetic beads. The enzyme comprising both methyltransferase activity and ATPase activity (e.g. truncated DNMT5) may be permanently immobilised on the support.

In particular, the invention provides products comprising DNMT in a permanently immobilised form which are suitable for use in a method of the invention. Thus, the invention provides DNMT permanently immobilised on a solid support, typically wherein the solid support is an inert support.

The invention provides DNMT permanently immobilised to a solid and/or inert support. Permanent immobilisation of the DNMT may be by any appropriate means (e.g. covalent or non-covalent), non-limiting examples of which are described herein. In some embodiments the solid and/or inert support may be selected from a surface, bead or membrane, or a plurality of any of the above. The surface may, for example, be the surface of a microplate well, or the surface of a microfluidic chip, or chamber or zone thereof. Wherein the support is a bead or plurality thereof, the bead(s) may be any suitable type of bead, examples of which are known in the art and commercially available, such as magnetic beads or agarose beads. Wherein the support is a surface of a microfluidic chip, or chamber or zone thereof, a polymerase may also be immobilised (permanently or temporarily, preferably permanently) to a surface of a microfluidic chip, or chamber or zone thereof. The polymerase may be immobilised to the surface of the same chamber or zone of the microfluidic chip, or to a different chamber or zone thereof. Immobilisation of the DNMT and the polymerase to the surface of different chambers or zone of a microfluidic chip, this may allow the primers and/or polynucleotide (e.g. dsDNA) substrate to be moved across the polymerase and DNMT in sequence, e.g. by injection or fluid flow.

The immobilised DNMT may be any enzyme comprising both methyltransferase activity and ATPase activity, such as a truncated DNMT. The enzyme may be a DNMT5 or a truncated DNMT5. Such enzymes comprising both methyltransferase activity and ATPase activity, truncated DNMTs, DNMT5s and truncated DNMT5s are described herein. Any and all disclosure herein in relation to products for use in the methods and applications of the invention, e.g. to DNMT permanently immobilised to a solid and/or inert support apply equally and without reservation to embodiments in which the DNMT is a truncated DNMT, DNMT5 or a truncated DNMT5, such as described herein. By way of non-limiting example, the invention provides DNMT5 permanently immobilised to a solid and/or inert support such as the surface of a microplate well, or the surface of a microfluidic chip, or chamber or zone thereof, with magnetic beads being particularly preferred. The DNMT5 may comprise or consist of an amino acid sequence having at least 60% identity, preferably at least 80% identity (e.g. at least 90%, at least 95%, at least 97% or more, up to 100% identity) to SEQ ID NO: 1 or 3, particularly SEQ ID NO: 3 as described herein, with the DNMT5 of SEQ ID NO: 3 or 1 being particularly preferred. The DNMT5 may be as defined herein. In particular, the DNMT5 may comprise or consist of an amino acid sequence of any one of SEQ ID NOs: 23 to 34, typically an amino acid sequence of any one of SEQ ID NOs: 28 or 30 to 34, preferably any one of SEQ ID NOs: 25 to 34, particularly any one of SEQ ID NOs: 28 to 34.

The invention further comprises a kit comprising an enzyme comprising both methyltransferase activity and ATPase activity (a DNMT as defined herein) for use in a method of the invention. A kit of the invention may comprise an enzyme comprising both methyltransferase activity and ATPase activity, wherein optionally the enzyme is as defined herein. The enzyme may be labelled or tagged, optionally wherein a label is present at the N- and/or C-terminal of the enzyme, preferably at the C-terminal. The enzyme may be immobilised on an inert support. Optionally said kit may further comprise instructions for use.

Thus, the invention provides a kit comprising DNMT, which optionally may further comprise one or more of (i) SAM; (ii) ATP and/or dATP; (iii) a DNA polymerase; (iv) a DNA cleaving enzyme; (v) one or more oligonucleotide primers; (vi) Mg²⁺ cations; (vii) a proteinase, such as proteinase K; (viii) one or more buffer; (ix) a ligase and/or (x) control DNA. Any of the SAM, ATP, dATP, DNA polymerase, DNA cleaving enzyme, primers, Mg²⁺ cations, proteinase, ligase and/or control DNA may be separately or together lyophilized. Optionally said kit may further comprise instructions for use.

The enzyme comprising both methyltransferase activity and ATPase activity (DNMT) in a kit of the invention may be permanently immobilised on any suitable solid and/or inert support, such as those described herein, e.g. on beads (with magnetic beads being particularly preferred), or on the surface of a microfluidic chip or a chamber or zone thereof. Alternatively or in addition, the DNMT within said kit may preferably be a DNMT5, such as a DNMT5 as described herein. Any and all disclosure herein in relation to kits of the invention applies equally and without reservation to embodiments in which the DNMT is DNMT5, such as a DNMT5 as described herein. The DNMT5 may comprise or consist of an amino acid sequence having at least 60%, preferably at least 80% identity (e.g. at least 90%, at least 95%, at least 97% or more, up to 100% identity) to SEQ ID NO: 1 or 3 as described herein, particularly SEQ ID NO: 3, with the DNMT5 of SEQ ID NO: 3 or 1 being particularly preferred. The DNMT5 may be as defined herein. In particular, the DNMT5 may comprise or consist of an amino acid sequence of any one of SEQ ID NOs: 23 to 34, typically an amino acid sequence of any one of SEQ ID NOs: 28 or 30 to 34, preferably any one of SEQ ID NOs: 25 to 34, particularly any one of SEQ ID NOs: 28 to 34.

### Sequence Homology

An amino acid modification according to the invention may be a substitution, deletion, addition or other modification, including post-translational modification, unless the relevant disclosure explicitly says otherwise. Preferably said modifications are amino acid substitutions. In other words, the amino acid at a specified position within the antigen of the invention is substituted by a naturally occurring or non-naturally occurring amino acid that is different to the amino acid present at that position in the sequence from which the antigen of the invention is derived. Alternatively, the amino acid at a specified position within the antigen of the invention may be modified post-translationally. Post-translational modifications include glycosylation's, acetylation's, acylation's, de-aminations, phosphorylisations, isoprenylisations, glycosyl phosphatidyl inositolisations and further modifications known to a person skilled in the art.

The modification of one or more amino acid position as described herein may be performed, for example, by specific mutagenesis, or any other method known in the art. In embodiments in which one or more amino acid position is substituted relative to the corresponding antigen from which the antigen of the invention is derived, the substitution may be a conservative substitution or a non-conservative substitution. A conservative substitution is defined as substitution by an amino acid pertaining to the same physiochemical group to the amino acid present in the antigen from which the antigen of the invention is derived. A non-conservative amino acid substitution is defined as substitution by an amino acid pertaining to a different physiochemical group to the amino acid present in the antigen from which the antigen of the invention is derived. In more detail, amino acids are, in principle, divided into different physiochemical groups. Aspartate and glutamate belong to the negatively charged amino acids. Histidine, arginine and lysine belong to the positively charged amino acids. Asparagine, glutamine, serine, threonine, cysteine and tyrosine belong to the polar amino acids. Glycine, alanine, valine, leucine, isoleucine, methionine, proline, phenylalanine and tryptophan belong to the non-polar amino acids. Aromatic side groups are to be found among the amino acids histidine, phenylalanine, tyrosine and tryptophan. Thus, as a non-limiting example, a conservative substation may involve the substitution of a non-polar amino acid by another non-polar amino acid, such as substituting leucine with isoleucine. As another non-limiting example, a non-conservative substitution may involve the substation of a non-polar amino acid (e.g., leucine) with a negatively charged amino acid (e.g., aspartate), a positively-charged amino acid (e.g. arginine), or a polar amino acid (e.g. asparagine).

Conventional methods for determining amino acid sequence identity are known in the art. The terms “sequence identity” and “sequence homology” are considered synonymous in this specification.

By way of example, a polypeptide of interest may comprise an amino acid sequence having at least 70, 75, 80, 82, 84, 86, 88, 90, 92, 94, 96, 98, 99 or 100% amino acid sequence identity with the amino acid sequence of a reference polypeptide.

There are many established algorithms available to align two amino acid sequences. Typically, one sequence acts as a reference sequence, to which test sequences may be compared. The sequence comparison algorithm calculates the percentage sequence identity for the test sequence(s) relative to the reference sequence, based on the designated program parameters. Alignment of amino acid sequences for comparison may be conducted, for example, by computer implemented algorithms (e.g., GAP, BESTFIT, FASTA or TFASTA), or BLAST and BLAST 2.0 algorithms.

The BLOSUM62 table shown below is an amino acid substitution matrix derived from about 2,000 local multiple alignments of protein sequence segments, representing highly conserved regions of more than 500 groups of related proteins (Henikoff & Henikoff, Proc. Natl. Acad. Sci. USA 89:10915-10919, 1992; incorporated herein by reference). Amino acids are indicated by the standard one-letter codes. The percent identity is calculated as:

$$\frac{Total number of identical matches}{[ length of the longer sequence plus the number of gaps} \times 100$$

Introduced into the longer sequence in order to align the two sequences]

BLOSUM62 table

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

In a homology comparison, the identity may exist over a region of the sequences that is at least 10 amino acid residues in length (e.g., at least 15, 20, 30, 40, 50, 75, 100, 150, 200, 250, 300, 350, 400, 450, 500 or 520 amino acid residues in length)—e.g., up to the entire length of the reference sequence.

Substantially homologous polypeptides have one or more amino acid substitutions, deletions, or additions. In many embodiments, those changes are of a minor nature, for example, involving only conservative amino acid substitutions. Conservative substitutions are those made by replacing one amino acid with another amino acid within the following groups: Basic: arginine, lysine, histidine; Acidic: glutamic acid, aspartic acid; Polar: glutamine, asparagine; Hydrophobic: leucine, isoleucine, valine; Aromatic: phenylalanine, tryptophan, tyrosine; Small: glycine, alanine, serine, threonine, methionine. Substantially homologous polypeptides also encompass those comprising other substitutions that do not significantly affect the folding or activity of the polypeptide; small deletions, typically of 1 to about 30 amino acids (such as 1-10, or 1-5 amino acids); and small amino- or carboxyl-terminal extensions, such as an amino-terminal methionine residue, a small linker peptide of up to about 20-25 residues, or an affinity tag.

### Sequence Information

Where an initial Met amino acid residue or a corresponding initial codon is indicated in any of the following amino acid SEQ ID NOs, said residue/codon is optional.
SEQ ID NO 1: amino acid sequence for DNMT5 from Cryptococcus neoformans (UniProt Accession No. J9VI03 version 2, accessed 8 Jul. 2023)
SEQ ID NO 2: nucleic acid sequence for DNMT5 from Cryptococcus neoformans
SEQ ID NO 3: amino acid sequence for DNMT5 from Cryptococcus tepidarius
SEQ ID NO 4: nucleic acid sequence for DNMT5 from Cryptococcus tepidarius
SEQ ID NO 5: DNA sequence 1
SEQ ID NO 6: P257
SEQ ID NO 7: P258
SEQ ID NO 8: T7 for-Cy3
SEQ ID NO 9: T7 for-Atto647
SEQ ID NO 10: T7rev-Cy3
SEQ ID NO 11: T7rev-Atto647
SEQ ID NO 12: methylated top strand of DNA sequence 2
SEQ ID NO 13: unmethylated bottom strand of DNA sequence 2 with biotin
SEQ ID NO: 14: amino acid sequence for C-terminal His-tagged DNMT5 from Cryptococcus neoformans
SEQ ID NO: 15: Ascl recognition sequence
SEQ ID NO: 16: EcoRI recognition sequence
SEQ ID NO 17: methylated bottom strand of DNA sequence 2 with biotin
SEQ ID NO 18: T7F
SEQ ID NO 19: methylated SEPT9 sequence
SEQ ID NO 20: unmethylated SEPT9 sequence
SEQ IS NO 21: SEPT9 forward primer
SEQ ID NO 22: SEPT9 reverse primer
SEQ ID NO 23: NeoFLAGHis variant
SEQ ID NO 24: Variant 2
SEQ ID NO 25: Variant 3
SEQ ID NO 26: Variant 4
SEQ ID NO 27: Variant 5
SEQ ID NO 28: Variant 6
SEQ ID NO 29: Variant 7
SEQ ID NO 30: Variant 8
SEQ ID NO 31: Variant 9
SEQ ID NO 32: Variant 10
SEQ ID NO 33: Variant 11
SEQ ID NO 34: Variant 12
SEQ ID NO: 35: MT1 consensus sequence
SEQ ID NO: 36: MT23 consensus sequence
SEQ ID NO: 37: MT4 consensus sequence
SEQ ID NO: 38: MT5 consensus sequence
SEQ ID NO: 39: MT6 consensus sequence
SEQ ID NO: 40: MT7 consensus sequence
SEQ ID NO: 41: MT7B consensus sequence
SEQ ID NO: 42: MT8 consensus sequence
SEQ ID NO: 43: MT8B consensus sequence
SEQ ID NO: 44: MT9 consensus sequence
SEQ ID NO: 45: MT10 consensus sequence
SEQ ID NO: 46: ATP11B consensus sequence
SEQ ID NO: 47: ATP12 consensus sequence
SEQ ID NO: 48: ATP13 consensus sequence
SEQ ID NO: 49: ATP14 consensus sequence
SEQ ID NO: 50: ATP14B consensus sequence
SEQ ID NO: 51: ATP15 consensus sequence
SEQ ID NO: 52: ATP15B consensus sequence
SEQ ID NO: 53: MT1 sequence DNMT5 exemplar 1
SEQ ID NO: 54: MT23 sequence DNMT5 exemplar 1
SEQ ID NO: 55: MT4 sequence DNMT5 exemplar 1
SEQ ID NO: 56: MT5 sequence DNMT5 exemplar 1
SEQ ID NO: 57: MT6 sequence DNMT5 exemplar 1
SEQ ID NO: 58: MT7 sequence DNMT5 exemplar 1
SEQ ID NO: 59: MT7B sequence DNMT5 exemplar 1
SEQ ID NO: 60: MT8 sequence DNMT5 exemplar 1
SEQ ID NO: 61: MT8B sequence DNMT5 exemplar 1
SEQ ID NO: 62: MT9 sequence DNMT5 exemplar 1
SEQ ID NO: 63: MT10 sequence DNMT5 exemplar 1
SEQ ID NO: 64: ATP11B sequence DNMT5 exemplar 1
SEQ ID NO: 65: ATP12 sequence DNMT5 exemplar 1
SEQ ID NO: 66: ATP13 sequence DNMT5 exemplar 1
SEQ ID NO: 67: ATP14 sequence DNMT5 exemplar 1
SEQ ID NO: 68: ATP14B sequence DNMT5 exemplar 1
SEQ ID NO: 69: ATP15 sequence DNMT5 exemplar 1
SEQ ID NO: 70: ATP15B sequence DNMT5 exemplar 1
SEQ ID NO: 71: MT1 sequence DNMT5 exemplar 2
SEQ ID NO: 72: MT23 sequence DNMT5 exemplar 2
SEQ ID NO: 73: MT4 sequence DNMT5 exemplar 2
SEQ ID NO: 74: MT5 sequence DNMT5 exemplar 2
SEQ ID NO: 75: MT6 sequence DNMT5 exemplar 2
SEQ ID NO: 76: MT7 sequence DNMT5 exemplar 2
SEQ ID NO: 77: MT7B sequence DNMT5 exemplar 2
SEQ ID NO: 78: MT8 sequence DNMT5 exemplar 2
SEQ ID NO: 79: MT8B sequence DNMT5 exemplar 2
SEQ ID NO: 80: MT9 sequence DNMT5 exemplar 2
SEQ ID NO: 81: MT10 sequence DNMT5 exemplar 2
SEQ ID NO: 82: ATP11B sequence DNMT5 exemplar 2
SEQ ID NO: 83: ATP12 sequence DNMT5 exemplar 2
SEQ ID NO: 84: ATP13 sequence DNMT5 exemplar 2
SEQ ID NO: 85: ATP14 sequence DNMT5 exemplar 2
SEQ ID NO: 86: ATP14B sequence DNMT5 exemplar 2
SEQ ID NO: 87: ATP15 sequence DNMT5 exemplar 2
SEQ ID NO: 88: ATP15B sequence DNMT5 exemplar 2
SEQ ID NO: 89: MT1 sequence DNMT5 exemplar 3
SEQ ID NO: 90: MT23 sequence DNMT5 exemplar 3
SEQ ID NO: 91: MT4 sequence DNMT5 exemplar 3
SEQ ID NO: 92: MT5 sequence DNMT5 exemplar 3
SEQ ID NO: 93: MT6 sequence DNMT5 exemplar 3
SEQ ID NO: 94: MT7 sequence DNMT5 exemplar 3
SEQ ID NO: 95: MT7B sequence DNMT5 exemplar 3
SEQ ID NO: 96: MT8 sequence DNMT5 exemplar 3
SEQ ID NO: 97: MT8B sequence DNMT5 exemplar 3
SEQ ID NO: 98: MT9 sequence DNMT5 exemplar 3
SEQ ID NO: 99: MT10 sequence DNMT5 exemplar 3
SEQ ID NO: 100: ATP11B sequence DNMT5 exemplar 3
SEQ ID NO: 101: ATP12 sequence DNMT5 exemplar 3
SEQ ID NO: 102: ATP13 sequence DNMT5 exemplar 3
SEQ ID NO: 103: ATP14 sequence DNMT5 exemplar 3
SEQ ID NO: 104: ATP14B sequence DNMT5 exemplar 3
SEQ ID NO: 105: ATP15 sequence DNMT5 exemplar 3
SEQ ID NO: 106: ATP15B sequence DNMT5 exemplar 3
SEQ ID NO: 107: MT1 sequence DNMT5 exemplar 4
SEQ ID NO: 108: MT23 sequence DNMT5 exemplar 4
SEQ ID NO: 109: MT4 sequence DNMT5 exemplar 4
SEQ ID NO: 110: MT5 sequence DNMT5 exemplar 4
SEQ ID NO: 111: MT6 sequence DNMT5 exemplar 4
SEQ ID NO: 112: MT7 sequence DNMT5 exemplar 4
SEQ ID NO: 113: MT7B sequence DNMT5 exemplar 4
SEQ ID NO: 114: MT8 sequence DNMT5 exemplar 4
SEQ ID NO: 115: MT8B sequence DNMT5 exemplar 4
SEQ ID NO: 116: MT9 sequence DNMT5 exemplar 4
SEQ ID NO: 117: MT10 sequence DNMT5 exemplar 4
SEQ ID NO: 118: ATP11B sequence DNMT5 exemplar 4
SEQ ID NO: 119: ATP12 sequence DNMT5 exemplar 4
SEQ ID NO: 120: ATP13 sequence DNMT5 exemplar 4
SEQ ID NO: 121: ATP14 sequence DNMT5 exemplar 4
SEQ ID NO: 122: ATP14B sequence DNMT5 exemplar 4
SEQ ID NO: 123: ATP15 sequence DNMT5 exemplar 4
SEQ ID NO: 124: ATP15B sequence DNMT5 exemplar 4
SEQ ID NO: 125: MT1 sequence DNMT5 exemplar 5
SEQ ID NO: 126: MT23 sequence DNMT5 exemplar 5
SEQ ID NO: 127: MT4 sequence DNMT5 exemplar 5
SEQ ID NO: 128: MT5 sequence DNMT5 exemplar 5
SEQ ID NO: 129: MT6 sequence DNMT5 exemplar 5
SEQ ID NO: 130: MT7 sequence DNMT5 exemplar 5
SEQ ID NO: 131: MT7B sequence DNMT5 exemplar 5
SEQ ID NO: 132: MT8 sequence DNMT5 exemplar 5
SEQ ID NO: 133: MT8B sequence DNMT5 exemplar 5
SEQ ID NO: 134: MT9 sequence DNMT5 exemplar 5
SEQ ID NO: 135: MT10 sequence DNMT5 exemplar 5
SEQ ID NO: 136: ATP11B sequence DNMT5 exemplar 5
SEQ ID NO: 137: ATP12 sequence DNMT5 exemplar 5
SEQ ID NO: 138: ATP13 sequence DNMT5 exemplar 5
SEQ ID NO: 139: ATP14 sequence DNMT5 exemplar 5
SEQ ID NO: 140: ATP14B sequence DNMT5 exemplar 5
SEQ ID NO: 141: ATP15 sequence DNMT5 exemplar 5
SEQ ID NO: 142: ATP15B sequence DNMT5 exemplar 5
SEQ ID NO: 143: MT1 sequence DNMT5 exemplar 6
SEQ ID NO: 144: MT23 sequence DNMT5 exemplar 6
SEQ ID NO: 145: MT4 sequence DNMT5 exemplar 6
SEQ ID NO: 146: MT5 sequence DNMT5 exemplar 6
SEQ ID NO: 147: MT6 sequence DNMT5 exemplar 6
SEQ ID NO: 148: MT7 sequence DNMT5 exemplar 6
SEQ ID NO: 149: MT7B sequence DNMT5 exemplar 6
SEQ ID NO: 150: MT8 sequence DNMT5 exemplar 6
SEQ ID NO: 151: MT8B sequence DNMT5 exemplar 6
SEQ ID NO: 152: MT9 sequence DNMT5 exemplar 6
SEQ ID NO: 153: MT10 sequence DNMT5 exemplar 6
SEQ ID NO: 154: ATP11B sequence DNMT5 exemplar 6
SEQ ID NO: 155: ATP12 sequence DNMT5 exemplar 6
SEQ ID NO: 156: ATP13 sequence DNMT5 exemplar 6
SEQ ID NO: 157: ATP14 sequence DNMT5 exemplar 6
SEQ ID NO: 158: skipped
SEQ ID NO: 159: ATP15 sequence DNMT5 exemplar 6
SEQ ID NO: 160: ATP15B sequence DNMT5 exemplar 6
SEQ ID NO: 161: skipped
SEQ ID NO: 162: skipped
SEQ ID NO: 163: MT4 sequence DNMT5 exemplar 7
SEQ ID NO: 164: MT5 sequence DNMT5 exemplar 7
SEQ ID NO: 165: MT6 sequence DNMT5 exemplar 7
SEQ ID NO: 166: MT7 sequence DNMT5 exemplar 7
SEQ ID NO: 167: MT7B sequence DNMT5 exemplar 7
SEQ ID NO: 168: MT8 sequence DNMT5 exemplar 7
SEQ ID NO: 169: MT8B sequence DNMT5 exemplar 7
SEQ ID NO: 170: MT9 sequence DNMT5 exemplar 7
SEQ ID NO: 171: MT10 sequence DNMT5 exemplar 7
SEQ ID NO: 172: ATP11B sequence DNMT5 exemplar 7
SEQ ID NO: 173: ATP12 sequence DNMT5 exemplar 7
SEQ ID NO: 174: ATP13 sequence DNMT5 exemplar 7
SEQ ID NO: 175: ATP14 sequence DNMT5 exemplar 7
SEQ ID NO: 176: ATP14B sequence DNMT5 exemplar 7
SEQ ID NO: 177: ATP15 sequence DNMT5 exemplar 7
SEQ ID NO: 178: ATP15B sequence DNMT5 exemplar 7
SEQ ID NO: 179: MT1 sequence DNMT5 exemplar 8
SEQ ID NO: 180: MT23 sequence DNMT5 exemplar 8
SEQ ID NO: 181: MT4 sequence DNMT5 exemplar 8
SEQ ID NO: 182: MT5 sequence DNMT5 exemplar 8
SEQ ID NO: 183: MT6 sequence DNMT5 exemplar 8
SEQ ID NO: 184: MT7 sequence DNMT5 exemplar 8
SEQ ID NO: 185: MT7B sequence DNMT5 exemplar 8
SEQ ID NO: 186: MT8 sequence DNMT5 exemplar 8
SEQ ID NO: 187: MT8B sequence DNMT5 exemplar 8
SEQ ID NO: 188: MT9 sequence DNMT5 exemplar 8
SEQ ID NO: 189: MT10 sequence DNMT5 exemplar 8
SEQ ID NO: 190: ATP11B sequence DNMT5 exemplar 8
SEQ ID NO: 191: ATP12 sequence DNMT5 exemplar 8
SEQ ID NO: 192: ATP13 sequence DNMT5 exemplar 8
SEQ ID NO: 193: ATP14 sequence DNMT5 exemplar 8
SEQ ID NO: 194: ATP14B sequence DNMT5 exemplar 8
SEQ ID NO: 195: skipped
SEQ ID NO: 196: skipped
SEQ ID NO: 197: MT1 sequence DNMT5 exemplar 9
SEQ ID NO: 198: MT23 sequence DNMT5 exemplar 9
SEQ ID NO: 199: MT4 sequence DNMT5 exemplar 9
SEQ ID NO: 200: MT5 sequence DNMT5 exemplar 9
SEQ ID NO: 201: MT6 sequence DNMT5 exemplar 9
SEQ ID NO: 202: MT7 sequence DNMT5 exemplar 9
SEQ ID NO: 203: MT7B sequence DNMT5 exemplar 9
SEQ ID NO: 204: MT8 sequence DNMT5 exemplar 9
SEQ ID NO: 205: MT8B sequence DNMT5 exemplar 9
SEQ ID NO: 206: MT9 sequence DNMT5 exemplar 9
SEQ ID NO: 207: MT10 sequence DNMT5 exemplar 9
SEQ ID NO: 208: ATP11B sequence DNMT5 exemplar 9
SEQ ID NO: 209: ATP12 sequence DNMT5 exemplar 9
SEQ ID NO: 210: ATP13 sequence DNMT5 exemplar 9
SEQ ID NO: 211: ATP14 sequence DNMT5 exemplar 9
SEQ ID NO: 212: ATP14B sequence DNMT5 exemplar 9
SEQ ID NO: 213: ATP15 sequence DNMT5 exemplar 9
SEQ ID NO: 214: ATP15B sequence DNMT5 exemplar 9
SEQ ID NO: 215: MT1 sequence DNMT5 exemplar 10
SEQ ID NO: 216: MT23 sequence DNMT5 exemplar 10
SEQ ID NO: 217: MT4 sequence DNMT5 exemplar 10
SEQ ID NO: 218: MT5 sequence DNMT5 exemplar 10
SEQ ID NO: 219: MT6 sequence DNMT5 exemplar 10
SEQ ID NO: 220: MT7 sequence DNMT5 exemplar 10
SEQ ID NO: 221: MT7B sequence DNMT5 exemplar 10
SEQ ID NO: 222: MT8 sequence DNMT5 exemplar 10
SEQ ID NO: 223: MT8B sequence DNMT5 exemplar 10
SEQ ID NO: 224: MT9 sequence DNMT5 exemplar 10
SEQ ID NO: 225: MT10 sequence DNMT5 exemplar 10
SEQ ID NO: 226: ATP11B sequence DNMT5 exemplar 10
SEQ ID NO: 227: ATP12 sequence DNMT5 exemplar 10
SEQ ID NO: 228: ATP13 sequence DNMT5 exemplar 10
SEQ ID NO: 229: ATP14 sequence DNMT5 exemplar 10
SEQ ID NO: 230: ATP14B sequence DNMT5 exemplar 10
SEQ ID NO: 231: ATP15 sequence DNMT5 exemplar 10
SEQ ID NO: 232: ATP15B sequence DNMT5 exemplar 10
SEQ ID NO: 233: MT1 sequence DNMT5 exemplar 11
SEQ ID NO: 234: MT23 sequence DNMT5 exemplar 11
SEQ ID NO: 235: MT4 sequence DNMT5 exemplar 11
SEQ ID NO: 236: MT5 sequence DNMT5 exemplar 11
SEQ ID NO: 237: MT6 sequence DNMT5 exemplar 11
SEQ ID NO: 238: MT7 sequence DNMT5 exemplar 11
SEQ ID NO: 239: MT7B sequence DNMT5 exemplar 11
SEQ ID NO: 240: MT8 sequence DNMT5 exemplar 11
SEQ ID NO: 241: MT8B sequence DNMT5 exemplar 11
SEQ ID NO: 242: MT9 sequence DNMT5 exemplar 11
SEQ ID NO: 243: MT10 sequence DNMT5 exemplar 11
SEQ ID NO: 244: ATP11B sequence DNMT5 exemplar 11
SEQ ID NO: 245: ATP12 sequence DNMT5 exemplar 11
SEQ ID NO: 246: ATP13 sequence DNMT5 exemplar 11
SEQ ID NO: 247: ATP14 sequence DNMT5 exemplar 11
SEQ ID NO: 248: ATP14B sequence DNMT5 exemplar 11
SEQ ID NO: 249: ATP15 sequence DNMT5 exemplar 11
SEQ ID NO: 250: ATP15B sequence DNMT5 exemplar 11
SEQ ID NO: 251: MT1 sequence DNMT5 exemplar 12
SEQ ID NO: 252: MT23 sequence DNMT5 exemplar 12
SEQ ID NO: 253: MT4 sequence DNMT5 exemplar 12
SEQ ID NO: 254: MT5 sequence DNMT5 exemplar 12
SEQ ID NO: 255: MT6 sequence DNMT5 exemplar 12
SEQ ID NO: 256: MT7 sequence DNMT5 exemplar 12
SEQ ID NO: 257: MT7B sequence DNMT5 exemplar 12
SEQ ID NO: 258: MT8 sequence DNMT5 exemplar 12
SEQ ID NO: 259: MT8B sequence DNMT5 exemplar 12
SEQ ID NO: 260: MT9 sequence DNMT5 exemplar 12
SEQ ID NO: 261: MT10 sequence DNMT5 exemplar 12
SEQ ID NO: 262: ATP11B sequence DNMT5 exemplar 12
SEQ ID NO: 263: ATP12 sequence DNMT5 exemplar 12
SEQ ID NO: 264: ATP13 sequence DNMT5 exemplar 12
SEQ ID NO: 265: ATP14 sequence DNMT5 exemplar 12
SEQ ID NO: 266: ATP14B sequence DNMT5 exemplar 12
SEQ ID NO: 267: ATP15 sequence DNMT5 exemplar 12
SEQ ID NO: 268: ATP15B sequence DNMT5 exemplar 12
SEQ ID NO: 269: MT1 sequence DNMT5 exemplar 13
SEQ ID NO: 270: MT23 sequence DNMT5 exemplar 13
SEQ ID NO: 271: MT4 sequence DNMT5 exemplar 13
SEQ ID NO: 272: MT5 sequence DNMT5 exemplar 13
SEQ ID NO: 273: MT6 sequence DNMT5 exemplar 13
SEQ ID NO: 274: MT7 sequence DNMT5 exemplar 13
SEQ ID NO: 275: MT7B sequence DNMT5 exemplar 13
SEQ ID NO: 276: MT8 sequence DNMT5 exemplar 13
SEQ ID NO: 277: MT8B sequence DNMT5 exemplar 13
SEQ ID NO: 278: MT9 sequence DNMT5 exemplar 13
SEQ ID NO: 279: MT10 sequence DNMT5 exemplar 13
SEQ ID NO: 280: ATP11B sequence DNMT5 exemplar 13
SEQ ID NO: 281: ATP12 sequence DNMT5 exemplar 13
SEQ ID NO: 282: ATP13 sequence DNMT5 exemplar 13
SEQ ID NO: 283: ATP14 sequence DNMT5 exemplar 13
SEQ ID NO: 284: ATP14B sequence DNMT5 exemplar 13
SEQ ID NO: 285: ATP15 sequence DNMT5 exemplar 13
SEQ ID NO: 286: ATP15B sequence DNMT5 exemplar 13
SEQ ID NO: 287: MT1 sequence DNMT5 exemplar 14
SEQ ID NO: 288: MT23 sequence DNMT5 exemplar 14
SEQ ID NO: 289: MT4 sequence DNMT5 exemplar 14
SEQ ID NO: 290: MT5 sequence DNMT5 exemplar 14
SEQ ID NO: 291: MT6 sequence DNMT5 exemplar 14
SEQ ID NO: 292: MT7 sequence DNMT5 exemplar 14
SEQ ID NO: 293: MT7B sequence DNMT5 exemplar 14
SEQ ID NO: 294: MT8 sequence DNMT5 exemplar 14
SEQ ID NO: 295: MT8B sequence DNMT5 exemplar 14
SEQ ID NO: 296: MT9 sequence DNMT5 exemplar 14
SEQ ID NO: 297: MT10 sequence DNMT5 exemplar 14
SEQ ID NO: 298: ATP11B sequence DNMT5 exemplar 14
SEQ ID NO: 299: ATP12 sequence DNMT5 exemplar 14
SEQ ID NO: 300: ATP13 sequence DNMT5 exemplar 14
SEQ ID NO: 301: ATP14 sequence DNMT5 exemplar 14
SEQ ID NO: 302: ATP14B sequence DNMT5 exemplar 14
SEQ ID NO: 303: ATP15 sequence DNMT5 exemplar 14
SEQ ID NO: 304: ATP15B sequence DNMT5 exemplar 14
SEQ ID NO: 305: MT1 sequence DNMT5 exemplar 15
SEQ ID NO: 306: MT23 sequence DNMT5 exemplar 15
SEQ ID NO: 307: MT4 sequence DNMT5 exemplar 15
SEQ ID NO: 308: MT5 sequence DNMT5 exemplar 15
SEQ ID NO: 309: MT6 sequence DNMT5 exemplar 15
SEQ ID NO: 310: MT7 sequence DNMT5 exemplar 15
SEQ ID NO: 311: MT7B sequence DNMT5 exemplar 15
SEQ ID NO: 312: MT8 sequence DNMT5 exemplar 15
SEQ ID NO: 313: MT8B sequence DNMT5 exemplar 15
SEQ ID NO: 314: MT9 sequence DNMT5 exemplar 15
SEQ ID NO: 315: MT10 sequence DNMT5 exemplar 15
SEQ ID NO: 316: ATP11B sequence DNMT5 exemplar 15
SEQ ID NO: 317: ATP12 sequence DNMT5 exemplar 15
SEQ ID NO: 318: ATP13 sequence DNMT5 exemplar 15
SEQ ID NO: 319: ATP14 sequence DNMT5 exemplar 15
SEQ ID NO: 320: ATP14B sequence DNMT5 exemplar 15
SEQ ID NO: 321: ATP15 sequence DNMT5 exemplar 15
SEQ ID NO: 322: ATP15B sequence DNMT5 exemplar 15
SEQ ID NO: 323: MT1 sequence DNMT5 exemplar 16
SEQ ID NO: 324: MT23 sequence DNMT5 exemplar 16
SEQ ID NO: 325: MT4 sequence DNMT5 exemplar 16
SEQ ID NO: 326: MT5 sequence DNMT5 exemplar 16
SEQ ID NO: 327: MT6 sequence DNMT5 exemplar 16
SEQ ID NO: 328: MT7 sequence DNMT5 exemplar 16
SEQ ID NO: 329: MT7B sequence DNMT5 exemplar 16
SEQ ID NO: 330: MT8 sequence DNMT5 exemplar 16
SEQ ID NO: 331: MT8B sequence DNMT5 exemplar 16
SEQ ID NO: 332: MT9 sequence DNMT5 exemplar 16
SEQ ID NO: 333: MT10 sequence DNMT5 exemplar 16
SEQ ID NO: 334: ATP11B sequence DNMT5 exemplar 16
SEQ ID NO: 335: ATP12 sequence DNMT5 exemplar 16
SEQ ID NO: 336: ATP13 sequence DNMT5 exemplar 16
SEQ ID NO: 337: ATP14 sequence DNMT5 exemplar 16
SEQ ID NO: 338: ATP14B sequence DNMT5 exemplar 16
SEQ ID NO: 339: ATP15 sequence DNMT5 exemplar 16
SEQ ID NO: 340: ATP15B sequence DNMT5 exemplar 16
SEQ ID NO: 341: MT1 sequence DNMT5 exemplar 17
SEQ ID NO: 342: MT23 sequence DNMT5 exemplar 17
SEQ ID NO: 343: MT4 sequence DNMT5 exemplar 17
SEQ ID NO: 344: MT5 sequence DNMT5 exemplar 17
SEQ ID NO: 345: MT6 sequence DNMT5 exemplar 17
SEQ ID NO: 346: MT7 sequence DNMT5 exemplar 17
SEQ ID NO: 347: MT7B sequence DNMT5 exemplar 17
SEQ ID NO: 348: MT8 sequence DNMT5 exemplar 17
SEQ ID NO: 349: MT8B sequence DNMT5 exemplar 17
SEQ ID NO: 350: MT9 sequence DNMT5 exemplar 17
SEQ ID NO: 351: MT10 sequence DNMT5 exemplar 17
SEQ ID NO: 352: ATP11B sequence DNMT5 exemplar 17
SEQ ID NO: 353: ATP12 sequence DNMT5 exemplar 17
SEQ ID NO: 354: ATP13 sequence DNMT5 exemplar 17
SEQ ID NO: 355: ATP14 sequence DNMT5 exemplar 17
SEQ ID NO: 356: ATP14B sequence DNMT5 exemplar 17
SEQ ID NO: 357: ATP15 sequence DNMT5 exemplar 17
SEQ ID NO: 358: ATP15B sequence DNMT5 exemplar 17
SEQ ID NO: 359: MT1 sequence DNMT5 exemplar 18
SEQ ID NO: 360: MT23 sequence DNMT5 exemplar 18
SEQ ID NO: 361: MT4 sequence DNMT5 exemplar 18
SEQ ID NO: 362: MT5 sequence DNMT5 exemplar 18
SEQ ID NO: 363: MT6 sequence DNMT5 exemplar 18
SEQ ID NO: 364: MT7 sequence DNMT5 exemplar 18
SEQ ID NO: 365: MT7B sequence DNMT5 exemplar 18
SEQ ID NO: 366: MT8 sequence DNMT5 exemplar 18
SEQ ID NO: 367: MT8B sequence DNMT5 exemplar 18
SEQ ID NO: 368: MT9 sequence DNMT5 exemplar 18
SEQ ID NO: 369: MT10 sequence DNMT5 exemplar 18
SEQ ID NO: 370: ATP11B sequence DNMT5 exemplar 18
SEQ ID NO: 371: ATP12 sequence DNMT5 exemplar 18
SEQ ID NO: 372: ATP13 sequence DNMT5 exemplar 18
SEQ ID NO: 373: ATP14 sequence DNMT5 exemplar 18
SEQ ID NO: 374: ATP14B sequence DNMT5 exemplar 18
SEQ ID NO: 375: ATP15 sequence DNMT5 exemplar 18
SEQ ID NO: 376: ATP15B sequence DNMT5 exemplar 18
SEQ ID NO: 377: MT1 sequence DNMT5 exemplar 19
SEQ ID NO: 378: MT23 sequence DNMT5 exemplar 19
SEQ ID NO: 379: MT4 sequence DNMT5 exemplar 19
SEQ ID NO: 380: MT5 sequence DNMT5 exemplar 19
SEQ ID NO: 381: MT6 sequence DNMT5 exemplar 19
SEQ ID NO: 382: MT7 sequence DNMT5 exemplar 19
SEQ ID NO: 383: MT7B sequence DNMT5 exemplar 19
SEQ ID NO: 384: MT8 sequence DNMT5 exemplar 19
SEQ ID NO: 385: MT8B sequence DNMT5 exemplar 19
SEQ ID NO: 386: MT9 sequence DNMT5 exemplar 19
SEQ ID NO: 387: MT10 sequence DNMT5 exemplar 19
SEQ ID NO: 388: ATP11B sequence DNMT5 exemplar 19
SEQ ID NO: 389: ATP12 sequence DNMT5 exemplar 19
SEQ ID NO: 390: ATP13 sequence DNMT5 exemplar 19
SEQ ID NO: 391: ATP14 sequence DNMT5 exemplar 19
SEQ ID NO: 392: ATP14B sequence DNMT5 exemplar 19
SEQ ID NO: 393: ATP15 sequence DNMT5 exemplar 19
SEQ ID NO: 394: ATP15B sequence DNMT5 exemplar 19
SEQ ID NO: 395: MT1 sequence DNMT5 exemplar 20
SEQ ID NO: 396: MT23 sequence DNMT5 exemplar 20
SEQ ID NO: 397: MT4 sequence DNMT5 exemplar 20
SEQ ID NO: 398: MT5 sequence DNMT5 exemplar 20
SEQ ID NO: 399: MT6 sequence DNMT5 exemplar 20
SEQ ID NO: 400: MT7 sequence DNMT5 exemplar 20
SEQ ID NO: 401: MT7B sequence DNMT5 exemplar 20
SEQ ID NO: 402: MT8 sequence DNMT5 exemplar 20
SEQ ID NO: 403: MT8B sequence DNMT5 exemplar 20
SEQ ID NO: 404: MT9 sequence DNMT5 exemplar 20
SEQ ID NO: 405: MT10 sequence DNMT5 exemplar 20
SEQ ID NO: 406: ATP11B sequence DNMT5 exemplar 20
SEQ ID NO: 407: ATP12 sequence DNMT5 exemplar 20
SEQ ID NO: 408: ATP13 sequence DNMT5 exemplar 20
SEQ ID NO: 409: ATP14 sequence DNMT5 exemplar 20
SEQ ID NO: 410: ATP14B sequence DNMT5 exemplar 20
SEQ ID NO: 411: ATP15 sequence DNMT5 exemplar 20
SEQ ID NO: 412: ATP15B sequence DNMT5 exemplar 20
SEQ ID NO: 413: MT1 sequence DNMT5 exemplar 21
SEQ ID NO: 414: MT23 sequence DNMT5 exemplar 21
SEQ ID NO: 415: MT4 sequence DNMT5 exemplar 21
SEQ ID NO: 416: MT5 sequence DNMT5 exemplar 21
SEQ ID NO: 417: MT6 sequence DNMT5 exemplar 21
SEQ ID NO: 418: MT7 sequence DNMT5 exemplar 21
SEQ ID NO: 419: MT7B sequence DNMT5 exemplar 21
SEQ ID NO: 420: MT8 sequence DNMT5 exemplar 21
SEQ ID NO: 421: MT8B sequence DNMT5 exemplar 21
SEQ ID NO: 422: MT9 sequence DNMT5 exemplar 21
SEQ ID NO: 423: MT10 sequence DNMT5 exemplar 21
SEQ ID NO: 424: ATP11B sequence DNMT5 exemplar 21
SEQ ID NO: 425: ATP12 sequence DNMT5 exemplar 21
SEQ ID NO: 426: ATP13 sequence DNMT5 exemplar 21
SEQ ID NO: 427: ATP14 sequence DNMT5 exemplar 21
SEQ ID NO: 428: skipped
SEQ ID NO: 429: ATP15 sequence DNMT5 exemplar 21
SEQ ID NO: 430: ATP15B sequence DNMT5 exemplar 21
SEQ ID NO: 431: MT1 sequence from Cryptococcus neoformans
SEQ ID NO: 432: MT23 sequence from Cryptococcus neoformans
SEQ ID NO: 433: MT4 sequence from Cryptococcus neoformans
SEQ ID NO: 434: MT5 sequence from Cryptococcus neoformans
SEQ ID NO: 435: MT6 sequence from Cryptococcus neoformans
SEQ ID NO: 436: MT7 sequence from Cryptococcus neoformans
SEQ ID NO: 437: MT7B sequence from Cryptococcus neoformans
SEQ ID NO: 438: MT8 sequence from Cryptococcus neoformans
SEQ ID NO: 439: MT8B sequence from Cryptococcus neoformans
SEQ ID NO: 440: MT9 sequence from Cryptococcus neoformans
SEQ ID NO: 441: MT10 sequence from Cryptococcus neoformans
SEQ ID NO: 442: ATP11B sequence from Cryptococcus neoformans
SEQ ID NO: 443: ATP12 sequence from Cryptococcus neoformans
SEQ ID NO: 444: ATP13 sequence from Cryptococcus neoformans
SEQ ID NO: 445: ATP14 sequence from Cryptococcus neoformans
SEQ ID NO: 446: ATP14B sequence from Cryptococcus neoformans
SEQ ID NO: 447: ATP15 sequence from Cryptococcus neoformans
SEQ ID NO: 448: ATP15B sequence from Cryptococcus neoformans
SEQ ID NO: 449: MT1 sequence from Cryptococcus tepidarius
SEQ ID NO: 450: MT23 sequence from Cryptococcus tepidarius
SEQ ID NO: 451: MT4 sequence from Cryptococcus tepidarius
SEQ ID NO: 452: MT5 sequence from Cryptococcus tepidarius
SEQ ID NO: 453: MT6 sequence from Cryptococcus tepidarius
SEQ ID NO: 454: MT7 sequence from Cryptococcus tepidarius
SEQ ID NO: 455: MT7B sequence from Cryptococcus tepidarius
SEQ ID NO: 456: MT8 sequence from Cryptococcus tepidarius
SEQ ID NO: 457: MT8B sequence from Cryptococcus tepidarius
SEQ ID NO: 458: MT9 sequence from Cryptococcus tepidarius
SEQ ID NO: 459: MT10 sequence from Cryptococcus tepidarius
SEQ ID NO: 460: ATP11B sequence from Cryptococcus tepidarius
SEQ ID NO: 461: ATP12 sequence from Cryptococcus tepidarius
SEQ ID NO: 462: ATP13 sequence from Cryptococcus tepidarius
SEQ ID NO: 463: ATP14 sequence from Cryptococcus tepidarius
SEQ ID NO: 464: ATP14B sequence from Cryptococcus tepidarius
SEQ ID NO: 465: ATP15 sequence from Cryptococcus tepidarius
SEQ ID NO: 466: ATP15B sequence from Cryptococcus tepidarius

SEQ ID NO 1: amino acid sequence for DNMT5 from Cryptococcus neoformans
(UniProt Accession No. J9VI03 version 2, accessed 08 Jul. 2023)
MTTALTFGGGLFKDNTKFDIDMRGTADGAVNGGNIPNSQSQKRKRASPSPEIESEEDGDDWYEIDYIADSRVIRR
KGRQILQYLIHWAGYAVHERTWEDEDGIGGEDCALVQEFYRKNPGKPRLSPSSVRKEVKLARMVEVVITTRRIDG
KSRAASSTDQPSPHRLGITSPQANNIGGEDPNPSLTRRPVRSTVSEIAKRPTSKKVHPNKKCKASSDDESDFVFE
EGEWDEDEDDDNDVDFRSSEDDEDDEQERSAEEPESDEEIIKPAKKTKSSLPKAKLRPKPANLGGFVTGVRPLNQ
GLDIKAAVRNMSDDLPPISDIEAMFDHLVSRIPDIVELVRQLNGRKLRVATMCSGTESPLLALNMIAKAIKAQHG
LTLAFEHVESCEIEPFKQAYIERNFTPPILFRDVTELGKKRAHTAYGSMVDVPGDVDILIAGTSCVDYSNLNNVQ
QDIDANGESGRTFRGMLQWVKKHQPPIVILENVCNAPWDKVVEYFGQIDYDAQYTRLDTKEFYIPHTRTRVYLFA
TPSSSESDDLPEKWAQTVKDLRRPWSSPFEAFLLHTDDPNIHRARLELASARAQTDGTSRKTTDWNRCESRHQRA
RQDEALGLLRPLTSWQEAGVCKGLDWTWNDWLLAQTERVVDLLEISTLRMAKDGIDSGFKACIWNVSQNVDRQTG
SSKTALAPCLTPNMIPWVTIRGGPVTGREALALQGIPVRELLLTSENEDQLADLAGNAMTTTVVGSAMIAALKVA
CHKITEGANPEKEAALILEKEAVDDEQVANRIIGEDYLEHHDLDLAKVTKSNLSEILDLACRSSRHCQCEGQSGT
APNILECQECSYRACKSCGGRPEHVYAPCANQRVEPAEFEKRFKGLLPMRVRIAGLTDQCLNAVRKAAEKSNKGS
VNDNDWQLWSTALLEGIHDAEFRFRYLKRQSTWTAVYEARRAMLSLVLRNQIPEWRLTIKAPASEPNNSQLRALL
LHPVARLQIDIAGQDVLCGPWELCIPSMKTIDIEITGKGELLPSWQASLGLQGPFANTTRWSEVEISLQAEDENT
LDRKLSGTYQLLPRCGQAMSSLHKKRPDLSDDGLPQLYFFLDPTRCGESREDRYVFSTSTERLDYGTERPVIARL
DSHWREGNEKQRKVKLDVSGAWVKCPEAHLTAIGGDDIAVVANDAAANEIHRDRATFAIPSSASAISASLTTEGC
SHAMALLSCRVPLDPTHSESMWRRGAWAEIDLSHQGNTTFANLAWITERLPPLDGLKNWAHIADDVSEHVCERCA
PRPPKIHWIKREGKANKKGNKTKSTIIAFEDKLEAGQYEHALKHRPSPFVVQLRLDQDIGSFRIGLNIVSLAHRA
LSRLPPTTSEHKISLSWRLTPGHVTESPQPRRVFILPSNKQDPENSQPEAFKLPLRKEQLRSLWWMLEQEKATGK
THTFVEEEISESLLPAVGWRAEGKAERPVMVRGGVIADQVGYGKTVISIALVAQTLSLPAPEPATPGLIDLKATL
IVVPGHLSKQWPNEIARFTGSMFKVIVIQGMKDLQEKTIAELGKADIIVMASEIFESDVYWSRLEYLSAQPREWL
HDTQGGRFFCDRLDAAMESLVSQTKILKEKGSEAAMRAMEDKKKSLVDNVGSKKEVHTAVNFGKRMKGQAYRDKH
DSDSKAKPITKEELERWEASEDEDDDENSKTYIPIPKFHSFTGSESIFSASVKKDYKLLPNPVLHMFRFRRVIAD
EFTYLQKKSLAAVLRLSSSYRWILSGTPPVSDFAAIRSIATFMGIHLGVEDDGEGDVQYQKARAKDQTQAEKFHA
FREVHSRAWHNRRDELAQEFLNVFVRQNIAEIEDIPTVEHIHTFKLPASEGAVYLELEHHLQALEMQARKETKEK
NVSQGDRNARLEEALSDSKTAEEALLKRCCHFTLDLSDKTQDAKSAQEACDHITSARARQLLACQEDLSRSVNQA
IALHGWIKKKGGFSKNDDERQPFAEWIAFSSNISKHQGDIEAARILLKVIEKCGVKDGNIPPSPSDKQSPSIASG
AKMDDVKWQLREQTHLLRKLVKELVARVRSLRFFEVVRKIQKGKSDAQIVLESSECGHKPSTNPDIEMAILSCCG
HVACHKCMRKAAASQRCVKSGECQAAVRPTNIVKVSSLGVEGELSSGRYGAKLEHLVNLIHSIPKNERVLVFLQW
EDLAGKVSEALSAGRIPHVTLSGSAKSRANTLDRFQSTNADSARVLLLKMNDASAAGSNLTTANHAVELGPLFTN
SLFNYRAVETQAIGRVRRYGQQKKVHIHRLLALDTIDMTIFNARRTELKEKTDWEEIPQEEYKGRGSSISMTNEK
RTPTLTVKSNPFKRSSSWALASSFRSKKRSMEARDAEGVSDDDENSELSDII
SEQ ID NO 2: nucleic acid sequence for DNMT5 from Cryptococcus neoformans
5′ATGACTACTGCACTTACATTTGGCGGCGGTCTTTTCAAAGACAACACCAAGTTCGACATTGACATGAGGGGAA
CAGCAGACGGAGCCGTAAATGGGGGAAATATCCCAAATAGTCAGTCACAGAAAAGAAAACGTGCGTCTCCTTCGC
CGGAGATAGAGTCAGAAGAGGATGGCGACGACTGGTATGAAATTGACTACATTGCCGATTCGCGAGTTATCAGAA
GAAAAGGACGCCAAATCTTACAGTATCTCATCCACTGGGCCGGCTATGCTGTGCACGAGAGAACGTGGGAGGACG
AGGATGGTATTGGAGGAGAAGACTGTGCACTTGTTCAAGAATTTTATCGAAAGAATCCCGGAAAACCAAGGCTTT
CGCCCTCTTCTGTCAGGAAAGAAGTAAAATTAGCGCGCATGGTAGAGGTCGTCATCACTACGCGGAGGATCGACG
GAAAGAGCAGAGCCGCCTCCTCAACAGACCAGCCATCCCCTCATCGCCTTGGCATAACAAGTCCCCAAGCGAACA
ACATTGGTGGGGAAGACCCGAATCCATCCCTCACAAGGCGCCCTGTTCGGTCTACCGTCTCGGAAATAGCCAAGA
GACCAACTTCAAAGAAGGTGCATCCGAATAAAAAGTGTAAGGCGTCTAGCGATGATGAAAGCGACTTCGTCTTTG
AAGAAGGTGAATGGGACGAGGACGAGGACGATGACAATGATGTGGATTTCAGATCGAGTGAAGATGATGAAGATG
ATGAACAGGAGAGATCTGCGGAGGAGCCTGAAAGCGATGAGGAAATAATAAAACCTGCGAAAAAGACAAAGTCTT
CTTTGCCCAAGGCGAAATTACGTCCCAAACCAGCGAATCTTGGGGGTTTCGTCACAGGTGTGAGACCTCTCAACC
AGGGACTGGATATCAAAGCGGCTGTAAGGAACATGAGTGACGATCTACCTCCAATAAGCGACATCGAAGCCATGT
TCGATCACCTTGTTTCCAGGATCCCGGACATAGTTGAACTGGTCAGGCAGCTGAATGGGCGAAAACTCCGGGTAG
CTACAATGTGCTCCGGCACCGAGTCCCCTCTTCTTGCTCTCAACATGATCGCAAAAGCAATCAAAGCTCAACATG
GCCTTACATTGGCGTTTGAACACGTTTTTTCTTGTGAGATTGAACCATTCAAGCAGGCTTATATTGAGCGCAACT
TCACCCCTCCTATCCTATTCAGGGATGTAACTGAACTGGGAAAGAAAAGAGCTCATACAGCGTATGGCTCGATGG
TTGACGTACCTGGTGATGTTGATATACTTATTGCTGGTACTTCCTGCGTCGATTACTCCAATTTGAATAACGTCC
AGCAGGATATTGACGCCAATGGCGAATCTGGAAGGACCTTTCGTGGCATGTTACAATGGGTCAAAAAGCATCAGC
CCCCAATCGTCATTTTGGAAAACGTCTGTAACGCGCCATGGGATAAGGTTGTTGAATACTTTGGACAGATTGATT
ATGACGCTCAGTATACACGACTTGATACTAAAGAGTTTTATATTCCCCATACTCGAACTCGAGTTTACCTTTTCG
CCACTCCCTCATCTTCTGAATCAGACGATCTTCCGGAGAAATGGGCCCAGACCGTGAAGGATCTTCGTCGCCCAT
GGTCTTCACCGTTCGAAGCATTCTTACTTCATACAGATGATCCCAACATACATCGCGCTCGTCTCGAATTGGCCT
CTGCGCGCGCGCAGACAGATGGTACTTCTCGCAAAACTACCGACTGGAATAGATGCGAATCAAGACATCAGCGTG
CTAGACAAGACGAAGCCCTGGGCTTATTGAGACCTCTGACTTCCTGGCAAGAAGCGGGTGTGTGTAAGGGACTGG
ATTGGACATGGAATGACTGGCTACTTGCCCAGACAGAAAGGGTAGTCGATCTCTTGGAGATTTCCACCTTGCGGA
TGGCAAAGGACGGCATTGACTCTGGTTTCAAAGCTTGCATTTGGAATGTCAGTCAAAACGTTGATCGACAGACCG
GGTCGTCAAAGACAGCCCTTGCACCTTGCCTGACACCCAACATGATTCCCTGGGTCACTATCCGTGGTGGCCCTG
TCACTGGACGCGAGGCTCTAGCACTTCAAGGCATCCCCGTTCGAGAGCTCCTTCTTACAAGTGAGAACGAAGATC
AATTGGCTGATCTGGCGGGCAATGCCATGACCACCACTGTTGTCGGTTCTGCAATGATTGCTGCGCTCAAGGTGG
CATGTCACAAGATCACCGAGGGCGCTAACCCTGAAAAAGAAGCCGCTTTGATTCTTGAAAAGGAAGCAGTTGACG
ATGAACAAGTTGCTAACCGGATCATCGGCGAAGATTATCTCGAGCATCATGATCTCGACCTTGCCAAAGTCACCA
AATCCAATCTATCTGAGATCCTCGATTTGGCTTGTCGAAGTTCAAGGCACTGTCAATGTGAAGGGCAGTCTGGCA
CTGCTCCGAACATACTTGAGTGTCAAGAGTGCAGTTATCGTGCCTGTAAATCGTGTGGAGGCAGACCTGAGCATG
TCTATGCGCCTTGTGCCAATCAGAGGGTTGAGCCAGCGGAATTTGAGAAAAGATTTAAGGGCTTGCTTCCCATGC
GGGTACGGATTGCTGGCTTAACTGACCAATGTCTAAATGCTGTCAGGAAAGCAGCTGAAAAGTCTAACAAAGGCA
GCGTTAATGATAATGATTGGCAATTATGGAGTACAGCACTTCTAGAAGGAATACACGATGCTGAATTCAGGTTCC
GATATCTCAAAAGACAAAGCACCTGGACGGCAGTTTACGAAGCTCGCCGTGCTATGCTTAGTCTTGTCCTTCGCA
ATCAAATACCGGAATGGCGACTGACCATCAAGGCACCGGCATCCGAGCCGAATAACAGCCAACTTCGAGCACTTC
TTCTTCATCCGGTAGCTCGACTTCAAATTGATATCGCCGGCCAAGATGTCCTGTGCGGTCCTTGGGAACTTTGCA
TCCCCTCTATGAAGACTATCGACATTGAGATTACGGGTAAGGGCGAATTGTTGCCTTCTTGGCAAGCTTCGCTTG
GTCTCCAAGGTCCCTTCGCCAACACCACTCGATGGTCCGAAGTCGAAATTTCCCTCCAAGCGGAAGATGAGAATA
CGCTTGACAGAAAATTATCTGGTACCTATCAGCTTCTTCCCCGATGCGGTCAGGCAATGTCATCTCTTCATAAGA
AGAGGCCCGATCTTTCGGACGATGGTCTTCCTCAGCTCTACTTTTTCCTTGATCCTACCAGGTGTGGTGAATCCC
GTGAAGACAGATATGTATTTTCTACAAGTACTGAAAGATTGGACTATGGCACGGAGCGGCCTGTGATTGCTCGAT
TGGATTCCCACTGGCGGGAAGGCAATGAGAAACAGAGGAAGGTGAAGCTGGATGTCTCTGGCGCATGGGTGAAAT
GTCCCGAAGCCCACCTGACCGCCATAGGTGGGGATGATATCGCTGTCGTTGCCAATGATGCTGCGGCAAACGAAA
TCCATCGTGACAGGGCCACCTTTGCCATCCCTTCATCGGCTTCTGCCATATCAGCATCTTTGACAACTGAAGGTT
GTTCTCATGCAATGGCACTTCTGTCTTGTCGCGTTCCGCTTGATCCTACTCATTCGGAGAGTATGTGGCGACGAG
GAGCCTGGGCAGAAATCGACTTGTCACATCAAGGCAATACTACTTTTGCCAATTTAGCCTGGATCACCGAGAGAT
TGCCCCCTCTTGATGGCTTAAAAAACTGGGCCCATATTGCCGACGATGTATCCGAGCATGTTTGCGAGAGATGCG
CCCCAAGACCACCTAAGATTCACTGGATAAAAAGGGAAGGCAAGGCTAACAAAAAGGGAAACAAGACGAAGAGTA
CCATCATTGCGTTCGAAGATAAGCTAGAAGCTGGGCAGTATGAGCATGCTCTCAAGCATCGGCCCTCGCCCTTTG
TTGTCCAGCTTAGGCTCGATCAGGATATAGGTTCGTTCCGTATCGGTCTCAACATTGTTTCCCTTGCCCATCGTG
CACTCTCGCGACTCCCGCCTACCACGTCCGAACATAAAATCAGTCTTTCTTGGCGTCTCACCCCGGGGCATGTGA
CCGAAAGCCCACAACCTCGCCGCGTCTTTATCCTGCCAAGCAATAAACAAGATCCTGAAAACTCTCAACCCGAAG
CATTCAAATTACCCCTTCGTAAAGAACAGTTGCGATCCTTGTGGTGGATGCTAGAGCAAGAAAAGGCAACGGGGA
AAACCCACACGTTTGTTGAAGAAGAGATTTCTGAGTCCTTGCTTCCTGCTGTAGGGTGGAGAGCAGAGGGTAAGG
CTGAGAGGCCCGTCATGGTCCGCGGAGGTGTCATTGCGGATCAGGTCGGTTACGGCAAAACAGTCATCTCCATCG
CTCTGGTGGCGCAGACCTTGTCCCTTCCTGCGCCTGAGCCTGCAACCCCCGGCTTGATTGACCTGAAAGCGACGC
TTATTGTTGTGCCTGGTCATCTTAGTAAACAATGGCCCAACGAGATAGCGCGATTTACCGGCAGCATGTTCAAGG
TCATTGTCATCCAGGGGATGAAAGATTTACAGGAGAAGACTATCGCTGAACTTGGCAAAGCTGATATCATCGTCA
TGGCGTCAGAAATTTTCGAGTCTGACGTCTATTGGTCTCGACTCGAGTACCTCTCTGCCCAGCCTCGAGAATGGC
TCCATGACACCCAGGGTGGAAGATTTTTCTGTGACAGGCTTGACGCAGCTATGGAAAGTCTTGTGTCGCAGACGA
AAATCTTGAAAGAAAAAGGCAGCGAGGCGGCGATGAGGGCTATGGAGGATAAGAAGAAAAGTTTGGTAGATAATG
TCGGGTCTAAGAAGGAAGTACACACTGCCGTTAACTTCGGCAAGAGAATGAAAGGACAAGCGTATAGAGACAAAC
ATGATAGTGATTCCAAAGCGAAACCCATTACCAAGGAGGAGCTTGAGCGATGGGAGGCTTCTGAAGATGAGGATG
ATGACGAGAATAGCAAGACTTACATCCCTATACCCAAATTTCACAGCTTCACGGGCTCAGAATCAATTTTCAGTG
CTTCGGTCAAAAAGGATTACAAGTTGTTACCCAATCCGGTTCTCCATATGTTTCGATTCCGTCGAGTCATTGCAG
ATGAATTCACTTATCTTCAGAAAAAGAGCCTTGCAGCCGTTTTACGCCTTTCTTCTTCATACCGATGGATTCTGT
CTGGCACTCCGCCTGTCAGCGATTTTGCAGCAATCAGAAGTATCGCAACATTCATGGGTATACACCTTGGTGTGG
AGGATGATGGCGAGGGCGATGTCCAGTATCAGAAGGCTCGCGCAAAAGACCAGACCCAAGCCGAGAAGTTCCATG
CTTTTAGGGAGGTTCATTCACGAGCCTGGCATAATCGACGAGATGAACTGGCACAGGAGTTCTTAAACGTTTTTG
TGCGCCAGAATATCGCGGAGATTGAGGATATACCCACTGTCGAGCATATCCATACTTTCAAACTGCCAGCTTCAG
AAGGTGCTGTATACCTCGAACTAGAGCATCATCTCCAAGCTTTGGAAATGCAAGCACGCAAGGAGACGAAGTTTA
AAAATGTCAGTCAAGGTGATCGCAACGCTAGGCTTGAAGAGGCCTTGTCCGATTCGAAAACGGCAGAAGAAGCCT
TACTCAAACGATGCTGTCATTTCACTCTTGATTTGTCTGATAAGACTCAGGACGCAAAGTCTGCCCAAGAAGCCT
GTGATCACATTACCAGCGCTCGAGCTAGGCAATTACTCGCTTGTCAAGAAGATCTTTCTCGGTCAGTCAATCAGG
CAATAGCTTTGCATGGATGGATTAAGAAGAAGGGGGGATTTAGCAAGAATGATGACGAACGTCAACCATTTGCTG
AGTGGATTGCATTCTCCTCCAACATCTCGAAACATCAAGGAGACATCGAAGCTGCACGCATACTACTCAAGGTGA
TAGAGAAGTGTGGCGTAAAGGACGGCAATATCCCGCCTTCCCCTTCAGATAAACAGTCTCCGTCAATAGCCAGTG
GTGCCAAAATGGATGACGTCAAATGGCAGCTCCGAGAGCAGACCCATCTTCTTCGTAAACTTGTCAAGGAATTGG
TGGCGAGAGTCAGATCATTACGCTTCTTTGAGGTAGTCAGGAAAATCCAGAAAGGCAAGAGCGATGCCCAAATTG
TACTTGAATCTTCGGAATGTGGACACAAGCCATCCACCAATCCAGATATCGAAATGGCTATTCTGTCTTGTTGTG
GTCATGTCGCGTGTCATAAGTGCATGCGTAAGGCAGCCGCTTCTCAACGATGTGTTAAGTCCGGAGAATGCCAAG
CGGCAGTGCGACCGACCAACATAGTCAAGGTAAGCTCCCTGGGCGTTGAAGGAGAGCTTTCCTCAGGGCGGTATG
GTGCCAAACTAGAGCATCTAGTCAACCTCATCCATTCTATCCCCAAAAATGAGCGGGTGTTAGTATTCCTTCAAT
GGGAAGACCTCGCAGGCAAGGTCTCTGAAGCTCTGTCTGCAGGCAGAATTCCACACGTCACGCTATCCGGTTCTG
CCAAGTCACGAGCAAATACCCTTGACCGTTTCCAGTCCACAAATGCCGATAGTGCACGAGTACTGCTTCTCAAGA
TGAATGATGCAAGTGCCGCTGGGTCTAATCTGACCACTGCCAACCATGCTGTCTTCCTCGGCCCCTTATTCACCA
ATTCTTTATTCAACTATCGTGCCGTAGAAACACAAGCCATCGGTCGAGTGCGAAGGTACGGACAACAGAAAAAAG
TTCATATTCATCGGCTGTTGGCCCTCGACACCATTGACATGACCATTTTCAACGCGAGGAGGACTGAGCTCAAAG
AAAAGACAGATTGGGAAGAAATACCCCAAGAGGAGTACAAAGGAAGAGGCTCATCAATTAGCATGACAAACGAGA
AAAGGACGCCAACATTGACAGTCAAGAGTAATCCATTCAAACGCAGTTCTAGCTGGGCGTTAGCATCATCTTTCA
GAAGTAAGAAACGATCGATGGAGGCAAGGGATGCTGAAGGTGTAAGCGACGATGATGAGAATTCAGAACTTTCGG
ATATCATA
SEQ ID NO 3: amino acid sequence for DNMT5 from Cryptococcus tepidarius
MAVTQSSTRSNRTPRRAAAACVSYVDISDEEMNEDDMILVKKRKRAVNIAAMFRSQVKSQAEASSSRPLRHSTQP
QRFGSSTPEKALAKESRLLNESSNQPSITSMFSWQSKAQMILPSSTPNSQSTRSQQTAASTPKKGETQSNAKAFG
SGVKVQLRASISSKKSKVQSDDDDDFAASESDDDEDFESENEEEEKSEEEEYEEEDDYDEKPKGKKAKTSVTPMT
NKKSSGGWGANKIKGPKKAPVKLGNFKTGVKPLMTKDGKAVVKSANMNEDLPPIADVEEMFDHLTSRIPDIVKVV
QRLNGRKLRVATMCSGTESPLLALKMIAKAIERQHGLSFEFDHVESCEIEPFKQAYIERNFAPPILERDVTELGR
KQAHTAYGSIVKVPGDVDILIAGTSCVDYSGLNNEKQDIDANGESGRTFRGMLQWVNKHRPPIVILENVCSAPWD
KIVEYFGQVDYDANYCRLDTKEYYIPHTRTRVYLFATPAHKGDVSHLPEKWVSTLSDLKRPWSASFEAFLLPSDD
PNIHRARLDLAAARFQNDGTQRKPTDWNRCESRHARARQEELLGVLRPLTSWKEAGVCKGLDWTWNDWLLSQTER
VVDLLDISTLRMAAQNDVDSGYKACIWNVSQNVDRQGTGSRTALAPCLTPNMIPWVTIRGGPVTGREALSLQGIP
VRELLLTSENEDQLADLAGNAMTSTVVGASMLSALVLAVDKLAPGEGLESGTTKAEDDGEAKKHHIVGEEYLEEH
ELDLAKVTSANLVEIMDLAERSSRHCQCEGQSGTVTPIDECTLCGYRACKTCGGRPDHAYSPCRHVRVEPREFEK
QFKELLPMRVNIAGVTKASLERVRKRAEAIGKGSVVENDWNLWSEALLEGVTDAEFRFRYLKRQNVWTAVYEAPH
ATLELVLRSTPEWLLTIKSPSSEAVNSRLRTLLLHPVARMRENLTSQDVLCGPWELCVPSKQSFDILITGEGELV
PTWHASIGLQGKHQGTMRWSELRISVPPEVDMALDRKVSGRYPLLPRCGQAMASLHKKQPTQEDEGLSPLFFFLD
PDRVGESSDDTYVFTTSIERLEYGTERAIIAKLESKWRESDKIESKVKLNVRGGWVVCDEVHLTAVGGSDIAIVD
CINDAHRDTATYSFPRDIPAKLTSDCSQSLALLSVQVPLDSSHAETMWRKDDWGEIDLQHEGNTTFANLAWITER
LPSLDRFKEWSELSAVDTSGSPCSRCSPLPPTIHWVRQAGNLNSKGHKTKATIIAFEDRQEASRYEHALKNRPSA
FTVQLRLDNQVGSLRIGLNIASLAHRAFSRLPASQGGVKLSWRLTQDKIADLPRPPREFILPSNKRDPEYAHPKS
FLLPLRKEQLRSLWWMLRQEAAEGKTHTFVEEEISEAMLPSLGWRAEGKAERPVMVRGGVIADQVGYGKTIISLA
LVAETKDDPAPEPAPAGLIDLKATLIVVPGHLSKQWPSEITRFTGKLFKTIVIQSMRELNALNIADLSKADIIVM
ASEVLDSDAYWERFEYLSAQPEDWLSDQQGGREFADRLNTAMAALRDQTRLLMNDPEEAVENMKTLHERAREDAA
KKKKELKVVNFGKRLKGAAYRDKYDADQTLKKSKSAGTSKSKMWEVSEDEEEDEVEEDVAPPKLVERKASGKDSY
SSSSVRANYKLLSAPVMHMERFRRVIADEFTYLEKKPLNALLKLSSCYKWVLSGTPPVGDFPAIRSIASEMGLHL
GVEDDGEGSKSTQIARTKELTQAEKFHAFREMHSSAWHNRRDDLAQLFLNTFVRQNIAEIDEIPTVEHVHEVKLP
AAEGAVYLELEHHLQALEMQARKETKFKNVTQGDRNARLEEALTDSTTAEEALLKRSCHFTLDLNDRKRDAKSAK
EACSFIAGKRRVQLMGCEDDLVNTINLAVGLHAFVRENNGWTNAERQPFKEWVELSFDETKHQGDIEAARRLIAI
LQNRCGELDGIPNAPADSRGIKKLAKAKPAEVENMKWELREQTHLLRRLSKELVARVRSLRFFEVVRKLQLNGPE
AKEILESSVCDHRPSTHPDVEMAVLSCCGHVACHNCMIQAADNQRCPSPSTCHAAIRHTNIVKVKTLGVEGELSS
GRYGAKLQRLVDLIKAIPKRERVLVELQWEDLAHKVSEALTDGGISHLTLSGSVKQRANTLDSFQSDTASSRVLL
LKMQDASAAGSNLTTANHAIFLGPLFVDTLHRYRQAETQAIGRIRRYGQLKHVHIHRLLALDTIDMTIYQNRTRE
LQSKSDYIPIPIEVYVPRGKPSSRSSEGSAQSVMEVDGMIDVDRPSSPPA
SEQ ID NO 4: nucleic acid sequence for DNMT5 from Cryptococcus tepidarius
5′ATGGCAGTCACTCAGAGCTCGACTCGTTCCAATAGGACCCCTCGCAGAGCCGCTGCTGCTTGTGTCAGCTACG
TCGATATCTCAGACGAGGAAATGAATGAAGACGACATGATCTTGGTTAAAAAGCGAAAACGGGCCGTCAATATCG
CGGCGATGTTCCGCAGTCAAGTCAAGTCCCAAGCCGAAGCGTCGTCTTCAAGGCCTTTGAGGCATTCTACTCAAC
CTCAGCGCTTTGGTTCCTCAACTCCAGAGAAAGCTTTGGCGAAAGAGTCAAGGTTATTGAATGAATCCTCCAACC
AGCCCAGTATAACCTCCATGTTCTCTTGGCAATCCAAAGCGCAGATGATTTTACCATCATCAACTCCAAATAGTC
AATCAACGCGTTCTCAGCAAACAGCTGCGTCAACACCGAAAAAAGGTGAAACACAAAGTAATGCCAAGGCGTTCG
GGTCTGGAGTAAAGGTTCAATTGAGGGCATCAATATCCAGCAAAAAATCCAAGGTGCAATCTGATGATGATGATG
ACTTTGCGGCAAGTGAATCGGATGACGATGAAGATTTTGAATCAGAAAATGAAGAGGAGGAGAAAAGCGAGGAGG
AAGAATATGAGGAAGAAGATGACTATGATGAGAAACCCAAAGGAAAAAAAGCAAAGACATCAGTGACGCCTATGA
CCAACAAGAAATCATCAGGAGGTTGGGGTGCGAACAAAATCAAAGGCCCCAAAAAAGCACCAGTGAAGCTTGGCA
ATTTCAAAACCGGTGTTAAGCCTTTAATGACCAAAGATGGAAAGGCAGTTGTCAAAAGCGCCAATATGAACGAGG
ATCTCCCACCTATTGCTGATGTCGAGGAGATGTTCGATCACCTTACATCCAGGATACCAGATATCGTCAAAGTGG
TTCAACGTCTCAACGGTCGTAAACTTCGTGTCGCGACGATGTGCTCTGGTACTGAGTCGCCTCTATTGGCATTAA
AAATGATTGCCAAGGCTATCGAACGGCAGCACGGTTTATCGTTTGAGTTCGATCACGTTTTCTCATGCGAGATTG
AACCTTTCAAACAAGCTTACATCGAACGTAATTTTGCTCCTCCCATTCTTTTCCGTGATGTTACTGAACTCGGAC
GCAAGCAAGCGCATACTGCTTATGGCTCAATTGTCAAAGTTCCTGGTGATGTTGATATTCTTATCGCTGGTACAT
CATGTGTAGACTATTCTGGCCTGAACAACGAGAAACAAGATATCGATGCCAATGGTGAATCAGGTCGAACATTTC
GTGGTATGCTTCAATGGGTGAACAAACATCGCCCGCCCATTGTCATTCTGGAAAATGTATGTAGTGCGCCTTGGG
ACAAAATAGTGGAGTACTTTGGACAAGTTGATTACGACGCAAATTACTGCAGGCTTGACACTAAGGAGTATTACA
TTCCTCACACTCGAACTCGTGTCTACCTTTTCGCCACTCCAGCCCATAAAGGCGACGTGTCTCATCTACCCGAAA
AGTGGGTTTCGACTTTGTCCGACCTCAAACGTCCATGGTCGGCGTCATTTGAGGCATTTCTTTTGCCTTCTGACG
ATCCTAACATTCATCGAGCTCGACTCGACCTTGCTGCTGCTCGATTCCAAAACGACGGGACACAAAGGAAACCAA
CAGACTGGAATCGTTGCGAATCTCGACATGCTCGAGCCCGTCAAGAAGAATTGTTGGGCGTGCTCCGCCCTCTTA
CGTCATGGAAAGAGGCAGGTGTATGTAAAGGGTTGGATTGGACATGGAATGACTGGCTTCTCTCCCAAACAGAGC
GAGTTGTAGATCTGTTGGATATCTCAACATTGCGCATGGCTGCCCAAAATGACGTCGACTCCGGATACAAAGCTT
GCATTTGGAACGTCAGTCAAAACGTTGATCGACAAGGTACCGGAAGTCGAACTGCATTGGCGCCTTGTTTGACAC
CCAATATGATCCCGTGGGTAACGATTCGAGGTGGTCCCGTCACAGGTCGAGAGGCTTTGTCGCTTCAGGGTATAC
CGGTCCGTGAACTGCTCCTTACCAGCGAGAATGAGGATCAGCTTGCAGATCTCGCTGGTAATGCCATGACCTCGA
CAGTCGTCGGCGCGTCAATGCTTTCAGCACTAGTGCTGGCTGTGGACAAGCTCGCACCAGGTGAGGGTCTCGAGA
GTGGAACGACCAAAGCTGAGGACGATGGTGAGGCAAAGAAGCATCATATCGTAGGCGAAGAATACCTTGAAGAAC
ATGAGCTTGATCTCGCCAAAGTCACTAGTGCCAACCTGGTTGAGATTATGGATCTGGCTGAGCGTAGCTCTCGTC
ATTGTCAGTGCGAAGGTCAGTCAGGCACCGTTACGCCCATCGACGAATGTACATTGTGCGGCTATCGAGCTTGTA
AAACTTGTGGTGGCCGCCCTGATCATGCCTATTCTCCTTGCAGACATGTCCGCGTTGAGCCGAGGGAGTTTGAGA
AGCAGTTCAAAGAGCTTTTACCGATGCGTGTGAATATTGCTGGAGTGACTAAGGCATCGCTAGAAAGGGTTCGTA
AACGTGCGGAAGCTATTGGGAAGGGTTCAGTGGTCGAAAACGATTGGAATCTTTGGAGCGAGGCGCTCTTGGAGG
GTGTAACCGACGCAGAATTCAGATTCAGATATTTGAAACGTCAAAATGTTTGGACTGCGGTTTACGAAGCACCAC
ATGCGACATTGGAGCTTGTGCTACGCTCCACTCCCGAATGGCTTTTGACTATCAAATCACCTTCGTCTGAAGCTG
TGAATAGTCGCCTTCGTACTCTGCTTCTTCACCCCGTCGCCAGAATGCGATTCAATTTGACTAGCCAGGACGTAC
TTTGTGGCCCCTGGGAACTTTGTGTTCCTTCCAAACAGTCTTTCGACATATTAATCACTGGCGAAGGCGAACTCG
TCCCAACCTGGCATGCGTCGATCGGTCTTCAGGGCAAACATCAGGGGACGATGCGTTGGTCTGAACTCAGAATCT
CGGTTCCTCCTGAAGTCGATATGGCTCTCGATCGTAAGGTGTCAGGTCGATATCCCCTTCTCCCTCGATGTGGTC
AAGCCATGGCATCTCTACACAAGAAACAACCGACACAGGAGGATGAGGGTCTATCACCACTATTCTTCTTCCTCG
ACCCAGATCGCGTGGGTGAGTCGTCCGACGATACCTATGTATTCACCACATCAATCGAACGTCTTGAGTACGGAA
CTGAACGTGCGATTATCGCGAAGCTAGAGTCCAAGTGGCGCGAGTCTGACAAAATCGAGAGCAAAGTCAAGCTCA
ACGTCCGCGGTGGATGGGTAGTGTGTGACGAAGTGCACCTAACGGCTGTGGGAGGTAGTGATATCGCCATTGTGG
ATTGTATCAATGACGCCCATCGAGACACAGCCACATACTCATTCCCTCGGGATATCCCTGCAAAATTGACATCGG
ATTGTAGTCAATCATTGGCTCTCTTGTCGGTCCAAGTTCCTTTGGACTCTTCTCATGCTGAAACTATGTGGCGGA
AAGATGATTGGGGTGAGATTGATCTTCAGCACGAAGGGAACACGACGTTTGCCAATCTTGCTTGGATCACTGAAC
GTCTTCCTTCGCTTGACCGGTTCAAAGAATGGAGCGAGCTCTCAGCGGTGGATACCTCCGGTTCGCCCTGTTCTC
GTTGCTCGCCTCTCCCGCCAACGATTCATTGGGTCAGGCAAGCGGGAAATCTTAATTCCAAAGGCCACAAAACCA
AGGCGACCATCATCGCCTTTGAGGATCGGCAAGAAGCTAGTCGCTATGAGCATGCACTTAAAAATCGTCCTTCAG
CGTTCACTGTTCAGCTACGCCTCGACAACCAGGTTGGGTCTTTGCGAATTGGCTTAAACATTGCCTCTCTGGCGC
ATCGTGCATTTTCTCGTTTGCCCGCTTCTCAGGGCGGAGTCAAACTCTCTTGGCGTCTTACACAGGACAAAATTG
CCGACCTCCCTCGACCACCTCGGGAGTTTATCTTGCCTAGCAACAAACGAGACCCCGAATATGCTCATCCAAAGT
CGTTCCTCTTACCGCTTCGTAAAGAGCAGCTTCGCTCCCTTTGGTGGATGTTGCGTCAGGAGGCAGCCGAGGGCA
AAACTCACACATTTGTTGAGGAGGAAATCTCAGAGGCCATGTTGCCATCTCTTGGATGGCGAGCGGAAGGTAAAG
CAGAGCGACCAGTCATGGTGCGTGGAGGTGTGATCGCCGATCAAGTCGGTTACGGAAAGACTATTATCTCTCTCG
CTCTGGTCGCTGAGACAAAAGATGATCCCGCTCCAGAACCCGCTCCAGCTGGTTTGATTGACCTCAAAGCCACGC
TCATCGTTGTTCCTGGCCACTTGAGTAAACAATGGCCGAGTGAGATCACCCGATTCACGGGAAAGCTGTTTAAGA
CGATTGTCATCCAAAGTATGCGAGAACTCAACGCTCTCAACATTGCAGATCTTTCCAAAGCCGACATTATCGTTA
TGGCCTCGGAGGTGCTCGATTCAGATGCGTATTGGGAGAGATTCGAATATCTTTCAGCTCAGCCAGAGGACTGGT
TGTCCGATCAACAAGGTGGACGATTTTTCGCTGATCGCTTGAACACTGCTATGGCGGCGTTGCGCGATCAGACGA
GGTTGCTCATGAATGACCCGGAGGAAGCAGTAGAGAACATGAAAACCCTGCACGAGCGAGCTCGGGAAGACGCAG
CAAAAAAGAAAAAGGAACTCAAGGTCGTCAACTTTGGGAAACGCCTAAAGGGTGCCGCATATCGAGACAAATATG
ACGCTGATCAGACCTTAAAGAAATCCAAATCAGCTGGCACCTCAAAGAGCAAGATGTGGGAGGTATCTGAAGATG
AAGAAGAAGACGAAGTGGAGGAGGATGTTGCTCCTCCAAAGCTAGTCTTTCGCAAAGCGTCTGGTAAAGACTCGT
ATTCCAGCAGTTCGGTCCGTGCAAACTACAAGCTTCTTAGCGCACCCGTCATGCACATGTTTAGATTCCGACGGG
TTATTGCCGATGAATTTACGTATCTCGAAAAGAAGCCCTTGAATGCTCTTCTCAAGCTCTCTTCTTGCTACAAAT
GGGTACTTTCCGGAACTCCACCCGTGGGAGACTTCCCAGCGATCCGCAGCATCGCTTCCTTCATGGGACTTCATC
TCGGTGTAGAGGACGACGGCGAAGGCAGCAAATCGACCCAGATTGCCCGCACAAAAGAACTCACACAAGCCGAAA
AGTTCCACGCTTTCCGTGAAATGCATTCATCGGCTTGGCATAACCGTCGCGATGACCTTGCGCAATTGTTCTTGA
ACACGTTCGTCCGTCAAAATATCGCAGAGATTGATGAGATCCCAACAGTTGAGCATGTTCATGAAGTCAAGCTTC
CTGCCGCTGAAGGTGCCGTTTACCTCGAGCTTGAGCACCACCTTCAAGCACTCGAGATGCAAGCACGTAAAGAAA
CCAAATTCAAAAACGTCACGCAAGGTGACCGAAACGCGCGTTTGGAAGAGGCCTTGACCGATTCGACAACTGCTG
AAGAGGCTCTTCTCAAGCGAAGTTGTCATTTCACACTCGACCTGAATGATCGAAAGCGAGATGCAAAGTCAGCCA
AGGAAGCGTGTAGTTTCATTGCAGGAAAACGTAGGGTCCAATTGATGGGCTGCGAAGACGACTTGGTGAATACTA
TCAACCTTGCTGTTGGCCTTCATGCTTTTGTTCGTTTCAACAATGGCTGGACTAATGCAGAACGGCAACCGTTTA
AAGAGTGGGTGGAGCTCTCATTTGATGAGACCAAACATCAGGGCGACATCGAGGCCGCACGTCGATTGATTGCTA
TCCTCCAGAACAGATGTGGCTTCTTAGACGGTATCCCAAACGCACCTGCTGATTCTCGTGGCATTAAAAAGTTGG
CAAAGGCAAAACCGGCCGAAGTAGAGAATATGAAATGGGAGTTGCGAGAGCAGACTCATTTACTTCGACGATTAT
CCAAGGAACTCGTTGCCCGTGTTAGGTCTCTTCGATTCTTTGAAGTAGTGCGTAAACTTCAATTGAATGGTCCCG
AAGCCAAAGAAATCCTTGAGTCTTCAGTTTGCGACCATCGTCCATCGACACATCCAGACGTCGAAATGGCCGTAC
TTTCATGCTGCGGTCACGTTGCTTGTCATAATTGTATGATTCAAGCTGCCGACAACCAGCGTTGTCCATCACCCT
CAACTTGTCACGCTGCCATTCGACACACCAATATAGTCAAAGTCAAGACGCTGGGTGTCGAGGGCGAACTCTCGT
CTGGTCGATATGGAGCAAAACTTCAACGCCTCGTTGATTTGATCAAGGCAATTCCAAAGAGAGAACGTGTTTTGG
TCTTTTTGCAATGGGAAGATCTCGCCCACAAAGTCTCGGAAGCTCTGACTGACGGTGGTATTTCCCACCTCACGC
TCAGCGGCAGTGTCAAGCAACGAGCCAACACTCTGGATTCTTTCCAATCCGATACCGCATCGTCGCGCGTTCTCT
TGCTCAAAATGCAGGACGCATCAGCTGCTGGATCCAACTTGACAACTGCCAATCACGCCATCTTCTTGGGACCTT
TGTTTGTCGATACACTTCATCGATACCGACAAGCAGAAACTCAGGCGATCGGTCGAATCCGTCGTTATGGTCAAT
TGAAACATGTTCATATTCACCGTCTCTTGGCTCTTGACACGATTGATATGACAATTTACCAGAACAGGACGAGAG
AATTACAATCCAAATCAGACTATATCCCGATCCCTATTGAGGTGTACGTCCCCAGAGGCAAGCCGTCCTCCAGAT
CGAGTGAGGGATCAGCCCAGTCCGTTATGGAAGTTGATGGTATGATTGACGTTGATAGACCATCGAGTCCTCCTG
CA
SEQ ID NO: 5:
DNA sequence 1
CCGGTAGGGGCCCATTATAGCCGAATTCGGCGCGCCAGGTGGCACTTTTCGGGGAAATGTGCGCGGAACCCCTAT
TTGTTTATTTTTCTAAATACATTCAAATATGTATCCGCTCATGAGACAATAACCCTGATAAATGC
EcoR1 site = italics
AscI site = underlined
SEQ ID NO: 6: P257
GCGAAATTAATACGACTCACTATAGGGAGCCGGTAGGGGCCCATTATAGCCGAATTCGG[5mC]GCGCCAGGTGG
SEQ ID NO: 7: P258
CAAAAAACCCCTCAAGACCCGTTTAGGCATTTATCAGGGTTATTGTCTCATGAGCGGATAC
SEQ ID NO: 8: T7for-Cy3
GCGAAATTAATACGACTCACTATAGGGAG
wherein the Cy3 is present at the 5′ end:
Cy3-GCGAAATTAATACGACTCACTATAGGGAG
SEQ ID NO: 9: T7for-Atto647
GCGAAATTAATACGACTCACTATAGGGAG
wherein the Atto647 is present at the 5′ end:
Atto647-GCGAAATTAATACGACTCACTATAGGGAG
SEQ ID NO: 10: T7rev-Cy3
CAAAAAACCCCTCAAGACCCGTTTAG
wherein the Cy3 is present at the 5′ end:
Cy3-CAAAAAACCCCTCAAGACCCGTTTAG
SEQ ID NO: 11: T7rev-Atto647
CAAAAAACCCCTCAAGACCCGTTTAG
wherein the Atto647 is present at the 5′ end:
Atto647-CAAAAAACCCCTCAAGACCCGTTTAG
SEQ ID NO: 12: methylated top strand of DNA sequence 2
GGGCGGCGACCTCGCGGGTTTTCGCTATTTATGAAAATCGTTTCCGGTTTAAGGCGTTTGTCATAACTTAATGTT
TTTCCGTTCTTCTTCGTCGACGGGCTCCC
5mC bases are underlined
SEQ ID NO: 13: unmethylated bottom strand of DNA sequence 2 with biotin
GGGAGCCCGTCGACGAAGAAGAACGGAAAAACATTAAGTTATGACAAACGCCTTAAACCGGAAACGATTTTCATA
AATAGCGAAAACCCGCGAGGTCGCCGCCC
wherein a biotin group is attached to the 5′ end
SEQ ID NO: 14: amino acid sequence for C-terminal His-tagged DNMT5 from
Cryptococcus neoformans
MTTALTFGGGLFKDNTKFDIDMRGTADGAVNGGNIPNSQSQKRKRASPSPEIESEEDGDDWYEIDYIADSRVIRR
KGRQILQYLIHWAGYAVHERTWEDEDGIGGEDCALVQEFYRKNPGKPRLSPSSVRKEVKLARMVEVVITTRRIDG
KSRAASSTDQPSPHRLGITSPQANNIGGEDPNPSLTRRPVRSTVSEIAKRPTSKKVHPNKKCKASSDDESDEVFE
EGEWDEDEDDDNDVDERSSEDDEDDEQERSAEEPESDEEIIKPAKKTKSSLPKAKLRPKPANLGGFVTGVRPLNQ
GLDIKAAVRNMSDDLPPISDIEAMEDHLVSRIPDIVELVRQLNGRKLRVATMCSGTESPLLALNMIAKAIKAQHG
LTLAFEHVESCEIEPFKQAYIERNFTPPILFRDVTELGKKRAHTAYGSMVDVPGDVDILIAGTSCVDYSNLNNVQ
QDIDANGESGRTFRGMLQWVKKHQPPIVILENVCNAPWDKVVEYFGQIDYDAQYTRLDTKEFYIPHTRTRVYLFA
TPSSSESDDLPEKWAQTVKDLRRPWSSPFEAFLLHTDDPNIHRARLELASARAQTDGTSRKTTDWNRCESRHQRA
RQDEALGLLRPLTSWQEAGVCKGLDWTWNDWLLAQTERVVDLLEISTLRMAKDGIDSGFKACIWNVSQNVDRQTG
SSKTALAPCLTPNMIPWVTIRGGPVTGREALALQGIPVRELLLTSENEDQLADLAGNAMTTTVVGSAMIAALKVA
CHKITEGANPEKEAALILEKEAVDDEQVANRIIGEDYLEHHDLDLAKVTKSNLSEILDLACRSSRHCQCEGQSGT
APNILECQECSYRACKSCGGRPEHVYAPCANQRVEPAEFEKRFKGLLPMRVRIAGLTDQCLNAVRKAAEKSNKGS
VNDNDWQLWSTALLEGIHDAEFRFRYLKRQSTWTAVYEARRAMLSLVLRNQIPEWRLTIKAPASEPNNSQLRALL
LHPVARLQIDIAGQDVLCGPWELCIPSMKTIDIEITGKGELLPSWQASLGLQGPFANTTRWSEVEISLQAEDENT
LDRKLSGTYQLLPRCGQAMSSLHKKRPDLSDDGLPQLYFFLDPTRCGESREDRYVESTSTERLDYGTERPVIARL
DSHWREGNEKQRKVKLDVSGAWVKCPEAHLTAIGGDDIAVVANDAAANEIHRDRATFAIPSSASAISASLTTEGC
SHAMALLSCRVPLDPTHSESMWRRGAWAEIDLSHQGNTTFANLAWITERLPPLDGLKNWAHIADDVSEHVCERCA
PRPPKIHWIKREGKANKKGNKTKSTIIAFEDKLEAGQYEHALKHRPSPFVVQLRLDQDIGSFRIGLNIVSLAHRA
LSRLPPTTSEHKISLSWRLTPGHVTESPQPRRVFILPSNKQDPENSQPEAFKLPLRKEQLRSLWWMLEQEKATGK
THTFVEEEISESLLPAVGWRAEGKAERPVMVRGGVIADQVGYGKTVISIALVAQTLSLPAPEPATPGLIDLKATL
IVVPGHLSKQWPNEIARFTGSMFKVIVIQGMKDLQEKTIAELGKADIIVMASEIFESDVYWSRLEYLSAQPREWL
HDTQGGRFFCDRLDAAMESLVSQTKILKEKGSEAAMRAMEDKKKSLVDNVGSKKEVHTAVNFGKRMKGQAYRDKH
DSDSKAKPITKEELERWEASEDEDDDENSKTYIPIPKFHSFTGSESIFSASVKKDYKLLPNPVLHMFRFRRVIAD
EFTYLQKKSLAAVLRLSSSYRWILSGTPPVSDFAAIRSIATFMGIHLGVEDDGEGDVQYQKARAKDQTQAEKFHA
FREVHSRAWHNRRDELAQEFLNVFVRQNIAEIEDIPTVEHIHTFKLPASEGAVYLELEHHLQALEMQARKETKEK
NVSQGDRNARLEEALSDSKTAEEALLKRCCHFTLDLSDKTQDAKSAQEACDHITSARARQLLACQEDLSRSVNQA
IALHGWIKKKGGFSKNDDERQPFAEWIAFSSNISKHQGDIEAARILLKVIEKCGVKDGNIPPSPSDKQSPSIASG
AKMDDVKWQLREQTHLLRKLVKELVARVRSLRFFEVVRKIQKGKSDAQIVLESSECGHKPSTNPDIEMAILSCCG
HVACHKCMRKAAASQRCVKSGECQAAVRPTNIVKVSSLGVEGELSSGRYGAKLEHLVNLIHSIPKNERVLVFLQW
EDLAGKVSEALSAGRIPHVTLSGSAKSRANTLDRFQSTNADSARVLLLKMNDASAAGSNLTTANHAVFLGPLFTN
SLENYRAVETQAIGRVRRYGQQKKVHIHRLLALDTIDMTIFNARRTELKEKTDWEEIPQEEYKGRGSSISMTNEK
RTPTLTVKSNPFKRSSSWALASSFRSKKRSMEARDAEGVSDDDENSELSDIIGGGSHHHHHH
SEQ ID NO: 15: AscI recognition sequence
5′-GG|CGCGCC-3′
where the | mark indicates the site of cleavage on dsDNA
SEQ ID NO: 16: EcoRI recognition sequence
5′-G|AATTC-3′
where the | mark indicates the site of cleavage on dsDNA
SEQ ID 17: methylated bottom strand of DNA sequence 2 with biotin
GGGAGCCCGTCGACGAAGAAGAACGGAAAAACATTAAGTTATGACAAACGCCTTAAACCGGAAACGATTTTCATA
AATAGCGAAAACCCGCGAGGTCGCCGCCC
wherein a biotin group is attached at the 5′end, and 5mC bases are underlined
SEQ ID NO 18: T7F
5′ GCGAAATTAATACGACTCACTATAGGGAG
SEQ ID NO 19: methylated SEPT9 sequence
5′CCGCCTACGTGGGGGACCCTGTTAGGGGCACCXGXGTAGACCCTGXGXGCCCTCACAGGACCCTGTGCTXGTT
CTGXGCACTGCXGCCTGGGTTTCCTTCCTTTTATTGTTGTTTGTGTTTGCCAAGCGACAG
Where X indicates 5-mC, bold indicates the priming site for the SEQ ID NO
22, and underline is the priming site for SEQ ID NO 21
SEQ ID NO 20: unmethylated SEPT9 sequence
5′CCGCCTACGTGGGGGACCCTGTTAGGGGCACCCGCGTAGACCCTGCGCGCCCTCACAGGACCCTGTGCTCGTT
CTGCGCACTGCCGCCTGGGTTTCCTTCCTTTTATTGTTGTTTGTGTTTGCCAAGCGACAG
Where bold indicates the priming site for the SEQ ID NO 22, and underline is
the priming site for SEQ ID NO 21
SEQ IS NO 21: SEPT9 forward primer
5′ ACCTTCTTCGTTACCTTCTTCACCAACAATGAAATAATCCTGTCGCTTGGCAAACACAAACAACAATAAAAG
SEQ ID NO 22: SEPT9 reverse primer
5′ ACCACAACGGTTTCCCTCTAGAAATAATTTTGTTTAACCCGCCTACGTGGGGGACCCT
SEQ ID NO 23: NeoFLAGHis variant
MTTALTFGGGLFKDNTKEDIDMRGTADGAVNGGNIPNSQSQKRKRASPSPEIESEEDGDDWYEIDYIADSRVIRR
KGRQILQYLIHWAGYAVHERTWEDEDGIGGEDCALVQEFYRKNPGKPRLSPSSVRKEVKLARMVEVVITTRRIDG
KSRAASSTDQPSPHRLGITSPQANNIGGEDPNPSLTRRPVRSTVSEIAKRPTSKKVHPNKKCKASSDDESDEVFE
EGEWDEDEDDDNDVDERSSEDDEDDEQERSAEEPESDEEIIKPAKKTKSSLPKAKLRPKPANLGGFVTGVRPLNQ
GLDIKAAVRNMSDDLPPISDIEAMEDHLVSRIPDIVELVRQLNGRKLRVATMCSGTESPLLALNMIAKAIKAQHG
LTLAFEHVESCEIEPFKQAYIERNFTPPILFRDVTELGKKRAHTAYGSMVDVPGDVDILIAGTSCVDYSNLNNVQ
QDIDANGESGRTFRGMLQWVKKHQPPIVILENVCNAPWDKVVEYFGQIDYDAQYTRLDTKEFYIPHTRTRVYLFA
TPSSSESDDLPEKWAQTVKDLRRPWSSPFEAFLLHTDDPNIHRARLELASARAQTDGTSRKTTDWNRCESRHQRA
RQDEALGLLRPLTSWQEAGVCKGLDWTWNDWLLAQTERVVDLLEISTLRMAKDGIDSGFKACIWNVSQNVDRQTG
SSKTALAPCLTPNMIPWVTIRGGPVTGREALALQGIPVRELLLTSENEDQLADLAGNAMTTTVVGSAMIAALKVA
CHKITEGANPEKEAALILEKEAVDDEQVANRIIGEDYLEHHDLDLAKVTKSNLSEILDLACRSSRHCQCEGQSGT
APNILECQECSYRACKSCGGRPEHVYAPCANQRVEPAEFEKRFKGLLPMRVRIAGLTDQCLNAVRKAAEKSNKGS
VNDNDWQLWSTALLEGIHDAEFRFRYLKRQSTWTAVYEARRAMLSLVLRNQIPEWRLTIKAPASEPNNSQLRALL
LHPVARLQIDIAGQDVLCGPWELCIPSMKTIDIEITGKGELLPSWQASLGLQGPFANTTRWSEVEISLQAEDENT
LDRKLSGTYQLLPRCGQAMSSLHKKRPDLSDDGLPQLYFFLDPTRCGESREDRYVESTSTERLDYGTERPVIARL
DSHWREGNEKQRKVKLDVSGAWVKCPEAHLTAIGGDDIAVVANDAAANEIHRDRATFAIPSSASAISASLTTEGC
SHAMALLSCRVPLDPTHSESMWRRGAWAEIDLSHQGNTTFANLAWITERLPPLDGLKNWAHIADDVSEHVCERCA
PRPPKIHWIKREGKANKKGNKTKSTIIAFEDKLEAGQYEHALKHRPSPFVVQLRLDQDIGSFRIGLNIVSLAHRA
LSRLPPTTSEHKISLSWRLTPGHVTESPQPRRVFILPSNKQDPENSQPEAFKLPLRKEQLRSLWWMLEQEKATGK
THTFVEEEISESLLPAVGWRAEGKAERPVMVRGGVIADQVGYGKTVISIALVAQTLSLPAPEPATPGLIDLKATL
IVVPGHLSKQWPNEIARFTGSMFKVIVIQGMKDLQEKTIAELGKADIIVMASEIFESDVYWSRLEYLSAQPREWL
HDTQGGRFFCDRLDAAMESLVSQTKILKEKGSEAAMRAMEDKKKSLVDNVGSKKEVHTAVNFGKRMKGQAYRDKH
DSDSKAKPITKEELERWEASEDEDDDENSKTYIPIPKFHSFTGSESIFSASVKKDYKLLPNPVLHMFRFRRVIAD
EFTYLQKKSLAAVLRLSSSYRWILSGTPPVSDFAAIRSIATFMGIHLGVEDDGEGDVQYQKARAKDQTQAEKFHA
FREVHSRAWHNRRDELAQEFLNVFVRQNIAEIEDIPTVEHIHTEKLPASEGAVYLELEHHLQALEMQARKETKEK
NVSQGDRNARLEEALSDSKTAEEALLKRCCHFTLDLSDKTQDAKSAQEACDHITSARARQLLACQEDLSRSVNQA
IALHGWIKKKGGFSKNDDERQPFAEWIAFSSNISKHQGDIEAARILLKVIEKCGVKDGNIPPSPSDKQSPSIASG
AKMDDVKWQLREQTHLLRKLVKELVARVRSLRFFEVVRKIQKGKSDAQIVLESSECGHKPSTNPDIEMAILSCCG
HVACHKCMRKAAASQRCVKSGECQAAVRPTNIVKVSSLGVEGELSSGRYGAKLEHLVNLIHSIPKNERVLVELQW
EDLAGKVSEALSAGRIPHVTLSGSAKSRANTLDRFQSTNADSARVLLLKMNDASAAGSNLTTANHAVELGPLFTN
SLFNYRAVETQAIGRVRRYGQQKKVHIHRLLALDTIDMTIFNARRTELKEKTDWEEIPQEEYKGRGSSISMTNEK
RTPTLTVKSNPFKRSSSWALASSERSKKRSMEARDAEGVSDDDENSELSDIIGGGSDYKDDDDKHHHHHH
SEQ ID NO 24: Variant 2
MGDDWYEIDYIADSRVIRRKGRQILQYLIHWAGYAVHERTWEDEDGIGGEDCALVQEFYRKNPGKPRLSPSSVRK
EVKLARMVEVVITTRRIDGKSRAASSTDQPSPHRLGITSPQANNIGGEDPNPSLTRRPVRSTVSEIAKRPTSKKV
HPNKKCKASSDDESDEVFEEGEWDEDEDDDNDVDERSSEDDEDDEQERSAEEPESDEEIIKPAKKTKSSLPKAKL
RPKPANLGGFVTGVRPLNQGLDIKAAVRNMSDDLPPISDIEAMEDHLVSRIPDIVELVRQLNGRKLRVATMCSGT
ESPLLALNMIAKAIKAQHGLTLAFEHVESCEIEPFKQAYIERNFTPPILFRDVTELGKKRAHTAYGSMVDVPGDV
DILIAGTSCVDYSNLNNVQQDIDANGESGRTFRGMLQWVKKHQPPIVILENVCNAPWDKVVEYFGQIDYDAQYTR
LDTKEFYIPHTRTRVYLFATPSSSESDDLPEKWAQTVKDLRRPWSSPFEAFLLHTDDPNIHRARLELASARAQTD
GTSRKTTDWNRCESRHQRARQDEALGLLRPLTSWQEAGVCKGLDWTWNDWLLAQTERVVDLLEISTLRMAKDGID
SGFKACIWNVSQNVDRQTGSSKTALAPCLTPNMIPWVTIRGGPVTGREALALQGIPVRELLLTSENEDQLADLAG
NAMTTTVVGSAMIAALKVACHKITEGANPEKEAALILEKEAVDDEQVANRIIGEDYLEHHDLDLAKVTKSNLSEI
LDLACRSSRHCQCEGQSGTAPNILECQECSYRACKSCGGRPEHVYAPCANQRVEPAEFEKRFKGLLPMRVRIAGL
TDQCLNAVRKAAEKSNKGSVNDNDWQLWSTALLEGIHDAEFRFRYLKRQSTWTAVYEARRAMLSLVLRNQIPEWR
LTIKAPASEPNNSQLRALLLHPVARLQIDIAGQDVLCGPWELCIPSMKTIDIEITGKGELLPSWQASLGLQGPFA
NTTRWSEVEISLQAEDENTLDRKLSGTYQLLPRCGQAMSSLHKKRPDLSDDGLPQLYFELDPTRCGESREDRYVE
STSTERLDYGTERPVIARLDSHWREGNEKQRKVKLDVSGAWVKCPEAHLTAIGGDDIAVVANDAAANEIHRDRAT
FAIPSSASAISASLTTEGCSHAMALLSCRVPLDPTHSESMWRRGAWAEIDLSHQGNTTFANLAWITERLPPLDGL
KNWAHIADDVSEHVCERCAPRPPKIHWIKREGKANKKGNKTKSTIIAFEDKLEAGQYEHALKHRPSPFVVQLRLD
QDIGSFRIGLNIVSLAHRALSRLPPTTSEHKISLSWRLTPGHVTESPQPRRVFILPSNKQDPENSQPEAFKLPLR
KEQLRSLWWMLEQEKATGKTHTFVEEEISESLLPAVGWRAEGKAERPVMVRGGVIADQVGYGKTVISIALVAQTL
SLPAPEPATPGLIDLKATLIVVPGHLSKQWPNEIARFTGSMFKVIVIQGMKDLQEKTIAELGKADIIVMASEIFE
SDVYWSRLEYLSAQPREWLHDTQGGRFFCDRLDAAMESLVSQTKILKEKGSEAAMRAMEDKKKSLVDNVGSKKEV
HTAVNFGKRMKGQAYRDKHDSDSKAKPITKEELERWEASEDEDDDENSKTYIPIPKFHSFTGSESIFSASVKKDY
KLLPNPVLHMERERRVIADEFTYLQKKSLAAVLRLSSSYRWILSGTPPVSDFAAIRSIATEMGIHLGVEDDGEGD
VQYQKARAKDQTQAEKFHAFREVHSRAWHNRRDELAQEFLNVFVRQNIAEIEDIPTVEHIHTFKLPASEGAVYLE
LEHHLQALEMQARKETKEKNVSQGDRNARLEEALSDSKTAEEALLKRCCHFTLDLSDKTQDAKSAQEACDHITSA
RARQLLACQEDLSRSVNQAIALHGWIKKKGGFSKNDDERQPFAEWIAFSSNISKHQGDIEAARILLKVIEKCGVK
DGNIPPSPSDKQSPSIASGAKMDDVKWQLREQTHLLRKLVKELVARVRSLRFFEVVRKIQKGKSDAQIVLESSEC
GHKPSTNPDIEMAILSCCGHVACHKCMRKAAASQRCVKSGECQAAVRPTNIVKVSSLGVEGELSSGRYGAKLEHL
VNLIHSIPKNERVLVELQWEDLAGKVSEALSAGRIPHVTLSGSAKSRANTLDRFQSTNADSARVLLLKMNDASAA
GSNLTTANHAVELGPLFTNSLENYRAVETQAIGRVRRYGQQKKVHIHRLLALDTIDMTIFNARRTELKEKTDWEE
IPQEEYKGRGSSISMTNEKRTPTLTVKSNPFKRSSSWALASSFRSKKRSMEARDAEGVSDDDENSELSDIIGGGS
DYKDDDDKHHHHHH
SEQ ID NO 25: Variant 3
MGGFVTGVRPLNQGLDIKAAVRNMSDDLPPISDIEAMEDHLVSRIPDIVELVRQLNGRKLRVATMCSGTESPLLA
LNMIAKAIKAQHGLTLAFEHVESCEIEPFKQAYIERNFTPPILERDVTELGKKRAHTAYGSMVDVPGDVDILIAG
TSCVDYSNLNNVQQDIDANGESGRTFRGMLQWVKKHQPPIVILENVCNAPWDKVVEYFGQIDYDAQYTRLDTKEF
YIPHTRTRVYLFATPSSSESDDLPEKWAQTVKDLRRPWSSPFEAFLLHTDDPNIHRARLELASARAQTDGTSRKT
TDWNRCESRHQRARQDEALGLLRPLTSWQEAGVCKGLDWTWNDWLLAQTERVVDLLEISTLRMAKDGIDSGFKAC
IWNVSQNVDRQTGSSKTALAPCLTPNMIPWVTIRGGPVTGREALALQGIPVRELLLTSENEDQLADLAGNAMTTT
VVGSAMIAALKVACHKITEGANPEKEAALILEKEAVDDEQVANRIIGEDYLEHHDLDLAKVTKSNLSEILDLACR
SSRHCQCEGQSGTAPNILECQECSYRACKSCGGRPEHVYAPCANQRVEPAEFEKRFKGLLPMRVRIAGLTDQCLN
AVRKAAEKSNKGSVNDNDWQLWSTALLEGIHDAEFRFRYLKRQSTWTAVYEARRAMLSLVLRNQIPEWRLTIKAP
ASEPNNSQLRALLLHPVARLQIDIAGQDVLCGPWELCIPSMKTIDIEITGKGELLPSWQASLGLQGPFANTTRWS
EVEISLQAEDENTLDRKLSGTYQLLPRCGQAMSSLHKKRPDLSDDGLPQLYFFLDPTRCGESREDRYVESTSTER
LDYGTERPVIARLDSHWREGNEKQRKVKLDVSGAWVKCPEAHLTAIGGDDIAVVANDAAANEIHRDRATFAIPSS
ASAISASLTTEGCSHAMALLSCRVPLDPTHSESMWRRGAWAEIDLSHQGNTTFANLAWITERLPPLDGLKNWAHI
ADDVSEHVCERCAPRPPKIHWIKREGKANKKGNKTKSTIIAFEDKLEAGQYEHALKHRPSPFVVQLRLDQDIGSF
RIGLNIVSLAHRALSRLPPTTSEHKISLSWRLTPGHVTESPQPRRVFILPSNKQDPENSQPEAFKLPLRKEQLRS
LWWMLEQEKATGKTHTFVEEEISESLLPAVGWRAEGKAERPVMVRGGVIADQVGYGKTVISIALVAQTLSLPAPE
PATPGLIDLKATLIVVPGHLSKQWPNEIARFTGSMFKVIVIQGMKDLQEKTIAELGKADIIVMASEIFESDVYWS
RLEYLSAQPREWLHDTQGGRFFCDRLDAAMESLVSQTKILKEKGSEAAMRAMEDKKKSLVDNVGSKKEVHTAVNF
GKRMKGQAYRDKHDSDSKAKPITKEELERWEASEDEDDDENSKTYIPIPKFHSFTGSESIFSASVKKDYKLLPNP
VLHMFRFRRVIADEFTYLQKKSLAAVLRLSSSYRWILSGTPPVSDFAAIRSIATEMGIHLGVEDDGEGDVQYQKA
RAKDQTQAEKFHAFREVHSRAWHNRRDELAQEFLNVFVRQNIAEIEDIPTVEHIHTEKLPASEGAVYLELEHHLQ
ALEMQARKETKFKNVSQGDRNARLEEALSDSKTAEEALLKRCCHFTLDLSDKTQDAKSAQEACDHITSARARQLL
ACQEDLSRSVNQAIALHGWIKKKGGFSKNDDERQPFAEWIAFSSNISKHQGDIEAARILLKVIEKCGVKDGNIPP
SPSDKQSPSIASGAKMDDVKWQLREQTHLLRKLVKELVARVRSLRFFEVVRKIQKGKSDAQIVLESSECGHKPST
NPDIEMAILSCCGHVACHKCMRKAAASQRCVKSGECQAAVRPTNIVKVSSLGVEGELSSGRYGAKLEHLVNLIHS
IPKNERVLVFLQWEDLAGKVSEALSAGRIPHVTLSGSAKSRANTLDRFQSTNADSARVLLLKMNDASAAGSNLTT
ANHAVFLGPLFTNSLFNYRAVETQAIGRVRRYGQQKKVHIHRLLALDTIDMTIFNARRTELKEKTDWEEIPQEEY
KGRGSSISMTNEKRTPTLTVKSNPFKRSSSWALASSFRSKKRSMEARDAEGVSDDDENSELSDIIGGGSDYKDDD
DKHHHHHH
SEQ ID NO 26: Variant 4
MGLDIKAAVRNMSDDLPPISDIEAMEDHLVSRIPDIVELVRQLNGRKLRVATMCSGTESPLLALNMIAKAIKAQH
GLTLAFEHVESCEIEPFKQAYIERNFTPPILFRDVTELGKKRAHTAYGSMVDVPGDVDILIAGTSCVDYSNLNNV
QQDIDANGESGRTFRGMLQWVKKHQPPIVILENVCNAPWDKVVEYFGQIDYDAQYTRLDTKEFYIPHTRTRVYLF
ATPSSSESDDLPEKWAQTVKDLRRPWSSPFEAFLLHTDDPNIHRARLELASARAQTDGTSRKTTDWNRCESRHQR
ARQDEALGLLRPLTSWQEAGVCKGLDWTWNDWLLAQTERVVDLLEISTLRMAKDGIDSGFKACIWNVSQNVDRQT
GSSKTALAPCLTPNMIPWVTIRGGPVTGREALALQGIPVRELLLTSENEDQLADLAGNAMTTTVVGSAMIAALKV
ACHKITEGANPEKEAALILEKEAVDDEQVANRIIGEDYLEHHDLDLAKVTKSNLSEILDLACRSSRHCQCEGQSG
TAPNILECQECSYRACKSCGGRPEHVYAPCANQRVEPAEFEKRFKGLLPMRVRIAGLTDQCLNAVRKAAEKSNKG
SVNDNDWQLWSTALLEGIHDAEFRFRYLKRQSTWTAVYEARRAMLSLVLRNQIPEWRLTIKAPASEPNNSQLRAL
LLHPVARLQIDIAGQDVLCGPWELCIPSMKTIDIEITGKGELLPSWQASLGLQGPFANTTRWSEVEISLQAEDEN
TLDRKLSGTYQLLPRCGQAMSSLHKKRPDLSDDGLPQLYFFLDPTRCGESREDRYVESTSTERLDYGTERPVIAR
LDSHWREGNEKQRKVKLDVSGAWVKCPEAHLTAIGGDDIAVVANDAAANEIHRDRATFAIPSSASAISASLTTEG
CSHAMALLSCRVPLDPTHSESMWRRGAWAEIDLSHQGNTTFANLAWITERLPPLDGLKNWAHIADDVSEHVCERC
APRPPKIHWIKREGKANKKGNKTKSTIIAFEDKLEAGQYEHALKHRPSPFVVQLRLDQDIGSFRIGLNIVSLAHR
ALSRLPPTTSEHKISLSWRLTPGHVTESPQPRRVFILPSNKQDPENSQPEAFKLPLRKEQLRSLWWMLEQEKATG
KTHTFVEEEISESLLPAVGWRAEGKAERPVMVRGGVIADQVGYGKTVISIALVAQTLSLPAPEPATPGLIDLKAT
LIVVPGHLSKQWPNEIARFTGSMFKVIVIQGMKDLQEKTIAELGKADIIVMASEIFESDVYWSRLEYLSAQPREW
LHDTQGGRFFCDRLDAAMESLVSQTKILKEKGSEAAMRAMEDKKKSLVDNVGSKKEVHTAVNFGKRMKGQAYRDK
HDSDSKAKPITKEELERWEASEDEDDDENSKTYIPIPKFHSFTGSESIFSASVKKDYKLLPNPVLHMFRFRRVIA
DEFTYLQKKSLAAVLRLSSSYRWILSGTPPVSDFAAIRSIATFMGIHLGVEDDGEGDVQYQKARAKDQTQAEKFH
AFREVHSRAWHNRRDELAQEFLNVFVRQNIAEIEDIPTVEHIHTFKLPASEGAVYLELEHHLQALEMQARKETKE
KNVSQGDRNARLEEALSDSKTAEEALLKRCCHFTLDLSDKTQDAKSAQEACDHITSARARQLLACQEDLSRSVNQ
AIALHGWIKKKGGFSKNDDERQPFAEWIAFSSNISKHQGDIEAARILLKVIEKCGVKDGNIPPSPSDKQSPSIAS
GAKMDDVKWQLREQTHLLRKLVKELVARVRSLRFFEVVRKIQKGKSDAQIVLESSECGHKPSTNPDIEMAILSCC
GHVACHKCMRKAAASQRCVKSGECQAAVRPTNIVKVSSLGVEGELSSGRYGAKLEHLVNLIHSIPKNERVLVELQ
WEDLAGKVSEALSAGRIPHVTLSGSAKSRANTLDRFQSTNADSARVLLLKMNDASAAGSNLTTANHAVELGPLFT
NSLENYRAVETQAIGRVRRYGQQKKVHIHRLLALDTIDMTIFNARRTELKEKTDWEEIPQEEYKGRGSSISMTNE
KRTPTLTVKSNPFKRSSSWALASSFRSKKRSMEARDAEGVSDDDENSELSDIIGGGSDYKDDDDKHHHHHH
SEQ ID NO 27: Variant 5
MTTALTFGGGLFKDNTKFDIDMRGTADGAVNGGNIPNSQSQKRKRASPSPEIESEEDGDDWYEIDYIADSRVIRR
KGRQILQYLIHWAGYAVHERTWEDEDGIGGEDCALVQEFYRKNPGKPRLSPSSVRKEVKLARMVEVVITTRRIDG
KSRAASSTDQPSPHRLGITSPQANNIGGEDPNPSLTRRPVRSTVSEIAKRPTSKKVHPNKKCKASSDDESDFVFE
EGEWDEDEDDDNDVDERSSEDDEDDEQERSAEEPESDEEIIKPAKKTKSSLPKAKLRPKPANLGGFVTGVRPLNQ
GLDIKAAVRNMSDDLPPISDIEAMEDHLVSRIPDIVELVRQLNGRKLRVATMCSGTESPLLALNMIAKAIKAQHG
LTLAFEHVFSCEIEPFKQAYIERNFTPPILFRDVTELGKKRAHTAYGSMVDVPGDVDILIAGTSCVDYSNLNNVQ
QDIDANGESGRTFRGMLQWVKKHQPPIVILENVCNAPWDKVVEYFGQIDYDAQYTRLDTKEFYIPHTRTRVYLFA
TPSSSESDDLPEKWAQTVKDLRRPWSSPFEAFLLHTDDPNIHRARLELASARAQTDGTSRKTTDWNRCESRHQRA
RQDEALGLLRPLTSWQEAGVCKGLDWTWNDWLLAQTERVVDLLEISTLRMAKDGIDSGFKACIWNVSQNVDRQTG
SSKTALAPCLTPNMIPWVTIRGGPVTGREALALQGIPVRELLLTSENEDQLADLAGNAMTTTVVGSAMIAALKVA
CHKITEGANPEKEAALILEKEAVDDEQVANRIIGEDYLEHHDLDLAKVTKSNLSEILDLACRSSRHCQCEGQSGT
APNILECQECSYRACKSCGGRPEHVYAPCANQRVEPAEFEKRFKGLLPMRVRIAGLTDQCLNAVRKAAEKSNKGS
VNDNDWQLWSTALLEGIHDAEFRFRYLKRQSTWTAVYEARRAMLSLVLRNQIPEWRLTIKAPASEPNNSQLRALL
LHPVARLQIDIAGQDVLCGPWELCIPSMKTIDIEITGKGELLPSWQASLGLQGPFANTTRWSEVEISLQAEDENT
LDRKLSGTYQLLPRCGQAMSSLHKKRPDLSDDGLPQLYFFLDPTRCGESREDRYVESTSTERLDYGTERPVIARL
DSHWREGNEKQRKVKLDVSGAWVKCPEAHLTAIGGDDIAVVANDAAANEIHRDRATFAIPSSASAISASLTTEGC
SHAMALLSCRVPLDPTHSESMWRRGAWAEIDLSHQGNTTFANLAWITERLPPLDGLKNWAHIADDVSEHVCERCA
PRPPKIHWIKREGKANKKGNKTKSTIIAFEDKLEAGQYEHALKHRPSPFVVQLRLDQDIGSFRIGLNIVSLAHRA
LSRLPPTTSEHKISLSWRLTPGHVTESPQPRRVFILPSNKQDPENSQPEAFKLPLRKEQLRSLWWMLEQEKATGK
THTFVEEEISESLLPAVGWRAEGKAERPVMVRGGVIADQVGYGKTVISIALVAQTLSLPAPEPATPGLIDLKATL
IVVPGHLSKQWPNEIARFTGSMFKVIVIQGMKDLQEKTIAELGKADIIVMASEIFESDVYWSRLEYLSAQPREWL
HDTQGGRFFCDRLDAAMESLVSQTKILKEKGSEAAMRAMEDKKKSLVDNVGSKKEVHTAVNEGKRMKGQAYRDKH
DSDSKAKPITKEELERWEASEDEDDDENSKTYIPIPKFHSFTGSESIFSASVKKDYKLLPNPVLHMFRFRRVIAD
EFTYLQKKSLAAVLRLSSSYRWILSGTPPVSDFAAIRSIATFMGIHLGVEDDGEGDVQYQKARAKDQTQAEKFHA
FREVHSRAWHNRRDELAQEFLNVFVRQNIAEIEDIPTVEHIHTFKLPASEGAVYLELEHHLQALEMQARKETKFK
NVSQGDRNARLEEALSDSKTAEEALLKRCCHFTLDLSDKTQDAKSAQEACDHITSARARQLLACQEDLSRSVNQA
IALHGWIKKKGGFSKNDDERQPFAEWIAFSSNISKHQGDIEAARILLKVIEKCGVKDGNIPPSPSDKQSPSIASG
AKMDDVKWQLREQTHLLRKLVKELVARVRSLRFFEVVRKIQKGKSDAQIVLESSECGHKPSTNPDIEMAILSCCG
HVACHKCMRKAAASQRCVKSGECQAAVRPTNIVKVSSLGVEGELSSGRYGAKLEHLVNLIHSIPKNERVLVFLQW
EDLAGKVSEALSAGRIPHVTLSGSAKSRANTLDRFQSTNADSARVLLLKMNDASAAGSNLTTANHAVELGPLFTN
SLENYRAVETQAIGRVRRYGQQKKVHIHRLLALDTIDMTIFNARRTELKEKTDWEEIPQEEYKGRGSSISMTNEK
RTPTLTVKSNPGGGSDYKDDDDKHHHHHH
SEQ ID NO 28: Variant 6
MGDDWYEIDYIADSRVIRRKGRQILQYLIHWAGYAVHERTWEDEDGIGGEDCALVQEFYRKNPGKPRLSPSSVRK
EVKLARMVEVVITTRRIDGKSRAASSTDQPSPHRLGITSPQANNIGGEDPNPSLTRRPVRSTVSEIAKRPTSKKV
HPNKKCKASSDDESDEVFEEGEWDEDEDDDNDVDERSSEDDEDDEQERSAEEPESDEEIIKPAKKTKSSLPKAKL
RPKPANLGGFVTGVRPLNQGLDIKAAVRNMSDDLPPISDIEAMEDHLVSRIPDIVELVRQLNGRKLRVATMCSGT
ESPLLALNMIAKAIKAQHGLTLAFEHVESCEIEPFKQAYIERNFTPPILFRDVTELGKKRAHTAYGSMVDVPGDV
DILIAGTSCVDYSNLNNVQQDIDANGESGRTFRGMLQWVKKHQPPIVILENVCNAPWDKVVEYFGQIDYDAQYTR
LDTKEFYIPHTRTRVYLFATPSSSESDDLPEKWAQTVKDLRRPWSSPFEAFLLHTDDPNIHRARLELASARAQTD
GTSRKTTDWNRCESRHQRARQDEALGLLRPLTSWQEAGVCKGLDWTWNDWLLAQTERVVDLLEISTLRMAKDGID
SGFKACIWNVSQNVDRQTGSSKTALAPCLTPNMIPWVTIRGGPVTGREALALQGIPVRELLLTSENEDQLADLAG
NAMTTTVVGSAMIAALKVACHKITEGANPEKEAALILEKEAVDDEQVANRIIGEDYLEHHDLDLAKVTKSNLSEI
LDLACRSSRHCQCEGQSGTAPNILECQECSYRACKSCGGRPEHVYAPCANQRVEPAEFEKRFKGLLPMRVRIAGL
TDQCLNAVRKAAEKSNKGSVNDNDWQLWSTALLEGIHDAEFRFRYLKRQSTWTAVYEARRAMLSLVLRNQIPEWR
LTIKAPASEPNNSQLRALLLHPVARLQIDIAGQDVLCGPWELCIPSMKTIDIEITGKGELLPSWQASLGLQGPFA
NTTRWSEVEISLQAEDENTLDRKLSGTYQLLPRCGQAMSSLHKKRPDLSDDGLPQLYFFLDPTRCGESREDRYVE
STSTERLDYGTERPVIARLDSHWREGNEKQRKVKLDVSGAWVKCPEAHLTAIGGDDIAVVANDAAANEIHRDRAT
FAIPSSASAISASLTTEGCSHAMALLSCRVPLDPTHSESMWRRGAWAEIDLSHQGNTTFANLAWITERLPPLDGL
KNWAHIADDVSEHVCERCAPRPPKIHWIKREGKANKKGNKTKSTIIAFEDKLEAGQYEHALKHRPSPFVVQLRLD
QDIGSFRIGLNIVSLAHRALSRLPPTTSEHKISLSWRLTPGHVTESPQPRRVFILPSNKQDPENSQPEAFKLPLR
KEQLRSLWWMLEQEKATGKTHTFVEEEISESLLPAVGWRAEGKAERPVMVRGGVIADQVGYGKTVISIALVAQTL
SLPAPEPATPGLIDLKATLIVVPGHLSKQWPNEIARFTGSMFKVIVIQGMKDLQEKTIAELGKADIIVMASEIFE
SDVYWSRLEYLSAQPREWLHDTQGGREFCDRLDAAMESLVSQTKILKEKGSEAAMRAMEDKKKSLVDNVGSKKEV
HTAVNFGKRMKGQAYRDKHDSDSKAKPITKEELERWEASEDEDDDENSKTYIPIPKFHSFTGSESIFSASVKKDY
KLLPNPVLHMFRFRRVIADEFTYLQKKSLAAVLRLSSSYRWILSGTPPVSDFAAIRSIATFMGIHLGVEDDGEGD
VQYQKARAKDQTQAEKFHAFREVHSRAWHNRRDELAQEFLNVFVRQNIAEIEDIPTVEHIHTFKLPASEGAVYLE
LEHHLQALEMQARKETKFKNVSQGDRNARLEEALSDSKTAEEALLKRCCHFTLDLSDKTQDAKSAQEACDHITSA
RARQLLACQEDLSRSVNQAIALHGWIKKKGGFSKNDDERQPFAEWIAFSSNISKHQGDIEAARILLKVIEKCGVK
DGNIPPSPSDKQSPSIASGAKMDDVKWQLREQTHLLRKLVKELVARVRSLRFFEVVRKIQKGKSDAQIVLESSEC
GHKPSTNPDIEMAILSCCGHVACHKCMRKAAASQRCVKSGECQAAVRPTNIVKVSSLGVEGELSSGRYGAKLEHL
VNLIHSIPKNERVLVFLQWEDLAGKVSEALSAGRIPHVTLSGSAKSRANTLDRFQSTNADSARVLLLKMNDASAA
GSNLTTANHAVFLGPLFTNSLENYRAVETQAIGRVRRYGQQKKVHIHRLLALDTIDMTIFNARRTELKEKTDWEE
IPQEEYKGRGSSISMTNEKRTPTLTVKSNPGGGSDYKDDDDKHHHHHH
SEQ ID NO 29: Variant 7
MGGFVTGVRPLNQGLDIKAAVRNMSDDLPPISDIEAMEDHLVSRIPDIVELVRQLNGRKLRVATMCSGTESPLLA
LNMIAKAIKAQHGLTLAFEHVESCEIEPFKQAYIERNFTPPILERDVTELGKKRAHTAYGSMVDVPGDVDILIAG
TSCVDYSNLNNVQQDIDANGESGRTFRGMLQWVKKHQPPIVILENVCNAPWDKVVEYFGQIDYDAQYTRLDTKEF
YIPHTRTRVYLFATPSSSESDDLPEKWAQTVKDLRRPWSSPFEAFLLHTDDPNIHRARLELASARAQTDGTSRKT
TDWNRCESRHQRARQDEALGLLRPLTSWQEAGVCKGLDWTWNDWLLAQTERVVDLLEISTLRMAKDGIDSGFKAC
IWNVSQNVDRQTGSSKTALAPCLTPNMIPWVTIRGGPVTGREALALQGIPVRELLLTSENEDQLADLAGNAMTTT
VVGSAMIAALKVACHKITEGANPEKEAALILEKEAVDDEQVANRIIGEDYLEHHDLDLAKVTKSNLSEILDLACR
SSRHCQCEGQSGTAPNILECQECSYRACKSCGGRPEHVYAPCANQRVEPAEFEKRFKGLLPMRVRIAGLTDQCLN
AVRKAAEKSNKGSVNDNDWQLWSTALLEGIHDAEFRFRYLKRQSTWTAVYEARRAMLSLVLRNQIPEWRLTIKAP
ASEPNNSQLRALLLHPVARLQIDIAGQDVLCGPWELCIPSMKTIDIEITGKGELLPSWQASLGLQGPFANTTRWS
EVEISLQAEDENTLDRKLSGTYQLLPRCGQAMSSLHKKRPDLSDDGLPQLYFFLDPTRCGESREDRYVESTSTER
LDYGTERPVIARLDSHWREGNEKQRKVKLDVSGAWVKCPEAHLTAIGGDDIAVVANDAAANEIHRDRATFAIPSS
ASAISASLTTEGCSHAMALLSCRVPLDPTHSESMWRRGAWAEIDLSHQGNTTFANLAWITERLPPLDGLKNWAHI
ADDVSEHVCERCAPRPPKIHWIKREGKANKKGNKTKSTIIAFEDKLEAGQYEHALKHRPSPFVVQLRLDQDIGSE
RIGLNIVSLAHRALSRLPPTTSEHKISLSWRLTPGHVTESPQPRRVFILPSNKQDPENSQPEAFKLPLRKEQLRS
LWWMLEQEKATGKTHTFVEEEISESLLPAVGWRAEGKAERPVMVRGGVIADQVGYGKTVISIALVAQTLSLPAPE
PATPGLIDLKATLIVVPGHLSKQWPNEIARFTGSMFKVIVIQGMKDLQEKTIAELGKADIIVMASEIFESDVYWS
RLEYLSAQPREWLHDTQGGRFFCDRLDAAMESLVSQTKILKEKGSEAAMRAMEDKKKSLVDNVGSKKEVHTAVNE
GKRMKGQAYRDKHDSDSKAKPITKEELERWEASEDEDDDENSKTYIPIPKFHSFTGSESIFSASVKKDYKLLPNP
VLHMFRFRRVIADEFTYLQKKSLAAVLRLSSSYRWILSGTPPVSDFAAIRSIATFMGIHLGVEDDGEGDVQYQKA
RAKDQTQAEKFHAFREVHSRAWHNRRDELAQEFLNVFVRQNIAEIEDIPTVEHIHTFKLPASEGAVYLELEHHLQ
ALEMQARKETKFKNVSQGDRNARLEEALSDSKTAEEALLKRCCHFTLDLSDKTQDAKSAQEACDHITSARARQLL
ACQEDLSRSVNQAIALHGWIKKKGGFSKNDDERQPFAEWIAFSSNISKHQGDIEAARILLKVIEKCGVKDGNIPP
SPSDKQSPSIASGAKMDDVKWQLREQTHLLRKLVKELVARVRSLRFFEVVRKIQKGKSDAQIVLESSECGHKPST
NPDIEMAILSCCGHVACHKCMRKAAASQRCVKSGECQAAVRPTNIVKVSSLGVEGELSSGRYGAKLEHLVNLIHS
IPKNERVLVFLQWEDLAGKVSEALSAGRIPHVTLSGSAKSRANTLDRFQSTNADSARVLLLKMNDASAAGSNLTT
ANHAVFLGPLFTNSLENYRAVETQAIGRVRRYGQQKKVHIHRLLALDTIDMTIFNARRTELKEKTDWEEIPQEEY
KGRGSSISMTNEKRTPTLTVKSNPGGGSDYKDDDDKHHHHHH
SEQ ID NO 30: Variant 8
MGLDIKAAVRNMSDDLPPISDIEAMFDHLVSRIPDIVELVRQLNGRKLRVATMCSGTESPLLALNMIAKAIKAQH
GLTLAFEHVESCEIEPFKQAYIERNFTPPILFRDVTELGKKRAHTAYGSMVDVPGDVDILIAGTSCVDYSNLNNV
QQDIDANGESGRTFRGMLQWVKKHQPPIVILENVCNAPWDKVVEYFGQIDYDAQYTRLDTKEFYIPHTRTRVYLF
ATPSSSESDDLPEKWAQTVKDLRRPWSSPFEAFLLHTDDPNIHRARLELASARAQTDGTSRKTTDWNRCESRHQR
ARQDEALGLLRPLTSWQEAGVCKGLDWTWNDWLLAQTERVVDLLEISTLRMAKDGIDSGFKACIWNVSQNVDRQT
GSSKTALAPCLTPNMIPWVTIRGGPVTGREALALQGIPVRELLLTSENEDQLADLAGNAMTTTVVGSAMIAALKV
ACHKITEGANPEKEAALILEKEAVDDEQVANRIIGEDYLEHHDLDLAKVTKSNLSEILDLACRSSRHCQCEGQSG
TAPNILECQECSYRACKSCGGRPEHVYAPCANQRVEPAEFEKRFKGLLPMRVRIAGLTDQCLNAVRKAAEKSNKG
SVNDNDWQLWSTALLEGIHDAEFRFRYLKRQSTWTAVYEARRAMLSLVLRNQIPEWRLTIKAPASEPNNSQLRAL
LLHPVARLQIDIAGQDVLCGPWELCIPSMKTIDIEITGKGELLPSWQASLGLQGPFANTTRWSEVEISLQAEDEN
TLDRKLSGTYQLLPRCGQAMSSLHKKRPDLSDDGLPQLYFFLDPTRCGESREDRYVESTSTERLDYGTERPVIAR
LDSHWREGNEKQRKVKLDVSGAWVKCPEAHLTAIGGDDIAVVANDAAANEIHRDRATFAIPSSASAISASLTTEG
CSHAMALLSCRVPLDPTHSESMWRRGAWAEIDLSHQGNTTFANLAWITERLPPLDGLKNWAHIADDVSEHVCERC
APRPPKIHWIKREGKANKKGNKTKSTIIAFEDKLEAGQYEHALKHRPSPFVVQLRLDQDIGSFRIGLNIVSLAHR
ALSRLPPTTSEHKISLSWRLTPGHVTESPQPRRVFILPSNKQDPENSQPEAFKLPLRKEQLRSLWWMLEQEKATG
KTHTFVEEEISESLLPAVGWRAEGKAERPVMVRGGVIADQVGYGKTVISIALVAQTLSLPAPEPATPGLIDLKAT
LIVVPGHLSKQWPNEIARFTGSMFKVIVIQGMKDLQEKTIAELGKADIIVMASEIFESDVYWSRLEYLSAQPREW
LHDTQGGRFFCDRLDAAMESLVSQTKILKEKGSEAAMRAMEDKKKSLVDNVGSKKEVHTAVNFGKRMKGQAYRDK
HDSDSKAKPITKEELERWEASEDEDDDENSKTYIPIPKFHSFTGSESIFSASVKKDYKLLPNPVLHMFRFRRVIA
DEFTYLQKKSLAAVLRLSSSYRWILSGTPPVSDFAAIRSIATFMGIHLGVEDDGEGDVQYQKARAKDQTQAEKFH
AFREVHSRAWHNRRDELAQEFLNVFVRQNIAEIEDIPTVEHIHTFKLPASEGAVYLELEHHLQALEMQARKETKE
KNVSQGDRNARLEEALSDSKTAEEALLKRCCHFTLDLSDKTQDAKSAQEACDHITSARARQLLACQEDLSRSVNQ
AIALHGWIKKKGGFSKNDDERQPFAEWIAFSSNISKHQGDIEAARILLKVIEKCGVKDGNIPPSPSDKQSPSIAS
GAKMDDVKWQLREQTHLLRKLVKELVARVRSLRFFEVVRKIQKGKSDAQIVLESSECGHKPSTNPDIEMAILSCC
GHVACHKCMRKAAASQRCVKSGECQAAVRPTNIVKVSSLGVEGELSSGRYGAKLEHLVNLIHSIPKNERVLVFLQ
WEDLAGKVSEALSAGRIPHVTLSGSAKSRANTLDRFQSTNADSARVLLLKMNDASAAGSNLTTANHAVELGPLFT
NSLENYRAVETQAIGRVRRYGQQKKVHIHRLLALDTIDMTIFNARRTELKEKTDWEEIPQEEYKGRGSSISMTNE
KRTPTLTVKSNPGGGSDYKDDDDKHHHHHH
SEQ ID NO 31: Variant 9
MTTALTFGGGLFKDNTKFDIDMRGTADGAVNGGNIPNSQSQKRKRASPSPEIESEEDGDDWYEIDYIADSRVIRR
KGRQILQYLIHWAGYAVHERTWEDEDGIGGEDCALVQEFYRKNPGKPRLSPSSVRKEVKLARMVEVVITTRRIDG
KSRAASSTDQPSPHRLGITSPQANNIGGEDPNPSLTRRPVRSTVSEIAKRPTSKKVHPNKKCKASSDDESDEVFE
EGEWDEDEDDDNDVDFRSSEDDEDDEQERSAEEPESDEEIIKPAKKTKSSLPKAKLRPKPANLGGFVTGVRPLNQ
GLDIKAAVRNMSDDLPPISDIEAMEDHLVSRIPDIVELVRQLNGRKLRVATMCSGTESPLLALNMIAKAIKAQHG
LTLAFEHVESCEIEPFKQAYIERNFTPPILFRDVTELGKKRAHTAYGSMVDVPGDVDILIAGTSCVDYSNLNNVQ
QDIDANGESGRTFRGMLQWVKKHQPPIVILENVCNAPWDKVVEYFGQIDYDAQYTRLDTKEFYIPHTRTRVYLFA
TPSSSESDDLPEKWAQTVKDLRRPWSSPFEAFLLHTDDPNIHRARLELASARAQTDGTSRKTTDWNRCESRHQRA
RQDEALGLLRPLTSWQEAGVCKGLDWTWNDWLLAQTERVVDLLEISTLRMAKDGIDSGFKACIWNVSQNVDRQTG
SSKTALAPCLTPNMIPWVTIRGGPVTGREALALQGIPVRELLLTSENEDQLADLAGNAMTTTVVGSAMIAALKVA
CHKITEGANPEKEAALILEKEAVDDEQVANRIIGEDYLEHHDLDLAKVTKSNLSEILDLACRSSRHCQCEGQSGT
APNILECQECSYRACKSCGGRPEHVYAPCANQRVEPAEFEKRFKGLLPMRVRIAGLTDQCLNAVRKAAEKSNKGS
VNDNDWQLWSTALLEGIHDAEFRFRYLKRQSTWTAVYEARRAMLSLVLRNQIPEWRLTIKAPASEPNNSQLRALL
LHPVARLQIDIAGQDVLCGPWELCIPSMKTIDIEITGKGELLPSWQASLGLQGPFANTTRWSEVEISLQAEDENT
LDRKLSGTYQLLPRCGQAMSSLHKKRPDLSDDGLPQLYFFLDPTRCGESREDRYVESTSTERLDYGTERPVIARL
DSHWREGNEKQRKVKLDVSGAWVKCPEAHLTAIGGDDIAVVANDAAANEIHRDRATFAIPSSASAISASLTTEGC
SHAMALLSCRVPLDPTHSESMWRRGAWAEIDLSHQGNTTFANLAWITERLPPLDGLKNWAHIADDVSEHVCERCA
PRPPKIHWIKREGKANKKGNKTKSTIIAFEDKLEAGQYEHALKHRPSPFVVQLRLDQDIGSFRIGLNIVSLAHRA
LSRLPPTTSEHKISLSWRLTPGHVTESPQPRRVFILPSNKQDPENSQPEAFKLPLRKEQLRSLWWMLEQEKATGK
THTFVEEEISESLLPAVGWRAEGKAERPVMVRGGVIADQVGYGKTVISIALVAQTLSLPAPEPATPGLIDLKATL
IVVPGHLSKQWPNEIARFTGSMFKVIVIQGMKDLQEKTIAELGKADIIVMASEIFESDVYWSRLEYLSAQPREWL
HDTQGGRFFCDRLDAAMESLVSQTKILKEKGSEAAMRAMEDKKKSLVDNVGSKKEVHTAVNFGKRMKGQAYRDKH
DSDSKAKPITKEELERWEASEDEDDDENSKTYIPIPKFHSFTGSESIFSASVKKDYKLLPNPVLHMFRFRRVIAD
EFTYLQKKSLAAVLRLSSSYRWILSGTPPVSDFAAIRSIATFMGIHLGVEDDGEGDVQYQKARAKDQTQAEKFHA
FREVHSRAWHNRRDELAQEFLNVFVRQNIAEIEDIPTVEHIHTFKLPASEGAVYLELEHHLQALEMQARKETKEK
NVSQGDRNARLEEALSDSKTAEEALLKRCCHFTLDLSDKTQDAKSAQEACDHITSARARQLLACQEDLSRSVNQA
IALHGWIKKKGGFSKNDDERQPFAEWIAFSSNISKHQGDIEAARILLKVIEKCGVKDGNIPPSPSDKQSPSIASG
AKMDDVKWQLREQTHLLRKLVKELVARVRSLRFFEVVRKIQKGKSDAQIVLESSECGHKPSTNPDIEMAILSCCG
HVACHKCMRKAAASQRCVKSGECQAAVRPTNIVKVSSLGVEGELSSGRYGAKLEHLVNLIHSIPKNERVLVELQW
EDLAGKVSEALSAGRIPHVTLSGSAKSRANTLDRFQSTNADSARVLLLKMNDASAAGSNLTTANHAVFLGPLFTN
SLFNYRAVETQAIGRVRRYGQQKKVHIHRLLALDTIDMTIFNARRTELKEKGGGSDYKDDDDKHHHHHH
SEQ ID NO 32: Variant 10
MGDDWYEIDYIADSRVIRRKGRQILQYLIHWAGYAVHERTWEDEDGIGGEDCALVQEFYRKNPGKPRLSPSSVRK
EVKLARMVEVVITTRRIDGKSRAASSTDQPSPHRLGITSPQANNIGGEDPNPSLTRRPVRSTVSEIAKRPTSKKV
HPNKKCKASSDDESDFVFEEGEWDEDEDDDNDVDFRSSEDDEDDEQERSAEEPESDEEIIKPAKKTKSSLPKAKI
RPKPANLGGFVTGVRPLNQGLDIKAAVRNMSDDLPPISDIEAMEDHLVSRIPDIVELVRQLNGRKLRVATMCSGT
ESPLLALNMIAKAIKAQHGLTLAFEHVESCEIEPFKQAYIERNFTPPILFRDVTELGKKRAHTAYGSMVDVPGDV
DILIAGTSCVDYSNLNNVQQDIDANGESGRTFRGMLQWVKKHQPPIVILENVCNAPWDKVVEYFGQIDYDAQYTR
LDTKEFYIPHTRTRVYLFATPSSSESDDLPEKWAQTVKDLRRPWSSPFEAFLLHTDDPNIHRARLELASARAQTD
GTSRKTTDWNRCESRHQRARQDEALGLLRPLTSWQEAGVCKGLDWTWNDWLLAQTERVVDLLEISTLRMAKDGID
SGFKACIWNVSQNVDRQTGSSKTALAPCLTPNMIPWVTIRGGPVTGREALALQGIPVRELLLTSENEDQLADLAG
NAMTTTVVGSAMIAALKVACHKITEGANPEKEAALILEKEAVDDEQVANRIIGEDYLEHHDLDLAKVTKSNLSEI
LDLACRSSRHCQCEGQSGTAPNILECQECSYRACKSCGGRPEHVYAPCANQRVEPAEFEKRFKGLLPMRVRIAGL
TDQCLNAVRKAAEKSNKGSVNDNDWQLWSTALLEGIHDAEFRFRYLKRQSTWTAVYEARRAMLSLVLRNQIPEWR
LTIKAPASEPNNSQLRALLLHPVARLQIDIAGQDVLCGPWELCIPSMKTIDIEITGKGELLPSWQASLGLQGPFA
NTTRWSEVEISLQAEDENTLDRKLSGTYQLLPRCGQAMSSLHKKRPDLSDDGLPQLYFELDPTRCGESREDRYVF
STSTERLDYGTERPVIARLDSHWREGNEKQRKVKLDVSGAWVKCPEAHLTAIGGDDIAVVANDAAANEIHRDRAT
FAIPSSASAISASLTTEGCSHAMALLSCRVPLDPTHSESMWRRGAWAEIDLSHQGNTTFANLAWITERLPPLDGL
KNWAHIADDVSEHVCERCAPRPPKIHWIKREGKANKKGNKTKSTIIAFEDKLEAGQYEHALKHRPSPFVVQLRLD
QDIGSFRIGLNIVSLAHRALSRLPPTTSEHKISLSWRLTPGHVTESPQPRRVFILPSNKQDPENSQPEAFKLPLR
KEQLRSLWWMLEQEKATGKTHTFVEEEISESLLPAVGWRAEGKAERPVMVRGGVIADQVGYGKTVISIALVAQTL
SLPAPEPATPGLIDLKATLIVVPGHLSKQWPNEIARFTGSMFKVIVIQGMKDLQEKTIAELGKADIIVMASEIFE
SDVYWSRLEYLSAQPREWLHDTQGGRFFCDRLDAAMESLVSQTKILKEKGSEAAMRAMEDKKKSLVDNVGSKKEV
HTAVNFGKRMKGQAYRDKHDSDSKAKPITKEELERWEASEDEDDDENSKTYIPIPKFHSFTGSESIFSASVKKDY
KLLPNPVLHMFRFRRVIADEFTYLQKKSLAAVLRLSSSYRWILSGTPPVSDFAAIRSIATEMGIHLGVEDDGEGD
VQYQKARAKDQTQAEKFHAFREVHSRAWHNRRDELAQEFLNVFVRQNIAEIEDIPTVEHIHTFKLPASEGAVYLE
LEHHLQALEMQARKETKFKNVSQGDRNARLEEALSDSKTAEEALLKRCCHFTLDLSDKTQDAKSAQEACDHITSA
RARQLLACQEDLSRSVNQAIALHGWIKKKGGFSKNDDERQPFAEWIAFSSNISKHQGDIEAARILLKVIEKCGVK
DGNIPPSPSDKQSPSIASGAKMDDVKWQLREQTHLLRKLVKELVARVRSLRFFEVVRKIQKGKSDAQIVLESSEC
GHKPSTNPDIEMAILSCCGHVACHKCMRKAAASQRCVKSGECQAAVRPTNIVKVSSLGVEGELSSGRYGAKLEHL
VNLIHSIPKNERVLVFLQWEDLAGKVSEALSAGRIPHVTLSGSAKSRANTLDRFQSTNADSARVLLLKMNDASAA
GSNLTTANHAVFLGPLFTNSLENYRAVETQAIGRVRRYGQQKKVHIHRLLALDTIDMTIFNARRTELKEKGGGSD
YKDDDDKHHHHHH
SEQ ID NO 33: Variant 11
MGGFVTGVRPLNQGLDIKAAVRNMSDDLPPISDIEAMEDHLVSRIPDIVELVRQLNGRKLRVATMCSGTESPLLA
LNMIAKAIKAQHGLTLAFEHVESCEIEPFKQAYIERNFTPPILFRDVTELGKKRAHTAYGSMVDVPGDVDILIAG
TSCVDYSNLNNVQQDIDANGESGRTFRGMLQWVKKHQPPIVILENVCNAPWDKVVEYFGQIDYDAQYTRLDTKEF
YIPHTRTRVYLFATPSSSESDDLPEKWAQTVKDLRRPWSSPFEAFLLHTDDPNIHRARLELASARAQTDGTSRKT
TDWNRCESRHQRARQDEALGLLRPLTSWQEAGVCKGLDWTWNDWLLAQTERVVDLLEISTLRMAKDGIDSGFKAC
IWNVSQNVDRQTGSSKTALAPCLTPNMIPWVTIRGGPVTGREALALQGIPVRELLLTSENEDQLADLAGNAMTTT
VVGSAMIAALKVACHKITEGANPEKEAALILEKEAVDDEQVANRIIGEDYLEHHDLDLAKVTKSNLSEILDLACR
SSRHCQCEGQSGTAPNILECQECSYRACKSCGGRPEHVYAPCANQRVEPAEFEKRFKGLLPMRVRIAGLTDQCLN
AVRKAAEKSNKGSVNDNDWQLWSTALLEGIHDAEFRFRYLKRQSTWTAVYEARRAMLSLVLRNQIPEWRLTIKAP
ASEPNNSQLRALLLHPVARLQIDIAGQDVLCGPWELCIPSMKTIDIEITGKGELLPSWQASLGLQGPFANTTRWS
EVEISLQAEDENTLDRKLSGTYQLLPRCGQAMSSLHKKRPDLSDDGLPQLYFFLDPTRCGESREDRYVESTSTER
LDYGTERPVIARLDSHWREGNEKQRKVKLDVSGAWVKCPEAHLTAIGGDDIAVVANDAAANEIHRDRATFAIPSS
ASAISASLTTEGCSHAMALLSCRVPLDPTHSESMWRRGAWAEIDLSHQGNTTFANLAWITERLPPLDGLKNWAHI
ADDVSEHVCERCAPRPPKIHWIKREGKANKKGNKTKSTIIAFEDKLEAGQYEHALKHRPSPFVVQLRLDQDIGSF
RIGLNIVSLAHRALSRLPPTTSEHKISLSWRLTPGHVTESPQPRRVFILPSNKQDPENSQPEAFKLPLRKEQLRS
LWWMLEQEKATGKTHTFVEEEISESLLPAVGWRAEGKAERPVMVRGGVIADQVGYGKTVISIALVAQTLSLPAPE
PATPGLIDLKATLIVVPGHLSKQWPNEIARFTGSMFKVIVIQGMKDLQEKTIAELGKADIIVMASEIFESDVYWS
RLEYLSAQPREWLHDTQGGRFFCDRLDAAMESLVSQTKILKEKGSEAAMRAMEDKKKSLVDNVGSKKEVHTAVNF
GKRMKGQAYRDKHDSDSKAKPITKEELERWEASEDEDDDENSKTYIPIPKFHSFTGSESIFSASVKKDYKLLPNP
VLHMFRFRRVIADEFTYLQKKSLAAVLRLSSSYRWILSGTPPVSDFAAIRSIATFMGIHLGVEDDGEGDVQYQKA
RAKDQTQAEKFHAFREVHSRAWHNRRDELAQEFLNVFVRQNIAEIEDIPTVEHIHTEKLPASEGAVYLELEHHLQ
ALEMQARKETKFKNVSQGDRNARLEEALSDSKTAEEALLKRCCHFTLDLSDKTQDAKSAQEACDHITSARARQLL
ACQEDLSRSVNQAIALHGWIKKKGGFSKNDDERQPFAEWIAFSSNISKHQGDIEAARILLKVIEKCGVKDGNIPP
SPSDKQSPSIASGAKMDDVKWQLREQTHLLRKLVKELVARVRSLRFFEVVRKIQKGKSDAQIVLESSECGHKPST
NPDIEMAILSCCGHVACHKCMRKAAASQRCVKSGECQAAVRPTNIVKVSSLGVEGELSSGRYGAKLEHLVNLIHS
IPKNERVLVFLQWEDLAGKVSEALSAGRIPHVTLSGSAKSRANTLDRFQSTNADSARVLLLKMNDASAAGSNLTT
ANHAVELGPLFTNSLFNYRAVETQAIGRVRRYGQQKKVHIHRLLALDTIDMTIFNARRTELKEKGGGSDYKDDDD
KHHHHHH
SEQ ID NO 34: Variant 12
MGLDIKAAVRNMSDDLPPISDIEAMFDHLVSRIPDIVELVRQLNGRKLRVATMCSGTESPLLALNMIAKAIKAQH
GLTLAFEHVESCEIEPFKQAYIERNFTPPILFRDVTELGKKRAHTAYGSMVDVPGDVDILIAGTSCVDYSNLNNV
QQDIDANGESGRTFRGMLQWVKKHQPPIVILENVCNAPWDKVVEYFGQIDYDAQYTRLDTKEFYIPHTRTRVYLF
ATPSSSESDDLPEKWAQTVKDLRRPWSSPFEAFLLHTDDPNIHRARLELASARAQTDGTSRKTTDWNRCESRHQR
ARQDEALGLLRPLTSWQEAGVCKGLDWTWNDWLLAQTERVVDLLEISTLRMAKDGIDSGFKACIWNVSQNVDRQT
GSSKTALAPCLTPNMIPWVTIRGGPVTGREALALQGIPVRELLLTSENEDQLADLAGNAMTTTVVGSAMIAALKV
ACHKITEGANPEKEAALILEKEAVDDEQVANRIIGEDYLEHHDLDLAKVTKSNLSEILDLACRSSRHCQCEGQSG
TAPNILECQECSYRACKSCGGRPEHVYAPCANQRVEPAEFEKRFKGLLPMRVRIAGLTDQCLNAVRKAAEKSNKG
SVNDNDWQLWSTALLEGIHDAEFRFRYLKRQSTWTAVYEARRAMLSLVLRNQIPEWRLTIKAPASEPNNSQLRAL
LLHPVARLQIDIAGQDVLCGPWELCIPSMKTIDIEITGKGELLPSWQASLGLQGPFANTTRWSEVEISLQAEDEN
TLDRKLSGTYQLLPRCGQAMSSLHKKRPDLSDDGLPQLYFFLDPTRCGESREDRYVESTSTERLDYGTERPVIAR
LDSHWREGNEKQRKVKLDVSGAWVKCPEAHLTAIGGDDIAVVANDAAANEIHRDRATFAIPSSASAISASLTTEG
CSHAMALLSCRVPLDPTHSESMWRRGAWAEIDLSHQGNTTFANLAWITERLPPLDGLKNWAHIADDVSEHVCERC
APRPPKIHWIKREGKANKKGNKTKSTIIAFEDKLEAGQYEHALKHRPSPFVVQLRLDQDIGSFRIGLNIVSLAHR
ALSRLPPTTSEHKISLSWRLTPGHVTESPQPRRVFILPSNKQDPENSQPEAFKLPLRKEQLRSLWWMLEQEKATG
KTHTFVEEEISESLLPAVGWRAEGKAERPVMVRGGVIADQVGYGKTVISIALVAQTLSLPAPEPATPGLIDLKAT
LIVVPGHLSKQWPNEIARFTGSMFKVIVIQGMKDLQEKTIAELGKADIIVMASEIFESDVYWSRLEYLSAQPREW
LHDTQGGRFFCDRLDAAMESLVSQTKILKEKGSEAAMRAMEDKKKSLVDNVGSKKEVHTAVNFGKRMKGQAYRDK
HDSDSKAKPITKEELERWEASEDEDDDENSKTYIPIPKFHSFTGSESIFSASVKKDYKLLPNPVLHMFRFRRVIA
DEFTYLQKKSLAAVLRLSSSYRWILSGTPPVSDFAAIRSIATFMGIHLGVEDDGEGDVQYQKARAKDQTQAEKFH
AFREVHSRAWHNRRDELAQEFLNVFVRQNIAEIEDIPTVEHIHTFKLPASEGAVYLELEHHLQALEMQARKETKE
KNVSQGDRNARLEEALSDSKTAEEALLKRCCHFTLDLSDKTQDAKSAQEACDHITSARARQLLACQEDLSRSVNQ
AIALHGWIKKKGGFSKNDDERQPFAEWIAFSSNISKHQGDIEAARILLKVIEKCGVKDGNIPPSPSDKQSPSIAS
GAKMDDVKWQLREQTHLLRKLVKELVARVRSLRFFEVVRKIQKGKSDAQIVLESSECGHKPSTNPDIEMAILSCC
GHVACHKCMRKAAASQRCVKSGECQAAVRPTNIVKVSSLGVEGELSSGRYGAKLEHLVNLIHSIPKNERVLVELQ
WEDLAGKVSEALSAGRIPHVTLSGSAKSRANTLDRFQSTNADSARVLLLKMNDASAAGSNLTTANHAVELGPLFT
NSLENYRAVETQAIGRVRRYGQQKKVHIHRLLALDTIDMTIFNARRTELKEKGGGSDYKDDDDKHHHHHH
SEQ ID NO: 35: MT1 consensus sequence
LXVATXCSGTXXPJXAL
SEQ ID NO: 36: MT23 consensus sequence
HLFSCEIEPFKQXXIXRNXXPXXXIFRDVXEL
SEQ ID NO: 37: MT4 consensus sequence
DILXAGCSCVDYSNXNX
SEQ ID NO: 38: MT5 consensus sequence
GESXRTFFAAJXXIXXXRP
SEQ ID NO: 39: MT6 consensus sequence
JVILENVXXAPW
SEQ ID NO: 40: MT7 consensus sequence
LDSKXXYLPQTRZRGYLXA
SEQ ID NO: 41: MT7B consensus sequence
WXXXXXRHXDXRXXXXLXXXXXPXSXKXXXNGXXXXXXXPSXSWXXX
SEQ ID NO: 42: MT8 consensus sequence
QXXRVXDXXDIXFXX
SEQ ID NO: 43: MT8B consensus sequence
DLXYKTXXJDXSQNVDRXXXXXXXXXXXX
SEQ ID NO: 44: MT9 consensus sequence
GXXGCITPSGXPX
SEQ ID NO: 45: MT10 consensus sequence
EXLALQGLPXDXLXXSTEXQXQLXDLAGNAMTXTVVGAXTLAAL
SEQ ID NO: 46: ATP11B consensus sequence
LRPXQXXXVXWMLZREXXXXXFXXXEIEEEVXXXLXXRXXGKAEX
SEQ ID NO: 47: ATP12 consensus sequence
RGGVXAHEXGYGKTVVXLALX
SEQ ID NO: 48: ATP13 consensus sequence
XATLXVVPXHIXXQWXXEXXKF
SEQ ID NO: 49: ATP14 consensus sequence
FARXVXDEXXYXXXXXJXXXVXXXXAXXKWLLSGTP
SEQ ID NO: 50: ATP14B consensus sequence
PGLPXXXXGP
SEQ ID NO: 51: ATP15 consensus sequence
XLLLBXXXETXAGSNLT
SEQ ID NO: 52: ATP15B consensus sequence
AXHVI FXXPXXXXXXXXXXXTXXQAXGRCXRXGQ
SEQ ID NO: 53: MT1 sequence DNMT5 exemplar 1
LRVVTMCSGTESPLLAL
SEQ ID NO: 54: MT23 sequence DNMT5 exemplar 1
HLFSAEIVPFKQAYIERNFHPPYLERDVSEL
SEQ ID NO: 55: MT4 sequence DNMT5 exemplar 1
DIVIAGFSCVDESALNN
SEQ ID NO: 56: MT5 sequence DNMT5 exemplar 1
GESGVTFYGIRSYIEKYRP
SEQ ID NO: 57: MT6 sequence DNMT5 exemplar 1
LIILENVKNAPW
SEQ ID NO: 58: MT7 sequence DNMT5 exemplar 1
IDTKTYYLPQTRERGYALC
SEQ ID NO: 59: MT7B sequence DNMT5 exemplar 1
WSRYQARHQSYRLDNKLGYKRPMTRFQDNGTCKMPDFVWQEW
SEQ ID NO: 60: MT8 sequence DNMT5 exemplar 1
QPERIWDTLDMNFLR
SEQ ID NO: 61: MT8B sequence DNMT5 exemplar 1
DLNFKERYLDLSQGIDREIDGR
SEQ ID NO: 62: MT9 sequence DNMT5 exemplar 1
GIMGCITPCGIPY
SEQ ID NO: 63: MT10 sequence DNMT5 exemplar 1
EALSLQGLPLDRLLLTKESQRELQDLAGNAMSSTVVGAATLSAL
SEQ ID NO: 64: ATP11B sequence DNMT5 exemplar 1
LRPEQLRSLSWMIEQEAEEPVPFEEEEVEEALLPFMMWRAEAKVTH
SEQ ID NO: 65: ATP12 sequence DNMT5 exemplar 1
RGGVLADEVGYGKTALILGLI
SEQ ID NO: 66: ATP13 sequence DNMT5 exemplar 1
KATLVVVPKMMVTQWKEEISKE
SEQ ID NO: 67: ATP14 sequence DNMT5 exemplar 1
FNRLVIDEFTYVSERKLGPVLNLKARSVWVLSGTP
SEQ ID NO: 68: ATP14B sequence DNMT5 exemplar 1
NPSTRLAN
SEQ ID NO: 69: ATP15 sequence DNMT5 exemplar 1
VLILNMGSESAAGINLQ
SEQ ID NO: 70: ATP15B sequence DNMT5 exemplar 1
ANHVIFLHPFLAKTQYDYESTMTQAIGRARREGQ
SEQ ID NO: 71: MT1 sequence DNMT5 exemplar 2
LRVATMCSGTESPLLAL
SEQ ID NO: 72: MT23 sequence DNMT5 exemplar 2
HLFSAEIDPFKQSYIQRNESPDIIFRDVNEL
SEQ ID NO: 73: MT4 sequence DNMT5 exemplar 2
DLLVVGFSCVDFSNLNV
SEQ ID NO: 74: MT5 sequence DNMT5 exemplar 2
GESGHTFFAVLRYMERCRP
SEQ ID NO: 75: MT6 sequence DNMT5 exemplar 2
LAVLENVSLAPW
SEQ ID NO: 76: MT7 sequence DNMT5 exemplar 2
VDTKNYYLPQTRERGYMLC
SEQ ID NO: 77: MT7B sequence DNMT5 exemplar 2
WTRYKARHMAYRMEEGLGNKRPLTKWQDNGTCQMPDFYWHDW
SEQ ID NO: 78: MT8 sequence DNMT5 exemplar 2
QTERVWDTLDVNYLR
SEQ ID NO: 79: MT8B sequence DNMT5 exemplar 2
DINYKTRVVDLSQGLDRELDHR
SEQ ID NO: 80: MT9 sequence DNMT5 exemplar 2
GVAGCLTPRGLHE
SEQ ID NO: 81: MT10 sequence DNMT5 exemplar 2
EALALQGIPIDRLLLGSDSQRALYDLAGNAMSSTVVGAAILSAL
SEQ ID NO: 82: ATP11B sequence DNMT5 exemplar 2
LRPEQLRSLTWMVSQEADDVPPFEEEEVVEALLPVINWRAEGRAST
SEQ ID NO: 83: ATP12 sequence DNMT5 exemplar 2
KGGILGDDVGYGKTAISLGLI
SEQ ID NO: 84: ATP13 sequence DNMT5 exemplar 2
KATLIIVPSHLVDQWAREITKE
SEQ ID NO: 85: ATP14 sequence DNMT5 exemplar 2
FNRLIIDEFTYSKARNYASALAIPARKKWILSGTP
SEQ ID NO: 86: ATP14B sequence DNMT5 exemplar 2
RDNERLKA
SEQ ID NO: 87: ATP15 sequence DNMT5 exemplar 2
VLILTLGDVTASGLNLQ
SEQ ID NO: 88: ATP15B sequence DNMT5 exemplar 2
ANHIIFYSPLVARSQYDYESGMAQAIGRCRRYGQ
SEQ ID NO: 89: MT1 sequence DNMT5 exemplar 3
LRVATVCSGTDAPIFAL
SEQ ID NO: 90: MT23 sequence DNMT5 exemplar 3
HVFSCEIEPFKQGFIRRNLPQCSLIERDIVEL
SEQ ID NO: 91: MT4 sequence DNMT5 exemplar 3
DIFFAGCSCVDYSNMNTN
SEQ ID NO: 92: MT5 sequence DNMT5 exemplar 3
GESARTFFAAVKLIAERRP
SEQ ID NO: 93: MT6 sequence DNMT5 exemplar 3
IVILENVSGAPW
SEQ ID NO: 94: MT7 sequence DNMT5 exemplar 3
LDSKNFYLPQTRQRGYLVA
SEQ ID NO: 95: MT7B sequence DNMT5 exemplar 3
WSLSSLRHIDARRRANLRHDDNP FSMKAMRGGQLIHMRPPDHSWRPV
SEQ ID NO: 96: MT8 sequence DNMT5 exemplar 3
AGPRILDLMDIVFAV
SEQ ID NO: 97: MT8B sequence DNMT5 exemplar 3
DLGYKTAMIDVSQNVDRNEFCRLGR
SEQ ID NO: 98: MT9 sequence DNMT5 exemplar 3
GIVGCVTPSGLPI
SEQ ID NO: 99: MT10 sequence DNMT5 exemplar 3
ETLALQGLPAEELVLSTETQAQQRDLAGNAMSVPVVGAATYAAI
SEQ ID NO: 100: ATP11B sequence DNMT5 exemplar 3
LRPSQRDAVKWMIQRERIPLNFIKREIEEEVVQPLNLRVAGKAEW
SEQ ID NO: 101: ATP12 sequence DNMT5 exemplar 3
RGGVVAHEIGYGKTVVTLALV
SEQ ID NO: 102: ATP13 sequence DNMT5 exemplar 3
SATLVIVPRHITDQWAKEIDKF
SEQ ID NO: 103: ATP14 sequence DNMT5 exemplar 3
FARIVWDECSYDDDKDSYIPVFVANAVANSKWLLSGTP
SEQ ID NO: 104: ATP14B sequence DNMT5 exemplar 3
PGLPAITEGP
SEQ ID NO: 105: ATP15 sequence DNMT5 exemplar 3
ILLLDIDNETSAGSNLT
SEQ ID NO: 106: ATP15B sequence DNMT5 exemplar 3
ANHVIFANPFVHHDEEHQSRTVRQARGRCVRIGQ
SEQ ID NO: 107: MT1 sequence DNMT5 exemplar 4
LNVATLCSGTDAPIFAL
SEQ ID NO: 108: MT23 sequence DNMT5 exemplar 4
HLFSCEIEPFKQGFIRRNLPPGTLIFRDVVEL
SEQ ID NO: 109: MT4 sequence DNMT5 exemplar 4
DILFAGCSCVDYSNMNQ
SEQ ID NO: 110: MT5 sequence DNMT5 exemplar 4
GESARTFFAAIKLITVIRP
SEQ ID NO: 111: MT6 sequence DNMT5 exemplar 4
LVILENVYNAPW
SEQ ID NO: 112: MT7 sequence DNMT5 exemplar 4
LDSKDFYLPQTRQRGYLVA
SEQ ID NO: 113: MT7B sequence DNMT5 exemplar 4
WALCSLRHADVRQRHGIRRDDNPFSLKAVRNSRLIFAAYPSHSWMQF
SEQ ID NO: 114: MT8 sequence DNMT5 exemplar 4
QVARVVDEMDIAFAA
SEQ ID NO: 115: MT8B sequence DNMT5 exemplar 4
DLGYKTCMIDVSQNVDRNDLVNGGR
SEQ ID NO: 116: MT9 sequence DNMT5 exemplar 4
GVVGCITPSGMPI
SEQ ID NO: 117: MT10 sequence DNMT5 exemplar 4
ETLALQGLPVDDLVVSTESQAQLRDLAGNAMTVTVVGAVTLAAL
SEQ ID NO: 118: ATP11B sequence DNMT5 exemplar 4
LRPSQREAVNWMLQRERVPLDFVKREVEEEVVAPLNLRVLGKAEW
SEQ ID NO: 119: ATP12 sequence DNMT5 exemplar 4
RGGVVAHEVGYGKTVVTLAVC
SEQ ID NO: 120: ATP13 sequence DNMT5 exemplar 4
SATLVVVPKHITAQWESEAAKF
SEQ ID NO: 121: ATP14 sequence DNMT5 exemplar 4
FARVVWDECSYDDDDNIRLFVAHAVANTKWLLSGTP
SEQ ID NO: 122: ATP14B sequence DNMT5 exemplar 4
PGLPAVTKGP
SEQ ID NO: 123: ATP15 sequence DNMT5 exemplar 4
ILLLDMDSETSAGSNLT
SEQ ID NO: 124: ATP15B sequence DNMT5 exemplar 4
ATRVIFASPYVHQDEEHQVRTVRQAKGRCIRTGQ
SEQ ID NO: 125: MT1 sequence DNMT5 exemplar 5
LNVATLCSGTDAPIFAL
SEQ ID NO: 126: MT23 sequence DNMT5 exemplar 5
HLFSCEIEPFKQGFIRRNLPQGTLIFRDVVEM
SEQ ID NO: 127: MT4 sequence DNMT5 exemplar 5
DILFAGCSCVDYSNMNS
SEQ ID NO: 128: MT5 sequence DNMT5 exemplar 5
GESSRTFFAAVKLISAMRP
SEQ ID NO: 129: MT6 sequence DNMT5 exemplar 5
AVILENVYGAPW
SEQ ID NO: 130: MT7 sequence DNMT5 exemplar 5
LDSKDYYLPQTRQRGYLVA
SEQ ID NO: 131: MT7B sequence DNMT5 exemplar 5
WALSSLRHYDVRHANGVREDDNPFSLKAMRHGRLIFASYPSHSWRRF
SEQ ID NO: 132: MT8 sequence DNMT5 exemplar 5
QPPRVTDLMDIVFAV
SEQ ID NO: 133: MT8B sequence DNMT5 exemplar 5
DLGYKTCMIDASQNVDRIHKIDAKN
SEQ ID NO: 134: MT9 sequence DNMT5 exemplar 5
GIVGCITPSGLPI
SEQ ID NO: 135: MT10 sequence DNMT5 exemplar 5
ETLSLQGLPVDELVISTETQANLRDLAGNAMTVTVVGAATLALL
SEQ ID NO: 136: ATP11B sequence DNMT5 exemplar 5
LRPSQERAVEWMLQRENAPLDFVKAEIEEEVVRPLNMRVVGKAEW
SEQ ID NO: 137: ATP12 sequence DNMT5 exemplar 5
KGGVVAHEIGYGKTVVMLALI
SEQ ID NO: 138: ATP13 sequence DNMT5 exemplar 5
SATLVVVPRHITDQWAKEATKF
SEQ ID NO: 139: ATP14 sequence DNMT5 exemplar 5
FARVIWDECSYDGNENVTLFVSNAVANAKWLLSGTP
SEQ ID NO: 140: ATP14B sequence DNMT5 exemplar 5
PGLPAVTDGP
SEQ ID NO: 141: ATP15 sequence DNMT5 exemplar 5
ILLLDIDSETSAGSNLT
SEQ ID NO: 142: ATP15B sequence DNMT5 exemplar 5
ANHVIFANPYVHPDEDHRARTVRQAKGRCVRMGQ
SEQ ID NO: 143: MT1 sequence DNMT5 exemplar 6
LRVVTMCSGTESPLLAL
SEQ ID NO: 144: MT23 sequence DNMT5 exemplar 6
HLFSAEIVHFKQAYIERNFHPPIIFRDVREL
SEQ ID NO: 145: MT4 sequence DNMT5 exemplar 6
DILIAGFSCVDESNLNR
SEQ ID NO: 146: MT5 sequence DNMT5 exemplar 6
GESGQTFEGILSYVAQHRP
SEQ ID NO: 147: MT6 sequence DNMT5 exemplar 6
LVVLENIKHAPW
SEQ ID NO: 148: MT7 sequence DNMT5 exemplar 6
VDTKNYYLPQTRARGYMLC
SEQ ID NO: 149: MT7B sequence DNMT5 exemplar 6
WSSYRIRHQDYRRTNQLGYRRPVTNFREGGLCRPIDWMDRSW
SEQ ID NO: 150: MT8 sequence DNMT5 exemplar 6
QPERVLDTIDADFLY
SEQ ID NO: 151: MT8B sequence DNMT5 exemplar 6
DNRYKLRVHDVSQGVDREADNR
SEQ ID NO: 152: MT9 sequence DNMT5 exemplar 6
GIVGCLTPCGMPY
SEQ ID NO: 153: MT10 sequence DNMT5 exemplar 6
ESLGLQGLPTYRLNLTRESQKELQDLAGNAMSSTVVAGAIASAL
SEQ ID NO: 154: ATP11B sequence DNMT5 exemplar 6
LRPDQLRSLAWMLSRESDGMTPFMEEEIDEAFMAPLDWRAEIKIIV
SEQ ID NO: 155: ATP12 sequence DNMT5 exemplar 6
RGGILADHVGFGKTAVVLGLL
SEQ ID NO: 156: ATP13 sequence DNMT5 exemplar 6
KATLIVVPETLFGQWRAEIEKF
SEQ ID NO: 157: ATP14 sequence DNMT5 exemplar 6
FQRMVVDEFTYLDQDKHTLLASIKARSKWLLSGTP
SEQ ID NO: 158: skipped
SEQ ID NO: 159: ATP15 sequence DNMT5 exemplar 6
ILLLRLGTKNAAGLNLQ
SEQ ID NO: 160: ATP15B sequence DNMT5 exemplar 6
ANHIIFFAPMSVHHPSEYQASVAQAMGRAIRWGQ
SEQ ID NO: 161: skipped
SEQ ID NO: 162: skipped
SEQ ID NO: 163: MT4 sequence DNMT5 exemplar 7
DIFVAGCSCVDESNLNS
SEQ ID NO: 164: MT5 sequence DNMT5 exemplar 7
GESSTTFLAALSYIIKVEP
SEQ ID NO: 165: MT6 sequence DNMT5 exemplar 7
IVILENVDSAPW
SEQ ID NO: 166: MT7 sequence DNMT5 exemplar 7
VNSRMYYMPQTRQRGYLVA
SEQ ID NO: 167: MT7B sequence DNMT5 exemplar 7
WGFSQVRHQSTREEAKLSLTARPFSRGIIVRGRMVDMQPPSRSWRSY
SEQ ID NO: 168: MT8 sequence DNMT5 exemplar 7
QPARVLDFIDIAYLQ
SEQ ID NO: 169: MT8B sequence DNMT5 exemplar 7
DLRYKTWVLDVSQNVDRSTVFGTN
SEQ ID NO: 170: MT9 sequence DNMT5 exemplar 7
GSIGCLTPSGQPI
SEQ ID NO: 171: MT10 sequence DNMT5 exemplar 7
ESLGLQGLPVDDLVLSTETQAQLQDLAGNAMTVTVIGSVILAAL
SEQ ID NO: 172: ATP11B sequence DNMT5 exemplar 7
LRDDQVLSVKWMLGRETCPAPFLEKEIEELVLPRLAARIIGRAIF
SEQ ID NO: 173: ATP12 sequence DNMT5 exemplar 7
RGGVAAHDIGYGKTVVTIALL
SEQ ID NO: 174: ATP13 sequence DNMT5 exemplar 7
KATLIIVPGHISTQWVAEFKKF
SEQ ID NO: 175: ATP14 sequence DNMT5 exemplar 7
FARIVVDEYSYQAPSTLSFVSSSLAFAKWLLSGTP
SEQ ID NO: 176: ATP14B sequence DNMT5 exemplar 7
PGLPSVTEGP
SEQ ID NO: 177: ATP15 sequence DNMT5 exemplar 7
VLMLNPSEVSCAGVNLT
SEQ ID NO: 178: ATP15B sequence DNMT5 exemplar 7
ANHVMFVSPFLEPDPYRRNQGMRQAVGRCVRQGQ
SEQ ID NO: 179: MT1 sequence DNMT5 exemplar 8
LAVATICSGTDAPIFAL
SEQ ID NO: 180: MT23 sequence DNMT5 exemplar 8
HVFSCEIEPFKQGFISRNMPPATVIFRDVVEL
SEQ ID NO: 181: MT4 sequence DNMT5 exemplar 8
DILFAGCSCVDYSNLNH
SEQ ID NO: 182: MT5 sequence DNMT5 exemplar 8
GESARTFFAAVKLIARTRP
SEQ ID NO: 183: MT6 sequence DNMT5 exemplar 8
AVILENVLGAPW
SEQ ID NO: 184: MT7 sequence DNMT5 exemplar 8
LDSKEFYLPQTRQRGYLVA
SEQ ID NO: 185: MT7B sequence DNMT5 exemplar 8
WNLCSIRHAIVRQKYGLRRDENPVSKKAMRNGRIIFANFPSHSWMRF
SEQ ID NO: 186: MT8 sequence DNMT5 exemplar 8
QVPRIPDLVDIVFAI
SEQ ID NO: 187: MT8B sequence DNMT5 exemplar 8
DLGYKTGMIDVSQNVDRTEYTRSGS
SEQ ID NO: 188: MT9 sequence DNMT5 exemplar 8
GIIGCITPSGLPV
SEQ ID NO: 189: MT10 sequence DNMT5 exemplar 8
EALALQGLPVDELVISTETQAQLRDLAGNAMTVTVVGAATLALI
SEQ ID NO: 190: ATP11B sequence DNMT5 exemplar 8
LRDSQKEAVKWMLRRELVPLDFIKCEIEEEVVSPLNMRVMGKAEW
SEQ ID NO: 191: ATP12 sequence DNMT5 exemplar 8
RGGVVAHEIGYGKTVVTLALI
SEQ ID NO: 192: ATP13 sequence DNMT5 exemplar 8
SATLVIVPKHITEQWANEAYKE
SEQ ID NO: 193: ATP14 sequence DNMT5 exemplar 8
FARVVWDECSYNDEEKIPLVIANVVANSKWLLSGTP
SEQ ID NO: 194: ATP14B sequence DNMT5 exemplar 8
PGLPPVTKGP
SEQ ID NO: 195: skipped
SEQ ID NO: 196: skipped
SEQ ID NO: 197: MT1 sequence DNMT5 exemplar 9
LNVATLCS
SEQ ID NO: 198: MT23 sequence DNMT5 exemplar 9
HLFSCEIEPFKQGFIRRNLPPGTLIFRDVVEL
SEQ ID NO: 199: MT4 sequence DNMT5 exemplar 9
DILFAGCSCVDYSNMNQ
SEQ ID NO: 200: MT5 sequence DNMT5 exemplar 9
GESARTFFAAIKLITVIRP
SEQ ID NO: 201: MT6 sequence DNMT5 exemplar 9
LVILENVYNAPW
SEQ ID NO: 202: MT7 sequence DNMT5 exemplar 9
LDSKEFYLPQTRQRGYLLA
SEQ ID NO: 203: MT7B sequence DNMT5 exemplar 9
WALCSLRHADVRQKHGIRRDDNPFSLKAMRNSRIIFAAYPSHSWMQF
SEQ ID NO: 204: MT8 sequence DNMT5 exemplar 9
QVARVVDFMDIAFAT
SEQ ID NO: 205: MT8B sequence DNMT5 exemplar 9
DLGFKTCMIDVSQNVDRNDLVNGGR
SEQ ID NO: 206: MT9 sequence DNMT5 exemplar 9
GIVGCITPSGLPI
SEQ ID NO: 207: MT10 sequence DNMT5 exemplar 9
ETLALQGLPVNDLIVSTETQAQLRDLAGNAMTVTVVGAVTLALL
SEQ ID NO: 208: ATP11B sequence DNMT5 exemplar 9
LRPSQKEAVNWMLQRERVALNFVKREIEEEVVAPLNLRVMGKAEW
SEQ ID NO: 209: ATP12 sequence DNMT5 exemplar 9
RGGVVAHEIGYGKTVVTLAVC
SEQ ID NO: 210: ATP13 sequence DNMT5 exemplar 9
SATLVIVPKHITAQWESEAAKF
SEQ ID NO: 211: ATP14 sequence DNMT5 exemplar 9
FARIVWDECSYDDDSNIRLEVANAVANAKWLLSGTP
SEQ ID NO: 212: ATP14B sequence DNMT5 exemplar 9
PGLPAVTKGP
SEQ ID NO: 213: ATP15 sequence DNMT5 exemplar 9
ILLLDMDSETSAGSNLT
SEQ ID NO: 214: ATP15B sequence DNMT5 exemplar 9
ASRVIFANPYVHGDEEHQVRTVRQARGRCIRTGQ
SEQ ID NO: 215: MT1 sequence DNMT5 exemplar 10
LNVATLCSGTDAPIFAL
SEQ ID NO: 216: MT23 sequence DNMT5 exemplar 10
HLFSCEIEPFKQGFIRRNLPHGTVVERDVIEM
SEQ ID NO: 217: MT4 sequence DNMT5 exemplar 10
DILFAGCSCVDYSNMNH
SEQ ID NO: 218: MT5 sequence DNMT5 exemplar 10
SESARTFFAAIKLITILRP
SEQ ID NO: 219: MT6 sequence DNMT5 exemplar 10
FVILENVYSAPW
SEQ ID NO: 220: MT7 sequence DNMT5 exemplar 10
LDSKDFYLPQTRQRGYLVA
SEQ ID NO: 221: MT7B sequence DNMT5 exemplar 10
WALSSLRHADARHQQGLKKDDNPFSLKAMRNNRIIHATYPSHSWRSF
SEQ ID NO: 222: MT8 sequence DNMT5 exemplar 10
QPARIIDLMDIIFAT
SEQ ID NO: 223: MT8B sequence DNMT5 exemplar 10
DLGYKTYMIDASQNVDRNTLVISDR
SEQ ID NO: 224: MT9 sequence DNMT5 exemplar 10
GIVGCITPSGLPV
SEQ ID NO: 225: MT10 sequence DNMT5 exemplar 10
ETLALQGLPVDEMVLSTETQGQLRDLAGNAMTVTVVGAATLALL
SEQ ID NO: 226: ATP11B sequence DNMT5 exemplar 10
LRPSQKSAVAWMLERERAQLNFVKCEIEEEVVPSLGIRVLGKAEW
SEQ ID NO: 227: ATP12 sequence DNMT5 exemplar 10
RGGIVAHEIGYGKTVVTLAVI
SEQ ID NO: 228: ATP13 sequence DNMT5 exemplar 10
TATLIVVPKHIAHQWEAEAAKF
SEQ ID NO: 229: ATP14 sequence DNMT5 exemplar 10
FARIVWDEYTYDGGDIIHLFVSNAVANAKWLLSGTP
SEQ ID NO: 230: ATP14B sequence DNMT5 exemplar 10
PGLPPITTGP
SEQ ID NO: 231: ATP15 sequence DNMT5 exemplar 10
VILLDMDSETSAGSNLT
SEQ ID NO: 232: ATP15B sequence DNMT5 exemplar 10
ATHLIFASPYVHYDPEHQIRTVQQARGRCIRTGQ
SEQ ID NO: 233: MT1 sequence DNMT5 exemplar 11
LNVATLCSGTDAPIFAL
SEQ ID NO: 234: MT23 sequence DNMT5 exemplar 11
HLFSCEIEPFKQGFIRRNLPHGTVVFRDVIEM
SEQ ID NO: 235: MT4 sequence DNMT5 exemplar 11
DILFAGCSCVDYSNMNH
SEQ ID NO: 236: MT5 sequence DNMT5 exemplar 11
SESARTFFAAIKLITILRP
SEQ ID NO: 237: MT6 sequence DNMT5 exemplar 11
FVILENVYSAPW
SEQ ID NO: 238: MT7 sequence DNMT5 exemplar 11
LDSKDFYLPQTRQRGYLVA
SEQ ID NO: 239: MT7B sequence DNMT5 exemplar 11
WALSSLRHADARHQQGLKKDDNPFSLKAMRNNRIIHATYPSHSWRSF
SEQ ID NO: 240: MT8 sequence DNMT5 exemplar 11
QPARIIDLMDIIFAT
SEQ ID NO: 241: MT8B sequence DNMT5 exemplar 11
DLGYKTYMIDASQNVDRNTLVISDR
SEQ ID NO: 242: MT9 sequence DNMT5 exemplar 11
GIVGCITPSGLPV
SEQ ID NO: 243: MT10 sequence DNMT5 exemplar 11
ETLALQGLPVDEMVLSTETQGQLRDLAGNAMTVTVVGAATLALL
SEQ ID NO: 244: ATP11B sequence DNMT5 exemplar 11
LRPSQKSAVAWMLERERAQLNFVKCEIEEEVVPSLGIRVLGKAEW
SEQ ID NO: 245: ATP12 sequence DNMT5 exemplar 11
RGGIVAHEIGYGKTVVTLAVI
SEQ ID NO: 246: ATP13 sequence DNMT5 exemplar 11
TATLIVVPKHIAHQWEAEAAKF
SEQ ID NO: 247: ATP14 sequence DNMT5 exemplar 11
FARIVWDEYTYDGGDIIHLEVSNAVANAKWLLSGTP
SEQ ID NO: 248: ATP14B sequence DNMT5 exemplar 11
PGLPPITTGP
SEQ ID NO: 249: ATP15 sequence DNMT5 exemplar 11
VILLDMDSETSAGSNLT
SEQ ID NO: 250: ATP15B sequence DNMT5 exemplar 11
ATHLIFASPYVHYDPEHQIRTVQQARGRCIRTGQ
SEQ ID NO: 251: MT1 sequence DNMT5 exemplar 12
LSVGTICSGTDAPIYAL
SEQ ID NO: 252: MT23 sequence DNMT5 exemplar 12
HHFSAEIEPAKQGFIRRNLDDIVIFRDVIEL
SEQ ID NO: 253: MT4 sequence DNMT5 exemplar 12
DILFAGCSCVDYSNMNH
SEQ ID NO: 254: MT5 sequence DNMT5 exemplar 12
SESAMTFFAAIKLITIIRP
SEQ ID NO: 255: MT6 sequence DNMT5 exemplar 12
FVILENVYSAPW
SEQ ID NO: 256: MT7 sequence DNMT5 exemplar 12
LDSKDYYLPQTRQRGYLVA
SEQ ID NO: 257: MT7B sequence DNMT5 exemplar 12
WALSSLRHADARHKNKLRRDDNPFSMKAMRNGKIVNAAYQSHSWMAF
SEQ ID NO: 258: MT8 sequence DNMT5 exemplar 12
QVARVIDLMDINFAV
SEQ ID NO: 259: MT8B sequence DNMT5 exemplar 12
DLGYKTYMIDASQNVDRNQLVTSGN
SEQ ID NO: 260: MT9 sequence DNMT5 exemplar 12
GIVGCITPSGLPV
SEQ ID NO: 261: MT10 sequence DNMT5 exemplar 12
EALALQGLPVDELVLSTETQSQLRDLAGNAMSLPVVGAATLALL
SEQ ID NO: 262: ATP11B sequence DNMT5 exemplar 12
LRPSQKLAVDWMLLRERASLRFAECEIEEEVVAPLGIRVMGKAEW
SEQ ID NO: 263: ATP12 sequence DNMT5 exemplar 12
RGGVVAHEIGYGKTVVSLALI
SEQ ID NO: 264: ATP13 sequence DNMT5 exemplar 12
SATLIIVPKHISQQWAAEAEKF
SEQ ID NO: 265: ATP14 sequence DNMT5 exemplar 12
FARIIWDEYTYDGGDLIHLEVSNAVANAKWLLSGTP
SEQ ID NO: 266: ATP14B sequence DNMT5 exemplar 12
PGLPPVTAGP
SEQ ID NO: 267: ATP15 sequence DNMT5 exemplar 12
ILLLDMDSETSAGSNLT
SEQ ID NO: 268: ATP15B sequence DNMT5 exemplar 12
ATHIIFANPYVHPDPEHQIRTVSQARGRCIRTGQ
SEQ ID NO: 269: MT1 sequence DNMT5 exemplar 13
LNVATLCSGTDAPIFAL
SEQ ID NO: 270: MT23 sequence DNMT5 exemplar 13
HLFSCEIEPFKQGFIRRNLPPGTLIFRDVVEL
SEQ ID NO: 271: MT4 sequence DNMT5 exemplar 13
DILFAGCSCVDYSNMNQ
SEQ ID NO: 272: MT5 sequence DNMT5 exemplar 13
GESARTFFAAIKLITVIRP
SEQ ID NO: 273: MT6 sequence DNMT5 exemplar 13
LVILENVYNAPW
SEQ ID NO: 274: MT7 sequence DNMT5 exemplar 13
LDSKDFYLPQTRQRGYLVA
SEQ ID NO: 275: MT7B sequence DNMT5 exemplar 13
WALCSLRHADVRQRHGIRRDDNPFSLKAMRNSRHIFAAYPSHSWMQF
SEQ ID NO: 276: MT8 sequence DNMT5 exemplar 13
QVARVADEMDIAFAA
SEQ ID NO: 277: MT8B sequence DNMT5 exemplar 13
DLGYKTCMIDVSQNVDRNDLVNGGR
SEQ ID NO: 278: MT9 sequence DNMT5 exemplar 13
GVVGCITPSGMPI
SEQ ID NO: 279: MT10 sequence DNMT5 exemplar 13
ETLALQGLPVDDLVVSTESQAQLRDLAGNAMTVTVVGAVTLAAL
SEQ ID NO: 280: ATP11B sequence DNMT5 exemplar 13
LRPSQREAVNWMLQRERVPLDFVKREVEEEVVAPLNLRVLGKAEW
SEQ ID NO: 281: ATP12 sequence DNMT5 exemplar 13
RGGVVAHEVGYGKTVVTLAVC
SEQ ID NO: 282: ATP13 sequence DNMT5 exemplar 13
SATLVVVPKHITAQWESEAAKF
SEQ ID NO: 283: ATP14 sequence DNMT5 exemplar 13
FARVVWDECSYDDDDNIRLEVAHAVANTKWLLSGTP
SEQ ID NO: 284: ATP14B sequence DNMT5 exemplar 13
PGLPAVTKGP
SEQ ID NO: 285: ATP15 sequence DNMT5 exemplar 13
ILLLDMDSETSAGSNLT
SEQ ID NO: 286: ATP15B sequence DNMT5 exemplar 13
ATRVIFASPYVHQDEEHQVRTVRQAKGRCIRTGQ
SEQ ID NO: 287: MT1 sequence DNMT5 exemplar 14
LNVATLCSGTDAPIFAL
SEQ ID NO: 288: MT23 sequence DNMT5 exemplar 14
HLFSCEIEPFKQGFIRRNLPPGTLIERDIVEL
SEQ ID NO: 289: MT4 sequence DNMT5 exemplar 14
DILFAGCSCVDYSAMNQ
SEQ ID NO: 290: MT5 sequence DNMT5 exemplar 14
GESARTFFAAIKLITVIRP
SEQ ID NO: 291: MT6 sequence DNMT5 exemplar 14
VVILENVYNAPW
SEQ ID NO: 292: MT7 sequence DNMT5 exemplar 14
LDSKDFYLPQTRQRGYLVA
SEQ ID NO: 293: MT7B sequence DNMT5 exemplar 14
WALCSLRHADVRQRHGIRRDDNPFSLKAMRNSRLIFAAYPSHSWMQF
SEQ ID NO: 294: MT8 sequence DNMT5 exemplar 14
QGARVVDFMDIAFSA
SEQ ID NO: 295: MT8B sequence DNMT5 exemplar 14
DLGYKTCMIDVSQNVDRNDLVTGGR
SEQ ID NO: 296: MT9 sequence DNMT5 exemplar 14
GLIGCITPSGMPI
SEQ ID NO: 297: MT10 sequence DNMT5 exemplar 14
ETLALQGLPVDDLVVSTESQAQLRDLAGNAMTVTVVGAVTLAAL
SEQ ID NO: 298: ATP11B sequence DNMT5 exemplar 14
LRPSQREAVNWMLQRERVPLDFVKREIEEEVVSPLNLRVLGKAEW
SEQ ID NO: 299: ATP12 sequence DNMT5 exemplar 14
RGGVVAHEVGYGKTVVTLAVC
SEQ ID NO: 300: ATP13 sequence DNMT5 exemplar 14
SATLVVVPRHITAQWESEAAKF
SEQ ID NO: 301: ATP14 sequence DNMT5 exemplar 14
FARIVWDECSYDDDDNIRLEVAHAVANAKWLLSGTP
SEQ ID NO: 302: ATP14B sequence DNMT5 exemplar 14
PGLPSVTKGP
SEQ ID NO: 303: ATP15 sequence DNMT5 exemplar 14
ILLLDMDSETSAGSNLT
SEQ ID NO: 304: ATP15B sequence DNMT5 exemplar 14
ATRVIFANPYVHQDEEHQARTVRQAKGRCIRTGQ
SEQ ID NO: 305: MT1 sequence DNMT5 exemplar 15
LNVATLCSGTDAPIFAL
SEQ ID NO: 306: MT23 sequence DNMT5 exemplar 15
HLFSCEIEPFKQGFIRRNLPPGTLIFRDVVEL
SEQ ID NO: 307: MT4 sequence DNMT5 exemplar 15
DILFAGCSCVDYSNMNQ
SEQ ID NO: 308: MT5 sequence DNMT5 exemplar 15
GESARTFFAAIKLITVIRP
SEQ ID NO: 309: MT6 sequence DNMT5 exemplar 15
LVILENVYNAPW
SEQ ID NO: 310: MT7 sequence DNMT5 exemplar 15
LDSKDFYLPQTRQRGYLVA
SEQ ID NO: 311: MT7B sequence DNMT5 exemplar 15
EWALCSLRHADVRQRHGIRGDDNPFSLKTMRNSRLIFAAYPSHSWMQF
SEQ ID NO: 312: MT8 sequence DNMT5 exemplar 15
QVARVVDFMDIAFAA
SEQ ID NO: 313: MT8B sequence DNMT5 exemplar 15
DLGYKTCMIDVSQNVDRNDLVNGGR
SEQ ID NO: 314: MT9 sequence DNMT5 exemplar 15
GVVGCITPSGMPI
SEQ ID NO: 315: MT10 sequence DNMT5 exemplar 15
ETLALQGLPVDDLVVSTESQAQLRDLAGNAMTVTVVGAVTLAAL
SEQ ID NO: 316: ATP11B sequence DNMT5 exemplar 15
LRPSQREAVKWMLQRERVPLDFVKREVEEEVVAPLNLRVLGKAEW
SEQ ID NO: 317: ATP12 sequence DNMT5 exemplar 15
RGGVVAHEVGYGKTVVTLAVC
SEQ ID NO: 318: ATP13 sequence DNMT5 exemplar 15
SATLVVVPKHITAQWESEAAKF
SEQ ID NO: 319: ATP14 sequence DNMT5 exemplar 15
FARVVWDECSYDDDDNIRLFVAHAVANAKWLLSGTP
SEQ ID NO: 320: ATP14B sequence DNMT5 exemplar 15
PGLPAVTKGP
SEQ ID NO: 321: ATP15 sequence DNMT5 exemplar 15
ILLLDMDSETSAGSNLT
SEQ ID NO: 322: ATP15B sequence DNMT5 exemplar 15
ATRVIFASPYVHQDEEHQVRTVRQAKGRCIRTGQ
SEQ ID NO: 323: MT1 sequence DNMT5 exemplar 16
LNVATLCSGTDAPIFAL
SEQ ID NO: 324: MT23 sequence DNMT5 exemplar 16
HLFSCEIEPFKQGFIRRNLPPDTLIFRDIVEL
SEQ ID NO: 325: MT4 sequence DNMT5 exemplar 16
DILFAGCSCVDYSNMNQ
SEQ ID NO: 326: MT5 sequence DNMT5 exemplar 16
GESARTFFAAIKLITIIRP
SEQ ID NO: 327: MT6 sequence DNMT5 exemplar 16
LVILENVYNAPW
SEQ ID NO: 328: MT7 sequence DNMT5 exemplar 16
LDSKDFYLPQTRQRGYLVA
SEQ ID NO: 329: MT7B sequence DNMT5 exemplar 16
WALCSLRHADVRQKHGIRRDDNPFSLKAMRNSRLVFAAYPSHSWMEF
SEQ ID NO: 330: MT8 sequence DNMT5 exemplar 16
QSARVVDFMDIAFSA
SEQ ID NO: 331: MT8B sequence DNMT5 exemplar 16
DLAYKTCMIDVSQNVDRNDLVNGGK
SEQ ID NO: 332: MT9 sequence DNMT5 exemplar 16
GLIGCITPSGLPI
SEQ ID NO: 333: MT10 sequence DNMT5 exemplar 16
ETLALQGLPVDDLVISTESQAQLRDLAGNAMTVTVVGAVTLAAL
SEQ ID NO: 334: ATP11B sequence DNMT5 exemplar 16
LRPSQKEAVNWMIQRERVPLEFVKREIEEEVVAPLNMRVLGKAEW
SEQ ID NO: 335: ATP12 sequence DNMT5 exemplar 16
RGGVVAHEVGYGKTVVTLALC
SEQ ID NO: 336: ATP13 sequence DNMT5 exemplar 16
SATLVVVPRHITAQWESEAAKF
SEQ ID NO: 337: ATP14 sequence DNMT5 exemplar 16
FARIVWDECSYDDDEKNVHLEVAHAVANAKWLLSGTP
SEQ ID NO: 338: ATP14B sequence DNMT5 exemplar 16
PGLPPVTKGP
SEQ ID NO: 339: ATP15 sequence DNMT5 exemplar 16
ILLLDMDEETSAGSNLT
SEQ ID NO: 340: ATP15B sequence DNMT5 exemplar 16
ATRVIFASPYVHQDEEHQVRTVRQAKGRCIRTGQ
SEQ ID NO: 341: MT1 sequence DNMT5 exemplar 17
LNVATLCSGTDAPIFAL
SEQ ID NO: 342: MT23 sequence DNMT5 exemplar 17
HLFSCEIEPFKQGFIRRNLPHGTLIFRDVVEL
SEQ ID NO: 343: MT4 sequence DNMT5 exemplar 17
DILFAGCSCVDYSNMNQ
SEQ ID NO: 344: MT5 sequence DNMT5 exemplar 17
GESARTFFAAIKLITALRP
SEQ ID NO: 345: MT6 sequence DNMT5 exemplar 17
LIILENVAGAPW
SEQ ID NO: 346: MT7 sequence DNMT5 exemplar 17
LDSKYFYLPQTRQRGYLVA
SEQ ID NO: 347: MT7B sequence DNMT5 exemplar 17
WSLCQLRHADTRRNKDLRRDDNPFSKKAMRNGRVI FASFPSHSWRPF
SEQ ID NO: 348: MT8 sequence DNMT5 exemplar 17
QVPRIVDLMDIVVAI
SEQ ID NO: 349: MT8B sequence DNMT5 exemplar 17
DLAFKTGMVDASQNVDRLPYVVGSG
SEQ ID NO: 350: MT9 sequence DNMT5 exemplar 17
GIVGCITPNGLPI
SEQ ID NO: 351: MT10 sequence DNMT5 exemplar 17
ETLALQGLPIDELVISTETQAQLRDLAGNAMSVTVVGAVTLALL
SEQ ID NO: 352: ATP11B sequence DNMT5 exemplar 17
LRASQKDAVNWMLQREQVPLDEVKSEIEEEVVAPLNVRIVGKAEW
SEQ ID NO: 353: ATP12 sequence DNMT5 exemplar 17
RGGVVAHEIGYGKTVVTLALL
SEQ ID NO: 354: ATP13 sequence DNMT5 exemplar 17
SATLVIVPKHITDQWAAEIVKF
SEQ ID NO: 355: ATP14 sequence DNMT5 exemplar 17
FARVVWDECSYDEGGLVPLFVANVVANAKWLLSGTP
SEQ ID NO: 356: ATP14B sequence DNMT5 exemplar 17
PGLPAVTKGP
SEQ ID NO: 357: ATP15 sequence DNMT5 exemplar 17
ILLLDMDSETSAGSNLT
SEQ ID NO: 358: ATP15B sequence DNMT5 exemplar 17
ATHVIFANPYVHNDEEHQARTIRQARGRCIRTGQ
SEQ ID NO: 359: MT1 sequence DNMT5 exemplar 18
LKVATLCSGTDAPIFAL
SEQ ID NO: 360: MT23 sequence DNMT5 exemplar 18
HLFSCEIEPEKQGFIRRNLGADTLIFRDVFEL
SEQ ID NO: 361: MT4 sequence DNMT5 exemplar 18
DILFAGCSCVDYSNMNM
SEQ ID NO: 362: MT5 sequence DNMT5 exemplar 18
GESARTFFSAIKLVSKLRP
SEQ ID NO: 363: MT6 sequence DNMT5 exemplar 18
TVMLENVLGAPW
SEQ ID NO: 364: MT7 sequence DNMT5 exemplar 18
LDTKHFYLPQTRKRGYLIA
SEQ ID NO: 365: MT7B sequence DNMT5 exemplar 18
WGESSLRHFDERRRHGLLLDENPFSLKAMRNGRLIYASYPSHSWKRL
SEQ ID NO: 366: MT8 sequence DNMT5 exemplar 18
LGARVVDSMDILFAA
SEQ ID NO: 367: MT8B sequence DNMT5 exemplar 18
DIGYKTMLADVSQNIDRSNKFLPVGG
SEQ ID NO: 368: MT9 sequence DNMT5 exemplar 18
GIIGCITPSGIPV
SEQ ID NO: 369: MT10 sequence DNMT5 exemplar 18
EALALQGLPADELVISTETQAQLRDLAGNAMTVTVVGAATLVLL
SEQ ID NO: 370: ATP11B sequence DNMT5 exemplar 18
LRPSQRDAVHWMMQRERSPVDFTKVEIEEEVVRPLNMRVMGKAEW
SEQ ID NO: 371: ATP12 sequence DNMT5 exemplar 18
RGGVVAHEIGYGKTVVMLALH
SEQ ID NO: 372: ATP13 sequence DNMT5 exemplar 18
SATLVVVPRHITKQWAKEAARF
SEQ ID NO: 373: ATP14 sequence DNMT5 exemplar 18
FARIIWDECSYDGNSTISLFVTNAIANSKWLISGTP
SEQ ID NO: 374: ATP14B sequence DNMT5 exemplar 18
PGLPSVIKGP
SEQ ID NO: 375: ATP15 sequence DNMT5 exemplar 18
VLLLDIESDTSAGSNLT
SEQ ID NO: 376: ATP15B sequence DNMT5 exemplar 18
ASHVIFANPFVHSDKEFQARTVRQARGRCIRLGQ
SEQ ID NO: 377: MT1 sequence DNMT5 exemplar 19
LRVVTMCSGTESPLLAL
SEQ ID NO: 378: MT23 sequence DNMT5 exemplar 19
HLFSAEIVPFKQAYIERNFHPRYLERDVEQL
SEQ ID NO: 379: MT4 sequence DNMT5 exemplar 19
DLVIAGFSCVDFSGLNN
SEQ ID NO: 380: MT5 sequence DNMT5 exemplar 19
GESGGTFWGIIRYAVAYRP
SEQ ID NO: 381: MT6 sequence DNMT5 exemplar 19
IVVLENIKNAPW
SEQ ID NO: 382: MT7 sequence DNMT5 exemplar 19
VDTKAYYLPQTRERGYMLC
SEQ ID NO: 383: MT7B sequence DNMT5 exemplar 19
WKKYQSRHQNYRSTQGLGFERPMSKFQDSGACQTPDFAWQTW
SEQ ID NO: 384: MT8 sequence DNMT5 exemplar 19
LPERIWDTIDVNFLR
SEQ ID NO: 385: MT8B sequence DNMT5 exemplar 19
DMNFKERCIELSQGVDREIDSR
SEQ ID NO: 386: MT9 sequence DNMT5 exemplar 19
GIIGCITPSGMPY
SEQ ID NO: 387: MT10 sequence DNMT5 exemplar 19
ESLALQGLPLDRLLLTRESSRDLQDLAGNAMSSTVVGAAVLSAL
SEQ ID NO: 388: ATP11B sequence DNMT5 exemplar 19
LRPEQLRSLSWMLEQEKDDIAPFVEEEVEEEILPSMMWRAEGKVVI
SEQ ID NO: 389: ATP12 sequence DNMT5 exemplar 19
RGGVLADDVGYGKTAITLGLI
SEQ ID NO: 390: ATP13 sequence DNMT5 exemplar 19
KATLVLVPQIVLEQWRLEIAKF
SEQ ID NO: 391: ATP14 sequence DNMT5 exemplar 19
FNRIVIDEFTYAGNERLAPLLALRARSKWILSGTP
SEQ ID NO: 392: ATP14B sequence DNMT5 exemplar 19
SQNTRLKV
SEQ ID NO: 393: ATP15 sequence DNMT5 exemplar 19
VVILNLGSETAAGLNLQ
SEQ ID NO: 394: ATP15B sequence DNMT5 exemplar 19
TNHVIFLSPMLAQTQYDYDSSMTQAIGRALRYGQ
SEQ ID NO: 395: MT1 sequence DNMT5 exemplar 20
MCSGTESPLLAL
SEQ ID NO: 396: MT23 sequence DNMT5 exemplar 20
HLFSAEIVPFKQAYIERNFHPPYIFRDVREL
SEQ ID NO: 397: MT4 sequence DNMT5 exemplar 20
DIVIAGFSCVDESALNN
SEQ ID NO: 398: MT5 sequence DNMT5 exemplar 20
GESGITFHAILQYIEKYRP
SEQ ID NO: 399: MT6 sequence DNMT5 exemplar 20
LAVLENVSHAPW
SEQ ID NO: 400: MT7 sequence DNMT5 exemplar 20
VDSKAYYLPQTRERGYAIC
SEQ ID NO: 401: MT7B sequence DNMT5 exemplar 20
WTRYQARHQSYRLNNKLGYKRPMTRYQDNGTCKMPDFVWHEW
SEQ ID NO: 402: MT8 sequence DNMT5 exemplar 20
QPERIWDTLDMNFLR
SEQ ID NO: 403: MT8B sequence DNMT5 exemplar 20
DLNFKERYLDLSQGIDREQDSR
SEQ ID NO: 404: MT9 sequence DNMT5 exemplar 20
GIMGCITPCGIPY
SEQ ID NO: 405: MT10 sequence DNMT5 exemplar 20
EALSLQGLPLDRLLLTKESQRELQDLAGNAMTSTVVGAATLSAL
SEQ ID NO: 406: ATP11B sequence DNMT5 exemplar 20
LRPEQLRSLRWMIQQEADDPMPFEQEEVEEALLPSMMWRAEAKVTA
SEQ ID NO: 407: ATP12 sequence DNMT5 exemplar 20
RGGVLADQVGYGKTALILGLI
SEQ ID NO: 408: ATP13 sequence DNMT5 exemplar 20
KATLIVVPRMMVKQWQSEITKE
SEQ ID NO: 409: ATP14 sequence DNMT5 exemplar 20
FSRLVVDEFTYVDQRKLGPVQKLKSRSVWVLSGTP
SEQ ID NO: 410: ATP14B sequence DNMT5 exemplar 20
MSSARLAS
SEQ ID NO: 411: ATP15 sequence DNMT5 exemplar 20
VLILNMGTESAAGINLQ
SEQ ID NO: 412: ATP15B sequence DNMT5 exemplar 20
ANHVIFLHPFLAKTQYDYESTMTQAIGRSRRFGQ
SEQ ID NO: 413: MT1 sequence DNMT5 exemplar 21
LRVVTMCSGTESPLLAM
SEQ ID NO: 414: MT23 sequence DNMT5 exemplar 21
HLFSSEIDPFKQAYIERNFQPPVLERDVKQL
SEQ ID NO: 415: MT4 sequence DNMT5 exemplar 21
DILIAGFSCVDFSVLNN
SEQ ID NO: 416: MT5 sequence DNMT5 exemplar 21
GESGQTFFAIIGYAKLHRP
SEQ ID NO: 417: MT6 sequence DNMT5 exemplar 21
LVVLENVKNAPW
SEQ ID NO: 418: MT7 sequence DNMT5 exemplar 21
VDTKHFYLPQTRERGYMVC
SEQ ID NO: 419: MT7B sequence DNMT5 exemplar 21
WLRYKIRHQDYRREKGLGHKRPLTNYQDGGTCRPPSWTDRHW
SEQ ID NO: 420: MT8 sequence DNMT5 exemplar 21
QVERVWDTIDMNFLR
SEQ ID NO: 421: MT8B sequence DNMT5 exemplar 21
DINYKLRIIDLSQGIDREIDGR
SEQ ID NO: 422: MT9 sequence DNMT5 exemplar 21
GVAGCITPCGMQY
SEQ ID NO: 423: MT10 sequence DNMT5 exemplar 21
ESLALQGLPLDRLTLTRESSRELQDLAGNAMSSTVVGSAILAAL
SEQ ID NO: 424: ATP11B sequence DNMT5 exemplar 21
LRPEQLRSLHWIRACESDDAPPFREEEVEEAVLGPLEWRVEAKVMB
SEQ ID NO: 425: ATP12 sequence DNMT5 exemplar 21
RGGVLADVVGFGKTALVLGTI
SEQ ID NO: 426: ATP13 sequence DNMT5 exemplar 21
KATLIVVPDILFDQWQSEIKKE
SEQ ID NO: 427: ATP14 sequence DNMT5 exemplar 21
FQRVIIDEFTYLETDKHALLSTIKARSRWLLSGTP
SEQ ID NO: 428: skipped
SEQ ID NO: 429: ATP15 sequence DNMT5 exemplar 21
VLILMLGSENAAGINLQ
SEQ ID NO: 430: ATP15B sequence DNMT5 exemplar 21
ANHVIFLAEMCAATAYDHESSMTQAIGRALRYGQ
SEQ ID NO: 431: MT1 sequence from Cryptococcus neoformans
LRVATMCSGTESPLLAL
SEQ ID NO: 432: MT23 sequence from Cryptococcus neoformans
HVFSCEIEPFKQAYIERNFTPPILERDVTEL
SEQ ID NO: 433: MT4 sequence from Cryptococcus neoformans
DILIAGTSCVDYSNLNN
SEQ ID NO: 434: MT5 sequence from Cryptococcus neoformans
GESGRTFRGMLQWVKKHQP
SEQ ID NO: 435: MT6 sequence from Cryptococcus neoformans
IVILENVCNAPW
SEQ ID NO: 436: MT7 sequence from Cryptococcus neoformans
LDTKEFYIPHTRTRVYLFA
SEQ ID NO: 437: MT7B sequence from Cryptococcus neoformans
WNRCESRHQRARQDEALGLLRPLTSWQEAGVCKGLDWTWNDW
SEQ ID NO: 438: MT8 sequence from Cryptococcus neoformans
QTERVVDLLEISTLR
SEQ ID NO: 439: MT8B sequence from Cryptococcus neoformans
DSGFKACIWNVSQNVDRQTGS
SEQ ID NO: 440: MT9 sequence from Cryptococcus neoformans
ALAPCLTPNMIPW
SEQ ID NO: 441: MT10 sequence from Cryptococcus neoformans
EALALQGIPVRELLLTSENEDQLADLAGNAMTTTVVGSAMIAAL
SEQ ID NO: 442: ATP11B sequence from Cryptococcus neoformans
LRKEQLRSLWWMLEQEKATGKTHTFVEEEISESLLPAVGWRAEGKAER
SEQ ID NO: 443: ATP12 sequence from Cryptococcus neoformans
RGGVIADQVGYGKTVISIALV
SEQ ID NO: 444: ATP13 sequence from Cryptococcus neoformans
KATLIVVPGHLSKQWPNEIARF
SEQ ID NO: 445: ATP14 sequence from Cryptococcus neoformans
FRRVIADEFTYLQKKSLAAVLRLSSSYRWILSGTP
SEQ ID NO: 446: ATP14B sequence from Cryptococcus neoformans
DVQYQKA
SEQ ID NO: 447: ATP15 sequence from Cryptococcus neoformans
VLLLKMNDASAAGSNLT
SEQ ID NO: 448: ATP15B sequence from Cryptococcus neoformans
ANHAVFLGPLFTNSLENYRAVETQAIGRVRRYGQ
SEQ ID NO: 449: MT1 sequence from Cryptococcus tepidarius
LRVATMCSGTESPLLAL
SEQ ID NO: 450: MT23 sequence from Cryptococcus tepidarius
HVFSCEIEPFKQAYIERNFAPPILERDVTEL
SEQ ID NO: 451: MT4 sequence from Cryptococcus tepidarius
DILIAGTSCVDYSGLNN
SEQ ID NO: 452: MT5 sequence from Cryptococcus tepidarius
GESGRTFRGMLQWVNKHRP
SEQ ID NO: 453: MT6 sequence from Cryptococcus tepidarius
IVILENVCSAPW
SEQ ID NO: 454: MT7 sequence from Cryptococcus tepidarius
LDTKEYYIPHTRTRVYLFA
SEQ ID NO: 455: MT7B sequence from Cryptococcus tepidarius
WNRCESRHARARQEELLGVLRPLTSWKEAGVCKGLDWTWNDW
SEQ ID NO: 456: MT8 sequence from Cryptococcus tepidarius
QTERVVDLLDISTLR
SEQ ID NO: 457: MT8B sequence from Cryptococcus tepidarius
DSGYKACIWNVSQNVDRQGTG
SEQ ID NO: 458: MT9 sequence from Cryptococcus tepidarius
ALAPCLTPNMIPW
SEQ ID NO: 459: MT10 sequence from Cryptococcus tepidarius
EALSLQGIPVRELLLTSENEDQLADLAGNAMTSTVVGASMLSAL
SEQ ID NO: 460: ATP11B sequence from Cryptococcus tepidarius
LRKEQLRSLWWMLRQEAAEGKTHTFVEEEISEAMLPSLGWRAEGKAER
SEQ ID NO: 461: ATP12 sequence from Cryptococcus tepidarius
RGGVIADQVGYGKTIISLALV
SEQ ID NO: 462: ATP13 sequence from Cryptococcus tepidarius
KATLIVVPGHLSKQWPSEITRF
SEQ ID NO: 463: ATP14 sequence from Cryptococcus tepidarius
FRRVIADEFTYLEKKPLNALLKLSSCYKWVLSGTP
SEQ ID NO: 464: ATP14B sequence from Cryptococcus tepidarius
SKSTQIA
SEQ ID NO: 465: ATP15 sequence from Cryptococcus tepidarius
VLLLKMQDASAAGSNLT
SEQ ID NO: 466: ATP15B sequence from Cryptococcus tepidarius
ANHAIFLGPLFVDTLHRYRQAETQAIGRIRRYGQ

### EXAMPLES

The invention is now described with reference to the Examples below. These are not limiting on the scope of the invention, and a person skilled in the art would be appreciate that suitable equivalents could be used within the scope of the present invention. Thus, the Examples may be considered component parts of the invention, and the individual aspects described therein may be considered as disclosed independently, or in any combination.

### Example 1. Activity of Dnmt5 from Takashimella Tepidaria/Cryptococcus Tepidarius and Cryptococcus Neoformans

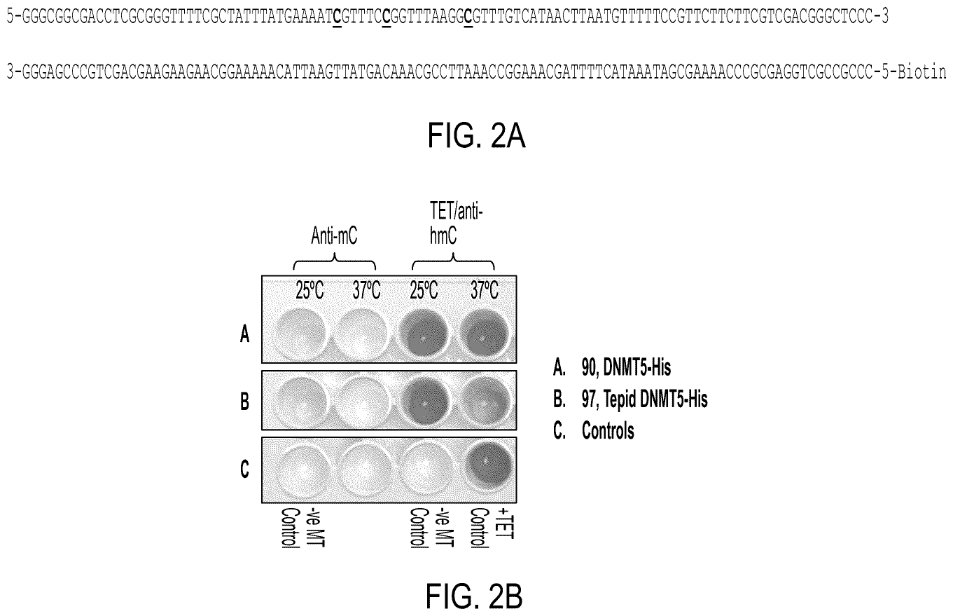

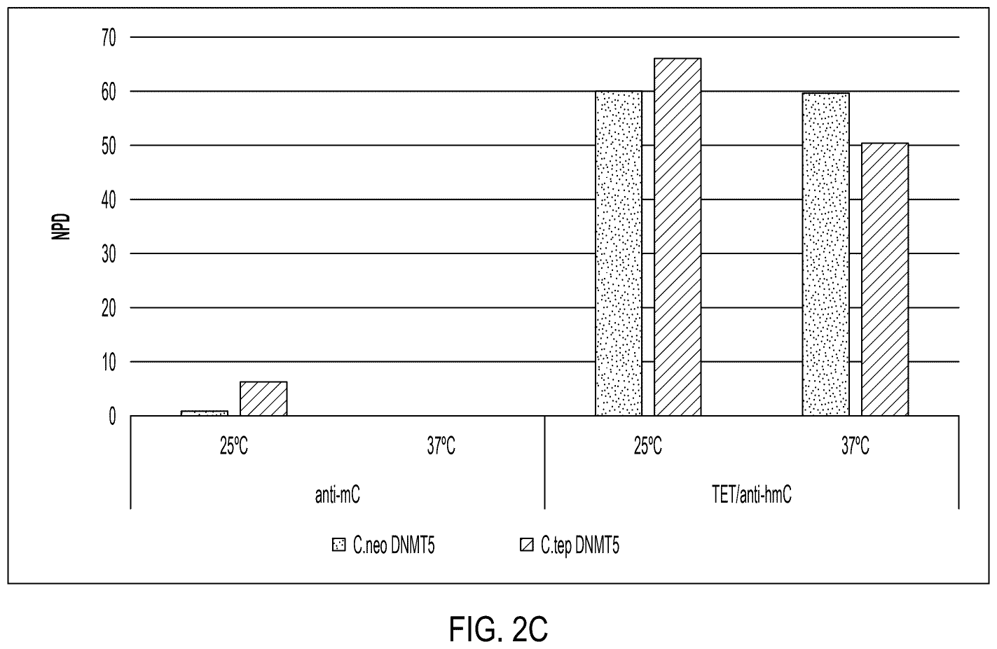

Different variants of DNMT5 were tested for methyltransferase activity as described in the following outlined scheme. DNMT5 proteins from Takashimella tepidaria/Cryptococcus tepidarius and Cryptococcus neoformans were incubated with a hemimethylated DNA substrate and an ELISA was used to determine whether the enzyme was able to copy the methyl group from the methylated strand to the unmethylated strand. Because the detection of methyl cytosines in DNA is weak when using 5 mC-specific antibodies, after the methyltransferase step the presence of the methyl cytosine was determined by first incubating the reaction product with a ten-eleven translocation methylcytosine dioxygenase (TET) enzyme that oxidises methylcytosine to hydroxymethylcytosine (hmC). This then allowed hmC to be detected with more specific anti-hmC reactive antibodies, resulting in a stronger ELISA signal (FIG. 2A). As controls, reactions without the methyltransferase (no MT), or with a substrate that was symmetrically-methylated (positive control) were used. Pixel density was determined and normalised against a well containing PBS-buffer only, and a graph plotted.

The hemimethylated substrate DNA comprised two DNA strands that were hybridised together—a first DNA strand (SEQ ID NO:12) that comprised a sequence with three methylated cytosine (mC) bases, and was not biotinylated—and a second DNA strand (SEQ ID NO: 13) that was the unmethylated reverse complement of the first and was labelled at its 5′ end with biotin. Thus, the action of the methyltransferase would copy the methylated cytosine residues from the unbiotinylated strand to the biotinylated strand, and the application of 0.1M NaOH would separate the two strands leaving only the biotinylated strand, together with any methyl cytosines copied from the first unbiotinylated strand, bound to the streptavidin plate.

A hemimethylated dsDNA substrate was generated by combining two oligos (SEQ ID: 12 and SEQ ID: 13; purchased from Biomers.net GmbH, Germany) in an equimolar ratio in PBS. 2 microL of SEQ 12: at 100 microM was combined with 2 microL SEQ ID: 13 at 100 microM and 10 microL 2×PBS, to a final volume 20 microL with the balancing volume made up with deionised water. This mixture was heated to 80° C. in a PCR machine, and then slowly cooled (1° C. per see) to 25° C., to generate the double stranded hemimethylated substrate at 10 microM, 675 ng/microL.

The DNMT5 assay conditions were as follows: 50 mM Tris-HCl PH 8, 25 mM NaCl, 10% Glycerol, 2 mM DTT, 1 mM ATP, 800 microM SAM, 1 mM MgCl2, 200 ng DNA substrate, 80 fmoles of DNMT5 enzyme in a final volume of 50 microL Reactions were incubated in parallel for 2.5 hours at 25° C. and 37° C. Following the reaction, the DNA was purified using the DNA Clean & Concentrator-5 kit (Zymo research, D4004) following manufacturer's instructions and eluted into 10 microL of water and quantified using a Qubit fluorometer. The resulting DNA was then treated with TET enzyme as follows: 100 ng of DNA eluted from the Zymo purification column was added to a TET reaction mix (50 mM HEPES, pH 6.5, 100 mM NaCl, 1 mM alpha-ketoglutarate, 1 mM DTT, 2 mM sodium ascorbate, 75 microM ammonium iron (II) sulfate, 1 microL of TET2 enzyme (NEB EM-seq kit catalogue E7120) to a final volume of 20 microL in water. The reaction was incubated at 37° C. for one hour. A symmetrically-methylated DNA substrate was used as a positive control, and a reaction without the DNMT5 from Cryptococcus neoformans was used as a no-methyltransferase control. The symmetrically-methylated control substrate was generated by combining two oligos (SEQ ID 12 and SEQ ID 17, purchased from Biomers.net GmbH, Germany) in an equimolar ratio in PBS as follows: 2 microL of SEQ ID 12 at 100 microM was combined with 2 microL SEQ ID 17 at 100 microM and 10 microL 2×PBS, to a final volume 20 microL in water). This was heated to 80° C. in a PCR machine then slowly cooled (1° C. per see) to 25° C., to generate the double stranded symmetrically-methylated dsDNA substrate at 10 microM, 675 ng/microL. This was diluted in water to a final concentration of 100 ng/microL for use in the assay.

The TET reaction was applied to a 96 well white Nunc MaxiSorp microtitre plate (Thermofisher catalogue no. 236105) pre-coated in 100 microL of 100 microg/ml streptavidin (NEB catalogue no. N7021) and blocked in 0.2% BSA in PBS. The wells were washed three times with 100 microL 0.1M NaOH to denature and remove the non-biotinylated strand, then three times with 200 microL PBS prior to the addition of the primary antibody. 5hmC was detected using 50 microL per well of a rabbit anti-hmC monoclonal antibody (clone RM236, Abcam catalogue no AB21428) diluted 1:5000 in PBS+0.1% BSA, and incubated for one hour at room temperature before washing three times with 200 microL PBS with 0.1% Tween20 and three times with 200 microL PBS. Subsequently, 50 microL per well of HRP-labelled anti-rabbit secondary antibody (Invitrogen, catalogue no 31460) diluted to 1:5000 in PBS+0.1% BSA was added to each well and incubated for one hour at room temperature, then washed with three times with 200 microL PBS with 0.1% Tween20 and three times with 200 microL PBS. The binding of the antibody complex was detected with a TMB-substrate (Life Technologies, catalogue no 002023) and was quenched with H₂SO₄ after approximately five minutes.

The microtitre plate was imaged (FIG. 2B) and the relative intensity of each well was calculated from the pixel density of the image using ImageJ. The data was normalised by deducting the pixel density from the methyltransferase-negative control from the pixel density of the experimental wells (NPD, normalised pixel density). This was used to plot a histogram to compare the reactivities of the Takashimella tepidaria/Cryptococcus tepidarius DNMT5 and DNMT5 from Cryptococcus neoformans at both 25° C. and 37° C. (FIG. 2C).

### Results

Both DNMT5 proteins from Takashimella tepidaria/Cryptococcus tepidarius and Cryptococcus neoformans were active methyltransferase enzymes at both temperatures tested. The control sample that was symmetrically-methylated showed the strongest signal as expected, and no activity was observed for the dsDNA control lacking the Cryptococcus neoformans methyltransferase. These data demonstrated that the DNMT5 proteins are methyltransferases and copied 5 mC from the unbiotinylated substrate strand to the biotinylated strand. Furthermore, the 5 mC groups could be further oxidised to hmC by TET enzymes, and the reaction detected by anti-hmC antibodies in an ELISA.

### Example 2. Protection of an Ascl Recognition Site in a Hemimethylated DNA Template by the Activity of DNMT5

The activity of DNMT5 on hemimethylated DNA was assessed by the methyl-sensitive restriction endonuclease Ascl. The restriction endonuclease Ascl cleaves dsDNA containing the recognition sequence 5′-GG|CGCGCC-3′ (where the | mark indicates the site of cleavage on dsDNA). Ascl is able to cleave this recognition sequence when the DNA is unmethylated, and also cuts hemimethylated DNA when the first cytosine in the recognition sequence is methylated (5′-GG|mCGCGCC-3′; mC depicts methylated cytosine). Ascl was also known to be inhibited when the first cytosine in the recognition sequence was symmetrically-methylated on both strands of the duplex. Treating a dsDNA sequence that contained a hemimethylated

Ascl site with a DNMT5 enzyme before restriction digestion would test whether DNMT5 was able to protect the restriction site from cleavage, provided that the first methyl cytosine in the recognition sequence, and its reverse complement, were methylated and inhibited the enzyme.

An assay was developed to distinguish the methylation state of an Ascl restriction site in hemimethylated DNA from an Ascl site in which both strands of the recognition site were methylated. For clarity, the data from one exemplary assay is illustrated schematically in FIG. 3A, and the corresponding experimental data shown in FIG. 3B.

FIG. 3A depicts a hemimethylated dsDNA substrate of 195 bp that is capable of digestion by Ascl, and generates two bands of 135 bp and 60 bp. By contrast, treatment of this DNA with DNMT5 prior to Ascl digestion protects the DNA from cleavage, resulting in a full-length dsDNA product.

FIG. 3B shows experimental data from an Ascl assay corresponding to FIG. 3A. The following methods were used:

1. Generating dsDNA that Contains a Hemimethylated Ascl Site

Plasmid DNA containing DNA sequence 1 (SEQ ID NO: 5) that contained a multiple cloning site containing EcoRI (italics) and Ascl (underlined) recognition sequences was amplified with DNA primers P257 and P258 (SEQ ID NOs: 6 and 7) in a thermocycler using Q5® High-Fidelity DNA Polymerase (NEB M0491). The PCR composition was 1× Q5® High-Fidelity Buffer (NEB), 0.2 mM dNTPs, 0.5 microM of each primer P257 and P258, 0.02 ng/microL DNA template, 0.02 U/microL Q5 polymerase, in a final volume of 50 microL. PCR conditions were 98° C. 30s, 26 cycles [98° C. 10s, 70° C. 30s, 72° C. 20s], 72° C. 1 min. The DNA was purified using the DNA Clean & Concentrator-5 kit (Zymo research, D4004) following manufacturer's instructions. DNA was quantified (115 ng/microL for ˜120 microL total volume).

2. Treatment of Hemimethylated DNA with DNMT5

DNMT5 at an approximate concentration of 8pmol/uL was incubated with 800 microM SAM (S-adenosylmethionine, NEB #B9003S), 1.25 mM ATP, 1× final concentration of 10× Jena labelling buffer (PCR-201S), 49fmol/microL of hemimethylated DNA from step 1, in a final volume of 80 microL. The reactions were incubated for 1 h at 25° C. and then stored at 8° C. until usage. For both negative control samples and samples treated with DNMT5, 12 microL were purified using DNA Clean & Concentrator-5 (Zymo #D4004) following manufacturer instructions, and quantified.

3. Digestion of dsDNA with Ascl

Between 15 to 30 ng of DNA sample was digested using 2U of Ascl (NEB #R0558S) in 1× Cutsmart buffer (NEB) with a final volume of 10 microL. The digestion was performed at 37° C. for 1h. 2 μl of DNA Loading Dye was added to the reaction mixture, which was then loaded on a 4% agarose gel stained with SYBR Safe and run for 30 minutes at 100V.

### Results

The results of a typical Ascl assay following these methods are shown in FIG. 3B. In the first sample (lane 1) the DNA substrate had not been treated with either enzyme, and therefore migrated at its full length of 195 bp. In the second sample (lane 2) the hemimethylated DNA was cut by Ascl, and therefore migrated as two bands of 135 bp and 60 bp. In the third sample (lane 3), the DNA was treated with DNMT5 but migrated as a full-length product because it had not treated with Ascl. In the fourth sample (lane 4), the DNA substrate had been exposed to DNMT5 prior to Ascl digestion, and migrated as a full-length product. These data indicated that the DNMT5 protected the hemimethylated dsDNA substrate from digestion by Ascl because the Ascl site was methylated on both strands.

### Example 3. Copying Methyl Cytosine Through Two H-E-M Cycles

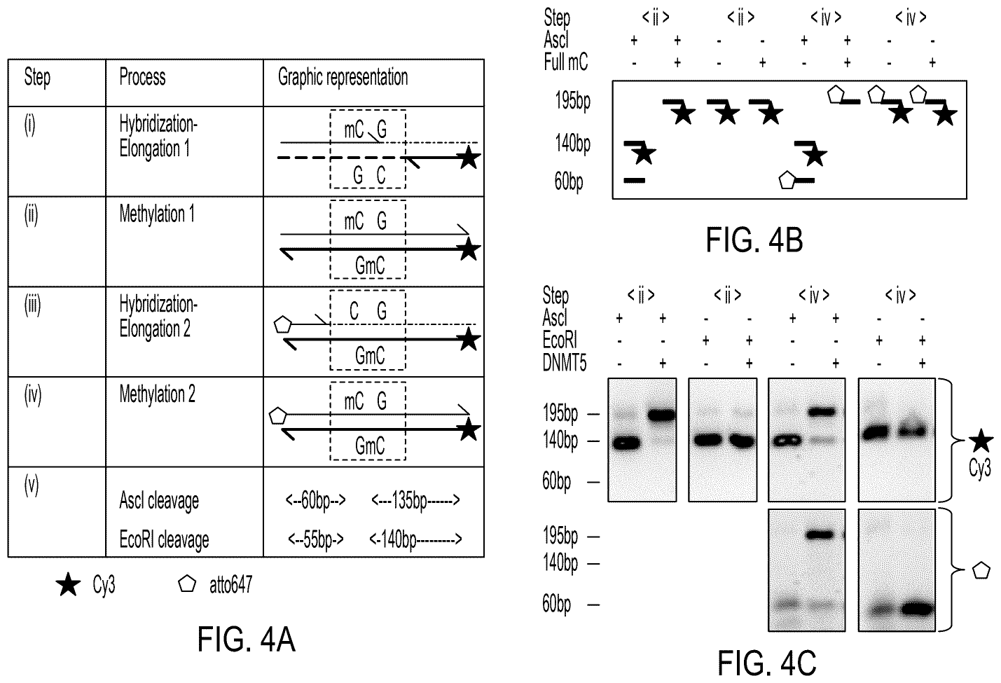

An experiment was designed to test whether a methyl cytosine base in a hemimethylated DNA substrate could be copied to its complementary strand, and then whether the copied methyl cytosine could subsequently act as a substrate for a second copying reaction into a newly synthesised DNA strand. In this experiment it was necessary to distinguish the original and copied DNA strands using DNA primers that had different fluorophore labels. For clarity, a schematic representation of the experiment is illustrated in FIG. 4A, and the expected results and actual experimental data are shown in FIGS. 4B and 4C respectively.

FIG. 4A depicts a four-step experiment to demonstrate the activity of DNMT5 in repeated H-E-M cycles, and is broken down as follows: (i) synthesis of a hemimethylated DNA substrate that contained a methylated cytosine group (top) and a fluorescently-labelled complementary unmethylated strand (bottom); (ii) copying of the methyl cytosine group (top strand) to the unmethylated (bottom strand) in the hemimethylated complex using DNMT5; (iii) synthesis of a new fluorescently-labelled (top) DNA strand that used the originally unmethylated DNA (bottom strand) as template; (iv) copying of the methyl cytosine group to the newly synthesised top strand of DNA using DNMT5. In (v) a representation is depicted of the expected banding pattern when DNA was digested with either Ascl or EcoRI restriction enzymes. A star symbol represents a Cy3 fluorophore that labelled the unmethylated DNA strand in the original hemimethylated complex, and a pentagon symbol represents an Atto647 fluorophore that labelled the new DNA strand that was synthesised using the Cy3 labelled DNA as template. The dotted box represents the Ascl recognition site (5′GG|CGCGCC3′), and for clarity, the letters within the box represent the first CG motif of the Ascl site on the top DNA strand, and its reverse complement on the bottom strand. mC depicts methyl cytosine.

FIG. 4B depicts the expected results from the four-step process illustrated in FIG. 4A that starts in (i) with a DNA substrate of the same sequence as the one used in Example 2. As before, Ascl digested both unmethylated and hemimethylated DNA products to form 2 fragments of 60 bp and 135 bp, but was inhibited when the Ascl recognition site was methylated on both strands, resulting in no digestion of the 195 bp DNA substrate. By contrast, EcoRI cut all DNA substrates (to two fragments of 140 bp and 55 bp) regardless of the methylation status of the DNA, and served as a control. Fluorophore labelling allowed each strand to be monitored independently for restriction digestion.

FIG. 4C shows experimental data corresponding to the schematic illustrations in FIGS. 4A and 4B. The following methods were used:

(i) Generation of dsDNA Containing a Hemimethylated Ascl Site in which One Strand is Labelled with Cy3.

A two-step method was used to generate a hemimethylated Ascl site in dsDNA in which the unmethylated strand was labelled with a Cy3 fluorescent dye. In the first step the method of Example 2 step (1) was used with no modification. The PCR product produced was quantified (14 ng/microL in ˜30 microL).

In the second step, the product from the first amplification was diluted to approximately 1 ng/microL and further amplified through a single cycle of DNA denaturation and DNA synthesis with Phusion® High-Fidelity DNA Polymerase (NEB M0530S) using P257 primer (Seq ID No:6) and T7rev-Cy3 (SEQ ID NO: 10). The composition of this reaction was as follows: 1× Buffer, 0.2 mM dNTPs, 0.5 microM of each primer, 1 ng/microL DNA template, 0.02 U/microL Phusion polymerase in a final volume of 8×50 microL. The amplification conditions were 98° C. 30s, 64° C. 30s, 72° C. 1 min. The PCR products from this second step were purified and quantified (80 ng/microL for ˜12 microL total volume).

(ii) Treating the Hemimethylated Ascl Site with DNMT5

The DNA from the previous step was treated with DNMT5 according to the method in Example 2 step (2). In addition, a negative control that lacked DNMT5 was set up in an identical manner. Reactions were incubated for 1 h at 25° C. and then stored at 8° C. until usage.

Prior to restriction digestion (step (v) below) samples were purified using DNA Clean & Concentrator-5 (Zymo #D4004) following manufacturer instructions.

(iii) Synthesis of a New DNA Strand that Uses the Originally Unmethylated DNA as Template.

The primer T7 for-Atto647 primer (SEQ ID NO: 9, final concentration of 1 microM) was added to unpurified samples from the previous step (ii, above) and a new DNA strand synthesised in a single cycle of PCR with the following conditions: 95° C. 40s, 64° C. 1 min, 68° C. 3 min, and then stored at 4° C. In this reaction, the use of the T7 for-Atto647 primer (SEQ ID NO: 9) allowed the new unmethylated DNA to be distinguished from the template DNA which was labelled in step (ii) with a Cy3 dye.

(iv) Copying the DNA Methylation from the Originally Unmethylated DNA Template into the Newly Synthesised DNA Strand.

10.2 uL of unpurified DNA from the previous step (iii, above) was incubated with DNMT5 under the same conditions as for the previous DNMT5 treatment of step (ii), in a reaction with a final volume of 20 μL. The reactions were incubated for 1 h at 25° C. and then stored at 8° C. until usage. The samples were purified as previously and quantified.

(v) Treating the DNA Samples from Steps (ii) and (iv) with Ascl and/or EcoRI.

Between 15 and 30 ng of DNA sample was digested using 2U of Ascl (NEB #R0558S) and/or EcoRI-hf (NEB #R3101S) in 1× Cutsmart buffer (NEB) with a final volume of 10 microL. The digestion was performed at 37° C. for 1h. 10 microL of each sample was mixed with 2 microL of 30% glycerol without any dye. The samples were run on a 4% agarose gel and run at 90V for 1 h. The expected results are shown in FIG. 4B.

### Results

At the end of the first round of Hybridization-Extension-Methylation (H-E-M), the sample from step (ii), treated with DNMT5 and digested with EcoRI, generated a 140 bp Cy3 labelled band (FIG. 4C). This demonstrated that the polymerase extension reaction had been successful and generated a dsDNA, because the EcoRI site was not present on the Cy3 primer. When subjected to Ascl digestion, this same sample generated a major band at 195 bp, indicating that it was protected from restriction digestion. As a control, the equivalent sample from step (ii) that had not treated with DNMT5 was unprotected from cleavage by Ascl and produced a major band at 135 bp. These data demonstrated that treatment with DNMT5 in this first round of H-E-M did protect the substrate from digestion by Ascl through the copying of the methylation pattern to the newly synthesised strand. A similar result was generated in step (iv). The sample treated with DNMT5 was cleaved by EcoRI, indicating the second elongation step had been successful and generated dsDNA labelled with Atto647. This same sample was very poorly cleaved by Ascl, while the DNMT5 negative sample was fully digested by Ascl.

In summary, in step (ii) DNMT5 protected the originally unmethylated (bottom) DNA strand (labelled with Cy3) from digestion. In (iv) neither the Cy3 strand, nor the newly synthesised Atto647-labelled strand complementary to the Cy3-labelled strand were digested by Ascl when the DNA was treated with DNMT5. These data demonstrated that a methylation mark had been copied from a template strand to a newly synthesised strand using DNMT5, and then copied further from the first synthesised strand to another new synthesised strand, in a second round of amplification.

### Example 4. Two Cycles of H-E-M PCR Starting with a Methylated dsDNA Template

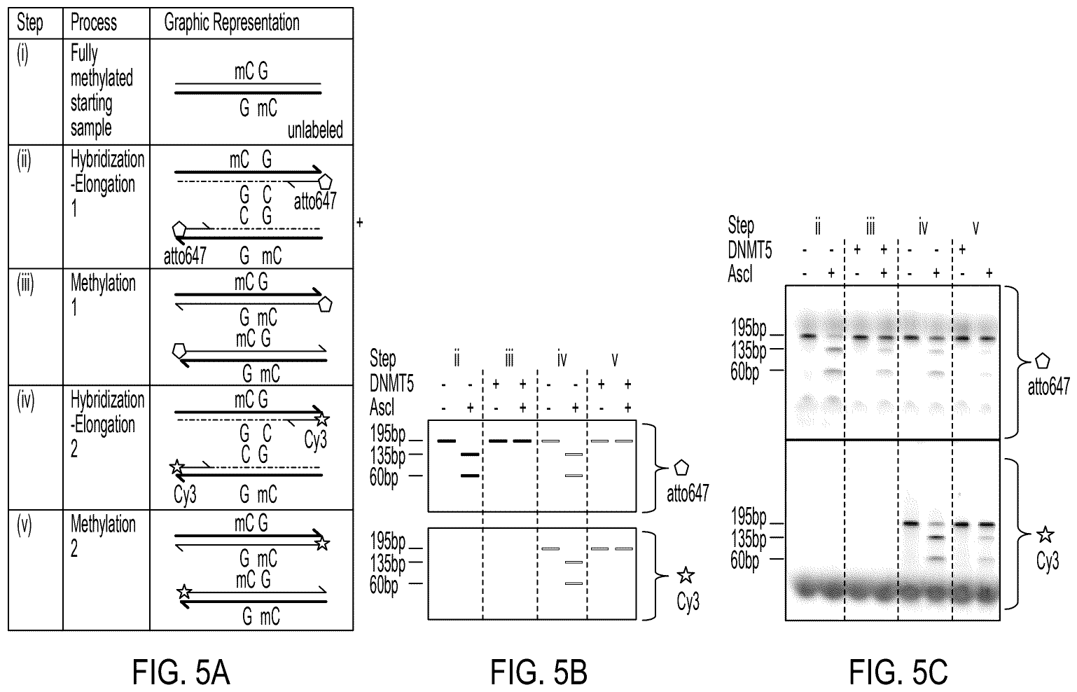

An experiment was designed to test whether a typical format for a PCR reaction, in which the substrate DNA is double stranded, and where both strands of DNA are amplified simultaneously, was compatible with the cycles of H-E-M using DNMT5, and without the need for purification of the DNA between cycles. Given that the DNMT5 was expected to be denatured at 94° C., it was not obvious that cycles of H-E-M would work in the absence of clean-up steps. Like in Example 3, in this experiment it was also necessary to distinguish the original and copied DNA strands using DNA primers that had different fluorophore labels. In this case, both copies of the forward and reverse strands were labelled with one fluorophore in the first cycle, and with a different fluorophore in the second cycle. For clarity, a schematic representation of the experiment is illustrated in FIG. 5A, and the expected results and actual experimental data are shown in FIGS. 5B and 5C respectively.

FIG. 5A depicts a four-step experiment to demonstrate the activity of DNMT5 on a dsDNA template in repeated H-E-M PCR cycles, and is broken down as follows: (i) synthesis of dsDNA substrate that contained an Ascl site with methylated cytosine groups on both strands; (ii) hybridisation of Atto647-labelled primers T7 for-Atto647 (SEQ ID NO:9) and T7rev-Atto647 (SEQ ID NO:11) and extension to generate labelled complementary copies of both original strands of DNA; (iii) methylation of the newly-synthesised hemimethylated DNA by DNMT5 to generate symmetrically-methylated dsDNA; (iv) hybridisation of Cy3-labelled primers T7 for-Cy3 (SEQ ID NO:8) and T7rev-Cy3 (SEQ ID NO:10) and extension to amplify the DNA from step (iii); and a second methylation reaction on the hemimethylated DNA from step (iii) to produce symmetrically-methylated dsDNA copies for a second time on the same sample.

FIG. 5B depicts the expected results from the four-step process illustrated in FIG. 5A in which the dsDNA in step (i) is a DNA substrate of the same sequence as the one used in Example 2. As before, Ascl digested both unmethylated and hemimethylated DNA products to form 2 fragments of 60 bp and 135 bp, but was inhibited when the Ascl recognition site was methylated on both strands, resulting in no digestion of the 195 bp DNA substrate. Fluorophore labelling allowed the two H-E-M cycles to be monitored independently for restriction digestion.

FIG. 5C shows experimental data corresponding to the schematic illustrations in FIGS. 5A and 5B. The following methods were used:

(i) Generation of dsDNA in which the Ascl Site is Methylated on Both Strands

A plasmid DNA containing DNA sequence 1 (SEQ ID NO: 5) that contains a multiple cloning site containing EcoRI (italics) and Ascl (underlined) was amplified with DNA primers P257 and P258 (SEQ ID NOs: 6 and 7) in a thermocycler using Q5® High-Fidelity DNA Polymerase (NEB M0491). The PCR composition was 1× Q5® High-Fidelity Buffer (NEB), 0.2 mM dNTPs, 0.5 microM of each primer P257 and P258, 0.02 ng/microL DNA template, 0.02 U/microL Q5 polymerase, in a final volume of 50 microL. PCR conditions were 98° C. 30s, 26 cycles [98° C. 10s, 70° C. 30s, 72° C. 20s], 72° C. 1 min. The DNA was purified using the DNA Clean & Concentrator-5 kit (Zymo research, D4004) following manufacturer's instructions.

The purified hemimethylated DNA (323fmol/microL) was mixed with DNMT5 at an approximate concentration of 75fmol/microL and was incubated with 800 microM SAM, 1.25 mM ATP, 1× final concentration of 10× Cutsmart buffer (NEB) in a final volume of 25 microL. The reaction was incubated for 1 h at 25° C. and then stored at 8° C. until usage. The dsDNA was purified as previously and quantified.

(ii) Synthesis of Complementary DNA to Both Methylated Strands of the dsDNA Template

Primers T7 for-Atto647 (SEQ ID NOs: 9) and T7rev-Atto647 SEQ ID NO: 11), both at a final concentration of 2 microM) were directly added to 150 ng of the methylated dsDNA sample generated in (i) together with 0.2 mM dNTPs, and 1× final concentration of 10× Jena labelling buffer (PCR-201S) and 0.5 units of the Jena Taq polymerase. The sample was submitted to a single cycle of PCR with the following conditions: 95° C. 75s, 64° C. 1 min, 68° C. 2 min30, storage at 4° C. The Atto647-labelled primers allowed the newly synthesised unmethylated DNA to be distinguished from the unlabelled template DNA. Samples were purified as previously described and quantified.

(iii) Copying the DNA Methylation from the Original dsDNA Template into the Newly Synthesised DNA Strands.

10 μL of unpurified DNA from step (ii) was incubated with DNMT5 at an approximate concentration of 75fmol/uL, 800 microM SAM, 1.25 mM ATP, 1× final concentration of 10× Jena labelling buffer in a final volume of 20 microL. The reaction was incubated for 1 h at 25° C. and then stored at 8° C. until usage. A sample was purified as previously and quantified while an identical sample was used for subsequent steps.

(iv) Repeating the Cycle of H-E-M a Second Time from a Complementary Methylated dsDNA Template

Primers T7 for-cy3 (SEQ ID NOs: 8) and T7rev-cy3 (SEQ ID NO: 10), both at a final concentration of 2 microM) and 0.5 units of the Taq polymerase were directly added to 20 μL of the reaction from step (iii). The sample was submitted to a single cycle of PCR with the following conditions: 95° C. 75s, 64° C. 1 min, 68° C. 2 min30, storage at 4° C. In this reaction, the use of the Cy3 labelled primers allowed the new unmethylated DNA to be distinguished from the template DNA which was unlabelled or labelled with Atto647. A sample was purified as previously described and quantified.

(vi) Copying the DNA Methylation into the Newly Synthesised DNA Strands.

20 microL of the unpurified DNA mixture from step (v) was incubated with DNMT5 at an approximate concentration of 75fmol/microL, 800 microM SAM, 1.25 mM ATP, 1× final concentration of 10× Jena labelling buffer in a final volume of 21.5 microL. The reaction was incubated for 1 h at 25° C. and then stored at 8° C. until usage. The sample was purified as previously and quantified.

(vii) Treating DNA Samples with Ascl.

Between 15 to 30 ng of DNA sample was digested using 2U of Ascl (NEB #R0558S) in 1× Cutsmart buffer (NEB) with a final volume of 10 microL. The digestion was performed at 37° C. for 1h. 10 microL of each sample was mixed with 2 microL of 30% glycerol without any dye. The samples were run on a 4% agarose gel and run at 90V for 1h.

### Results

This experiment comprised a dsDNA substrate that contained methylated cytosines on both DNA strands, and was amplified in a method analogous to a typical PCR reaction. As shown in FIG. 5C, after the first round of hybridisation and extension (H-E) and before the first Methylation (M) reaction, the Ascl restriction enzyme cleaved the sample generating two main products of 135 bp and 60 bp. A fainter band, at 195 bp, suggested that the reactions were not complete under the conditions used. Following DNMT5 treatment, the sample treated with DNMT5 and digested with Ascl generated a main protected band at 195 bp and 2 fainter secondary bands at 135 and 60 bp. This result indicated that incubation of the Atto-647 labelled hemimethylated dsDNA with DNMT5 protected the newly synthesised strands via the copying of the methyl-C into the daughter strands.

The exact same observation was made through the second round of H-E-M when observing the Cy3 labelling.

From these results, it can be concluded that new hemimethylated dsDNA was generated, and which could be tracked through two cycles of H-E-M using firstly the Atto647 dye, and subsequently with Cy3 in a second H-E-M cycle. In both cases, the methyl cytosine was copied from the parent strands to the daughter and grand-daughter strands and/or from daughter to grand-daughter strands. These data demonstrated that cycles of H-E-M were compatible with cycles of PCR when using the product of the invention.

### Example 5. DNMT5 is Functional when Tethered to a Bead

A variant of DNMT5 from Cryptococcus neoformans bearing a C-terminal His tag (SEQ ID NO: 14) at concentration of 0.2 mg/ml was diluted 1:5 in binding buffer (20 mM Tris pH8.0, 500 mM NaCl) to a total volume of 100 microL and added to a solution containing ProteinA-coated magnetic beads (Cytiva catalog no 28967056) conjugated with an anti-His antibody (Invitrogen, catalog no MA5-33032), and incubated at 25° C., with shaking, for one hour to allow the His-tagged DNMT5 protein to associate with the beads. The beads were separated from the mixture by placing in a Cytiva MagRack 6 magnet, and the supernatant was removed. The beads were washed three times in binding buffer then twice in Equilibration Buffer (20 mM Tris pH8.0, 150 mM NaCl). Both supernatant and the beads were analysed by SDS-PAGE to confirm the presence of DNMT5 on the beads. 5 microL samples were prepared by adding 5 microL loading dye (Invitrogen catalogue no. B0007) 2 microL reducing agent (Invitrogen catalogue no. B0009) and made to a final volume of 20 microL with water. The samples were incubated at 85° C. for 5 minutes before loading on to a precast polyacrylamide gel (Invitrogen catalogue no. NW04125). The gel was run at a constant voltage of 200V for 25 minutes with MES buffer (Invitrogen catalogue no. B0002). The gel was stained in Coomassie Instant Blue (Abcam, catalogue no AB119211) ProteinA beads coupled with the same anti-His antibody but without DNMT5 were used as a negative control, and these control beads were washed and equilibrated in the same manner as the DNMT5 conjugated beads. To determine the activity of the DNMT5-coated beads, or control beads without DNMT5, the beads were removed from the equilibration buffer using the Cytiva MagRack 6 magnet.

To assay the activity of the methyltransferase attached to the magnetic beads, an ELISA-based experiment was conducted as follows. A hemimethylated dsDNA substrate was generated by combining two oligos (SEQ ID: 12 and SEQ ID: 13; purchased from Biomers.net GmbH, Germany) in an equimolar ratio in PBS. 2 microL of SEQ 12:10 at 100 microM was combined with 2 microL SEQ ID: 13 at 100 microM and 10 microL 2×PBS, to a final volume 20 microL with the balancing volume made up with deionised water. This mixture was heated to 80° C. in a PCR machine, and then slowly cooled (1° C. per see) to 25° C., to generate the double stranded hemimethylated substrate at 10 microM, 675 ng/microL. This substrate was diluted in water to a final concentration of 100 ng/microL for use in the assay. The DNMT5-immobilised or control beads were then resuspended with the following component mixture: 50 mM Tris-HCl PH 8, 25 mM NaCl, 10% Glycerol, 2 mM DTT, 1 mM ATP, 800 microM SAM, 1 mM MgCl₂, 200 ng DNA substrate, in a final volume of 50 microL. The reaction was incubated for 2.5 hours at 25° C., with shaking at 700 rpm in an Eppendorf ThermoMixer C.

Following the DNMT5 reaction the beads were separated from the mixture by placing in a Cytiva MagRack 6 magnet, and the supernatant was removed as before. The supernatant was applied to a 96 well white Nunc MaxiSorp microtitre plate (Thermofisher catalogue no. 236105) pre-coated in 100 microL of 100 microG/ml streptavidin (NEB catalogue no. N7021) and blocked in 0.2% BSA in PBS. The wells were washed three times with 100 microL 0.1M NaOH then three times with 200 microL PBS prior to the addition of the primary antibody. The presence of 5 mC on the immobilised DNA was detected using 50 microL per well of a mouse anti-mC monoclonal antibody (clone 33D3, Abcam catalogue no AB10805) diluted 1:5000 in PBS+0.1% BSA+100 microg/ml salmon sperm DNA (Invitrogen, catalogue no 15632-011). The reaction was incubated for one hour at room temperature before washing three times with 200 microL PBS with 0.1% Tween20 and three times with 200 microL PBS. This followed by adding 50 microL per well of HRP-labelled anti-mouse secondary antibody (Abcam, catalogue no. AB6789) diluted to 1:50000 in PBS+0.1% BSA. This was incubated for one hour at room temperature then washed with three times with 200 microL PBS with 0.1% Tween20 and three times with 200 microL PBS. The binding of the antibody complex was detected with a TMB-substrate (Life Technologies, catalogue no 002023) and was quenched with H₂SO₄ after approximately ten minutes.

### Results.

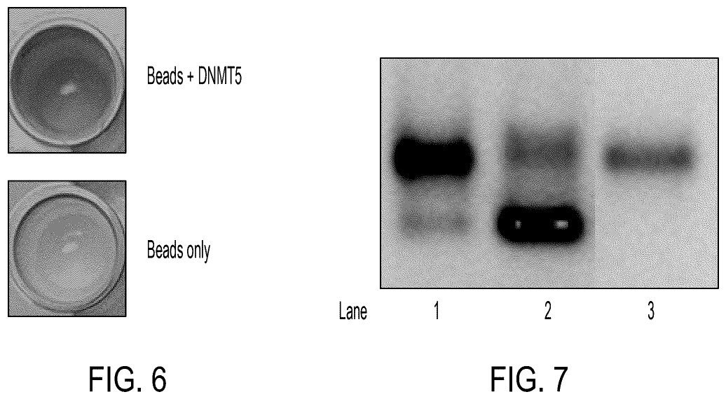

In this assay the substrate DNA comprised two DNA strands that were hybridised together—a first DNA strand (SEQ ID NO: 12) that comprised a sequence with three methylated cytosine (mC) bases, and was not biotinylated—and a second DNA strand (SEQ ID NO: 13) that was the unmethylated reverse complement of the first and was labelled at its 5′ end with biotin. Thus, the action of the methyltransferase would copy the methylated cytosine residues from the unbiotinylated strand to the biotinylated strand, and the application of 0.1M NaOH would separate the two strands leaving only the biotinylated strand, together with any methyl cytosines copied from the first unbiotinylated strand, bound to the streptavidin plate. In FIG. 6, it can be seen that a strong positive signal was observed from the well containing the DNA that had been treated with DNMT5 conjugated beads. By contrast, no signal was detected from the beads-only negative control that lacked DNMT5. These data demonstrated that a DNMT could be tethered to a solid support, such as a magnetic bead, and retained methyltransferase activity. These data support the potential of a tethered DNMT protein in the methods of the invention.

### Example 6. Archival Copying, Storage and Recovery of Data in DNA Using a Coupled Methyltransferase-Polymerase Reaction

Data is typically encoded into DNA as the canonical four bases, but in addition, synthetic DNA can be made with patterns of epigenetic (5 mC) modifications. It is possible to generate a method of data storage in DNA by one of several methods such as selective synthetic oligo annealing to a synthetic single-stranded template, coupled with CpG methylation; the synthesis and ligation of pre-methylated DNA fragments; or other suitable techniques.

The resulting DNA can be stored as the master archive of the data. When this data is required to be accessed, or transferred, without depleting the archive, a small dilute sample of the archive material can be taken. The methylated DNA in this sample is then amplified by a method of the present invention, such as using a polymerase-methyltransferase process described in Example 4 herein. The methylated DNA in the sample is copied by denaturation, primer annealing and extension to generate hemimethylated DNA. The methyl groups on the original strands are then copied onto the newly synthesized strands using the DNMT enzyme. A further round of denaturation, primer annealing, extension then yields new hemimethylated DNA, and subsequently these methyl groups are copied back onto the new copies of the original strands. Further rounds of this process are performed until the methylated DNA has been faithfully amplified. The data encoded within this amplified DNA can then be read using a variety of standard methods without depleting the original archive material.

### Example 7. DNMT5 Requires a Cation-Containing Buffer for Activity

DNMT5 is unique among DNMTs as it requires both SAM and ATP as cofactors for functional activity.

The method of Example 2 was repeated to test the activity of DNMT5 in the presence of 50 mM EDTA.

### Results.

As shown in FIG. 7, DNMT5 was not active in the presence of EDTA (lane 2). This suggests that chelating agents, which are described in the art in conventional methylation protocols using DNMT1, cannot be used in methods using DNMT5.

### Example 8. Optimisation of Mg²⁺within the buffer for a one-pot reaction

Having demonstrated that DNMT5 requires a cation-containing buffer for activity, the cofactor requirements of a DNA polymerase were assessed in order to improve the conditions for a one-pot H-E-M reaction. ATP and/or SAM and/or MgSO₄ was added to a primer extension experiment comprising 10 ng hemimethylated DNA substrate (prepared in Example 2(1)), 1× ThermoPol buffer (NEB B9004), forward primer (SEQ ID NO: 18, T7F), reverse primer (SEQ ID NO: 10, T7R-Cy3), Taq DNA polymerase (NEB M0267) and dNTPs. Reactions were incubated for 30 seconds at 95° C., 58° C. for 15 seconds, 68° C. for 30 seconds to allow a single DNA primer extension. 2 μL of loading dye was added to the reaction, which was then loaded on a 3% agarose gel and run for 30 minutes at 100V. The gel was imaged using Cy3 fluorescence from the reverse primer.

### Results.

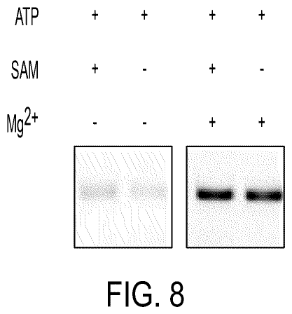

FIG. 8 shows that in the absence of additional Mg²⁺, primer extension, was inhibited by the addition of ATP. These effects were recovered by supplementing the reaction with additional Mg²⁺ (indicated by the +signs).

### Example 9. N- and C-Terminal Truncated Forms of C. neoformans DNMT5 are Able to Methylate Template DNA

To investigate whether full-length DNMT5 is required for methylation protection, a range of N- and/or C-terminally truncated fragments of C. neoformans DNMT5 were generated and tested for their activity in the Ascl methylation assay according to the procedure in Example 2.

The relative positions of the N- and C-terminal amino acid residues of the different DNMT5 variants are listed in the Table below, with residue numbering corresponding to the full-size enzyme sequence as shown in SEQ ID NO: 1. For the NeoFLAGHis variant (SEQ ID NO: 23) both the FLAG and His tag epitopes were appended to the C-terminal amino acids of DNMT5; the other variants also contain both tags on the C-terminal amino acid residue shown in the Table. The N-terminal positions relative to SEQ ID NO: 1 are indicated, and for each variant bearing an N-terminal truncation, an additional Methionine residue was encoded as the first position of the mature protein.

Table. Different truncations of the Cryptococcus neoformans
DNMT5 used in our Ascl assay. The amino acid
residue numbers are indicated.

| Variant | SEQ ID NO: | N-terminus | C-terminus | Tags |
|---|---|---|---|---|
| NeoFLAGHis | 23 | 1 | 2377 | FLAG, His |
| 2 | 24 | 58 | 2377 | FLAG, His |
| 3 | 25 | 289 | 2377 | FLAG, His |
| 4 | 26 | 301 | 2377 | FLAG, His |
| 5 | 27 | 1 | 2336 | FLAG, His |
| 6 | 28 | 58 | 2336 | FLAG, His |
| 7 | 29 | 289 | 2336 | FLAG, His |
| 8 | 30 | 301 | 2336 | FLAG, His |
| 9 | 31 | 1 | 2301 | FLAG, His |
| 10 | 32 | 58 | 2301 | FLAG, His |
| 11 | 33 | 289 | 2301 | FLAG, His |
| 12 | 34 | 301 | 2301 | FLAG, His |

A hemimethylated template containing an Ascl site was generated according to the procedure in Example 2 (1) and then labelled with a fluorophore as follows: 1 μL of the PCR product from Example 2 (1) was used as a template for a second round of amplification with DNA primers P257 (SEQ ID NO: 6) and T7rev-Atto647 (SEQ ID NO: 11), 1× Q5® High-Fidelity Buffer (NEB), dNTPs, DNA template, and Q5 polymerase. PCR conditions were 98° C. 30s, 26 cycles [98° C. 10s, 70° C. 30s, 72° C. 20s], 72° C. 1 min. Amplified DNA was purified using the Monarch® PCR & DNA Cleanup Kit following manufacturer's instructions.

The activity of the different variants was tested using the Ascl assay described in Example 2. After digestion, 2 μL of DNA Loading Dye was added to the reaction mixture, which was then loaded on a 4% agarose gel stained with SYBR Safe and run for 30 minutes at 100V.

### Results.

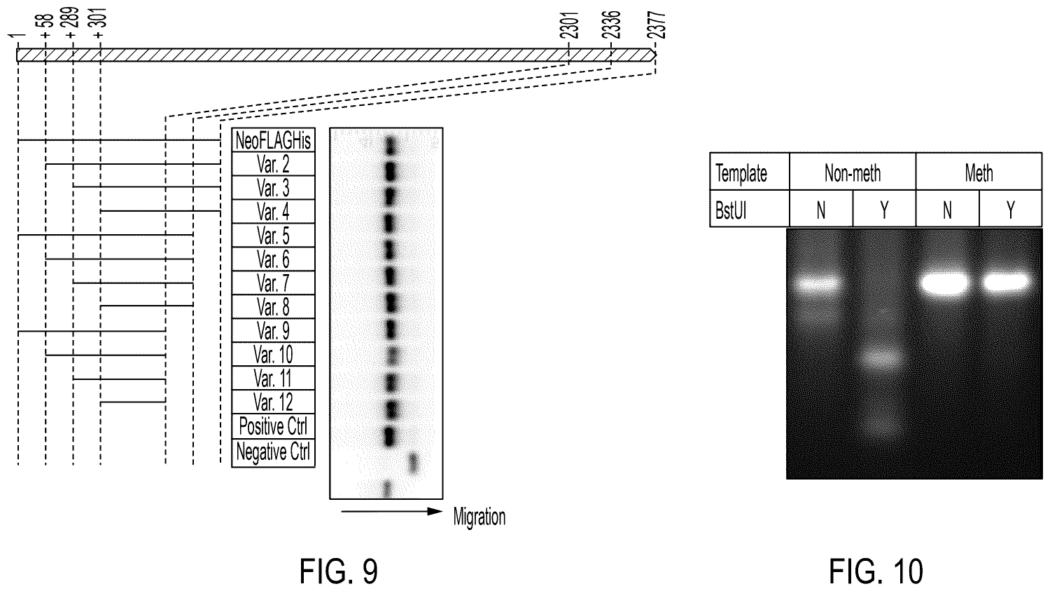

As shown in FIG. 9, provided that the MT domain and SNF2 ATPase domain remain functionally intact, the DNMT5 fragments, i.e. the truncated DNMT5s, were able to function in the Ascl assay.

These data indicate that functional fragments of DNMT5 may be used in H-E-M assays according to the present invention.

### Example 10. Removal of DNMT5 During Repeated H-E-M Cycles in a One-Pot Reaction

As shown in Example 5, DNMT5 is functional when tethered to beads.

The method of Example 4 is repeated, using DNMT5 tethered to beads as per Example 5, rather than in solution. At the end of step (iii), the tethered DNMT5 is pulled down and removed from the reaction vessel prior to the H-E-M cycle being repeated. Removal of the DNMT5 facilitates the next round of H-E-M. Tethered DNMT5 is added to the reaction vessel prior to the state of step (iii) of the second H-E-M cycle. Further H-E-M cycles can be conducted with the removal and addition of tethered DNMT5 in the same way. Removal of the DNMT5 after step (iii) facilitates the next round of H-E-M.

### Example 11. 5 Cycles of H-E-M in a One-Pot Reaction Successfully Amplifies Human DNA Template Sequence

Human SEPT9, an important biomarker for colorectal cancer, and which contains 7 potential sites for CpG methylation was subjected to 5 H-E-M cycles as follows:

Methylated and non-methylated synthetic oligonucleotide primers (Biomers.net GmbH, Germany) were synthesised corresponding to the SEPT9 promoter sequence in which the CpGs were either methylated or non-methylated (SEQ ID NO: 19 and SEQ ID NO: 20).

Hemimethylated and non-methylated SEPT9 dsDNA substrates were generated using a single primer extension by hybridising an oligonucleotide primer SEQ ID NO: 21 to SEQ ID NO: 19 or SEQ ID NO: 20 in a 50 uL reaction with Taq DNA polymerase comprising 10UM SEQ ID NO: 21, 20 uM SEQ ID NO: 19 or SEQ ID NO: 20, 25 uL 2× Taq Mastermix (NEB M0270). These samples were then incubated at 95° C., and then cooled to 60° C. for 15 seconds followed by 68° C. for 1 minute, and then purified.

One-pot reactions containing DNMT5 and Klenow were set up to test multiple cycles of H-E-M with the hemi-methylated or non-methylated SEPT9 dsDNA substrates, as described in Example 4. Because the SEPT9 DNA sequence contains two pairs of CpGs on each strand with the sequence CGCG, it was possible to use the methyl-sensitive restriction enzyme BstUl to assess methylation protection during multiple cycles of H-E-M, similar to how the Ascl site was used in Example 2 above.

In this Example, after 5 cycles of H-E-M with Klenow were carried out, with the DNMT5 removed between H-E-M cycles. Following sample purification, 5 μL of eluted DNA was digested with 2U BstUI (NEB, R0518) in 1× CutSmart buffer to a final volume of 10 μL. The digestion was performed at 65° C. for 1 hour. 2 μL of loading dye was added to the reaction, which was then loaded on a 3% agarose gel and run for 60 minutes at 100V. The gel was imaged using SYBR Safe.

### Results.

As shown in FIG. 10, the hemimethylated SEPT9 template was successfully amplified and methylated and was protected from BstUI digest. However, where the starting material was the non-methylated SEPT9 DNA template, the DNA was still sensitive to digestion by BstUl. Furthermore, five cycles of HEM did not introduce any de novo methylation via DNMT5 in this method.

### Example 12. Detecting a Biomarker in a Biological Fluid Using Amplification of Methylated dsDNA

A sample of a biological fluid (e.g. blood) is obtained from a subject suspected of or at risk of having said medical condition (e.g. cancer). dsDNA from the sample is subjected to a H-E-M method to amplify a methylated biomarker, wherein methylation of the biomarker is known to be associated with a medical condition. For example, SEPT9 and colon cancer, as in Example 11 above.

Following amplification of the biomarker, methylation of the biomarker is detected using an appropriate technique, such as sodium bisulfite treatment or endonuclease digestion. Detection of the methylated biomarker is used to diagnose the presence of the medical condition with which the methylated biomarker is associated. Again, as per Example 11 above, detection of methylated SEPT9 using BstUI digest can be used to diagnose colon cancer.

1-48. (canceled)

49. A method of amplifying methylated DNA, comprising:
(a) copying a target sequence on one strand of a double stranded DNA (dsDNA) with a DNA polymerase and at least one target specific DNA oligonucleotide primer, wherein the dsDNA is symmetrically methylated and the copying produces a hemimethylated dsDNA substrate; and
(b) contacting the hemimethylated dsDNA substrate with an enzyme comprising methyltransferase activity and ATP hydrolysis (ATPase) activity,
wherein an amplification cycle comprising steps (a) and (b) is repeated at least once, and wherein the method produces an amplified methylated dsDNA product.

50. The method of claim 49, wherein the enzyme comprises a methyltransferase domain and/or an ATPase domain.

51. The method of claim 49, wherein the enzyme has at least 100-fold greater activity for hemimethylated DNA compared with unmethylated DNA.

52. The method of claim 49, wherein the amplified methylated dsDNA product is symmetrically methylated.

53. The method of claim 49, further comprising (c) removing or inactivating the enzyme from step (b).

54. The method of claim 49, further comprising (d) determining the presence of methylated DNA in the amplified methylated dsDNA product.

55. The method of claim 49, further comprising (d) adding sequencing adapters to the amplified methylated dsDNA product for sequencing.

56. The method of claim 49, wherein steps (a)-(b) are conducted in the same buffer.

57. The method of claim 49, wherein steps (a)-(b) are conducted in a cation-containing buffer.

58. The method of claim 57, wherein the cation-containing buffer comprises Mg²⁺ cations.

59. The method of claim 49, wherein the method excludes the addition of a chelating agent.

60. The method of claim 59, wherein the chelating agent is EDTA.

61. The method of claim 49, wherein step (a) further comprises an adapter.

62. The method of claim 61, wherein the adapter is methylated.

63. The method of claim 49, wherein the DNA polymerase comprises a high-fidelity DNA polymerase; or a strand displacing DNA polymerase.

64. The method of claim 63, wherein the polymerase comprises: Q5® DNA polymerase, Phusion® DNA polymerase, Pfu DNA polymerase, Klenow DNA polymerase, Taq DNA polymerase, KAPA HiFi DNA polymerase, phi29 DNA polymerase, Bst DNA polymerase, or Bsu DNA polymerase.

65. The method of claim 49, wherein at least one target specific primer or an adapter added to (a) comprise a tag or a barcode at the 5′ and/or 3′ end.

66. The method of claim 65, wherein the tag comprises a His-tag, biotin, CBD, MBP, strep-tag or SNAP-tag; an antibody, a fluorescent label, a dye, a SAM analogue or a chemiluminescent label.

67. The method of claim 49, wherein the enzyme in (b) is a DNMT5.

68. The method of claim 49, wherein (b) further comprises a methyl donor group, wherein the methyl donor group is S-adenosyl methionine (SAM) or a SAM analog.

69. The method of claim 49, further comprising immobilizing on a solid substrate at least one of the following components: the target dsDNA, the primer, the polymerase, the enzyme, or the amplified dsDNA product.

70. The method of claim 49, wherein the enzyme in (b) has de novo methylation activity that is less than de novo methylation activity of a DNMT1.

71. The method of claim 49, wherein the enzyme in (b) has no detectable de novo methylation activity.

72. The method of claim 49, wherein the enzyme in (b) is thermofunctional at a temperature of up to 37C.

73. The method of claim 49, wherein the enzyme in (b) comprises the sequence of SEQ ID NO: 1 or a sequence having at least 80% identity thereto, or SEQ ID NO: 3 or a sequence having at least 80% identity thereto.

74. The method of claim 49, wherein steps (a) and (b) are performed in a single reaction vessel and/or at the same time.

75. The method of claim 49, wherein the amplified methylated dsDNA product is used in next-generation sequencing and/or epigenetic analysis.

76. A DNMT5 truncated at the C-terminus and/or N-terminus, which comprises methyltransferase activity and ATPase activity.

77. A method comprising:
determining the methylation status of a target sequence in a biological fluid, comprising amplifying methylated double-stranded DNA (dsDNA) from the biological fluid to produce an amplified symmetrically methylated dsDNA product,
wherein the method comprises:
(a) copying a target sequence on one strand of the dsDNA from the biological fluid with a DNA polymerase and at least one target specific DNA oligonucleotide primer, wherein the dsDNA is symmetrically methylated and the copying produces a hemimethylated dsDNA substrate; and
(b) contacting the hemimethylated dsDNA substrate with an enzyme comprising methyltransferase activity and ATP hydrolysis (ATPase) activity,
wherein an amplification cycle comprising steps (a) and (b) is repeated at least once, and wherein the method produces an amplified symmetrically methylated dsDNA product, and
wherein the methylation status is associated with a medical condition.

78. A kit comprising:
(i) an enzyme comprising methyltransferase activity and ATP hydrolysis (ATPase) activity,
(ii) one or more of: S-adenosyl methionine (SAM) or a SAM analog, ATP, dATP, a DNA polymerase, a DNA cleaving enzyme, one or more buffers, one or more oligonucleotide primers; Mg²⁺ cations, and
(iii) instructions for use.
